(19) United States
(12) Patent Application Publication
(10) Pub. No.: US 2026/0139251 A1
(43) Pub. Date: May 21, 2026

(54) RNAi Agents for Inhibiting Expression of Mucin 5AC (MUC5AC), Compositions Thereof, and Methods of Use

(71) Applicant: Arrowhead Pharmaceuticals, Inc., Pasadena, CA (US)

(72) Inventors: Erik W. Bush, Verona, WI (US); Anthony Nicholas, Oregon, WI (US); Casi M. Schienebeck, Deerfield, WI (US)

(21) Appl. No.: 19/275,121

(22) Filed: Jul. 21, 2025

Related U.S. Application Data
(63) Continuation of application No. 17/824,841, filed on May 25, 2022, now Pat. No. 12,365,897.
(60) Provisional application No. 63/194,370, filed on May 28, 2021.

(51) Int. Cl. C12N 15/113; A61P 11/12

(52) U.S. Cl. CPC C12N 15/113; A61P 11/12; C12N 2310/14; C12N 2310/315; C12N 2310/317; C12N 2310/321

(57) ABSTRACT

Described are RNAi agents, compositions that include RNAi agents, and methods for inhibition of a Mucin 5AC (MUC5AC) gene. The MUC5AC RNAi agents and RNAi agent conjugates disclosed herein inhibit the expression of an MUC5AC gene. Pharmaceutical compositions that include one or more MUC5AC RNAi agents, optionally with one or more additional therapeutics, are also described. Delivery of the described MUC5AC RNAi agents to pulmonary epithelial cells, in vivo, provides for inhibition of MUC5AC gene expression and a reduction in MUC5AC production, which can provide a therapeutic benefit to subjects, including human subjects, for the treatment of various diseases including mucoobstructive lung disease such as severe asthma and various cancers.

### CROSS REFERENCE TO RELATED APPLICATIONS

This application is a continuation of U.S. patent application Ser. No. 17/824,841, filed on May 25, 2022, which claims priority from U.S. Provisional Patent Application Ser. No. 63/194,370, filed on May 28, 2021, the contents of which are incorporated herein by reference in its entirety.

### SEQUENCE LISTING

This application contains a Sequence Listing (in compliance with Standard ST26), which has been submitted in xml format and is hereby incorporated by reference in its entirety. The xml sequence listing file is named 30659-US2_SeqListing.xml, created Jun. 3, 2025, and is 6,411,920 bytes in size.

### FIELD OF THE INVENTION

The present disclosure relates to RNA interference (RNAi) agents, e.g., double stranded RNAi agents, for inhibition of Mucin 5AC (“MUC5AC”) gene expression, compositions that include MUC5AC RNAi agents, and methods of use thereof. BACKGROUND

MUC5AC is a transcriptionally regulated secreted mucin expressed in airway epithelia of the lung and in other mucosal tissues (for example, gastrointestinal, urogenital, eye, and ear) (Lillehoj et al, Int Rev Cell Mol Biol, 2013). In airways, MUC5AC and MUC5B are the major gel-forming mucins. MUC5B is constitutively expressed and is required for mucociliary clearance (Roy et al., Nature 2014). Normal subjects have a relatively higher expression of MUC5B versus MUC5AC in the trachea and proximal airways, with this ratio further increasing in distal airways with expression of MUC5AC almost undetectable in distal and terminal bronchioles (Okuda et al., AJRCCM 2019). Typically expressed at low levels in the airway, MUC5AC expression can be robustly induced by external stress stimuli like pro-inflammatory mediators (including, for example, type 2 cytokines: IL-4, IL-9, TL-17, TL-23 and IL-13), noxious inhaled substances (for example, cigarette smoke, acrolein, toxic gases), viral infections, and allergens. The resulting mucus hypersecretion and hyperconcentration is understood to be a common pathogenic mechanism linked to airway obstruction in severe asthma and other mucoobstructive lung diseases such as cystic fibrosis (CF), chronic obstructive pulmonary disease (COPD), non-CF bronchiectasis (NCFB), and primary ciliary dyskinesia (PCD) (Boucher, NEJM 2019). In asthma, COPD, and NCFB patients, exaggerated expression and secretion of MUC5AC results in a narrowing of airway lumen, airway obstruction, and exacerbations (Dunican et al., JCI 2017; Bonser et al., JCI 2016; Kesimer et al., NEJM 12017; Ramsey et al, AJRCCM 2019). A genome-wide association study (GWAS) identified a novel MUC5AC allele linked to increased MUC5AC expression and patients with moderate-to-severe asthma (Shrine et al., Lancet Respir Med 2019). Experimental evidence from MUC5AC-deficient mice demonstrated that MUC5AC-mediated airway plugging is a major contributor to airway hyperresponsiveness to allergens independent of inflammation and bronchoconstriction (Evans et al, Nat Commun, 2015). Current standard of care treatments for severe asthma and other mucoobstructive lung diseases include bronchodilators and anti-inflammatory therapeutics (such as corticosteroids and biologics), but currently available treatments do not directly address pathogenic mucin overexpression and hypersecretion. Alternative approaches that directly treat mucus hypersecretion and obstruction are needed.

Increased expression of MUC5AC has also been observed in malignancies such as lung adenocarcinomas, pancreatic cancer, salivary gland carcinoma, breast cancer, cholangiocarcinoma, ovarian cancer, and other tumors (Krishn et al., Carcinogenesis 2018), where it has been linked to migration and invasiveness of tumor cells. Loss-of-function mutations in MUC5AC and other mucin genes are significantly underrepresented in tumor cells, suggesting that mucin overexpression may shield tumors from recognition by immune cells (Gorlov et al., Cancer Genetics 2019). Tumor MUC5AC overexpression is associated with progression and poor survival in lung adenocarcinoma patients (Bauer et al., JCI Insight 2018). MUC5AC overexpression has also been linked to number of other conditions including: allergic rhinitis, chronic rhinosinusitis, otitis media. Barret's esophagus, pancreatitis, and inflammatory bowel disease (Krishn et al., Carcinogenesis 2018).

### SUMMARY

There exists a need for novel RNA interference (RNAi) agents (termed RNAi agents, RNAi triggers, or triggers), e.g., double stranded RNAi agents, that are able to selectively and efficiently inhibit the expression of a MUC5AC gene, including for use as a therapeutic or medicament. Further, there exists a need for compositions of novel MUC5AC-specific RNAi agents for the treatment of diseases or disorders associated with mucus hypersecretion and obstruction (referred to herein as “mucoobstructive” lung diseases and disorders) such as for example cystic fibrosis (CF), chronic obstructive pulmonary disease (COPD), non-CF bronchiectasis (NCFB), primary ciliary dyskinesia (PCD), and asthma, and/or diseases or disorders that can be mediated at least in part by a reduction in MUC5AC gene expression and/or MUC5AC protein levels.

The nucleotide sequences and chemical modifications of the MUC5AC RNAi agents disclosed herein, as well as their combination with certain specific targeting ligands suitable for selectively and efficiently delivering the MUC5AC RNAi agents in vivo, differ from what is known in the art. The MUC5AC RNAi agents disclosed herein provide for highly potent and efficient inhibition of the expression of a MUC5AC gene and have sequences suitable for use as a therapeutic for the treatment of diseases and disorders.

In general, the present disclosure features MUC5AC gene-specific RNAi agents, compositions that include MUC5AC RNAi agents, and methods for inhibiting expression of a MUC5AC gene in vitro and/or in vivo using the MUC5AC RNAi agents and compositions that include MUC5AC RNAi agents described herein. The MUC5AC RNAi agents described herein are able to selectively and efficiently decrease expression of a MUC5AC gene, and thereby reduce the expression of the MUC5AC protein, which can lead to a therapeutic benefit such as, for example, a reduction in mucoobstruction in the lung.

The described MUC5AC RNAi agents can be used in methods for therapeutic treatment (including preventative or prophylactic treatment) of symptoms and diseases including, but not limited to, mucoobstructive lung diseases (such as asthma, CF, COPD, NCFB, PCD), allergic bronchopulmonary aspergillosis, interstitial lung diseases, cancer (such as lung adenocarcinomas, pancreatic cancer, salivary gland carcinoma, breast cancer, cholangiocarcinoma, ovarian cancer, and other tumors), respiratory infections (such as respiratory syncytial virus, influenza, rhinovirus), otitis media, inflammatory bowel disease, gallstone disease, allergic rhinitis, chronic rhinosinusitis and nasal polyposis.

In one aspect, the disclosure features RNAi agents for inhibiting expression of a MUC5AC gene, wherein the RNAi agent includes a sense strand (also referred to as a passenger strand) and an antisense strand (also referred to as a guide strand). The sense strand and the antisense strand can be partially, substantially, or fully complementary to each other. The length of the RNAi agent sense strands described herein each can be 15 to 49 nucleotides in length. The length of the RNAi agent antisense strands described herein each can be 18 to 49 nucleotides in length. In some embodiments, the sense and antisense strands are independently 18 to 26 nucleotides in length. The sense and antisense strands can be either the same length or different lengths. In some embodiments, the sense and antisense strands are independently 21 to 26 nucleotides in length. In some embodiments, the sense and antisense strands are independently 21 to 24 nucleotides in length. In some embodiments, both the sense strand and the antisense strand are 21 nucleotides in length. In some embodiments, the antisense strands are independently 18, 19, 20, 21, 22, 23, 24, 25, 26, 27, 28, 29, or 30 nucleotides in length. In some embodiments, the sense strands are independently 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides in length. The RNAi agents described herein, upon delivery to a cell expressing MUC5AC, inhibit the expression of one or more MUC5AC gene variants in vivo and/or in vitro.

The MUC5AC RNAi agents disclosed herein target a human MUC5AC gene (see, e.g., SEQ ID NO:1). In some embodiments, the MUC5AC RNAi agents disclosed herein target a portion of a MUC5AC gene having the sequence of any of the sequences disclosed in Table 1.

In another aspect, the disclosure features compositions, including pharmaceutical compositions, that include one or more of the disclosed MUC5AC RNAi agents that are able to selectively and efficiently decrease expression of a MUC5AC gene. The compositions that include one or more MUC5AC RNAi agents described herein can be administered to a subject, such as a human or animal subject, for the treatment (including prophylactic treatment or inhibition) of symptoms and diseases associated with MUC5AC gene expression and/or MUC5AC protein levels.

Examples of MUC5AC RNAi agent sense strands and antisense strands that can be used in a MUC5AC RNAi agent are provided in Tables 3, 4, 5, 6, and 7. Examples of MUC5AC RNAi agent duplexes are provided in Tables 8A, 8B, 8C, 9, 10A, 10B, and 11. Examples of 19-nucleotide core stretch sequences that may consist of or may be included in the sense strands and antisense strands of certain MUC5AC RNAi agents disclosed herein, are provided in Table 2.

In another aspect, the disclosure features methods for delivering MUC5AC RNAi agents to epithelial cells in a subject, such as a mammal, in vivo. Also described herein are compositions for use in such methods. In some embodiments, disclosed herein are methods for delivering MUC5AC RNAi agents to pulmonary cells (epithelial cells, macrophages, smooth muscle, endothelial cells) to a subject in vivo. In some embodiments, the subject is a human subject.

The methods disclosed herein include the administration of one or more MUC5AC RNAi agents to a subject, e.g., a human or animal subject, by any suitable means known in the art. The pharmaceutical compositions disclosed herein that include one or more MUC5AC RNAi agents can be administered in a number of ways depending upon whether local or systemic treatment is desired. Administration can be, but is not limited to, for example, intravenous, intraarterial, subcutaneous, intraperitoneal, subdermal (e.g., via an implanted device), and intraparenchymal administration. In some embodiments, the pharmaceutical compositions described herein are administered by inhalation (such as dry powder inhalation or aerosol inhalation), intranasal administration, intratracheal administration, or oropharyngeal aspiration administration.

In some embodiments, it is desired that the MUC5AC RNAi agents described herein inhibit the expression of an MUC5AC gene in the pulmonary epithelium, for which the administration is by inhalation (e.g., by an inhaler device, such as a metered-dose inhaler, or a nebulizer such as a jet or vibrating mesh nebulizer, or a soft mist inhaler).

The one or more MUC5AC RNAi agents can be delivered to target cells or tissues using any oligonucleotide delivery technology known in the art. In some embodiments, a MUC5AC RNAi agent is delivered to cells or tissues by covalently linking the RNAi agent to a targeting group. In some embodiments, the targeting group can include a cell receptor ligand, such as an integrin targeting ligand. Integrins are a family of transmembrane receptors that facilitate cell-extracellular matrix (ECM) adhesion. In particular, integrin alpha-v-beta-6 (αvβ6) is an epithelial-specific integrin that is known to be a receptor for ECM proteins and the TGF-beta latency-associated peptide (LAP), and is expressed in various cells and tissues. Integrin αvβ6 is known to be highly upregulated in injured pulmonary epithelium. In some embodiments, the MUC5AC RNAi agents described herein are linked to an integrin targeting ligand that has affinity for integrin αvβ6. As referred to herein, an “αvβ6 integrin targeting ligand” is a compound that has affinity for integrin αvβ6, which can be utilized as a ligand to facilitate the targeting and delivery of an RNAi agent to which it is attached to the desired cells and/or tissues (i.e., to cells expressing integrin αvβ6). In some embodiments, multiple αvβ6 integrin targeting ligands or clusters of αvβ6 integrin targeting ligands are linked to a MUC5AC RNAi agent. In some embodiments, the MUC5AC RNAi agent-αvβ6 integrin targeting ligand conjugates are selectively internalized by lung epithelial cells, either through receptor-mediated endocytosis or by other means.

Examples of targeting groups useful for delivering MUC5AC RNAi agents that include αvβ6 integrin targeting ligands are disclosed, for example, in International Patent Application Publication No. WO 2018/085415 and International Patent Application Publication No. WO 2019/089765, the contents of each of which are incorporated by reference herein in their entirety.

A targeting group can be linked to the 3′ or 5′ end of a sense strand or an antisense strand of a MUC5AC RNAi agent. In some embodiments, a targeting group is linked to the 3′ or 5′ end of the sense strand. In some embodiments, a targeting group is linked to the 5′ end of the sense strand. In some embodiments, a targeting group is linked internally to a nucleotide on the sense strand and/or the antisense strand of the RNAi agent. In some embodiments, a targeting group is linked to the RNAi agent via a linker.

In another aspect, the disclosure features compositions that include one or more MUC5AC RNAi agents that have the duplex structures disclosed in Tables 8A, 8B, 8C, 9, 10A, 10B, and 11.

The use of MUC5AC RNAi agents provides methods for therapeutic (including prophylactic) treatment of diseases or disorders for which a reduction in MUC5AC gene expression and/or a reduction in MUC5AC protein levels can provide a therapeutic benefit. The MUC5AC RNAi agents disclosed herein can be used to treat various diseases, including mucoobstructive lung diseases (such as asthma, CF, COPD, NCFB, PCD), allergic bronchopulmonary aspergillosis, interstitial lung diseases, cancer (such as lung adenocarcinomas, pancreatic cancer, salivary gland carcinoma, breast cancer, cholangiocarcinoma, ovarian cancer, and other tumors), respiratory infections (such as respiratory syncytial virus, influenza, rhinovirus), otitis media, inflammatory bowel disease, gallstone disease, allergic rhinitis, chronic rhinosinusitis and nasal polyposis. In some embodiments, the MUC5AC RNAi agents disclosed herein can be used to treat a mucoobstructive lung disease, such as severe asthma or COPD. MUC5AC RNAi agents can further be used to treat, for example, various cancers. Such methods of treatment include administration of a MUC5AC RNAi agent to a human being or animal having elevated or enhanced MUC5AC gene expression and/or MUC5AC protein levels above what is desired.

One aspect described herein is an RNAi agent for inhibiting expression of a MUC5AC gene, comprising:
(i) an antisense strand that is between 18 and 49 nucleotides in length that includes a nucleotide sequence at least partially complementary to a corresponding stretch of contiguous nucleotides of the MUC5AC gene transcript (SEQ ID NO: 1); and
(ii) a sense strand comprising a nucleotide sequence that is at least partially complementary to the antisense strand;
   wherein the RNAi agent sense strand is optionally further linked to a targeting ligand, and wherein RNAi agent is capable of inhibiting expression of a MUC5AC gene.

Another aspect described herein is an RNAi agent for inhibiting expression of a MUC5AC gene, comprising:
(i) an antisense strand comprising at least 17 contiguous nucleotides differing by 0 or 1 nucleotides from any one of the sequences provided in Table 3; and
(ii) a sense strand comprising a nucleotide sequence that is at least partially complementary to the antisense strand;
   wherein the RNAi agent sense strand is optionally further linked to a targeting ligand, and wherein RNAi agent is capable of inhibiting expression of a MUC5AC gene.

In some embodiments, a MUC5AC RNAi agent disclosed herein includes an antisense strand that consists of, consists essentially of, or comprises a sequence differing by 0 or 1 nucleobases from the nucleotide sequence (5′→3′) UUGUAGUAGUCGCAGAACAGC (SEQ ID NO: 1525). In some embodiments, a MUC5AC RNAi agent disclosed herein includes an antisense strand that consists of, consists essentially of, or comprises a nucleotide sequence differing by no more than 1 nucleotide from the nucleotide sequence (5′→3′) UUGUAGUAGUCGCAGAACAGC (SEQ ID NO: 1525), wherein all or substantially all of the nucleotides are modified nucleotides. In some embodiments, a MUC5AC RNAi agent disclosed herein includes an antisense strand that consists of, consists essentially of, or comprises a nucleobase sequence differing by 0 or 1 nucleobases from the nucleotide sequence (5′→3′) UUGUAGUAGUCGCAGAACAGC (SEQ ID NO: 1525), wherein SEQ ID NO: 1525 is located at positions 1-21 (5′→3′) of the antisense strand.

In some embodiments, a MUC5AC RNAi agent disclosed herein includes an antisense strand that consists of, consists essentially of, or comprises a modified nucleotide sequence differing by no more than 1 nucleotide from the nucleotide sequence (5′→3′) cPrpusUfsgsUfaGfuAfgUfcGfcAfgAfaCfaGfsc (SEQ ID NO: 1127), wherein a, c, g, and u represent 2′-O-methyl adenosine, cytidine, guanosine, and uridine, respectively; Af, Cf, Gf, and Uf represent 2′-fluoro adenosine, cytidine, guanosine, and uridine, respectively; cPrpu represents a 5′-cyclopropyl phosphonate-2′-O-methyluridine; and s represents a phosphorothioate linkage, and wherein the sense strand is at least substantially complementary to the antisense strand. As the person of ordinary skill in the art would clearly understand, the inclusion of a phosphorothioate linkage as shown in the modified nucleotide sequences disclosed herein replaces the phosphodiester linkage typically present in oligonucleotides (see, e.g., FIGS. 3A through 3J showing all internucleoside linkages). In some embodiments, a MUC5AC RNAi agent disclosed herein includes an antisense strand that consists of, consists essentially of, or comprises the nucleotide sequence (5′→3′) cPrpusUfsgsUfaGfuAfgUfcGfcAfgAfaCfaGfsc (SEQ ID NO: 1127), wherein a, c, g, and u represent 2′-O-methyl adenosine, cytidine, guanosine, and uridine, respectively; Af, Cf, Gf, and Uf represent 2′-fluoro adenosine, cytidine, guanosine, and uridine, respectively; cPrpu represents a 5′-cyclopropyl phosphonate-2′-O-methyluridine; and s represents a phosphorothioate linkage, and wherein the sense strand is at least substantially complementary to the antisense strand.

In some embodiments, a MUC5AC RNAi agent disclosed herein includes an antisense strand that consists of, consists essentially of, or comprises a modified nucleotide sequence differing by no more than 1 nucleotide from the nucleotide sequence (5′→3′) usUfsgsUfaGfuAfgUfcGfcAfgAfaCfaGfsc (SEQ ID NO: 1065), wherein a, c, g, and u represent 2′-O-methyl adenosine, cytidine, guanosine, and uridine, respectively; Af, Cf, Gf, and Uf represent 2′-fluoro adenosine, cytidine, guanosine, and uridine, respectively; and s represents a phosphorothioate linkage, and wherein the sense strand is at least substantially complementary to the antisense strand. In some embodiments, a MUC5AC RNAi agent disclosed herein includes an antisense strand that consists of, consists essentially of, or comprises the nucleotide sequence (5′→3′) usUfsgsUfaGfuAfgUfcGfcAfgAfaCfaGfsc (SEQ ID NO: 1065), wherein a, c, g, and u represent 2′-O-methyl adenosine, cytidine, guanosine, and uridine, respectively; Af, Cf, Gf, and Uf represent 2′-fluoro adenosine, cytidine, guanosine, and uridine, respectively; and s represents a phosphorothioate linkage, and wherein the sense strand is at least substantially complementary to the antisense strand.

In some embodiments, a MUC5AC RNAi agent disclosed herein includes an antisense strand that consists of, consists essentially of, or comprises a nucleobase sequence differing by 0 or 1 nucleobases from the nucleotide sequence (5′→3′) UUCUUGUUCAGGCAAAUCAGC (SEQ ID NO: 1535). In some embodiments, a MUC5AC RNAi agent disclosed herein includes an antisense strand that consists of, consists essentially of, or comprises a nucleotide sequence differing by no more than 1 nucleotide from the nucleotide sequence (5′→3′) UUCUUGUUCAGGCAAAUCAGC (SEQ ID NO: 1535), wherein all or substantially all of the nucleotides are modified nucleotides. In some embodiments, a MUC5AC RNAi agent disclosed herein includes an antisense strand that consists of, consists essentially of, or comprises a nucleobase sequence differing by 0 or 1 nucleobases from the nucleotide sequence (5′→3′) UUCUUGUUCAGGCAAAUCAGC (SEQ ID NO: 1535), wherein SEQ ID NO: 1535 is located at positions 1-21 (5′→3′) of the antisense strand.

In some embodiments, a MUC5AC RNAi agent disclosed herein includes an antisense strand that consists of, consists essentially of, or comprises a modified nucleotide sequence differing by no more than 1 nucleotide from the nucleotide sequence (5′→3′) usUfscsuuguucagGfcAfaAfucagsc (SEQ ID NO: 1166), wherein a, c, g, and u represent 2′-O-methyl adenosine, cytidine, guanosine, and uridine, respectively; Af, Cf, Gf, and Uf represent 2′-fluoro adenosine, cytidine, guanosine, and uridine, respectively; and s represents a phosphorothioate linkage, and wherein the sense strand is at least substantially complementary to the antisense strand. As the person of ordinary skill in the art would clearly understand, the inclusion of a phosphorothioate linkage as shown in the modified nucleotide sequences disclosed herein replaces the phosphodiester linkage typically present in oligonucleotides (see, e.g., FIGS. 3A through 3J showing all internucleoside linkages). In some embodiments, a MUC5AC RNAi agent disclosed herein includes an antisense strand that consists of, consists essentially of, or comprises the nucleotide sequence (5′→3′) usUfscsuuguucagGfcAfaAfucagsc (SEQ ID NO: 1166), wherein a, c, g, and u represent 2′-O-methyl adenosine, cytidine, guanosine, and uridine, respectively; Af, Cf, Gf, and Uf represent 2′-fluoro adenosine, cytidine, guanosine, and uridine, respectively; and s represents a phosphorothioate linkage, and wherein the sense strand is at least substantially complementary to the antisense strand.

In some embodiments, a MUC5AC RNAi agent disclosed herein includes an antisense strand that consists of, consists essentially of, or comprises a modified nucleotide sequence differing by no more than 1 nucleotide from the nucleotide sequence (5′→3′) cPrpuUfcuuguucagGfcAfaAfucagsc (SEQ ID NO: 1191), wherein a, c, g, and u represent 2′-O-methyl adenosine, cytidine, guanosine, and uridine, respectively; Af, Cf, Gf, and Uf represent 2′-fluoro adenosine, cytidine, guanosine, and uridine, respectively; cPrpu represents a 5′-cyclopropyl phosphonate-2′-O-methyluridine; and s represents a phosphorothioate linkage, and wherein the sense strand is at least substantially complementary to the antisense strand. As the person of ordinary skill in the art would clearly understand, the inclusion of a phosphorothioate linkage as shown in the modified nucleotide sequences disclosed herein replaces the phosphodiester linkage typically present in oligonucleotides (see, e.g., FIGS. 3A through 3J showing all internucleoside linkages). In some embodiments, a MUC5AC RNAi agent disclosed herein includes an antisense strand that consists of, consists essentially of, or comprises the nucleotide sequence (5′→3′) cPrpuUfcuuguucagGfcAfaAfucagsc (SEQ ID NO: 1191), wherein a, c, g, and u represent 2′-O-methyl adenosine, cytidine, guanosine, and uridine, respectively; Af, Cf, Gf, and Uf represent 2′-fluoro adenosine, cytidine, guanosine, and uridine, respectively; cPrpu represents a 5′-cyclopropyl phosphonate-2′-O-methyluridine; and s represents a phosphorothioate linkage, and wherein the sense strand is at least substantially complementary to the antisense strand.

In some embodiments, a MUC5AC RNAi agent disclosed herein includes an antisense strand that consists of, consists essentially of, or comprises a nucleotide sequence that differs by 0 or 1 nucleotides from one of the following nucleotide sequences (5′→3′):

(SEQ ID NO: 1525)
UUGUAGUAGUCGCAGAACAGC;
or
(SEQ ID NO: 1535)
UUCUUGUUCAGGCAAAUCAGC;

wherein the MUC5AC RNAi agent further includes a sense strand that is at least partially complementary to the antisense strand; and wherein all or substantially all of the nucleotides on both the antisense strand and the sense strand are modified nucleotides.

In some embodiments, a MUC5AC RNAi agent disclosed herein includes an antisense strand that consists of, consists essentially of, or comprises a nucleotide sequence that differs by 0 or 1 nucleotides from one of the following nucleotide sequences (5′→3′):

(SEQ ID NO: 1525)
UUGUAGUAGUCGCAGAACAGC;
or
(SEQ ID NO: 1535)
UUCUUGUUCAGGCAAAUCAGC;

wherein the MUC5AC RNAi agent further includes a sense strand that is at least partially complementary to the antisense strand; wherein all or substantially all of the nucleotides on both the antisense strand and the sense strand are modified nucleotides; and wherein the sense strand further includes inverted abasic residues at the 3′ terminal end and at the 5′ end of the nucleotide sequence, and the sense strand also includes a targeting ligand that is covalently linked to the 5′ terminal end, wherein the targeting ligand includes a compound having affinity for an integrin receptor.

In some embodiments, a MUC5AC RNAi agent disclosed herein includes an antisense strand that consists of, consists essentially of, or comprises a nucleotide sequence that differs by 0 or 1 nucleotides from one of the following nucleotide sequences (5′→3′):

(SEQ ID NO: 1525)
UUGUAGUAGUCGCAGAACAGC;
or
(SEQ ID NO: 1535)
UUCUUGUUCAGGCAAAUCAGC;

wherein the MUC5AC RNAi agent further includes a sense strand that is at least partially complementary to the antisense strand; wherein all or substantially all of the nucleotides on both the antisense strand and the sense strand are modified nucleotides; and wherein the sense strand further includes inverted abasic residues at the 3′ terminal end and at the 5′ end of the nucleotide sequence, and the sense strand also includes a targeting ligand that is covalently linked to the 5′ terminal end, wherein the targeting ligand includes a compound having affinity for an integrin receptor; and wherein the respective antisense strand sequence is located at positions 1-21 of the antisense strand.

In some embodiments, a MUC5AC RNAi agent disclosed herein includes an antisense strand and a sense strand, wherein the antisense strand and the sense strand consist of, consist essentially of, or comprise nucleotide sequences that differ by 0 or 1 nucleotides from one of the following nucleotide sequence (5′→3′) pairs:

(SEQ ID NO: 1525)
UUGUAGUAGUCGCAGAACAGC
and
(SEQ ID NO: 1617)
GCUGUUCUGCGACUACUACAA;
or
(SEQ ID NO: 1535)
UUCUUGUUCAGGCAAAUCAGC
and
(SEQ ID NO: 1632)
GCUGAUUUGCCUGAACAAGAA;

or wherein all or substantially all of the nucleotides on both the antisense strand and the sense strand are modified nucleotides.

In some embodiments, a MUC5AC RNAi agent disclosed herein includes an antisense strand and a sense strand, wherein the antisense strand and the sense strand consist of, consist essentially of, or comprise nucleotide sequences that differ by 0 or 1 nucleotides from one of the following nucleotide sequences (5′→3′) pairs:

(SEQ ID NO: 1525)
UUGUAGUAGUCGCAGAACAGC
and
(SEQ ID NO: 1617)
GCUGUUCUGCGACUACUACAA;
or
(SEQ ID NO: 1535)
UUCUUGUUCAGGCAAAUCAGC
and
(SEQ ID NO: 1632)
GCUGAUUUGCCUGAACAAGAA;

or wherein all or substantially all of the nucleotides on both the antisense strand and the sense strand are modified nucleotides; and wherein the sense strand further includes inverted abasic residues at the 3′ terminal end and at the 5′ end of the nucleotide sequence, and the sense strand also includes a targeting ligand that is covalently linked to the 5′ terminal end, wherein the targeting ligand includes a compound with affinity for an integrin receptor.

In some embodiments, a MUC5AC RNAi agent disclosed herein includes an antisense strand that consists of, consists essentially of, or comprises a modified nucleotide sequence that differs by 0 or 1 nucleotides from one of the following nucleotide sequences (5′→3′):

(SEQ ID NO: 1127)
cPrpusUfsgsUfaGfuAfgUfcGfcAfgAfaCfaGfsc;
(SEQ ID NO: 1065)
usUfsgsUfaGfuAfgUfcGfcAfgAfaCfaGfsc;
(SEQ ID NO: 1166)
usUfscsuuguucagGfcAfaAfucagsc;
(SEQ ID NO: 1191)
cPrpuUfcuuguucagGfcAfaAfucagsc;

wherein a, c, g, and u represent 2′-O-methyl adenosine, cytidine, guanosine, and uridine, respectively; Af, Cf, Gf, and Uf represent 2′-fluoro adenosine, cytidine, guanosine, and uridine, respectively; cPrpu represents a 5′-cyclopropyl phosphonate-2′-O-methyluridine; s represents a phosphorothioate linkage; and wherein the MUC5AC RNAi agent further includes the sense strand that is at least partially complementary to the antisense strand; and wherein all or substantially all of the nucleotides of the sense strand are modified nucleotides.

In some embodiments, a MUC5AC RNAi agent disclosed herein includes an antisense strand that consists of, consists essentially of, or comprises a modified nucleotide sequence that differs by 0 or 1 nucleotides from one of the following nucleotide sequences (5′→3′):

(SEQ ID NO: 1127)
cPrpusUfsgsUfaGfuAfgUfcGfcAfgAfaCfaGfsc;
(SEQ ID NO: 1065)
usUfsgsUfaGfuAfgUfcGfcAfgAfaCfaGfsc;
(SEQ ID NO: 1166)
usUfscsuuguucagGfcAfaAfucagsc;
(SEQ ID NO: 1191)
cPrpuUfcuuguucagGfcAfaAfucagsc;

wherein the MUC5AC RNAi agent further includes the sense strand that is at least partially complementary to the antisense strand; wherein all or substantially all of the nucleotides of the sense strand are modified nucleotides; wherein all or substantially all of the nucleotides on both the antisense strand and the sense strand are modified nucleotides; and wherein the sense strand further includes inverted abasic residues at the 3′ terminal end and at the 5′ end of the nucleotide sequence, and the sense strand also includes a targeting ligand that is covalently linked to the 5′ terminal end, wherein the targeting ligand includes a compound with affinity for an integrin receptor.

In some embodiments, a MUC5AC RNAi agent disclosed herein includes an antisense strand and a sense strand that consists of, consists essentially of, or comprises one of the following nucleotide sequence pairs (5′→3′):

(SEQ ID NO: 1127)
cPrpusUfsgsUfaGfuAfgUfcGfcAfgAfaCfaGfsc
and
(SEQ ID NO: 1265)
gscuguucuGfCfGfacuacuacaa;
(SEQ ID NO: 1065)
usUfsgsUfaGfuAfgUfcGfcAfgAfaCfaGfsc
and
(SEQ ID NO: 1265)
gscuguucuGfCfGfacuacuacaa;
(SEQ ID NO: 1166)
usUfscsuuguucagGfcAfaAfucagsc
and
(SEQ ID NO: 1315)
gscugauUfuGfcCfugaacaagaa;
and
(SEQ ID NO: 1191)
cPrpuUfcuuguucagGfcAfaAfucagsc
and
(SEQ ID NO: 1315)
gscugauUfuGfcCfugaacaagaa;

wherein a, c, g, and u represent 2′-O-methyl adenosine, cytidine, guanosine, and uridine, respectively; Af, Cf, Gf, and Uf represent 2′-fluoro adenosine, cytidine, guanosine, and uridine, respectively; cPrpu represents a 5′-cyclopropyl phosphonate-2′-O-methyluridine; and s represents a phosphorothioate linkage; and wherein the sense strand also includes a targeting ligand having affinity for an integrin receptor, wherein the targeting ligand is optionally linked at the 5′-end of the sense strand.

In some embodiments, a MUC5AC RNAi agent disclosed herein includes an antisense strand and a sense strand that consists of, consists essentially of, or comprises modified nucleotide sequences that differs by 0 or 1 nucleotides from one of the following sequence pairs (5′→3′):

(SEQ ID NO: 1127)
cPrpusUfsgsUfaGfuAfgUfcGfcAfgAfaCfaGfsc
and
(SEQ ID NO: 1491)
Tri-SM6.1-avb6-(TA14)gscuguucuGfCfGfacuacua
caas(invAb);
(SEQ ID NO: 1065)
usUfsgsUfaGfuAfgUfcGfcAfgAfaCfaGfsc
and
(SEQ ID NO: 1491)
Tri-SM6.1-avb6-(TA14)gscuguucuGfCfGfacuacua
caas(invAb);
(SEQ ID NO: 1166)
usUfscsuuguucagGfcAfaAfucagsc
and
(SEQ ID NO: 1513)
Tri-SM6.1-avb6-(TA14)gscugauUfuGfcCfugaacaa
gaas(invAb);
(SEQ ID NO: 1191)
cPrpuUfcuuguucagGfcAfaAfucagsc
and
(SEQ ID NO: 1513)
Tri-SM6.1-avb6-(TA14)gscugauUfuGfcCfugaacaa
gaas(invAb);

wherein a, c, g, and u represent 2′-O-methyl adenosine, cytidine, guanosine, and uridine, respectively; Af, Cf, Gf, and Uf represent 2′-fluoro adenosine, cytidine, guanosine, and uridine, respectively; cPrpu represents a 5′-cyclopropyl phosphonate-2′-O-methyluridine; Tri-SM6.1-αvβ6-(TA14) represents the tridentate αvβ6 epithelial cell targeting ligand with the chemical structure as shown in FIG. 1; (invAb) represents an inverted abasic deoxyribonucleotide (see also Table 11), and s represents a phosphorothioate linkage.

In some embodiments, a MUC5AC RNAi agent disclosed herein includes an antisense strand that includes a nucleobase sequence that differs by 0 or 1 nucleobases from the nucleotide sequences selected from the group consisting of 5′→3′):

(SEQ ID NO: 79)
UUGUAGUAGUCGCAGAACA;
and
(SEQ ID NO: 83)
UUCUUGUUCAGGCAAAUCA.

In some embodiments, a MUC5AC RNAi agent disclosed herein includes an antisense strand that includes a nucleobase sequence that differs by 0 or 1 nucleobases from the nucleotide sequences selected from the group consisting of (5′→3′):

(SEQ ID NO: 79)
UUGUAGUAGUCGCAGAACA;
and
(SEQ ID NO: 83)
UUCUUGUUCAGGCAAAUCA;

wherein all or substantially all of the nucleotides are modified nucleotides.

In some embodiments, a MUC5AC RNAi agent disclosed herein includes an antisense strand that includes a nucleobase sequence that differs by 0 or 1 nucleobases from the nucleotide sequences selected from the group consisting of (5′→3′):

(SEQ ID NO: 79)
UUGUAGUAGUCGCAGAACA;
and
(SEQ ID NO: 83)
UUCUUGUUCAGGCAAAUCA;

wherein all or substantially all of the nucleotides are modified nucleotides, and wherein SEQ ID NO:79 and SEQ ID NO: 83, respectively, is located at nucleotide positions 1-19 (5′→3′) of the antisense strand.

In some embodiments, a MUC5AC RNAi agent disclosed herein includes an antisense strand and a sense strand that each include a nucleobase sequences that differs by 0 or 1 nucleobases from the nucleotide sequence pairs selected from the group consisting of (5′→3′):

(SEQ ID NO: 79)
UUGUAGUAGUCGCAGAACA;
and
(SEQ ID NO: 568)
UGUUCUGCGACUACUACAA;
or
(SEQ ID NO: 83)
UUCUUGUUCAGGCAAAUCA
and
(SEQ ID NO: 572)
UGAUUUGCCUGAACAAGAA.

In some embodiments, a MUC5AC RNAi agent disclosed herein includes an antisense strand and a sense strand that each include a nucleobase sequences that differs by 0 or 1 nucleobases from the nucleotide sequence pairs selected from the group consisting of (5′→3′):

(SEQ ID NO: 79)
UUGUAGUAGUCGCAGAACA;
and
(SEQ ID NO: 568)
UGUUCUGCGACUACUACAA;
or
(SEQ ID NO: 83)
UUCUUGUUCAGGCAAAUCA
and
(SEQ ID NO: 572)
UGAUUUGCCUGAACAAGAA;

and
wherein all or substantially all of the nucleotides are modified nucleotides.

### Definitions

As used herein, the terms “oligonucleotide” and “polynucleotide” mean a polymer of linked nucleosides each of which can be independently modified or unmodified.

As used herein, an “RNAi agent” (also referred to as an “RNAi trigger”) means a composition that contains an RNA or RNA-like (e.g., chemically modified RNA) oligonucleotide molecule that is capable of degrading or inhibiting (e.g., degrades or inhibits under appropriate conditions) translation of targeted messenger RNA (mRNA) transcripts in a sequence specific manner. As used herein, RNAi agents may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that RNAi agents, as that term is used herein, operate primarily through the RNA interference mechanism, the disclosed RNAi agents are not bound by or limited to any particular pathway or mechanism of action. RNAi agents disclosed herein are comprised of a sense strand and an antisense strand, and include, but are not limited to: small (or short) interfering RNAs (siRNAs), double stranded RNAs (dsRNA), micro RNAs (miRNAs), short hairpin RNAs (shRNA), and dicer substrates. The antisense strand of the RNAi agents described herein is at least partially complementary to the mRNA being targeted (i.e., MUC5AC mRNA). RNAi agents can include one or more modified nucleotides and/or one or more non-phosphodiester linkages.

As used herein, the terms “silence,” “reduce,” “inhibit,” “down-regulate,” or “knockdown” when referring to expression of a given gene, mean that the expression of the gene, as measured by the level of RNA transcribed from the gene or the level of polypeptide, protein, or protein subunit translated from the mRNA in a cell, group of cells, tissue, organ, or subject in which the gene is transcribed, is reduced when the cell, group of cells, tissue, organ, or subject is treated with the RNAi agents described herein as compared to a second cell, group of cells, tissue, organ, or subject that has not or have not been so treated.

As used herein, the terms “sequence” and “nucleotide sequence” mean a succession or order of nucleobases or nucleotides, described with a succession of letters using standard nomenclature. Unless otherwise indicated, nucleotide sequences are written left to right in 5′ to 3′ orientation.

As used herein, a “base,” “nucleotide base,” or “nucleobase,” is a heterocyclic pyrimidine or purine compound that is a component of a nucleotide, and includes the primary purine bases adenine and guanine, and the primary pyrimidine bases cytosine, thymine, and uracil. A nucleobase may further be modified to include, without limitation, universal bases, hydrophobic bases, promiscuous bases, size-expanded bases, and fluorinated bases. (See, e.g., Modified Nucleosides in Biochemistry, Biotechnology and Medicine, Herdewijn, P. ed. Wiley-VCH, 2008). The synthesis of such modified nucleobases (including phosphoramidite compounds that include modified nucleobases) is known in the art.

As used herein, the term “nucleotide” has the same meaning as commonly understood in the art. Thus, the term “nucleotide” as used herein, refers to a glycoside comprising a sugar moiety, a base moiety and a covalently linked group (linkage group), such as a phosphate or phosphorothioate internucleoside linkage group, and covers both naturally occurring nucleotides, such as DNA or RNA, and non-naturally occurring nucleotides comprising modified sugar and/or base moieties, which are also referred to as nucleotide analogs or modified nucleotides herein. A single nucleotide may be referred to here as a monomer or unit.

As used herein, and unless otherwise indicated, the term “complementary,” when used to describe a first nucleobase or nucleotide sequence (e.g., RNAi agent sense strand or targeted mRNA) in relation to a second nucleobase or nucleotide sequence (e.g., RNAi agent antisense strand or a single-stranded antisense oligonucleotide), means the ability of an oligonucleotide or polynucleotide including the first nucleotide sequence to hybridize (form base pair hydrogen bonds under mammalian physiological conditions (or otherwise suitable in vivo or in vitro conditions)) and form a duplex or double helical structure under certain standard conditions with an oligonucleotide that includes the second nucleotide sequence. The person of ordinary skill in the art would be able to select the set of conditions most appropriate for a hybridization test. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification. For example, a and Af, as defined herein, are complementary to U (or T) and identical to A for the purposes of determining identity or complementarity.

As used herein, “perfectly complementary” or “fully complementary” means that in a hybridized pair of nucleobase or nucleotide sequence molecules, all (100%) of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “partially complementary” means that in a hybridized pair of nucleobase or nucleotide sequence molecules, at least 70%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “substantially complementary” means that in a hybridized pair of nucleobase or nucleotide sequence molecules, at least 85%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, the terms “complementary,” “fully complementary,” “partially complementary,” and “substantially complementary” are used with respect to the nucleobase or nucleotide matching between the sense strand and the antisense strand of an RNAi agent, or between the antisense strand of an RNAi agent and a sequence of an MUC5AC mRNA.

As used herein, the term “substantially identical” or “substantial identity,” as applied to a nucleic acid sequence means the nucleotide sequence (or a portion of a nucleotide sequence) has at least about 85% sequence identity or more, e.g., at least 90%, at least 95%, or at least 99% identity, compared to a reference sequence. Percentage of sequence identity is determined by comparing two optimally aligned sequences over a comparison window. The percentage is calculated by determining the number of positions at which the same type of nucleic acid base occurs in both sequences to yield the number of matched positions, dividing the number of matched positions by the total number of positions in the window of comparison and multiplying the result by 100 to yield the percentage of sequence identity. The inventions disclosed herein encompass nucleotide sequences substantially identical to those disclosed herein.

As used herein, the terms “treat,” “treatment,” and the like, mean the methods or steps taken to provide relief from or alleviation of the number, severity, and/or frequency of one or more symptoms of a disease in a subject. As used herein, “treat” and “treatment” may include the prevention, management, prophylactic treatment, and/or inhibition or reduction of the number, severity, and/or frequency of one or more symptoms of a disease in a subject.

As used herein, the phrase “introducing into a cell,” when referring to an RNAi agent, means functionally delivering the RNAi agent into a cell. The phrase “functional delivery,” means delivering the RNAi agent to the cell in a manner that enables the RNAi agent to have the expected biological activity, e.g., sequence-specific inhibition of gene expression.

Unless stated otherwise, use of the symbol [symbol] as used herein means that any group or groups may be linked thereto that is in accordance with the scope of the inventions described herein.

As used herein, the term “isomers” refers to compounds that have identical molecular formulae, but that differ in the nature or the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images are termed “enantiomers,” or sometimes optical isomers. A carbon atom bonded to four non-identical substituents is termed a “chiral center.”

As used herein, unless specifically identified in a structure as having a particular conformation, for each structure in which asymmetric centers are present and thus give rise to enantiomers, diastereomers, or other stereoisomeric configurations, each structure disclosed herein is intended to represent all such possible isomers, including their optically pure and racemic forms. For example, the structures disclosed herein are intended to cover mixtures of diastereomers as well as single stereoisomers.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

The person of ordinary skill in the art would readily understand and appreciate that the compounds and compositions disclosed herein may have certain atoms (e.g., N, O, or S atoms) in a protonated or deprotonated state, depending upon the environment in which the compound or composition is placed. Accordingly, as used herein, the structures disclosed herein envisage that certain functional groups, such as, for example, OH, SH, or NH, may be protonated or deprotonated. The disclosure herein is intended to cover the disclosed compounds and compositions regardless of their state of protonation based on the environment (such as pH), as would be readily understood by the person of ordinary skill in the art. Correspondingly, compounds described herein with labile protons or basic atoms should also be understood to represent salt forms of the corresponding compound. Compounds described herein may be in a free acid, free base, or salt form. Pharmaceutically acceptable salts of the compounds described herein should be understood to be within the scope of the invention.

As used herein, the term “linked” or “conjugated” when referring to the connection between two compounds or molecules means that two compounds or molecules are joined by a covalent bond. Unless stated, the terms “linked” and “conjugated” as used herein may refer to the connection between a first compound and a second compound either with or without any intervening atoms or groups of atoms.

As used herein, the term “including” is used to herein mean, and is used interchangeably with, the phrase “including but not limited to.” The term “or” is used herein to mean, and is used interchangeably with, the term “and/or,” unless the context clearly indicates otherwise.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Where a value is explicitly recited, it is to be understood that values which are about the same quantity or amount as the recited value are also within the scope of the disclosure. Where a combination is disclosed, each sub-combination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Other objects, features, aspects, and advantages of the invention will be apparent from the following detailed description, accompanying figures, and from the claims.

### BRIEF DESCRIPTION OF THE DRAWINGS

FIG. 1. Chemical structure representation of the tridentate αvβ6 epithelial cell targeting ligand referred to herein as Tri-SM6.1-αvβ6-(TA14).

FIG. 2. Chemical structure representation of the peptide αvβ6 epithelial cell targeting ligand referred to herein as αvβ6-pep1.

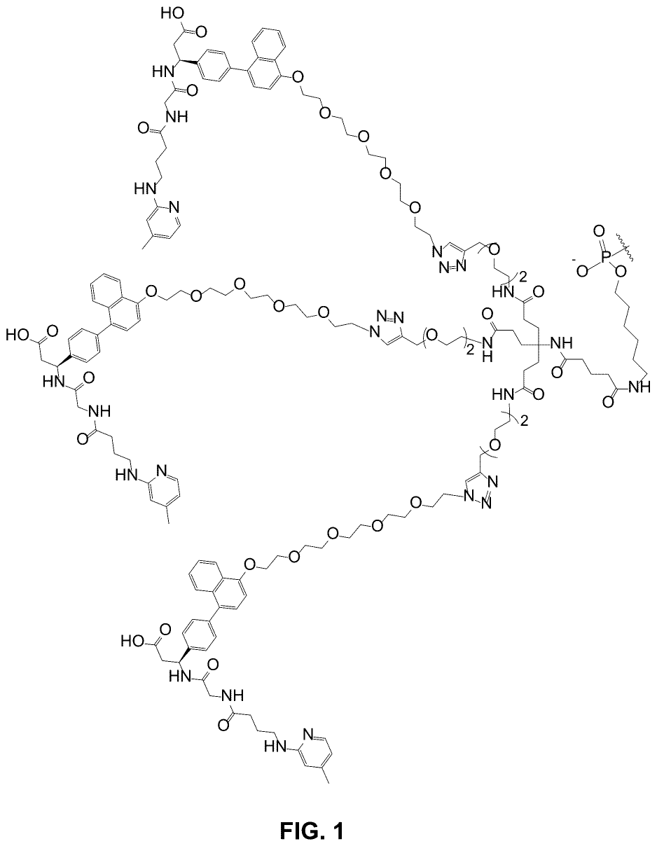

The following abbreviations are used in FIGS. 3A to 3J: a, c, g, i, and u are 2′-O-methyl modified nucleotides; Af, Cf, Gf, and Uf are 2′-fluoro modified nucleotides; o is a phosphodiester linkage; s is a phosphorothioate linkage; invAb is an inverted abasic residue (see, e.g., Table 11); cPrpu is a 5′-cyclopropyl phosphonate-2′-O-methyluridine modified nucleotide (see, e.g., Table 11); Tri-SM6.1-αvβ6-(TA14) is the tridentate αvβ6 epithelial cell targeting ligand having the structure shown in FIG. 1; and (TriAlk14) is the linking group as shown in Table 11, which is suitable for subsequent coupling to targeting ligands (See also, Example 1 herein).

FIG. 3A. Schematic diagram of the modified sense and antisense strands of the MUC5AC RNAi agent conjugate having the structure of AC000437 (see, e.g., Tables 9, 10, and 11), having a tridentate αvβ6 epithelial cell targeting ligand linked at the 5′ end of the sense strand.

FIG. 3B. Schematic diagram of the modified sense and antisense strands of the MUC5AC RNAi agent conjugate having the structure of AC000480 (see, e.g., Tables 9, 10, and 11), having a tridentate αvβ6 epithelial cell targeting ligand linked at the 5′ end of the sense strand.

FIG. 3C. Schematic diagram of the modified sense and antisense strands of the MUC5AC RNAi agent conjugate having the structure of AC000482 (see, e.g., Tables 9, 10, and 11), having a tridentate αvβ6 epithelial cell targeting ligand linked at the 5′ end of the sense strand.

FIG. 3D. Schematic diagram of the modified sense and antisense strands of the MUC5AC RNAi agent conjugate having the structure of AC001305 (see, e.g., Tables 9, 10, and 11), having a tridentate αvβ6 epithelial cell targeting ligand linked at the 5′ end of the sense strand.

FIG. 3E. Schematic diagram of the modified sense and antisense strands of the MUC5AC RNAi agent conjugate having the structure of AC001306 (see, e.g., Tables 9, 10, and 11), having a tridentate αvβ6 epithelial cell targeting ligand linked at the 5′ end of the sense strand.

FIG. 3F. Schematic diagram of the modified sense and antisense strands of the MUC5AC RNAi agent duplex having the structure of AD08089 (see, e.g., Tables 8 and 10), having a (TriAlk14) linker at the 5′ end of the sense strand.

FIG. 3G. Schematic diagram of the modified sense and antisense strands of the MUC5AC RNAi agent duplex having the structure of AD08174 (see, e.g., Tables 8 and 10), having a (TriAlk14) linker at the 5′ end of the sense strand.

FIG. 3H. Schematic diagram of the modified sense and antisense strands of the MUC5AC RNAi agent duplex having the structure of AD08173 (see, e.g., Tables 8 and 10), having a (TriAlk14) linker at the 5′ end of the sense strand.

FIG. 3I. Schematic diagram of the modified sense and antisense strands of the MUC5AC RNAi agent duplex having the structure of AD09240 (see, e.g., Tables 8 and 10), having a (TriAlk14) linker at the 5′ end of the sense strand.

FIG. 3J. Schematic diagram of the modified sense and antisense strands of the MUC5AC RNAi agent duplex having the structure of AD09241 (see, e.g., Tables 8 and 10), having a (TriAlk14) linker at the 5′ end of the sense strand.

### DETAILED DESCRIPTION

### RNAi Agents

Described herein are RNAi agents for inhibiting expression of a MUC5AC gene (referred to herein as MUC5AC RNAi agents or MUC5AC RNAi triggers). Each MUC5AC RNAi agent disclosed herein comprises a sense strand and an antisense strand. The sense strand can be 15 to 49 nucleotides in length. The antisense strand can be 18 to 49 nucleotides in length. The sense and antisense strands can be either the same length or they can be different lengths. In some embodiments, the sense and antisense strands are each independently 18 to 27 nucleotides in length. In some embodiments, both the sense and antisense strands are each 21-26 nucleotides in length. In some embodiments, the sense and antisense strands are each 21-24 nucleotides in length. In some embodiments, the sense and antisense strands are each independently 19-21 nucleotides in length. In some embodiments, the sense strand is about 19 nucleotides in length while the antisense strand is about 21 nucleotides in length. In some embodiments, the sense strand is about 21 nucleotides in length while the antisense strand is about 23 nucleotides in length. In some embodiments, a sense strand is 23 nucleotides in length and an antisense strand is 21 nucleotides in length. In some embodiments, both the sense and antisense strands are each 21 nucleotides in length. In some embodiments, the RNAi agent antisense strands are each independently 18, 19, 20, 21, 22, 23, 24, 25, 26, 27, 28, 29, or 30 nucleotides in length. In some embodiments, the RNAi agent sense strands are each independently 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides in length. The sense and antisense strands are annealed to form a duplex, and in some embodiments, a double-stranded RNAi agent has a duplex length of about 15, 16, 17, 18, 19, 20, 21, 22, 23 or 24 nucleotides.

Examples of nucleotide sequences used in forming MUC5AC RNAi agents are provided in Tables 2, 3, 4, 5, 6, 7, and 11. Examples of RNAi agent duplexes, that include the sense strand and antisense strand sequences in Tables 2, 3, 4, 5, 6, and 7 are shown in Tables 8A, 8B, 8C, 9, 10A, 10B, and 11.

In some embodiments, the region of perfect, substantial, or partial complementarity between the sense strand and the antisense strand is 15-26 (e.g., 15, 16, 17, 18, 19, 20, 21, 22, 23, 24, 25, or 26) nucleotides in length and occurs at or near the 5′ end of the antisense strand (e.g., this region may be separated from the 5′ end of the antisense strand by 0, 1, 2, 3, or 4 nucleotides that are not perfectly, substantially, or partially complementary).

A sense strand of the MUC5AC RNAi agents described herein includes at least 15 consecutive nucleotides that have at least 85% identity to a core stretch sequence (also referred to herein as a “core stretch” or “core sequence”) of the same number of nucleotides in an MUC5AC mRNA. In some embodiments, a sense strand core stretch sequence is 100% (perfectly) complementary or at least about 85% (substantially) complementary to a core stretch sequence in the antisense strand, and thus the sense strand core stretch sequence is typically perfectly identical or at least about 85% identical to a nucleotide sequence of the same length (sometimes referred to, e.g., as a target sequence) present in the MUC5AC mRNA target. In some embodiments, this sense strand core stretch is 15, 16, 17, 18, 19, 20, 21, 22, 23, or 24 nucleotides in length. In some embodiments, this sense strand core stretch is 17 nucleotides in length. In some embodiments, this sense strand core stretch is 19 nucleotides in length.

An antisense strand of a MUC5AC RNAi agent described herein includes at least 18 consecutive nucleotides that have at least 85% complementarity to a core stretch of the same number of nucleotides in an MUC5AC mRNA and to a core stretch of the same number of nucleotides in the corresponding sense strand. In some embodiments, an antisense strand core stretch is 100% (perfectly) complementary or at least about 85% (substantially) complementary to a nucleotide sequence (e.g., target sequence) of the same length present in the MUC5AC mRNA target. In some embodiments, this antisense strand core stretch is 15, 16, 17, 18, 19, 20, 21, 22, 23, or 24 nucleotides in length. In some embodiments, this antisense strand core stretch is 19 nucleotides in length. In some embodiments, this antisense strand core stretch is 17 nucleotides in length. In some embodiments, this antisense strand core stretch is 21 nucleotides in length. A sense strand core stretch sequence can be the same length as a corresponding antisense core stretch sequence or it can be a different length.

The MUC5AC RNAi agent sense and antisense strands anneal to form a duplex. A sense strand and an antisense strand of a MUC5AC RNAi agent can be partially, substantially, or fully complementary to each other. Within the complementary duplex region, the sense strand core stretch sequence is at least 85% complementary or 100% complementary to the antisense core stretch sequence. In some embodiments, the sense strand core stretch sequence contains a sequence of at least 15, at least 16, at least 17, at least 18, at least 19, at least 20, at least 21, at least 22, or at least 23 nucleotides that is at least 85% or 100% complementary to a corresponding 15, 16, 17, 18, 19, 20, 21, 22, 23, or 24 nucleotide sequence of the antisense strand core stretch sequence (i.e., the sense and antisense core stretch sequences of a MUC5AC RNAi agent have a region of at least 15, at least 16, at least 17, at least 18, at least 19, at least 20, at least 21, at least 22, at least 23, or at least 24 nucleotides that is at least 85% base paired or 100% base paired.)

In some embodiments, the antisense strand of a MUC5AC RNAi agent disclosed herein differs by 0, 1, 2, or 3 nucleotides from any of the antisense strand sequences in Table 2, Table 3, or Table 11. In some embodiments, the sense strand of a MUC5AC RNAi agent disclosed herein differs by 0, 1, 2, or 3 nucleotides from any of the sense strand sequences in Table 2, Table 4, Table 5, Table 6, Table 7, or Table 11.

In some embodiments, the sense strand and/or the antisense strand can optionally and independently contain an additional 1, 2, 3, 4, 5, or 6 nucleotides (extension) at the 3′ end, the 5′ end, or both the 3′ and 5′ ends of the core stretch sequences. The antisense strand additional nucleotides, if present, may or may not be complementary to the corresponding sequence in the MUC5AC mRNA. The sense strand additional nucleotides, if present, may or may not be identical to the corresponding sequence in the MUC5AC mRNA. The antisense strand additional nucleotides, if present, may or may not be complementary to the corresponding sense strand's additional nucleotides, if present.

As used herein, an extension comprises 1, 2, 3, 4, 5, or 6 nucleotides at the 5′ and/or 3′ end of the sense strand core stretch sequence and/or antisense strand core stretch sequence. The extension nucleotides on a sense strand may or may not be complementary to nucleotides, either core stretch sequence nucleotides or extension nucleotides, in the corresponding antisense strand. Conversely, the extension nucleotides on an antisense strand may or may not be complementary to nucleotides, either core stretch nucleotides or extension nucleotides, in the corresponding sense strand. In some embodiments, both the sense strand and the antisense strand of an RNAi agent contain 3′ and 5′ extensions. In some embodiments, one or more of the 3′ extension nucleotides of one strand base pairs with one or more 5′ extension nucleotides of the other strand. In other embodiments, one or more of 3′ extension nucleotides of one strand do not base pair with one or more 5′ extension nucleotides of the other strand. In some embodiments, a MUC5AC RNAi agent has an antisense strand having a 3′ extension and a sense strand having a 5′ extension. In some embodiments, the extension nucleotide(s) are unpaired and form an overhang. As used herein, an “overhang” refers to an extension or stretch of one or more unpaired nucleotides located at a terminal end of either the sense strand or the antisense strand that does not form part of the hybridized or duplexed portion of an RNAi agent disclosed herein.

In some embodiments, a MUC5AC RNAi agent comprises an antisense strand having a 3′ extension of 1, 2, 3, 4, 5, or 6 nucleotides in length. In other embodiments, a MUC5AC RNAi agent comprises an antisense strand having a 3′ extension of 1, 2, or 3 nucleotides in length. In some embodiments, one or more of the antisense strand extension nucleotides comprise nucleotides that are complementary to the corresponding MUC5AC mRNA sequence. In some embodiments, one or more of the antisense strand extension nucleotides comprise nucleotides that are not complementary to the corresponding MUC5AC mRNA sequence.

In some embodiments, a MUC5AC RNAi agent comprises a sense strand having a 3′ extension of 1, 2, 3, 4, or 5 nucleotides in length. In some embodiments, one or more of the sense strand extension nucleotides comprises adenosine, uracil, or thymidine nucleotides, AT dinucleotide, or nucleotides that correspond to or are the identical to nucleotides in the MUC5AC mRNA sequence. In some embodiments, the 3′ sense strand extension includes or consists of one of the following sequences, but is not limited to: T, UT, TT, UU, UUT, TTT, or TTTT (each listed 5′ to 3′).

A sense strand can have a 3′ extension and/or a 5′ extension. In some embodiments, a MUC5AC RNAi agent comprises a sense strand having a 5′ extension of 1, 2, 3, 4, 5, or 6 nucleotides in length. In some embodiments, one or more of the sense strand extension nucleotides comprise nucleotides that correspond to or are identical to nucleotides in the MUC5AC mRNA sequence.

Examples of sequences used in forming MUC5AC RNAi agents are provided in Tables 2, 3, 4, 5, 6, 7, and 11. In some embodiments, a MUC5AC RNAi agent antisense strand includes a sequence of any of the sequences in Tables 2, 3, or 11. In certain embodiments, a MUC5AC RNAi agent antisense strand comprises or consists of any one of the modified sequences in Table 3. In some embodiments, a MUC5AC RNAi agent antisense strand includes the sequence of nucleotides (from 5′ end→3′ end) 1-17, 2-17, 1-18, 2-18, 1-19, 2-19, 1-20, 2-20, 1-21, or 2-21, of any of the sequences in Table 2, Table 3, or Table 11. In some embodiments, a MUC5AC RNAi agent sense strand includes the sequence of any of the sequences in Tables 2, 4, 5, 6, or 7. In some embodiments, a MUC5AC RNAi agent sense strand includes the sequence of nucleotides (from 5′ end→3′ end) 1-18, 1-19, 1-20, 1-21, 2-19, 2-20, 2-21, 3-20, 3-21, or 4-21 of any of the sequences in Tables 2, 4, 5, 6, or 7. In certain embodiments, a MUC5AC RNAi agent sense strand comprises or consists of a modified sequence of any one of the modified sequences in Table 4, 5, 6, 7, or 11.

In some embodiments, the sense and antisense strands of the RNAi agents described herein contain the same number of nucleotides. In some embodiments, the sense and antisense strands of the RNAi agents described herein contain different numbers of nucleotides. In some embodiments, the sense strand 5′ end and the antisense strand 3′ end of an RNAi agent form a blunt end. In some embodiments, the sense strand 3′ end and the antisense strand 5′ end of an RNAi agent form a blunt end. In some embodiments, both ends of an RNAi agent form blunt ends. In some embodiments, neither end of an RNAi agent is blunt-ended. As used herein a “blunt end” refers to an end of a double stranded RNAi agent in which the terminal nucleotides of the two annealed strands are complementary (form a complementary base-pair).

In some embodiments, the sense strand 5′ end and the antisense strand 3′ end of an RNAi agent form a frayed end. In some embodiments, the sense strand 3′ end and the antisense strand 5′ end of an RNAi agent form a frayed end. In some embodiments, both ends of an RNAi agent form a frayed end. In some embodiments, neither end of an RNAi agent is a frayed end. As used herein a frayed end refers to an end of a double stranded RNAi agent in which the terminal nucleotides of the two annealed strands form a pair (i.e., do not form an overhang) but are not complementary (i.e. form a non-complementary pair). In some embodiments, one or more unpaired nucleotides at the end of one strand of a double stranded RNAi agent form an overhang. The unpaired nucleotides may be on the sense strand or the antisense strand, creating either 3′ or 5′ overhangs. In some embodiments, the RNAi agent contains: a blunt end and a frayed end, a blunt end and 5′ overhang end, a blunt end and a 3′ overhang end, a frayed end and a 5′ overhang end, a frayed end and a 3′ overhang end, two 5′ overhang ends, two 3′ overhang ends, a 5′ overhang end and a 3′ overhang end, two frayed ends, or two blunt ends. Typically, when present, overhangs are located at the 3′ terminal ends of the sense strand, the antisense strand, or both the sense strand and the antisense strand.

The MUC5AC RNAi agents disclosed herein may also be comprised of one or more modified nucleotides. In some embodiments, substantially all of the nucleotides of the sense strand and substantially all of the nucleotides of the antisense strand of the MUC5AC RNAi agent are modified nucleotides. The MUC5AC RNAi agents disclosed herein may further be comprised of one or more modified internucleoside linkages, e.g., one or more phosphorothioate linkages or phosphorodithioate linkages. In some embodiments, a MUC5AC RNAi agent contains one or more modified nucleotides and one or more modified internucleoside linkages. In some embodiments, a 2′-modified nucleotide is combined with modified internucleoside linkage.

In some embodiments, a MUC5AC RNAi agent is prepared or provided as a salt, mixed salt, or a free-acid. In some embodiments, a MUC5AC RNAi agent is prepared as a pharmaceutically acceptable salt. In some embodiments, a MUC5AC RNAi agent is prepared as a pharmaceutically acceptable sodium salt. Such forms that are well known in the art are within the scope of the inventions disclosed herein.

### Modified Nucleotides

Modified nucleotides, when used in various oligonucleotide constructs, can preserve activity of the compound in cells while at the same time increasing the serum stability of these compounds, and can also minimize the possibility of activating interferon activity in humans upon administration of the oligonucleotide construct.

In some embodiments, a MUC5AC RNAi agent contains one or more modified nucleotides. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides can include, but are not limited to, deoxyribonucleotides, nucleotide mimics, abasic nucleotides, 2′-modified nucleotides, inverted nucleotides, modified nucleobase-comprising nucleotides, bridged nucleotides, peptide nucleic acids (PNAs), 2′,3′-seco nucleotide mimics (unlocked nucleobase analogues), locked nucleotides, 3′-O-methoxy (2′ internucleoside linked) nucleotides, 2′-F-Arabino nucleotides, 5′-Me, 2′-fluoro nucleotide, morpholino nucleotides, vinyl phosphonate deoxyribonucleotides, vinyl phosphonate containing nucleotides, and cyclopropyl phosphonate containing nucleotides. 2′-modified nucleotides (i.e., a nucleotide with a group other than a hydroxyl group at the 2′ position of the five-membered sugar ring) include, but are not limited to, 2′-O-methyl nucleotides (also referred to as 2′-methoxy nucleotides), 2′-fluoro nucleotides (also referred to herein as 2′-deoxy-2′-fluoro nucleotides), 2′-deoxy nucleotides, 2′-methoxyethyl (2′-O-2-methoxylethyl) nucleotides (also referred to as 2′-MOE), 2′-amino nucleotides, and 2′-alkyl nucleotides. It is not necessary for all positions in a given compound to be uniformly modified. Conversely, more than one modification can be incorporated in a single MUC5AC RNAi agent or even in a single nucleotide thereof. The MUC5AC RNAi agent sense strands and antisense strands can be synthesized and/or modified by methods known in the art. Modification at one nucleotide is independent of modification at another nucleotide.

Modified nucleobases include synthetic and natural nucleobases, such as 5-substituted pyrinidines, 6-azapyrimidines and N-2, N-6 and 0-6 substituted purines, (e.g., 2-aminopropyladenine, 5-propynyluracil, or 5-propynylcytosine), 5-methylcytosine (5-me-C), 5-hydroxymethyl cytosine, inosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothynine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulflydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-m-ethyladenine, 8-azaguanine and 8-azaadenine, 7-deazaguanine, 7-deazaadenine, 3-deazaguanine, and 3-deazaadenine.

In some embodiments, the 5′ and/or 3′ end of the antisense strand can include abasic residues (Ab), which can also be referred to as an “abasic site” or “abasic nucleotide.” An abasic residue (Ab) is a nucleotide or nucleoside that lacks a nucleobase at the 1′ position of the sugar moiety. (See, e.g., U.S. Pat. No. 5,998,203). In some embodiments, an abasic residue can be placed internally in a nucleotide sequence. In some embodiments, Ab or AbAb can be added to the 3′ end of the antisense strand. In some embodiments, the 5′ end of the sense strand can include one or more additional abasic residues (e.g., (Ab) or (AbAb)). In some embodiments, UUAb, UAb, or Ab are added to the 3′ end of the sense strand. In some embodiments, an abasic (deoxyribose) residue can be replaced with a ribitol (abasic ribose) residue.

In some embodiments, all or substantially all of the nucleotides of an RNAi agent are modified nucleotides. As used herein, an RNAi agent wherein substantially all of the nucleotides present are modified nucleotides is an RNAi agent having four or fewer (i.e., 0, 1, 2, 3, or 4) nucleotides in both the sense strand and the antisense strand being ribonucleotides (i.e., unmodified). As used herein, a sense strand wherein substantially all of the nucleotides present are modified nucleotides is a sense strand having two or fewer (i.e., 0, 1, or 2) nucleotides in the sense strand being unmodified ribonucleotides. As used herein, an antisense sense strand wherein substantially all of the nucleotides present are modified nucleotides is an antisense strand having two or fewer (i.e., 0, 1, or 2) nucleotides in the antisense strand being unmodified ribonucleotides. In some embodiments, one or more nucleotides of an RNAi agent is an unmodified ribonucleotide. Chemical structures for certain modified nucleotides are set forth in Table 12 herein.

### Modified Internucleoside Linkages

In some embodiments, one or more nucleotides of a MUC5AC RNAi agent are linked by non-standard linkages or backbones (i.e., modified internucleoside linkages or modified backbones). Modified internucleoside linkages or backbones include, but are not limited to, phosphorothioate groups (represented herein as a lower case “s”), chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, a sense strand of a MUC5AC RNAi agent can contain 1, 2, 3, 4, 5, or 6 phosphorothioate linkages, an antisense strand of a MUC5AC RNAi agent can contain 1, 2, 3, 4, 5, or 6 phosphorothioate linkages, or both the sense strand and the antisense strand independently can contain 1, 2, 3, 4, 5, or 6 phosphorothioate linkages. In some embodiments, a sense strand of a MUC5AC RNAi agent can contain 1, 2, 3, or 4 phosphorothioate linkages, an antisense strand of a MUC5AC RNAi agent can contain 1, 2, 3, or 4 phosphorothioate linkages, or both the sense strand and the antisense strand independently can contain 1, 2, 3, or 4 phosphorothioate linkages.

In some embodiments, a MUC5AC RNAi agent sense strand contains at least two phosphorothioate internucleoside linkages. In some embodiments, the phosphorothioate internucleoside linkages are between the nucleotides at positions 1-3 from the 3′ end of the sense strand. In some embodiments, one phosphorothioate internucleoside linkage is at the 5′ end of the sense strand nucleotide sequence, and another phosphorothioate linkage is at the 3′ end of the sense strand nucleotide sequence. In some embodiments, two phosphorothioate internucleoside linkage are located at the 5′ end of the sense strand, and another phosphorothioate linkage is at the 3′ end of the sense strand. In some embodiments, the sense strand does not include any phosphorothioate internucleoside linkages between the nucleotides, but contains one, two, or three phosphorothioate linkages between the terminal nucleotides on both the 5′ and 3′ ends and the optionally present inverted abasic residue terminal caps. In some embodiments, the targeting ligand is linked to the sense strand via a phosphorothioate linkage.

In some embodiments, a MUC5AC RNAi agent antisense strand contains four phosphorothioate internucleoside linkages. In some embodiments, the four phosphorothioate internucleoside linkages are between the nucleotides at positions 1-3 from the 5′ end of the antisense strand and between the nucleotides at positions 19-21, 20-22, 21-23, 22-24, 23-25, or 24-26 from the 5′ end. In some embodiments, three phosphorothioate internucleoside linkages are located between positions 1-4 from the 5′ end of the antisense strand, and a fourth phosphorothioate internucleoside linkage is located between positions 20-21 from the 5′ end of the antisense strand. In some embodiments, a MUC5AC RNAi agent contains at least three or four phosphorothioate internucleoside linkages in the antisense strand.

### Capping Residues or Moieties

In some embodiments, the sense strand may include one or more capping residues or moieties, sometimes referred to in the art as a “cap,” a “terminal cap,” or a “capping residue.” As used herein, a “capping residue” is a non-nucleotide compound or other moiety that can be incorporated at one or more termini of a nucleotide sequence of an RNAi agent disclosed herein. A capping residue can provide the RNAi agent, in some instances, with certain beneficial properties, such as, for example, protection against exonuclease degradation. In some embodiments, inverted abasic residues (invAb) (also referred to in the art as “inverted abasic sites”) are added as capping residues (see Table 12). (See, e.g., F. Czauderna, Nucleic Acids Res., 2003, 31(11), 2705-16). Capping residues are generally known in the art, and include, for example, inverted abasic residues as well as carbon chains such as a terminal C₃H₇ (propyl), C₆H₁₃ (hexyl), or C₁₂H₂₅ (dodecyl) groups. In some embodiments, a capping residue is present at either the 5′ terminal end, the 3′ terminal end, or both the 5′ and 3′ terminal ends of the sense strand. In some embodiments, the 5′ end and/or the 3′ end of the sense strand may include more than one inverted abasic deoxyribose moiety as a capping residue.

In some embodiments, one or more inverted abasic residues (invAb) are added to the 3′ end of the sense strand. In some embodiments, one or more inverted abasic residues (invAb) are added to the 5′ end of the sense strand. In some embodiments, one or more inverted abasic residues or inverted abasic sites are inserted between the targeting ligand and the nucleotide sequence of the sense strand of the RNAi agent. In some embodiments, the inclusion of one or more inverted abasic residues or inverted abasic sites at or near the terminal end or terminal ends of the sense strand of an RNAi agent allows for enhanced activity or other desired properties of an RNAi agent.

In some embodiments, one or more inverted abasic residues (invAb) are added to the 5′ end of the sense strand. In some embodiments, one or more inverted abasic residues can be inserted between the targeting ligand and the nucleotide sequence of the sense strand of the RNAi agent. The inverted abasic residues may be linked via phosphate, phosphorothioate (e.g., shown herein as (invAb)s)), or other internucleoside linkages. In some embodiments, the inclusion of one or more inverted abasic residues at or near the terminal end or terminal ends of the sense strand of an RNAi agent may allow for enhanced activity or other desired properties of an RNAi agent. In some embodiments, an inverted abasic (deoxyribose) residue can be replaced with an inverted ribitol (abasic ribose) residue. In some embodiments, the 3′ end of the antisense strand core stretch sequence, or the 3′ end of the antisense strand sequence, may include an inverted abasic residue. The chemical structures for inverted abasic deoxyribose residues are shown in Table 12 below.

### MUC5AC RNAi Agents

The MUC5AC RNAi agents disclosed herein are designed to target specific positions on a MUC5AC gene (e.g., SEQ ID NO: 1 (NM_001304359.2)). As defined herein, an antisense strand sequence is designed to target a MUC5AC gene at a given position on the gene when the 5′ terminal nucleobase of the antisense strand is aligned with a position that is 19 nucleotides downstream (towards the 3′ end) from the position on the gene when base pairing to the gene. For example, as illustrated in Tables 1 and 2 herein, an antisense strand sequence designed to target a MUC5AC gene at position 3535 requires that when base pairing to the gene, the 5′ terminal nucleobase of the antisense strand is aligned with position 3553 of a MUC5AC gene.

As provided herein, a MUC5AC RNAi agent does not require that the nucleobase at position 1 (5′→3′) of the antisense strand be complementary to the gene, provided that there is at least 85% complementarity (e.g., at least 85, 86, 87, 88, 89, 90, 91, 92, 93, 94, 95, 96, 97, 98, 99, or 100% complementarity) of the antisense strand and the gene across a core stretch sequence of at least 16 consecutive nucleotides. For example, for a MUC5AC RNAi agent disclosed herein that is designed to target position 3535 of a MUC5AC gene, the 5′ terminal nucleobase of the antisense strand of the of the MUC5AC RNAi agent must be aligned with position 3553 of the gene; however, the 5′ terminal nucleobase of the antisense strand may be, but is not required to be, complementary to position 3553 of a MUC5AC gene, provided that there is at least 85% complementarity (e.g., at least 85, 86, 87, 88, 89, 90, 91, 92, 93, 94, 95, 96, 97, 98, 99, or 100% o complementarity) of the antisense strand and the gene across a core stretch sequence of at least 16 consecutive nucleotides. As shown by, among other things, the various examples disclosed herein, the specific site of binding of the gene by the antisense strand of the MUC5AC RNAi agent (e.g., whether the MUC5AC RNAi agent is designed to target a MUC5AC gene at position 3535, at position 4993, at position 15051, or at some other position) is an important factor to the level of inhibition achieved by the MUC5AC RNAi agent. (See also, Kamola et al., The siRNA Non-seed Region and Its Target Sequences are Auxiliary Determinants of Off-Target Effects, PLOS Computational Biology, 11(12), FIG. 1 (2015)).

In some embodiments, the MUC5AC RNAI agents disclosed herein target a MUC5AC gene at or near the positions of the MUC5AC sequence shown in Table 1. In some embodiments, the antisense strand of a MUC5AC RNAi agent disclosed herein includes a core stretch sequence that is fully, substantially, or at least partially complementary to a target MUC5AC 19-mer sequence disclosed in Table 1

TABLE 1
MUC5AC 19-mer mRNA Target Sequences (taken from
homo sapiens mucin 5AC, oligomeric mucus/gel-
forming (MUC5AC) gene transcript, GenBank
NM_001304359.2 (SEQ ID NO: 1))

| | | Corres- | Targeted |
|---|---|---|---|
| | | ponding | Gene |
| SEQ | MUC5AC 19-mer | Positions of | Position (as |
| ID | Target Sequences | Sequence on | referred |
| No. | (5′→3′) | SEQ ID NO: 1 | to herein) |
| 2 | GCUUCCACUACAAGACCUU | 304-322 | 304 |
| 3 | UGUGGAACCACGAUGACAG | 610-628 | 610 |
| 4 | GCAAGACCUCUGCUUCUGU | 923-941 | 923 |
| 5 | CACAGACUGCACCAACUGC | 1277-1295 | 1277 |
| 6 | CAGUGCCUUCACUGUACUG | 1445-1463 | 1445 |
| 7 | AGUGCCUUCACUGUACUGC | 1446-1464 | 1446 |
| 8 | CAGCGAGACCUGCCUGAAG | 1493-1511 | 1493 |
| 9 | GGGAAGUGUUCCUGAACCA | 1567-1585 | 1567 |
| 10 | AACGUCACCAUCUUCAGAC | 1617-1635 | 1617 |
| 11 | ACGUCACCAUCUUCAGACC | 1618-1636 | 1618 |
| 12 | UGUGGGAACUUCAACAGCA | 1758-1776 | 1758 |
| 13 | GGGAACUUCAACAGCAUCC | 1761-1779 | 1761 |
| 14 | UCCAGGCCGAUGACUUCCG | 1777-1795 | 1777 |
| 15 | CUUCUUCAACACCUUCAAG | 1832-1850 | 1832 |
| 16 | UUCAACACCUUCAAGACCC | 1836-1854 | 1836 |
| 17 | CCAACAUCAGGAACAGCUU | 1867-1885 | 1867 |
| 18 | CAUCAGGAACAGCUUCGAG | 1871-1889 | 1871 |
| 19 | AGUAUGCUCAGCACUGGUG | 1921-1939 | 1921 |
| 20 | ACCUACUACUCGAACUGCA | 2001-2019 | 2001 |
| 21 | UACUACUCGAACUGCAUGU | 2004-2022 | 2004 |
| 22 | ACAUCACCUGCAGUGUUGG | 2230-2248 | 2230 |
| 23 | CACCUGCAGUGUUGGCUUC | 2234-2252 | 2234 |
| 24 | UGGACAUGACCUGUUACAG | 2536-2554 | 2536 |
| 25 | AGAGCUACAGCUUCAACGG | 2797-2815 | 2797 |
| 26 | AGGGACCACCUGCUCCAAG | 2915-2933 | 2915 |
| 27 | CUGCUCCAAGGCCAUCAAG | 2924-2942 | 2924 |
| 28 | UGCUCCAAGGCCAUCAAGA | 2925-2943 | 2925 |
| 29 | CUCCAAGGCCAUCAAGAUU | 2927-2945 | 2927 |
| 30 | GACAAGAAGACCAGCAUCU | 3090-3108 | 3090 |
| 31 | AGACCAGCAUCUUCAUCAA | 3097-3115 | 3097 |
| 32 | ACCAGCAUCUUCAUCAACC | 3099-3117 | 3099 |
| 33 | CCUCAGCCCCGAGUUCAAG | 3116-3134 | 3116 |
| 34 | UGGGAACUUCGACGACAUC | 3155-3173 | 3155 |
| 35 | CAGAAGCAGUGCAGCAUCC | 3321-3339 | 3321 |
| 36 | CAGGCCUGCCAUGAAGUUU | 3475-3493 | 3475 |
| 37 | CCCUCUGUUCUGCGACUAC | 3530-3548 | 3530 |
| 38 | CCUCUGUUCUGCGACUACU | 3531-3549 | 3531 |
| 39 | UCUGUUCUGCGACUACUAC | 3533-3551 | 3533 |
| 40 | CUGUUCUGCGACUACUACA | 3534-3552 | 3534 |
| 41 | UGUUCUGCGACUACUACAA | 3535-3553 | 3535 |
| 42 | UCUUUGAUGAGGACAAGAU | 3694-3712 | 3694 |
| 43 | CUUUGAUGAGGACAAGAUG | 3695-3713 | 3695 |
| 44 | UUUGAUGAGGACAAGAUGC | 3696-3714 | 3696 |
| 45 | ACGUCAUCUACCACACGAC | 3910-3928 | 3910 |
| 46 | UGCUACAACUACCAGAUCA | 4443-4461 | 4443 |
| 47 | UACAACUACCAGAUCAGGG | 4446-4464 | 4446 |
| 48 | CUGAUUUGCCUGAACAAGA | 4992-5010 | 4992 |
| 49 | UGAUUUGCCUGAACAAGAA | 4993-5011 | 4993 |
| 50 | CACCCAUCUGCUACAACUA | 5020-5038 | 5020 |
| 51 | ACCCAUCUGCUACAACUAU | 5021-5039 | 5021 |
| 52 | UGCUACAACUAUGAGAUCC | 5028-5046 | 5028 |
| 53 | GCUACAACUAUGAGAUCCG | 5029-5047 | 5029 |
| 54 | GAUCCGCAUCCAGUGUUGC | 5042-5060 | 5042 |
| 55 | AAAGUGGUUCGACGUGGAC | 5297-5315 | 5297 |
| 56 | GUGGUUCGACGUGGACUUC | 5300-5318 | 5300 |
| 57 | GGUUCGACGUGGACUUCCC | 5302-5320 | 5302 |
| 58 | AAGGAAACCUACAACAACA | 5346-5364 | 5346 |
| 59 | AGGAAACCUACAACAACAU | 5347-5365 | 5347 |
| 60 | AAACCUACAACAACAUCAU | 5350-5368 | 5350 |
| 61 | AGAGGUGAGCAUCGAACAC | 5441-5459 | 5441 |
| 62 | GCAGGGACCCUUCAAGAUG | 5519-5537 | 5519 |
| 63 | AGAUGUGCCUCAACUACGA | 5533-5551 | 5533 |
| 64 | AUGUGCCUCAACUACGAGG | 5535-5553 | 5535 |
| 65 | ACCUCCUCUUGGCAGAAAU | 6777-6795 | 6777 |
| 66 | AGGACAACCACUUUGGUGA | 6798-6816 | 6798 |
| 67 | CUCUGCUCCUACAACUAGC | 6998-7016 | 6998 |
| 68 | ACCUCUGCUUCUACAACUA | 7980-7998 | 7980 |
| 69 | AUAACCAGCACAACUUCUG | 8448-8466 | 8448 |
| 70 | ACCAGAACAACCUCUGCUC | 8739-8757 | 8739 |
| 71 | CUACAACCAGCACAAUCUC | 9310-9328 | 9310 |
| 72 | UGGACCAAGUGGUUUGACA | 9729-9747 | 9729 |
| 73 | ACAACCAGCACAACUUCUG | 10206-10224 | 10206 |
| 74 | CAACCACUUUGGUGACAAG | 11014-11032 | 11014 |
| 75 | ACAACCAACACAACUUCUG | 11361-11379 | 11361 |
| 76 | CUCUGCUCCUACAACUAGC | 12965-12983 | 12965 |
| 77 | GAGAUCAUCUUCAACAACA | 15051-15069 | 15051 |
| 78 | AGAUCAUCUUCAACAACAA | 15052-15070 | 15052 |

Homo sapiens mucin 5AC, oligomeric mucus/gel-forming (MUC5AC) gene transcript, GenBank NM_001304359.2 (SEQ ID NO:1) (17,448 bases):

| | |
|---|---|
| 1 | ctcagaggct gctgagggac agggcactct tccccgccgt ccacacaatg agtgttggcc |
| 61 | ggaggaagct ggccctgctc tgggccctgg ctctcgctct ggcctgcacc cggcatacag |
| 121 | gccatgccca ggatggctcc tccgaatcca gctacaagca ccaccctgcc ctctctccta |
| 181 | tcgcccgggg gcccagcggg gtcccgctcc gtggggcgac tgtcttccca tctctgagga |
| 241 | ccatccctgt ggtacgagcc tccaacccgg cgcacaacgg gcgggtgtgc agcacctggg |
| 301 | gcagcttcca ctacaagacc ttcgacggcg acgtcttccg cttccccggc ctctgcaact |
| 361 | acgtgttctc cgagcactgc ggtgccgcct acgaggattt taacatccag ctacgccgca |
| 421 | gccaggagtc agcggccccc acgctgagca gggtcctcat gaaggtggat ggcgtggtca |
| 481 | tccagctgac caagggctcc gtcctggtca acggccaccc ggtcctgctg cccttcagcc |
| 541 | agtctggggt cctcattcag cagagcagca gctacaccaa ggtggaggcc aggctgggcc |
| 601 | ttgtcctcat gtggaaccac gatgacagcc tgctgctgga gctggacacc aaatacgcca |
| 661 | acaagacctg tgggctctgt ggggacttca acgggatgcc cgtggtcagc gagctcctct |
| 721 | cccacaacac caagctgaca cccatggaat tcgggaacct gcagaagatg gacgacccca |
| 781 | cggaccagtg tcaggaccct gtccctgaac ccccgaggaa ctgctccact ggctttggca |
| 841 | tctgtgagga gctcctgcac ggccagctgt tctctggctg cgtggccctg gtggacgtcg |
| 901 | gcagctacct ggaggcttgc aggcaagacc tctgcttctg tgaagacacc gacctgctca |
| 961 | gctgcgtctg ccacaccctt gccgagtact cccggcagtg cacccatgca ggggggttgc |
| 1021 | cccaggactg gcggggccct gacttctgcc cccagaagtg ccccaacaac atgcagtacc |
| 1081 | acgagtgccg ctccccctgc gcagacacct gctccaacca ggagcactcc cgggcctgtg |
| 1141 | aggaccactg tgtggccggc tgcttctgcc ctgaggggac ggtgcttgac gacatcggcc |
| 1201 | agaccggctg tgtccctgtg tcaaagtgtg cctgcgtcta caacggggct gcctatgccc |
| 1261 | caggggccac ctactccaca gactgcacca actgcacctg ctccggaggc cggtggagct |
| 1321 | gccaggaggt tccatgcccg ggtacctgct ctgtgcttgg aggtgcccac ttctcaacgt |
| 1381 | ttgacgggaa gcaatacacg gtgcacggcg actgcagcta tgtgctgacc aagccctgtg |
| 1441 | acagcagtgc cttcactgta ctggctgagc tgcgcaggtg cgggctgacg gacagcgaga |
| 1501 | cctgcctgaa gagcgtgaca ctgagcctgg atggggcgca gacggtggtg gtgatcaagg |
| 1561 | ccagtgggga agtgttcctg aaccagatct acacccagct gcccatctct gcagccaacg |
| 1621 | tcaccatctt cagaccctca accttcttca tcatcgccca gaccagcctg ggcctgcagc |
| 1681 | tgaacctgca gctggtgccc accatgcagc tgttcatgca gctggcgccc aagctccgtg |
| 1741 | ggcagacctg cggtctctgt gggaacttca acagcatcca ggccgatgac ttccggaccc |
| 1801 | tcagtggggt ggtggaggcc accgctgcgg ccttcttcaa caccttcaag acccaggccg |
| 1861 | cctgccccaa catcaggaac agcttcgagg acccctgctc tctgagcgtg gagaatgaga |
| 1921 | agtatgctca gcactggtgc tcgcagctga ccgatgccga cggccccttc ggccggtgcc |
| 1981 | atgctgccgt gaagccggga acctactact cgaactgcat gtttgacacc tgcaactgtg |
| 2041 | agcggagcga ggactgcctg tgcgccgcgc tgtcctccta cgtgcacgcc tgtgccgcca |
| 2101 | agggcgtgca gctcggcggc tggagggacg gcgtctgcac gaagcctatg accacttgcc |
| 2161 | ccaagtcaat gacgtaccac taccatgtca gcacctgcca gcccacctgc cgctccctga |
| 2221 | gcgaggggga catcacctgc agtgttggct tcatccccgt ggatggctgc atctgtccca |
| 2281 | agggcacctt cctggacgac acgggcaagt gtgtgcaggc cagcaactgt ccctgctacc |
| 2341 | acagaggctc catgatcccc aatggggagt cggtgcacga cagcggggct atctgcacct |
| 2401 | gcacacatgg gaagctgagc tgcatcggag gccaagcccc cgccccagtg tgtgctgcgc |
| 2461 | ccatggtgtt ctttgactgc cgaaatgcca cgcccgggga cacaggggct ggctgtcaga |
| 2521 | agagctgcca cacactggac atgacctgtt acagccccca gtgtgtgcct ggctgcgtgt |
| 2581 | gccccgacgg gctggtggcg gacggcgagg gcggctgcat cactgcggag gactgcccct |
| 2641 | gcgtgcacaa tgaggccagc taccgggccg gccagaccat ccgggtgggc tgcaacacct |
| 2701 | gcacctgtga cagcaggatg tggcggtgca cagatgaccc ctgcctggcc acctgcgccg |
| 2761 | tgtacgggga cggccactac ctcaccttcg acggacagag ctacagcttc aacggagact |
| 2821 | gcgagtacac gctggtgcag aaccactgtg gcgggaaaga cagcacccag gactcctttc |
| 2881 | gtgttgtcac cgagaacgtc ccctgcggca ccacagggac cacctgctcc aaggccatca |
| 2941 | agattttcct ggggggcttc gagctgaagc taagccatgg gaaggtggag gtgatcggga |
| 3001 | cggacgagag ccaggaggtg ccatacacca tccggcagat gggcatctac ctggtggtgg |
| 3061 | acaccgacat tggcctggtg ctgctgtggg acaagaagac cagcatcttc atcaacctca |
| 3121 | gccccgagtt caagggcagg gtctgcggcc tgtgtgggaa cttcgacgac atcgccgtta |
| 3181 | atgactttgc cacgcggagc cggtctgtgg tgggggacgt gctggagttt gggaacagct |
| 3241 | ggaagctctc cccctcctgc ccagatgccc tggcgcccaa ggacccctgc acggccaacc |
| 3301 | ccttccgcaa gtcctgggcc cagaagcagt gcagcatcct ccacggcccc accttcgccg |
| 3361 | cctgccacgc acacgtggag ccggccaggt actacgaggc ctgcgtgaac gacgcgtgcg |
| 3421 | cctgcgactc cgggggtgac tgcgagtgct tctgcacggc tgtggccgcc tacgcccagg |
| 3481 | cctgccatga agtaggcctg tgtgtgtcct ggcggacccc gagcatctgc cctctgttct |
| 3541 | gcgactacta caaccccgaa ggccagtgcg agtggcacta ccagccctgc ggggtgccct |
| 3601 | gcctgcgcac ctgccggaac ccccgtggag actgcctgcg ggacgtccgg ggcctggaag |
| 3661 | gctgctaccc caagtgccca ccagaggctc ccatctttga tgaggacaag atgcagtgtg |
| 3721 | tggccacctg cccaaccccg cctctgccac cacggtgcca cgtccatggg aagtcctacc |
| 3781 | ggccaggtgc agtggtgccc tcggacaaga actgccagtc ctgcctttgt acggagcgcg |
| 3841 | gcgtggagtg cacctacaaa gctgaggcct gtgtctgcac ctacaatgga cagcgcttcc |
| 3901 | acccagggga cgtcatctac cacacgacgg atggcacggg tggctgcatc tccgcccgct |
| 3961 | gcggggccaa cggcaccatt gagaggaggg tctacccctg cagccccacc acccctgtcc |
| 4021 | ccccaaccac cttctccttc tccacacccc cgcttgtcgt gagctccacg cacaccccca |
| 4081 | gcaatggccc aagcagcgcg cacacaggcc ctccgagcag cgcctggccc accacagcag |
| 4141 | gcacttctcc caggacgagg ctgcccacag cctctgcctc actgccgccg gtctgtgggg |
| 4201 | aaaagtgcct gtggtcgcca tggatggatg tcagccgccc tggacggggc acggacagcg |
| 4261 | gtgacttcga cacactggag aacctccgcg cccatgggta ccgggtgtgc gaatcaccca |
| 4321 | ggtcggtgga gtgccgagct gaggacgccc ccggagtgcc gctccgagcc ctggggcagc |
| 4381 | gtgtgcagtg cagcccggat gtggggctga cctgtcgtaa cagggagcag gcatcggggc |
| 4441 | tctgctacaa ctaccagatc agggtccagt gctgcacgcc cctaccctgc tccacctcta |
| 4501 | gcagtccagc ccagaccact cctccaacta cctccaagac cactgaaacc cgggcctcag |
| 4561 | gctcctcagc tcccagcagc acacctggca ccgtgtctct ctctacagcc aggacgacac |
| 4621 | ctgccccagg taccgctacc tctgtcaaaa aaactttctc aactcccagc cctccgccag |
| 4681 | tgccggcaac atcaacatca tccatgtcga ccacggcccc ggggacctct gtggtctcca |
| 4741 | gcaagcccac ccccacggag cccagcacat cctcctgcct gcaggagctt tgcacctgga |
| 4801 | ccgagtggat cgatggcagc taccctgctc ctggaataaa tggtggagat tttgacacat |
| 4861 | ttcaaaattt gagagacgaa ggatacacat tctgtgaaag tcctcgaagc gtgcagtgcc |
| 4921 | gggcagagag cttccccaac acgccgctgg cagacctggg gcaggacgtc atctgcagcc |
| 4981 | acacagaggg gctgatttgc ctgaacaaga accagctccc acccatctgc tacaactatg |
| 5041 | agatccgcat ccagtgttgc gagacggtga acgtgtgcag agacatcacc agactgccaa |
| 5101 | agaccgtcgc aacgacacgg ccgactccac atccaaccgg agctcagacc cagaccacct |
| 5161 | tcaccacaca catgccctcg gcctccacag agcaacccac ggcaacctcc aggggtgggc |
| 5221 | ccacagcaac cagcgtcaca cagggcaccc acaccacact agtcaccaga aactgtcatc |
| 5281 | cccggtgcac ctggacaaag tggttcgacg tggacttccc gtcccccgga ccccatggtg |
| 5341 | gagacaagga aacctacaac aacatcatca ggagtgggga aaaaatctgc cgccgacctg |
| 5401 | aggagatcac caggctccag tgccgagcca agagccaccc agaggtgagc atcgaacacc |
| 5461 | tgggccaggt ggtgcagtgc agccgggaag agggcctggt gtgccggaac caggaccagc |
| 5521 | agggaccctt caagatgtgc ctcaactacg aggtgcgtgt gctctgctgc gagaccccca |
| 5581 | gaggctgcca catgacctcc acacctggct ccacctctag cagtccagcc cagaccactc |
| 5641 | cttcaacaac ctccaagacc actgaaaccc aggcctcagg ctcctcagcc cccagcagca |
| 5701 | cacctggcac cgtgtctctc tctacagcca ggacgacacc tgccccaggt accgctacct |
| 5761 | ctgtcaaaaa aactttctca actcccagcc ctccgccagt gccggcaaca tcaacatcat |
| 5821 | ccatgtcgac cacggccccg gggacctctg tggtctccag caagcccacc cccacggagc |
| 5881 | ccagcacatc ctcctgcctg caggagcttt gcacctggac cgagtggatt gatggcagct |
| 5941 | accctgctcc tggaataaat ggtggagatt ttgacacatt tcaaaatttg agagacgaag |
| 6001 | gatacacatt ctgtgaaagt cctcgaagcg tgcagtgccg ggcagagagc ttccccaaca |
| 6061 | cgccgctggc agacctgggg caggacgtca tctgcagcca cacagagggg ctgatttgcc |
| 6121 | tgaacaagaa ccagctccca cccatctgct acaactatga gatccgcatc cagtgttgcg |
| 6181 | agacggtgaa cgtgtgcaga gacatcacca gaccgccaaa gaccgtcgca acgacacggc |
| 6241 | cgactccaca tccaaccgga gctcagaccc agaccacctt caccacacac atgccctcgg |
| 6301 | cctccacaga gcaacccacg gcaacctcca ggggtgggcc cacagcaacc agcgtcacac |
| 6361 | agggcaccca caccacacca gtcaccagaa actgtcatcc ccggtgcacc tggacaacgt |
| 6421 | ggttcgacgt ggacttcccg tcccccggac cccatggtgg agacaaggaa acctacaaca |
| 6481 | acatcatcag gagtggggaa aaaatctgcc gccgacctga ggagatcacc aggctccagt |
| 6541 | gccgagccaa gagccaccca gaggtgagca tcgaacacct gggccaggtg gtgcagtgca |
| 6601 | gccgggaaga gggcctggtg tgccggaacc aggaccagca gggacccttc aagatgtgcc |
| 6661 | tcaactacga ggtgcgtgtg ctctgctgcg agacccccaa aggctgcccc gtgacctcca |
| 6721 | cacctgtgac agctcctagc acccctagtg ggagagccac cagcccaact cagagcacct |
| 6781 | cctcttggca gaaatccagg acaaccactt tggtgacaac cagcacaacc tccactccac |
| 6841 | agaccagtac aacctatgcc catacaacca gcacaacctc tgctcctaca gccagaacaa |
| 6901 | cctctgctcc tacaaccaga acaacctctg cctctccagc cagcacaacc tctggtcctg |
| 6961 | gaaatactcc cagccctgtt cctaccacca gcacaatctc tgctcctaca actagcataa |
| 7021 | cctctgcccc tacaaccagc acaacctctg cccctacaag cagcacaacc tctggtcctg |
| 7081 | gaactactcc cagccctgtt cctaccacca gcataacctc tgcccctaca accagcacaa |
| 7141 | cctctgctcc tacaaccagc acaacctctg cccgtacaag cagcacaacc tctgccacta |
| 7201 | ccaccagcag aatctctggt cctgaaacta ctcccagccc tgttcctacc accagcacaa |
| 7261 | cctctgccac tacaaccagc acaacctcag ctcctacaac cagcacaacc tctgccccta |
| 7321 | caagcagcac aacctccagt ccacagacca gcacaacctc ggctcctaca accagcacaa |
| 7381 | cttctggtcc tggaactacc ccaagccctg ttcccacgac cagcacaacc tctgccccta |
| 7441 | caacaagaac aacttctgct cctaaaagca gcacaacctc tgccgctaca accagcacaa |
| 7501 | cctctggtcc tgaaactact cctagacctg ttcctaccac cagcacaacc tcttctccta |
| 7561 | caaccagcac aacctctgct cctacaacca gcacaacctc tgcttctaca accagcacaa |
| 7621 | cctctggtgc tggaactact cccagccctg ttcccaccac cagcacaacc tctgctccta |
| 7681 | caaccagcac aacctctgcc cctataagca gcacaacctc tgccactaca accagcacaa |
| 7741 | cctctggtcc tggaactact cccagccctg ttcctaccac gagcacaacc tctgctccta |
| 7801 | caaccagcac aacctctggt cctggaacta ctcccagtgc tgttcccacc accagcataa |
| 7861 | cctctgcacc tacaaccagc acaaactctg cccctataag cagcacaacc tctgccacta |
| 7921 | caaccagcag aatctctggt cctgaaacta ctcccagccc tgttcctacc gccagcacaa |
| 7981 | cctctgcttc tacaactagc acaacctctg gtcctggaac tactcccagc cctgttccta |
| 8041 | ccaccagcac aatctctgtt cctaccacca gcacaacttc tgcttctaca accagcacaa |
| 8101 | cctctgcttc tacaaccagc acaacctctg gtcctggaac tactcccagc cctgttccca |
| 8161 | ccaccagcac aacctctgct cccacaacaa gcacaacctc tgcccctaca accagcacaa |
| 8221 | tctcggcccc aacaaccagc acaacctctg ccactacaac cagcacgacc tctgctccta |
| 8281 | cacccagaag aacctcagcc cctacaacca gcacaatctc tgcctctacc accagcacaa |
| 8341 | cctctgcgac tacaaccagc acaacctctg ctactacaac cagcacaatc tctgccccta |
| 8401 | caaccagcac aactttgtct cctacaacca gcacaacctc tactactata accagcacaa |
| 8461 | cttctgcccc tataagcagc acaacttcca caccacagac cagcacaact tcggctccta |
| 8521 | caaccagcac aacttctggt cctggaacta cttcaagccc tgttcccacc accagcacaa |
| 8581 | cctctgcccc tacaaccagc acaacctctg cccctacaac cagaacaacc tctgtcccta |
| 8641 | caagcagcac aacctccact gctacaacca gcacaacctc tggccctgga actactccca |
| 8701 | gccctgttcc caccaccagt acaacctctg ctcctacaac cagaacaacc tctgctccta |
| 8761 | caaccagcac aacctctgcc cctacaacca gcacaacctc tgcccctaca agcagcacaa |
| 8821 | cctcagctac tacaaccagc acaatctctg ttcctacaac cagcacaact tctgttcctg |
| 8881 | gaactactcc cagccctgtt cctaccacca gcacaatctc tgttcctacc accagcacaa |
| 8941 | cttctgcttc tacaaccagc acaacctctg gtcctggaac tactcccagc cctgttccca |
| 9001 | ccaccagcac aacctctgct cccacaacaa gcacaacctc tgcccctaca accagcacaa |
| 9061 | tctcggcccc aacaaccagc acaccctctg cccctacaac cagcacaacc ttagctccta |
| 9121 | caaccagcac aacctctgcc cctacaacca gcacaacctc tacccctaca agcagcacaa |
| 9181 | cctcctctcc acagaccagc acaacctcgg cttctaccac cagcataact tctggtcctg |
| 9241 | gaactacccc aagccctgtt cccaccacca gcacaacctc tgctcctaca accagcacaa |
| 9301 | cctctgccgc tacaaccagc acaatctcgg ccccaacaac cagcacaacg tctgctccta |
| 9361 | caaccagcac aacctctgcc tctacagcca gcaaaacctc tggtcttgga actactccca |
| 9421 | gccctattcc taccaccagc acaacctctc ctcctacaac cagcacaact tctgcctcta |
| 9481 | cagccagcaa aacctctggt cctggaacca ctcccagccc tgttcccacc accagcacaa |
| 9541 | tctttgctcc tagaaccagc accacttctg cctctacaac cagcacaacc cctggtcctg |
| 9601 | gaaccactcc cagccccgtt cccaccacca gcacagcctc tgtttcaaag accagcacaa |
| 9661 | gccatgtttc catatccaag acaacccact cccaaccagt caccagagac tgtcatctcc |
| 9721 | ggtgcacctg gaccaagtgg tttgacatag acttcccatc ccctggaccc cacggcgggg |
| 9781 | acaaggaaac ctacaacaac atcatcagga gtggggaaaa aatctgccgc cgacctgagg |
| 9841 | agatcaccag gctccagtgc cgagccgaga gccacccgga ggtgagcatt gaacacctgg |
| 9901 | gccaggtggt gcagtgcagc cgtgaagagg gcctggtgtg ccggaaccag gaccagcagg |
| 9961 | gacccttcaa gatgtgcctc aactacgagg tgcgtgtgct ctgctgcgag acccctaaag |
| 10021 | gttgccccgt gacctccaca cctgtgacag ctcctagcac ccctagtggg agagccacca |
| 10081 | gcccaactca gagcacttcc tcttggcaga aatccaggac aaccactttg gtgacaacca |
| 10141 | gcacaacctc cactccacag accagcacaa cctctgctcc tacaaccagc acaacctctg |
| 10201 | ctcccacaac cagcacaact tctgccccta caaccagcac aacctccact ccacagacca |
| 10261 | gcatatcctc tgcccctaca agcagcacaa cctcggctcc tacaagcagc acaatctctg |
| 10321 | ctcgtacaac cagcataatc tctgccccta caaccagcac aacctcttcc cctacaacca |
| 10381 | gcacaacctc tgctactaca accagcacaa cctctgcccc tacaagcagc acaacctcca |
| 10441 | ctccacagac cagcaaaacc tcagctgcta caagcagcac aacctccggt tctggaacta |
| 10501 | ctcccagccc tgttaccacc accagcacag cctctgtttc aaagaccagc acaagccatg |
| 10561 | tttctgtatc caagacaacc cactcccaac cagtcaccag agactgtcat ccccggtgca |
| 10621 | cctggaccaa atggtttgat gtggactttc catcccctgg accccacggt ggggacaagg |
| 10681 | aaacctacaa caacatcatc aggagtgggg aaaaaatctg ccgccgacct gaggagatca |
| 10741 | ccaggctcca gtgccgagcc aagagccacc cggaggtgag catcgaacac ctgggccagg |
| 10801 | tggtgcagtg cagccgcgaa gagggcctgg tgtgccggaa ccaggaccag cagggaccct |
| 10861 | tcaagatgtg cctcaactac gaggtgcgtg tgctttgctg cgagaccccc aaaggctgcc |
| 10921 | ccgtgacctc cacatctgtg acagctccta gcacccctag tgggagagcc accagcccaa |
| 10981 | ctcagagcac ctcctcttgg cagaaatcca ggacaaccac tttggtgaca agcagcataa |
| 11041 | cctccactac acagaccagc acaacctctg cccctacaac tagcacaacc cctgcttcta |
| 11101 | tacccagcac aacctctgcc ccaacaacca gcacaacctc tgctcccaca acgagcacaa |
| 11161 | cttctgcccc tacaaccagc acaacctcca ctccacagac caccacatcc tctgccccta |
| 11221 | caagcagcac aacctcggct cctaccacca gcacaatctc tgcccctaca accagcacaa |
| 11281 | tctctgcccc tacaaccagc acaacctctg ctcccacagc cagcacaacg tcagctccta |
| 11341 | cgagcacttc ctcggctcct acaaccaaca caacctctgc ccctacaact agcactacct |
| 11401 | ctgctcccat aaccagcaca atctctgccc ctacaaccag cacaacctcc actccacaga |
| 11461 | ccagcacaat ctcttcccct acaaccagca caacctccac tccgcagacc agcacaacct |
| 11521 | cttcccctac aactagcaca acctcagctc ctacaaccag cacaacttct gcccctacaa |
| 11581 | ccagcacaac ctccactcca cagaccagca tatcctctgc ccctacaagc agcacaacct |
| 11641 | ctgctcctac agccagcaca atctctgccc ctacaaccag cacaacctct ttccatacaa |
| 11701 | ccagcacaac ctctccccct acaagcagca caagctccac tccacagacc agcaaaacct |
| 11761 | cagctgctac aagcagcaca acctccggtt ctggaactac tcccagcccc gttcccacca |
| 11821 | ccagcacagc ctctgtttca aagaccagca caagccatgt ttctgtatcc aagacaaccc |
| 11881 | actcccaacc agtcaccaga gactgtcatc cccggtgcac ctggaccaag tggtttgacg |
| 11941 | tggactttcc atcccctgga ccccacggtg gggacaagga aacctacaac aacatcatca |
| 12001 | ggagtgggga aaaaatctgc cgccgacctg aggagatcac caggctccag tgccgagccg |
| 12061 | agagccaccc ggaggtgagc atcgaacacc tgggccaggt ggtgcagtgc agccgggaag |
| 12121 | agggcctggt gtgccggaac caggaccagc agggaccctt caagatgtgc ctcaactacg |
| 12181 | aggtgcgtgt gctctgctgc gagaccccca aaggctgccc cgtgacctcc acacctgtga |
| 12241 | cagctcctag cacccctagt gggagagcca ccagcccaac tcagagcact tcctcttggc |
| 12301 | agaaatccag gacaaccact ttggtgacaa ccagcacaac ctccactcca cagaccagca |
| 12361 | caacctctgc ccctacaacc agcacaatcc ctgcttctac acccagcaca acctctgccc |
| 12421 | ctacaaccag cacaacctct gcccctacaa ccagcacgac ctcagctcct acacacagaa |
| 12481 | cgacttctgg tcctacaacc agcacaacct tggctcctac aaccagcaca acctctgctc |
| 12541 | caacaaccag cacaaactct gctcctacaa ccagcacaat ctctgcctct acaaccagca |
| 12601 | caatctctgc ccctacaacc agcacaatct cttcccctac aagcagcaca acctccactc |
| 12661 | cacagaccag caaaacctca gctgctacaa gcagcacaac ctccggttct ggaactactc |
| 12721 | caagccctgt tcccaccacc agcacaacct ctgcctctac aaccagcaca acttctgctc |
| 12781 | ctacaaccag cacaacctct ggtcctggaa ctactccaag ccctgttccc agcaccagta |
| 12841 | caacctctgc tgctacaacc agcacaacct ctgctcctac aaccagaaca acatctgctc |
| 12901 | ctacaagcag catgacctct ggtcctggaa ctactcccag ccctgttccc accaccagca |
| 12961 | caacctctgc tcctacaact agcacaacct ctggtcctgg aactactccc agccctgttc |
| 13021 | ccaccaccag cacaacctct gctcctataa ccagcacaac ctctggtcct ggaagtactc |
| 13081 | ccagccctgt tcccaccacc agcacaacct ctgctcctac aaccagcaca acctctgcct |
| 13141 | ctacagccag cacaacctct ggtcctggaa ctactcccag ccctgttccc accaccagca |
| 13201 | caacctctgc tcctacaacc agaacaacct ctgcctctac agccagcaca acctctggtc |
| 13261 | ctggaagtac tcccagccct gttcccacca ccagcacaac ctctgctcct acaaccagaa |
| 13321 | caacccctgc ctctacagcc agcacaacct ctggtcctgg aactactccc agccctgttc |
| 13381 | ccaccacaag cacaacctct gcttctacaa ccagcacaat ctctctccct acaaccagca |
| 13441 | caacctctgc tcctataacc agcatgacct ctggtcctgg aactactccc agccctgttc |
| 13501 | ccaccaccag cacaacctct gctcctacaa ccagcacaac ctctgcctct acagccagca |
| 13561 | caacctctgg tcctggaact actcccagcc ctgttcccac caccagcaca acctctgctc |
| 13621 | ctacaaccag cacaacctct gcctctacag ccagcacaac ctctggtcct ggaacttctc |
| 13681 | tcagccctgt tcccaccacg agcacaacct ctgctcctac aactagcaca acctctggtc |
| 13741 | ctggaactac tcccagccct gttcccacca ccagcacaac ctctgctcct acaaccagca |
| 13801 | cgacctctgg tcctggaact actcccagcc ccgttcccac caccagcaca acccctgttt |
| 13861 | caaagaccag cacaagccat ctttctgtat ccaagacaac ccactcccaa ccagtcacca |
| 13921 | gtgactgtca tcctctgtgc gcctggacaa agtggttcga cgtggacttc ccatcccctg |
| 13981 | gaccccacgg cggggacaag gaaacctaca acaacatcat caggagtggg gaaaaaatct |
| 14041 | gccgccgacc tgaggagatc accaggctcc agtgccgagc cgagagccac ccggaggtga |
| 14101 | acattgaaca cctgggtcag gtggtgcagt gcagccgtga agagggcctg gtgtgccgga |
| 14161 | accaggacca gcagggaccc ttcaagatgt gcctcaacta cgaggtgcgc gtgctctgct |
| 14221 | gcgagacccc cagaggctgc ccggtgacct ctgtgacccc atatgggact tctcctacca |
| 14281 | atgctctgta tccttccctg tctacttcca tggtatccgc ctccgtggca tccacctctg |
| 14341 | tggcatccag ctctgtggca tccagctctg tggcttactc cacccaaacc tgcttctgca |
| 14401 | acgtggctga ccggctctac cctgcaggat ccaccatata ccgccacaga gacctcgctg |
| 14461 | gccattgcta ttatgccctg tgtagccagg actgccaagt ggtcagaggg gttgacagtg |
| 14521 | actgtccgtc caccacgctg cctcctgccc cagccacgtc cccttcaata tccacctccg |
| 14581 | agcccgtcac tgagctggga tgcccaaatg cggttccccc cagaaagaaa ggtgagacct |
| 14641 | gggccacacc caactgctcc gaggccacct gtgagggcaa caacgtcatc tccctgcgcc |
| 14701 | cgcgcacgtg cccgagggtg gagaagccca cttgtgccaa cggctacccg gctgtgaagg |
| 14761 | tggctgacca agatggctgc tgccatcact accagtgcca gtgtgtgtgc agcggctggg |
| 14821 | gtgaccccca ctacatcacc ttcgacggca cctactacac cttcctggac aactgcacgt |
| 14881 | acgtgctggt gcagcagatt gtgcccgtgt atggccactt ccgcgtgctc gtcgacaact |
| 14941 | acttctgcgg tgcggaggac gggctctcct gcccgaggtc catcatcctg gagtaccacc |
| 15001 | aggaccgcgt ggtgctgacc cgcaagccag tccacggggt gatgacaaac gagatcatct |
| 15061 | tcaacaacaa ggtggtcagc cccggcttcc ggaaaaacgg catcgtggtc tcgcgcatcg |
| 15121 | gcgtcaagat gtacgcgacc atcccggagc tgggagtcca ggtcatgttc tccggcctca |
| 15181 | tcttctccgt ggaggtgccc ttcagcaagt ttgccaacaa caccgagggc cagtgcggca |
| 15241 | cttgcaccaa cgacaggaag gatgagtgcc gcacgcctag ggggacggtg gtcgcttcct |
| 15301 | gctccgagat gtccggcctc tggaacgtga gcatacccga ccagccagcc tgccaccggc |
| 15361 | ctcacccgac gcccaccacg gtcgggccca ccacagttgg gtctaccacg gtcgggccca |
| 15421 | ccacagttgg gtctaccacg gtcgggccca ccacaccgcc tgctccgtgc ctgccatcac |
| 15481 | ccatctgcca gctgattctg agcaaggtct ttgagccgtg ccacactgtg atccccccac |
| 15541 | tgctgttcta tgagggctgc gtctttgacc ggtgccacat gacggacctg gatgtggtgt |
| 15601 | gctccagcct ggagctgtac gcggcactct gtgcgtccca cgacatctgc atcgattgga |
| 15661 | gaggccggac cggccacatg tgcccattca cctgcccagc cgacaaggtg taccagccct |
| 15721 | gcggcccgag caacccctcc tactgctacg ggaatgacag cgccagcctc ggggctctgc |
| 15781 | cggaggccgg ccccatcacc gaaggctgct tctgtccgga gggcatgacc ctcttcagca |
| 15841 | ccagtgccca agtctgcgtg cccacgggct gccccaggtg tctggggccc cacggagagc |
| 15901 | cggtgaaggt gggccacacc gtcggcatgg actgccagga gtgcacgtgt gaggcggcca |
| 15961 | cgtggacgct gacctgccga cccaagctct gcccgctgcc ccctgcctgc cccctgcccg |
| 16021 | gcttcgtgcc tgtgcctgca gccccacagg ccggccagtg ctgcccccag tacagctgcg |
| 16081 | cctgcaacac cagccgctgc cccgcgcccg tgggctgtcc tgagggcgcc cgcgcgatcc |
| 16141 | cgacctacca ggagggggcc tgctgcccag tccaaaactg cagctggaca gtgtgcagca |
| 16201 | tcaacgggac cctgtaccag cccggcgccg tggtctcctc gagcctgtgc gaaacctgca |
| 16261 | ggtgtgagct gccgggtggc cccccatcgg acgcgtttgt ggtcagctgt gagacccaga |
| 16321 | tctgcaacac acactgccct gtgggcttcg agtaccagga gcagagcggg cagtgctgtg |
| 16381 | gcacctgtgt gcaggtcgcc tgtgtcacca acaccagcaa gagccccgcc cacctcttct |
| 16441 | accccggcga gacctggtca gacgcaggga accactgtgt gacccaccag tgtgagaagc |
| 16501 | accaggatgg gctcgtggtg gtcaccacga agaaggcgtg ccccccgctc agctgttctc |
| 16561 | tggacgaggc ccgcatgagc aaggacggct gctgccgctt ctgcccgccg cccccgcccc |
| 16621 | cgtaccagaa ccagtcgacc tgtgctgtgt accataggag cctgatcatc cagcagcagg |
| 16681 | gctgcagctc ctcggagccc gtgcgcctgg cttactgccg ggggaactgt ggggacagct |
| 16741 | cttccatgta ctcgctcgag ggcaacacgg tggagcacag gtgccagtgc tgccaggagc |
| 16801 | tgcggacctc gctgaggaat gtgaccctgc actgcaccga cggctccagc cgggccttca |
| 16861 | gctacaccga ggtggaagag tgcggctgca tgggccggcg gtgccctgcg ccgggcgaca |
| 16921 | cccagcactc ggaggaggcg gaacccgagc ccagccagga ggcagagagt gggagctggg |
| 16981 | agagaggcgt cccagtgtcc cccatgcact gaccagcact gccgccctcc tgacctccaa |
| 17041 | ggagaacctc ccatatgtcc tctgagctcg gcttccaagg ccagtggaac ttgtgcccct |
| 17101 | gtccaggcgg ctgcagcttt gaacacactg tccacgcccg ctttcttgtg gagggtgtgg |
| 17161 | gctatgggtc acctgctgcc tggaggaggg gcccttaccc accccgcctg cagccacctc |
| 17221 | tcaggaccag ccccggggct ggccgagctc ctctggccat gcatccagcc tgctgttctg |
| 17281 | gggacgtgag catcacctga gggtctcagg aatgacgctt ggacatggtg atcagctgcc |
| 17341 | tggtggctgc aggaggaaga acctcactcc tacctcagcc ctcagcctgc gctcccctcc |
| 17401 | tcagtacacg gccaatctgt tgcataaata cacttgagca ttttgcaa |

In some embodiments, a MUC5AC RNAi agent includes an antisense strand wherein position 19 of the antisense strand (5′43′) is capable of forming a base pair with position 1 of a 19-mer target sequence disclosed in Table 1. In some embodiments, a MUC5AC agent includes an antisense strand wherein position 1 of the antisense strand (5′43′) is capable of forming a base pair with position 19 of a 19-mer target sequence disclosed in Table 1.

In some embodiments, a MUC5AC agent includes an antisense strand wherein position 2 of the antisense strand (5′→3′) is capable of forming a base pair with position 18 of a 19-mer target sequence disclosed in Table 1. In some embodiments, a MUC5AC agent includes an antisense strand wherein positions 2 through 18 of the antisense strand (5′ 4 3′) are capable of forming base pairs with each of the respective complementary bases located at positions 18 through 2 of the 19-mer target sequence disclosed in Table 1.

For the RNAi agents disclosed herein, the nucleotide at position 1 of the antisense strand (from 5′ end→3′ end) can be perfectly complementary to a MUC5AC gene, or can be non-complementary to a MUC5AC gene. In some embodiments, the nucleotide at position 1 of the antisense strand (from 5′ end→3′ end) is a U, A, or dT. In some embodiments, the nucleotide at position 1 of the antisense strand (from 5′ end→3′ end) forms an A:U or U:A base pair with the sense strand.

In some embodiments, a MUC5AC RNAi agent antisense strand comprises the sequence of nucleotides (from 5′ end→3′ end) 2-18 or 2-19 of any of the antisense strand sequences in Table 2, Table 3, or Table 11. In some embodiments, a MUC5AC RNAi sense strand comprises the sequence of nucleotides (from 5′ end→3′ end) 1-17, 1-18, or 2-18 of any of the sense strand sequences in Table 2, Table 4, Table 5, Table 6, or Table 7.

In some embodiments, a MUC5AC RNAi agent is comprised of (i) an antisense strand comprising the sequence of nucleotides (from 5′ end→3′ end) 1-18, 1-19, or 2-19 of any of the antisense strand sequences in Table 2 or Table 3, and (ii) a sense strand comprising the sequence of nucleotides (from 5′ end→3′ end) 2-19, 1-19, 1-18, or 2-18 of any of the sense strand sequences in Table 2, Table 4, Table 5, Table 6, or Table 7.

In some embodiments, the MUC5AC RNAi agents include core 19-mer nucleotide sequences shown in the following Table 2.

TABLE 2
MUC5AC RNAi Agent Antisense Strand and Sense Strand Core Stretch Base Sequences
(N = any nucleobase; I = inosine (hypoxanthine nucleobase)

| | | | | Corresponding | |
|---|---|---|---|---|---|
| | Antisense Strand Base | | Sense Strand Base | Positions of | |
| SEQ | Sequence (5′→3′) | SEQ | Sequence (5′→3′) | Identified | Targeted |
| ID | (Shown as an Unmodified | ID | (Shown as an Unmodified | Sequence on | Gene |
| NO:. | Nucleotide Sequence) | NO:. | Nucleotide Sequence) | SEQ ID NO: 1 | Position |
| 79 | UUGUAGUAGUCGCAGAACA | 568 | UGUUCUGCGACUACUACAA | 3535-3553 | 3535 |
| 80 | NUGUAGUAGUCGCAGAACA | 569 | UGUUCUGCGACUACUACAN | 3535-3553 | 3535 |
| 81 | UUGUAGUAGUCGCAGAACN | 570 | NGUUCUGCGACUACUACAA | 3535-3553 | 3535 |
| 82 | NUGUAGUAGUCGCAGAACN | 571 | NGUUCUGCGACUACUACAN | 3535-3553 | 3535 |
| 83 | UUCUUGUUCAGGCAAAUCA | 572 | UGAUUUGCCUGAACAAGAA | 4993-5011 | 4993 |
| 84 | NUCUUGUUCAGGCAAAUCA | 573 | UGAUUUGCCUGAACAAGAN | 4993-5011 | 4993 |
| 85 | UUCUUGUUCAGGCAAAUCN | 574 | NGAUUUGCCUGAACAAGAA | 4993-5011 | 4993 |
| 86 | NUCUUGUUCAGGCAAAUCN | 575 | NGAUUUGCCUGAACAAGAN | 4993-5011 | 4993 |
| 87 | CUUGAUGGCCUUGGAGCAG | 576 | CUGCUCCAAGGCCAUCAAG | 2924-2942 | 2924 |
| 88 | UUUGAUGGCCUUGGAGCAG | 577 | CUGCUCCAAGGCCAUCAAA | 2924-2942 | 2924 |
| 89 | NUUGAUGGCCUUGGAGCAG | 578 | CUGCUCCAAGGCCAUCAAN | 2924-2942 | 2924 |
| 90 | UUUGAUGGCCUUGGAGCAN | 579 | NUGCUCCAAGGCCAUCAAA | 2924-2942 | 2924 |
| 91 | NUUGAUGGCCUUGGAGCAN | 580 | NUGCUCCAAGGCCAUCAAN | 2924-2942 | 2924 |
| 92 | AAUCUUGAUGGCCUUGGAG | 581 | CUCCAAGGCCAUCAAGAUU | 2927-2945 | 2927 |
| 93 | AAUCUUGAUGGCCUUGGAN | 582 | NUCCAAGGCCAUCAAGAUU | 2927-2945 | 2927 |
| 94 | UAUCUUGAUGGCCUUGGAG | 583 | CUCCAAGGCCAUCAAGAUA | 2927-2945 | 2927 |
| 95 | UAUCUUGAUGGCCUUGGAN | 584 | NUCCAAGGCCAUCAAGAUA | 2927-2945 | 2927 |
| 96 | NAUCUUGAUGGCCUUGGAG | 585 | CUCCAAGGCCAUCAAGAUN | 2927-2945 | 2927 |
| 97 | NAUCUUGAUGGCCUUGGAN | 586 | NUCCAAGGCCAUCAAGAUN | 2927-2945 | 2927 |
| 98 | CUUGAACUCGGGGCUGAGG | 587 | CCUCAGCCCCGAGUUCAAG | 3116-3134 | 3116 |
| 99 | UUUGAACUCGGGGCUGAGG | 588 | CCUCAGCCCCGAGUUCAAA | 3116-3134 | 3116 |
| 100 | NUUGAACUCGGGGCUGAGG | 589 | CCUCAGCCCCGAGUUCAAN | 3116-3134 | 3116 |
| 101 | UUUGAACUCGGGGCUGAGN | 590 | NCUCAGCCCCGAGUUCAAA | 3116-3134 | 3116 |
| 102 | NUUGAACUCGGGGCUGAGN | 591 | NCUCAGCCCCGAGUUCAAN | 3116-3134 | 3116 |
| 103 | GGAUGCUGCACUGCUUCUG | 592 | CAGAAGCAGUGCAGCAUCC | 3321-3339 | 3321 |
| 104 | UGAUGCUGCACUGCUUCUG | 593 | CAGAAGCAGUGCAGCAUCA | 3321-3339 | 3321 |
| 105 | NGAUGCUGCACUGCUUCUG | 594 | CAGAAGCAGUGCAGCAUCN | 3321-3339 | 3321 |
| 106 | UGAUGCUGCACUGCUUCUN | 595 | NAGAAGCAGUGCAGCAUCA | 3321-3339 | 3321 |
| 107 | NGAUGCUGCACUGCUUCUN | 596 | NAGAAGCAGUGCAGCAUCN | 3321-3339 | 3321 |
| 108 | UGAUGCUGCACUGCUUCUG | 597 | CAGAAGCAGUGCAICAUCA | 3321-3339 | 3321 |
| 109 | UGAUGCUGCACUGCUUCUN | 598 | NAGAAGCAGUGCAICAUCA | 3321-3339 | 3321 |
| 110 | NGAUGCUGCACUGCUUCUG | 599 | CAGAAGCAGUGCAICAUCN | 3321-3339 | 3321 |
| 111 | NGAUGCUGCACUGCUUCUN | 600 | NAGAAGCAGUGCAICAUCN | 3321-3339 | 3321 |
| 112 | GUAGUCGCAGAACAGAGGG | 601 | CCCUCUGUUCUGCGACUAC | 3530-3548 | 3530 |
| 113 | UUAGUCGCAGAACAGAGGG | 602 | CCCUCUGUUCUGCGACUAA | 3530-3548 | 3530 |
| 114 | UUAGUCGCAGAACAGAGGN | 603 | NCCUCUGUUCUGCGACUAA | 3530-3548 | 3530 |
| 115 | NUAGUCGCAGAACAGAGGG | 604 | CCCUCUGUUCUGCGACUAN | 3530-3548 | 3530 |
| 116 | NUAGUCGCAGAACAGAGGN | 605 | NCCUCUGUUCUGCGACUAN | 3530-3548 | 3530 |
| 117 | UUAGUCGCAGAACAGAGGG | 606 | CCCUCUGUUCUGCIACUAA | 3530-3548 | 3530 |
| 118 | UUAGUCGCAGAACAGAGGN | 607 | NCCUCUGUUCUGCIACUAA | 3530-3548 | 3530 |
| 119 | NUAGUCGCAGAACAGAGGG | 608 | CCCUCUGUUCUGCIACUAN | 3530-3548 | 3530 |
| 120 | NUAGUCGCAGAACAGAGGN | 609 | NCCUCUGUUCUGCIACUAN | 3530-3548 | 3530 |
| 121 | AGUAGUCGCAGAACAGAGG | 610 | CCUCUGUUCUGCGACUACU | 3531-3549 | 3531 |
| 122 | AGUAGUCGCAGAACAGAGN | 611 | NCUCUGUUCUGCGACUACU | 3531-3549 | 3531 |
| 123 | UGUAGUCGCAGAACAGAGG | 612 | CCUCUGUUCUGCGACUACA | 3531-3549 | 3531 |
| 124 | UGUAGUCGCAGAACAGAGN | 613 | NCUCUGUUCUGCGACUACA | 3531-3549 | 3531 |
| 125 | NGUAGUCGCAGAACAGAGG | 614 | CCUCUGUUCUGCGACUACN | 3531-3549 | 3531 |
| 126 | NGUAGUCGCAGAACAGAGN | 615 | NCUCUGUUCUGCGACUACN | 3531-3549 | 3531 |
| 127 | AGUAGUCGCAGAACAGAGG | 616 | CCUCUGUUCUICGACUACU | 3531-3549 | 3531 |
| 128 | AGUAGUCGCAGAACAGAGN | 617 | NCUCUGUUCUICGACUACU | 3531-3549 | 3531 |
| 129 | UGUAGUCGCAGAACAGAGG | 618 | CCUCUGUUCUICGACUACA | 3531-3549 | 3531 |
| 130 | UGUAGUCGCAGAACAGAGN | 619 | NCUCUGUUCUICGACUACA | 3531-3549 | 3531 |
| 131 | NGUAGUCGCAGAACAGAGG | 620 | CCUCUGUUCUICGACUACN | 3531-3549 | 3531 |
| 132 | NGUAGUCGCAGAACAGAGN | 621 | NCUCUGUUCUICGACUACN | 3531-3549 | 3531 |
| 133 | GUAGUAGUCGCAGAACAGA | 622 | UCUGUUCUGCGACUACUAC | 3533-3551 | 3533 |
| 134 | UUAGUAGUCGCAGAACAGA | 623 | UCUGUUCUGCGACUACUAA | 3533-3551 | 3533 |
| 135 | NUAGUAGUCGCAGAACAGA | 624 | UCUGUUCUGCGACUACUAN | 3533-3551 | 3533 |
| 136 | NUAGUAGUCGCAGAACAGN | 625 | NCUGUUCUGCGACUACUAN | 3533-3551 | 3533 |
| 137 | UGUAGUAGUCGCAGAACAG | 626 | CUGUUCUGCGACUACUACA | 3534-3552 | 3534 |
| 138 | NGUAGUAGUCGCAGAACAG | 627 | CUGUUCUGCGACUACUACN | 3534-3552 | 3534 |
| 139 | NGUAGUAGUCGCAGAACAN | 628 | NUGUUCUGCGACUACUACN | 3534-3552 | 3534 |
| 140 | AUAGUUGUAGCAGAUGGGU | 629 | ACCCAUCUGCUACAACUAU | 5021-5039 | 5021 |
| 141 | AUAGUUGUAGCAGAUGGGN | 630 | NCCCAUCUGCUACAACUAU | 5021-5039 | 5021 |
| 142 | UUAGUUGUAGCAGAUGGGU | 631 | ACCCAUCUGCUACAACUAA | 5021-5039 | 5021 |
| 143 | UUAGUUGUAGCAGAUGGGN | 632 | NCCCAUCUGCUACAACUAA | 5021-5039 | 5021 |
| 144 | NUAGUUGUAGCAGAUGGGU | 633 | ACCCAUCUGCUACAACUAN | 5021-5039 | 5021 |
| 145 | NUAGUUGUAGCAGAUGGGN | 634 | NCCCAUCUGCUACAACUAN | 5021-5039 | 5021 |
| 146 | GUCCACGUCGAACCACUUU | 635 | AAAGUGGUUCGACGUGGAC | 5297-5315 | 5297 |
| 147 | UUCCACGUCGAACCACUUU | 636 | AAAGUGGUUCGACGUGGAA | 5297-5315 | 5297 |
| 148 | UUCCACGUCGAACCACUUN | 637 | NAAGUGGUUCGACGUGGAA | 5297-5315 | 5297 |
| 149 | NUCCACGUCGAACCACUUU | 638 | AAAGUGGUUCGACGUGGAN | 5297-5315 | 5297 |
| 150 | NUCCACGUCGAACCACUUN | 639 | NAAGUGGUUCGACGUGGAN | 5297-5315 | 5297 |
| 151 | UUCCACGUCGAACCACUUU | 640 | AAAGUGGUUCGACIUGGAA | 5297-5315 | 5297 |
| 152 | UUCCACGUCGAACCACUUN | 641 | NAAGUGGUUCGACIUGGAA | 5297-5315 | 5297 |
| 153 | NUCCACGUCGAACCACUUU | 642 | AAAGUGGUUCGACIUGGAN | 5297-5315 | 5297 |
| 154 | NUCCACGUCGAACCACUUN | 643 | NAAGUGGUUCGACIUGGAN | 5297-5315 | 5297 |
| 155 | GAAGUCCACGUCGAACCAC | 644 | GUGGUUCGACGUGGACUUC | 5300-5318 | 5300 |
| 156 | UAAGUCCACGUCGAACCAC | 645 | GUGGUUCGACGUGGACUUA | 5300-5318 | 5300 |
| 157 | UAAGUCCACGUCGAACCAN | 646 | NUGGUUCGACGUGGACUUA | 5300-5318 | 5300 |
| 158 | NAAGUCCACGUCGAACCAC | 647 | GUGGUUCGACGUGGACUUN | 5300-5318 | 5300 |
| 159 | NAAGUCCACGUCGAACCAN | 648 | NUGGUUCGACGUGGACUUN | 5300-5318 | 5300 |
| 160 | UAAGUCCACGUCGAACCAC | 649 | GUGGUUCGACGUGIACUUA | 5300-5318 | 5300 |
| 161 | UAAGUCCACGUCGAACCAN | 650 | NUGGUUCGACGUGIACUUA | 5300-5318 | 5300 |
| 162 | NAAGUCCACGUCGAACCAC | 651 | GUGGUUCGACGUGIACUUN | 5300-5318 | 5300 |
| 163 | NAAGUCCACGUCGAACCAN | 652 | NUGGUUCGACGUGIACUUN | 5300-5318 | 5300 |
| 164 | GGGAAGUCCACGUCGAACC | 653 | GGUUCGACGUGGACUUCCC | 5302-5320 | 5302 |
| 165 | UGGAAGUCCACGUCGAACC | 654 | GGUUCGACGUGGACUUCCA | 5302-5320 | 5302 |
| 166 | UGGAAGUCCACGUCGAACN | 655 | NGUUCGACGUGGACUUCCA | 5302-5320 | 5302 |
| 167 | NGGAAGUCCACGUCGAACC | 656 | GGUUCGACGUGGACUUCCN | 5302-5320 | 5302 |
| 168 | NGGAAGUCCACGUCGAACN | 657 | NGUUCGACGUGGACUUCCN | 5302-5320 | 5302 |
| 169 | UGGAAGUCCACGUCGAACC | 658 | GGUUCGACGUGIACUUCCA | 5302-5320 | 5302 |
| 170 | UGGAAGUCCACGUCGAACN | 659 | NGUUCGACGUGIACUUCCA | 5302-5320 | 5302 |
| 171 | NGGAAGUCCACGUCGAACC | 660 | GGUUCGACGUGIACUUCCN | 5302-5320 | 5302 |
| 172 | NGGAAGUCCACGUCGAACN | 661 | NGUUCGACGUGIACUUCCN | 5302-5320 | 5302 |
| 173 | AGAUGCUGGUCUUCUUGUC | 662 | GACAAGAAGACCAGCAUCU | 3090-3108 | 3090 |
| 174 | AGAUGCUGGUCUUCUUGUN | 663 | NACAAGAAGACCAGCAUCU | 3090-3108 | 3090 |
| 175 | UGAUGCUGGUCUUCUUGUC | 664 | GACAAGAAGACCAGCAUCA | 3090-3108 | 3090 |
| 176 | UGAUGCUGGUCUUCUUGUN | 665 | NACAAGAAGACCAGCAUCA | 3090-3108 | 3090 |
| 177 | NGAUGCUGGUCUUCUUGUC | 666 | GACAAGAAGACCAGCAUCN | 3090-3108 | 3090 |
| 178 | NGAUGCUGGUCUUCUUGUN | 667 | NACAAGAAGACCAGCAUCN | 3090-3108 | 3090 |
| 179 | AGAUGCUGGUCUUCUUGUC | 668 | GACAAGAAGACCAICAUCU | 3090-3108 | 3090 |
| 180 | AGAUGCUGGUCUUCUUGUN | 669 | NACAAGAAGACCAICAUCU | 3090-3108 | 3090 |
| 181 | UGAUGCUGGUCUUCUUGUC | 670 | GACAAGAAGACCAICAUCA | 3090-3108 | 3090 |
| 182 | UGAUGCUGGUCUUCUUGUN | 671 | NACAAGAAGACCAICAUCA | 3090-3108 | 3090 |
| 183 | NGAUGCUGGUCUUCUUGUC | 672 | GACAAGAAGACCAICAUCN | 3090-3108 | 3090 |
| 184 | NGAUGCUGGUCUUCUUGUN | 673 | NACAAGAAGACCAICAUCN | 3090-3108 | 3090 |
| 185 | GGUUGAUGAAGAUGCUGGU | 674 | ACCAGCAUCUUCAUCAACC | 3099-3117 | 3099 |
| 186 | UGUUGAUGAAGAUGCUGGN | 675 | NCCAGCAUCUUCAUCAACC | 3099-3117 | 3099 |
| 187 | NGUUGAUGAAGAUGCUGGU | 676 | ACCAGCAUCUUCAUCAACN | 3099-3117 | 3099 |
| 188 | NGUUGAUGAAGAUGCUGGN | 677 | NCCAGCAUCUUCAUCAACN | 3099-3117 | 3099 |
| 189 | AUGUUGUUGUAGGUUUCCU | 678 | AGGAAACCUACAACAACAU | 5347-5365 | 5347 |
| 190 | AUGUUGUUGUAGGUUUCCN | 679 | NGGAAACCUACAACAACAU | 5347-5365 | 5347 |
| 191 | UUGUUGUUGUAGGUUUCCU | 680 | AGGAAACCUACAACAACAA | 5347-5365 | 5347 |
| 192 | UUGUUGUUGUAGGUUUCCN | 681 | NGGAAACCUACAACAACAA | 5347-5365 | 5347 |
| 193 | NUGUUGUUGUAGGUUUCCN | 682 | NGGAAACCUACAACAACAN | 5347-5365 | 5347 |
| 194 | AUGAUGUUGUUGUAGGUUU | 683 | AAACCUACAACAACAUCAU | 5350-5368 | 5350 |
| 195 | AUGAUGUUGUUGUAGGUUN | 684 | NAACCUACAACAACAUCAU | 5350-5368 | 5350 |
| 196 | UUGAUGUUGUUGUAGGUUU | 685 | AAACCUACAACAACAUCAA | 5350-5368 | 5350 |
| 197 | UUGAUGUUGUUGUAGGUUN | 686 | NAACCUACAACAACAUCAA | 5350-5368 | 5350 |
| 198 | NUGAUGUUGUUGUAGGUUN | 687 | NAACCUACAACAACAUCAN | 5350-5368 | 5350 |
| 199 | UUGAUGAAGAUGCUGGUCU | 688 | AGACCAGCAUCUUCAUCAA | 3097-3115 | 3097 |
| 200 | UUGAUGAAGAUGCUGGUCN | 689 | NGACCAGCAUCUUCAUCAA | 3097-3115 | 3097 |
| 201 | NUGAUGAAGAUGCUGGUCU | 690 | AGACCAGCAUCUUCAUCAN | 3097-3115 | 3097 |
| 202 | NUGAUGAAGAUGCUGGUCN | 691 | NGACCAGCAUCUUCAUCAN | 3097-3115 | 3097 |
| 203 | UGAUCUGGUAGUUGUAGCA | 692 | UGCUACAACUACCAGAUCA | 4443-4461 | 4443 |
| 204 | UGAUCUGGUAGUUGUAGCN | 693 | NGCUACAACUACCAGAUCA | 4443-4461 | 4443 |
| 205 | NGAUCUGGUAGUUGUAGCA | 694 | UGCUACAACUACCAGAUCN | 4443-4461 | 4443 |
| 206 | NGAUCUGGUAGUUGUAGCN | 695 | NGCUACAACUACCAGAUCN | 4443-4461 | 4443 |
| 207 | UGAUCUGGUAGUUGUAGCN | 696 | NGCUACAACUACCAIAUCA | 4443-4461 | 4443 |
| 208 | NGAUCUGGUAGUUGUAGCA | 697 | UGCUACAACUACCAIAUCN | 4443-4461 | 4443 |
| 209 | NGAUCUGGUAGUUGUAGCN | 698 | NGCUACAACUACCAIAUCN | 4443-4461 | 4443 |
| 210 | UGAUCUGGUAGUUGUAGCN | 699 | NGCUACAACUACCAIAUCA | 4443-4461 | 4443 |
| 211 | CCCUGAUCUGGUAGUUGUA | 700 | UACAACUACCAGAUCAGGG | 4446-4464 | 4446 |
| 212 | UCCUGAUCUGGUAGUUGUA | 701 | UACAACUACCAGAUCAGGA | 4446-4464 | 4446 |
| 213 | NCCUGAUCUGGUAGUUGUA | 702 | UACAACUACCAGAUCAGGN | 4446-4464 | 4446 |
| 214 | UCCUGAUCUGGUAGUUGUN | 703 | NACAACUACCAGAUCAGGA | 4446-4464 | 4446 |
| 215 | NCCUGAUCUGGUAGUUGUN | 704 | NACAACUACCAGAUCAGGN | 4446-4464 | 4446 |
| 216 | CCCUGAUCUGGUAGUUGUA | 705 | UACAACUACCAGAUCAIGG | 4446-4464 | 4446 |
| 217 | UCCUGAUCUGGUAGUUGUA | 706 | UACAACUACCAGAUCAIGA | 4446-4464 | 4446 |
| 218 | NCCUGAUCUGGUAGUUGUA | 707 | UACAACUACCAGAUCAIGN | 4446-4464 | 4446 |
| 219 | UCCUGAUCUGGUAGUUGUN | 708 | NACAACUACCAGAUCAIGA | 4446-4464 | 4446 |
| 220 | NCCUGAUCUGGUAGUUGUN | 709 | NACAACUACCAGAUCAIGN | 4446-4464 | 4446 |
| 221 | UAGUUGUAGCAGAUGGGUG | 710 | CACCCAUCUGCUACAACUA | 5020-5038 | 5020 |
| 222 | NAGUUGUAGCAGAUGGGUG | 711 | CACCCAUCUGCUACAACUN | 5020-5038 | 5020 |
| 223 | UAGUUGUAGCAGAUGGGUN | 712 | NACCCAUCUGCUACAACUA | 5020-5038 | 5020 |
| 224 | NAGUUGUAGCAGAUGGGUN | 713 | NACCCAUCUGCUACAACUN | 5020-5038 | 5020 |
| 225 | GCAACACUGGAUGCGGAUC | 714 | GAUCCGCAUCCAGUGUUGC | 5042-5060 | 5042 |
| 226 | UCAACACUGGAUGCGGAUC | 715 | GAUCCGCAUCCAGUGUUGA | 5042-5060 | 5042 |
| 227 | NCAACACUGGAUGCGGAUC | 716 | GAUCCGCAUCCAGUGUUGA | 5042-5060 | 5042 |
| 228 | UCAACACUGGAUGCGGAUN | 717 | NAUCCGCAUCCAGUGUUGN | 5042-5060 | 5042 |
| 229 | NCAACACUGGAUGCGGAUN | 718 | NAUCCGCAUCCAGUGUUGN | 5042-5060 | 5042 |
| 230 | GCAACACUGGAUGCGGAUC | 719 | GAUCCGCAUCCAGUIUUGC | 5042-5060 | 5042 |
| 231 | UCAACACUGGAUGCGGAUC | 720 | GAUCCGCAUCCAGUIUUGA | 5042-5060 | 5042 |
| 232 | NCAACACUGGAUGCGGAUC | 721 | GAUCCGCAUCCAGUIUUGA | 5042-5060 | 5042 |
| 233 | UCAACACUGGAUGCGGAUN | 722 | NAUCCGCAUCCAGUIUUGN | 5042-5060 | 5042 |
| 234 | NCAACACUGGAUGCGGAUN | 723 | NAUCCGCAUCCAGUIUUGN | 5042-5060 | 5042 |
| 235 | GUGUUCGAUGCUCACCUCU | 724 | AGAGGUGAGCAUCGAACAC | 5441-5459 | 5441 |
| 236 | UUGUUCGAUGCUCACCUCU | 725 | AGAGGUGAGCAUCGAACAA | 5441-5459 | 5441 |
| 237 | NUGUUCGAUGCUCACCUCU | 726 | AGAGGUGAGCAUCGAACAN | 5441-5459 | 5441 |
| 238 | UUGUUCGAUGCUCACCUCN | 727 | NGAGGUGAGCAUCGAACAA | 5441-5459 | 5441 |
| 239 | NUGUUCGAUGCUCACCUCN | 728 | NGAGGUGAGCAUCGAACAN | 5441-5459 | 5441 |
| 240 | GUGUUCGAUGCUCACCUCU | 729 | AGAGGUGAGCAUCIAACAC | 5441-5459 | 5441 |
| 241 | UUGUUCGAUGCUCACCUCU | 730 | AGAGGUGAGCAUCIAACAA | 5441-5459 | 5441 |
| 242 | NUGUUCGAUGCUCACCUCU | 731 | AGAGGUGAGCAUCIAACAN | 5441-5459 | 5441 |
| 243 | UUGUUCGAUGCUCACCUCN | 732 | NGAGGUGAGCAUCIAACAA | 5441-5459 | 5441 |
| 244 | NUGUUCGAUGCUCACCUCN | 733 | NGAGGUGAGCAUCIAACAN | 5441-5459 | 5441 |
| 245 | CAUCUUGAAGGGUCCCUGC | 734 | GCAGGGACCCUUCAAGAUG | 5519-5537 | 5519 |
| 246 | UAUCUUGAAGGGUCCCUGC | 735 | GCAGGGACCCUUCAAGAUA | 5519-5537 | 5519 |
| 247 | NAUCUUGAAGGGUCCCUGC | 736 | GCAGGGACCCUUCAAGAUN | 5519-5537 | 5519 |
| 248 | UAUCUUGAAGGGUCCCUGN | 737 | NCAGGGACCCUUCAAGAUA | 5519-5537 | 5519 |
| 249 | NAUCUUGAAGGGUCCCUGN | 738 | NCAGGGACCCUUCAAGAUN | 5519-5537 | 5519 |
| 250 | UCGUAGUUGAGGCACAUCU | 739 | AGAUGUGCCUCAACUACGA | 5533-5551 | 5533 |
| 251 | NCGUAGUUGAGGCACAUCU | 740 | AGAUGUGCCUCAACUACGN | 5533-5551 | 5533 |
| 252 | UCGUAGUUGAGGCACAUCN | 741 | NGAUGUGCCUCAACUACGA | 5533-5551 | 5533 |
| 253 | NCGUAGUUGAGGCACAUCN | 742 | NGAUGUGCCUCAACUACGN | 5533-5551 | 5533 |
| 254 | UCGUAGUUGAGGCACAUCU | 743 | AGAUGUGCCUCAACUACIA | 5533-5551 | 5533 |
| 255 | NCGUAGUUGAGGCACAUCU | 744 | AGAUGUGCCUCAACUACIN | 5533-5551 | 5533 |
| 256 | UCGUAGUUGAGGCACAUCN | 745 | NGAUGUGCCUCAACUACIA | 5533-5551 | 5533 |
| 257 | NCGUAGUUGAGGCACAUCN | 746 | NGAUGUGCCUCAACUACIN | 5533-5551 | 5533 |
| 258 | CCUCGUAGUUGAGGCACAU | 747 | AUGUGCCUCAACUACGAGG | 5535-5553 | 5535 |
| 259 | UCUCGUAGUUGAGGCACAU | 748 | AUGUGCCUCAACUACGAGA | 5535-5553 | 5535 |
| 260 | NCUCGUAGUUGAGGCACAU | 749 | AUGUGCCUCAACUACGAGN | 5535-5553 | 5535 |
| 261 | UCUCGUAGUUGAGGCACAN | 750 | NUGUGCCUCAACUACGAGA | 5535-5553 | 5535 |
| 262 | NCUCGUAGUUGAGGCACAN | 751 | NUGUGCCUCAACUACGAGN | 5535-5553 | 5535 |
| 263 | CCUCGUAGUUGAGGCACAU | 752 | AUGUGCCUCAACUACIAGG | 5535-5553 | 5535 |
| 264 | UCUCGUAGUUGAGGCACAU | 753 | AUGUGCCUCAACUACIAGA | 5535-5553 | 5535 |
| 265 | NCUCGUAGUUGAGGCACAU | 754 | AUGUGCCUCAACUACIAGN | 5535-5553 | 5535 |
| 266 | UCUCGUAGUUGAGGCACAN | 755 | NUGUGCCUCAACUACIAGA | 5535-5553 | 5535 |
| 267 | NCUCGUAGUUGAGGCACAN | 756 | NUGUGCCUCAACUACIAGN | 5535-5553 | 5535 |
| 268 | CUUCAGGCAGGUCUCGCUG | 757 | CAGCGAGACCUGCCUGAAG | 1493-1511 | 1493 |
| 269 | UUUCAGGCAGGUCUCGCUG | 758 | CAGCGAGACCUGCCUGAAA | 1493-1511 | 1493 |
| 270 | NUUCAGGCAGGUCUCGCUG | 759 | CAGCGAGACCUGCCUGAAN | 1493-1511 | 1493 |
| 271 | UUUCAGGCAGGUCUCGCUN | 760 | NAGCGAGACCUGCCUGAAA | 1493-1511 | 1493 |
| 272 | NUUCAGGCAGGUCUCGCUN | 761 | NAGCGAGACCUGCCUGAAN | 1493-1511 | 1493 |
| 273 | GUCUGAAGAUGGUGACGUU | 762 | AACGUCACCAUCUUCAGAC | 1617-1635 | 1617 |
| 274 | UUCUGAAGAUGGUGACGUU | 763 | AACGUCACCAUCUUCAGAA | 1617-1635 | 1617 |
| 275 | NUCUGAAGAUGGUGACGUU | 764 | AACGUCACCAUCUUCAGAN | 1617-1635 | 1617 |
| 276 | UUCUGAAGAUGGUGACGUN | 765 | NACGUCACCAUCUUCAGAA | 1617-1635 | 1617 |
| 277 | NUCUGAAGAUGGUGACGUN | 766 | NACGUCACCAUCUUCAGAN | 1617-1635 | 1617 |
| 278 | GGUCUGAAGAUGGUGACGU | 767 | ACGUCACCAUCUUCAGACC | 1618-1636 | 1618 |
| 279 | UGUCUGAAGAUGGUGACGU | 768 | ACGUCACCAUCUUCAGACA | 1618-1636 | 1618 |
| 280 | NGUCUGAAGAUGGUGACGU | 769 | ACGUCACCAUCUUCAGACN | 1618-1636 | 1618 |
| 281 | UGUCUGAAGAUGGUGACGN | 770 | NCGUCACCAUCUUCAGACA | 1618-1636 | 1618 |
| 282 | NGUCUGAAGAUGGUGACGN | 771 | NCGUCACCAUCUUCAGACN | 1618-1636 | 1618 |
| 283 | GGUCUGAAGAUGGUGACGU | 772 | ACGUCACCAUCUUCAIACC | 1618-1636 | 1618 |
| 284 | UGUCUGAAGAUGGUGACGU | 773 | ACGUCACCAUCUUCAIACA | 1618-1636 | 1618 |
| 285 | NGUCUGAAGAUGGUGACGU | 774 | ACGUCACCAUCUUCAIACN | 1618-1636 | 1618 |
| 286 | UGUCUGAAGAUGGUGACGN | 775 | NCGUCACCAUCUUCAIACA | 1618-1636 | 1618 |
| 287 | NGUCUGAAGAUGGUGACGN | 776 | NCGUCACCAUCUUCAIACN | 1618-1636 | 1618 |
| 288 | CGGAAGUCAUCGGCCUGGA | 777 | UCCAGGCCGAUGACUUCCG | 1777-1795 | 1777 |
| 289 | UGGAAGUCAUCGGCCUGGA | 778 | UCCAGGCCGAUGACUUCCA | 1777-1795 | 1777 |
| 290 | NGGAAGUCAUCGGCCUGGA | 779 | UCCAGGCCGAUGACUUCCN | 1777-1795 | 1777 |
| 291 | UGGAAGUCAUCGGCCUGGN | 780 | NCCAGGCCGAUGACUUCCA | 1777-1795 | 1777 |
| 292 | NGGAAGUCAUCGGCCUGGN | 781 | NCCAGGCCGAUGACUUCCN | 1777-1795 | 1777 |
| 293 | CUUGAAGGUGUUGAAGAAG | 782 | CUUCUUCAACACCUUCAAG | 1832-1850 | 1832 |
| 294 | UUUGAAGGUGUUGAAGAAG | 783 | CUUCUUCAACACCUUCAAA | 1832-1850 | 1832 |
| 295 | NUUGAAGGUGUUGAAGAAG | 784 | CUUCUUCAACACCUUCAAN | 1832-1850 | 1832 |
| 296 | UUUGAAGGUGUUGAAGAAN | 785 | NUUCUUCAACACCUUCAAA | 1832-1850 | 1832 |
| 297 | NUUGAAGGUGUUGAAGAAN | 786 | NUUCUUCAACACCUUCAAN | 1832-1850 | 1832 |
| 298 | UGCAGUUCGAGUAGUAGGU | 787 | ACCUACUACUCGAACUGCA | 2001-2019 | 2001 |
| 299 | NGCAGUUCGAGUAGUAGGU | 788 | ACCUACUACUCGAACUGCN | 2001-2019 | 2001 |
| 300 | UGCAGUUCGAGUAGUAGGN | 789 | NCCUACUACUCGAACUGCA | 2001-2019 | 2001 |
| 301 | NGCAGUUCGAGUAGUAGGN | 790 | NCCUACUACUCGAACUGCN | 2001-2019 | 2001 |
| 302 | UGCAGUUCGAGUAGUAGGU | 791 | ACCUACUACUCGAACUICA | 2001-2019 | 2001 |
| 303 | NGCAGUUCGAGUAGUAGGU | 792 | ACCUACUACUCGAACUICN | 2001-2019 | 2001 |
| 304 | UGCAGUUCGAGUAGUAGGN | 793 | NCCUACUACUCGAACUICA | 2001-2019 | 2001 |
| 305 | NGCAGUUCGAGUAGUAGGN | 794 | NCCUACUACUCGAACUICN | 2001-2019 | 2001 |
| 306 | CUUGGAGCAGGUGGUCCCU | 795 | AGGGACCACCUGCUCCAAG | 2915-2933 | 2915 |
| 307 | UUUGGAGCAGGUGGUCCCU | 796 | AGGGACCACCUGCUCCAAA | 2915-2933 | 2915 |
| 308 | NUUGGAGCAGGUGGUCCCU | 797 | AGGGACCACCUGCUCCAAN | 2915-2933 | 2915 |
| 309 | UUUGGAGCAGGUGGUCCCN | 798 | NGGGACCACCUGCUCCAAA | 2915-2933 | 2915 |
| 310 | NUUGGAGCAGGUGGUCCCN | 799 | NGGGACCACCUGCUCCAAN | 2915-2933 | 2915 |
| 311 | UCUUGAUGGCCUUGGAGCA | 800 | UGCUCCAAGGCCAUCAAGA | 2925-2943 | 2925 |
| 312 | NCUUGAUGGCCUUGGAGCA | 801 | UGCUCCAAGGCCAUCAAGN | 2925-2943 | 2925 |
| 313 | UCUUGAUGGCCUUGGAGCN | 802 | NGCUCCAAGGCCAUCAAGA | 2925-2943 | 2925 |
| 314 | NCUUGAUGGCCUUGGAGCN | 803 | NGCUCCAAGGCCAUCAAGN | 2925-2943 | 2925 |
| 315 | CUGUCAUCGUGGUUCCACA | 804 | UGUGGAACCACGAUGACAG | 610-628 | 610 |
| 316 | UUGUCAUCGUGGUUCCACA | 805 | UGUGGAACCACGAUGACAA | 610-628 | 610 |
| 317 | NUGUCAUCGUGGUUCCACA | 806 | UGUGGAACCACGAUGACAN | 610-628 | 610 |
| 318 | UUGUCAUCGUGGUUCCACN | 807 | NGUGGAACCACGAUGACAA | 610-628 | 610 |
| 319 | NUGUCAUCGUGGUUCCACN | 808 | NGUGGAACCACGAUGACAN | 610-628 | 610 |
| 320 | CUGUCAUCGUGGUUCCACA | 809 | UGUGGAACCACGAUIACAG | 610-628 | 610 |
| 321 | UUGUCAUCGUGGUUCCACA | 810 | UGUGGAACCACGAUIACAA | 610-628 | 610 |
| 322 | NUGUCAUCGUGGUUCCACA | 811 | UGUGGAACCACGAUIACAN | 610-628 | 610 |
| 323 | UUGUCAUCGUGGUUCCACN | 812 | NGUGGAACCACGAUIACAA | 610-628 | 610 |
| 324 | NUGUCAUCGUGGUUCCACN | 813 | NGUGGAACCACGAUIACAN | 610-628 | 610 |
| 325 | ACAGAAGCAGAGGUCUUGC | 814 | GCAAGACCUCUGCUUCUGU | 923-941 | 923 |
| 326 | ACAGAAGCAGAGGUCUUGN | 815 | NCAAGACCUCUGCUUCUGU | 923-941 | 923 |
| 327 | UCAGAAGCAGAGGUCUUGC | 816 | GCAAGACCUCUGCUUCUGA | 923-941 | 923 |
| 328 | UCAGAAGCAGAGGUCUUGN | 817 | NCAAGACCUCUGCUUCUGA | 923-941 | 923 |
| 329 | ACAGAAGCAGAGGUCUUGC | 818 | GCAAGACCUCUGCUUCUIU | 923-941 | 923 |
| 330 | ACAGAAGCAGAGGUCUUGN | 819 | NCAAGACCUCUGCUUCUIU | 923-941 | 923 |
| 331 | UCAGAAGCAGAGGUCUUGC | 820 | GCAAGACCUCUGCUUCUIA | 923-941 | 923 |
| 332 | UCAGAAGCAGAGGUCUUGN | 821 | NCAAGACCUCUGCUUCUIA | 923-941 | 923 |
| 333 | GCAGUUGGUGCAGUCUGUG | 822 | CACAGACUGCACCAACUGC | 1277-1295 | 1277 |
| 334 | UCAGUUGGUGCAGUCUGUG | 823 | CACAGACUGCACCAACUGA | 1277-1295 | 1277 |
| 335 | NCAGUUGGUGCAGUCUGUG | 824 | CACAGACUGCACCAACUGA | 1277-1295 | 1277 |
| 336 | UCAGUUGGUGCAGUCUGUN | 825 | NACAGACUGCACCAACUGN | 1277-1295 | 1277 |
| 337 | NCAGUUGGUGCAGUCUGUN | 826 | NACAGACUGCACCAACUGN | 1277-1295 | 1277 |
| 338 | GCAGUUGGUGCAGUCUGUG | 827 | CACAGACUGCACCAACUIC | 1277-1295 | 1277 |
| 339 | UCAGUUGGUGCAGUCUGUG | 828 | CACAGACUGCACCAACUIA | 1277-1295 | 1277 |
| 340 | NCAGUUGGUGCAGUCUGUG | 829 | CACAGACUGCACCAACUIA | 1277-1295 | 1277 |
| 341 | UCAGUUGGUGCAGUCUGUN | 830 | NACAGACUGCACCAACUIN | 1277-1295 | 1277 |
| 342 | NCAGUUGGUGCAGUCUGUN | 831 | NACAGACUGCACCAACUIN | 1277-1295 | 1277 |
| 343 | GCAGUACAGUGAAGGCACU | 832 | AGUGCCUUCACUGUACUGC | 1446-1464 | 1446 |
| 344 | UCAGUACAGUGAAGGCACU | 833 | AGUGCCUUCACUGUACUGA | 1446-1464 | 1446 |
| 345 | NCAGUACAGUGAAGGCACU | 834 | AGUGCCUUCACUGUACUGN | 1446-1464 | 1446 |
| 346 | UCAGUACAGUGAAGGCACN | 835 | NGUGCCUUCACUGUACUGA | 1446-1464 | 1446 |
| 347 | NCAGUACAGUGAAGGCACN | 836 | NGUGCCUUCACUGUACUGN | 1446-1464 | 1446 |
| 348 | GCAGUACAGUGAAGGCACU | 837 | AGUGCCUUCACUGUACUIC | 1446-1464 | 1446 |
| 349 | UCAGUACAGUGAAGGCACU | 838 | AGUGCCUUCACUGUACUIA | 1446-1464 | 1446 |
| 350 | NCAGUACAGUGAAGGCACU | 839 | AGUGCCUUCACUGUACUIN | 1446-1464 | 1446 |
| 351 | UCAGUACAGUGAAGGCACN | 840 | NGUGCCUUCACUGUACUIA | 1446-1464 | 1446 |
| 352 | NCAGUACAGUGAAGGCACN | 841 | NGUGCCUUCACUGUACUIN | 1446-1464 | 1446 |
| 353 | UGCUGUUGAAGUUCCCACA | 842 | UGUGGGAACUUCAACAGCA | 1758-1776 | 1758 |
| 354 | NGCUGUUGAAGUUCCCACA | 843 | UGUGGGAACUUCAACAGCN | 1758-1776 | 1758 |
| 355 | UGCUGUUGAAGUUCCCACN | 844 | NGUGGGAACUUCAACAGCA | 1758-1776 | 1758 |
| 356 | NGCUGUUGAAGUUCCCACN | 845 | NGUGGGAACUUCAACAGCN | 1758-1776 | 1758 |
| 357 | UGCUGUUGAAGUUCCCACA | 846 | UGUGGGAACUUCAACAICA | 1758-1776 | 1758 |
| 358 | NGCUGUUGAAGUUCCCACA | 847 | UGUGGGAACUUCAACAICN | 1758-1776 | 1758 |
| 359 | UGCUGUUGAAGUUCCCACN | 848 | NGUGGGAACUUCAACAICA | 1758-1776 | 1758 |
| 360 | NGCUGUUGAAGUUCCCACN | 849 | NGUGGGAACUUCAACAICN | 1758-1776 | 1758 |
| 361 | GGAUGCUGUUGAAGUUCCC | 850 | GGGAACUUCAACAGCAUCC | 1761-1779 | 1761 |
| 362 | UGAUGCUGUUGAAGUUCCC | 851 | GGGAACUUCAACAGCAUCA | 1761-1779 | 1761 |
| 363 | NGAUGCUGUUGAAGUUCCC | 852 | GGGAACUUCAACAGCAUCN | 1761-1779 | 1761 |
| 364 | UGAUGCUGUUGAAGUUCCN | 853 | NGGAACUUCAACAGCAUCA | 1761-1779 | 1761 |
| 365 | NGAUGCUGUUGAAGUUCCN | 854 | NGGAACUUCAACAGCAUCN | 1761-1779 | 1761 |
| 366 | GGAUGCUGUUGAAGUUCCC | 855 | GGGAACUUCAACAICAUCC | 1761-1779 | 1761 |
| 367 | UGAUGCUGUUGAAGUUCCC | 856 | GGGAACUUCAACAICAUCA | 1761-1779 | 1761 |
| 368 | NGAUGCUGUUGAAGUUCCC | 857 | GGGAACUUCAACAICAUCN | 1761-1779 | 1761 |
| 369 | UGAUGCUGUUGAAGUUCCN | 858 | NGGAACUUCAACAICAUCA | 1761-1779 | 1761 |
| 370 | NGAUGCUGUUGAAGUUCCN | 859 | NGGAACUUCAACAICAUCN | 1761-1779 | 1761 |
| 371 | GGGUCUUGAAGGUGUUGAA | 860 | UUCAACACCUUCAAGACCC | 1836-1854 | 1836 |
| 372 | UGGUCUUGAAGGUGUUGAA | 861 | UUCAACACCUUCAAGACCA | 1836-1854 | 1836 |
| 373 | NGGUCUUGAAGGUGUUGAA | 862 | UUCAACACCUUCAAGACCN | 1836-1854 | 1836 |
| 374 | UGGUCUUGAAGGUGUUGAN | 863 | NUCAACACCUUCAAGACCA | 1836-1854 | 1836 |
| 375 | NGGUCUUGAAGGUGUUGAN | 864 | NUCAACACCUUCAAGACCN | 1836-1854 | 1836 |
| 376 | GGGUCUUGAAGGUGUUGAA | 865 | UUCAACACCUUCAAIACCC | 1836-1854 | 1836 |
| 377 | UGGUCUUGAAGGUGUUGAA | 866 | UUCAACACCUUCAAIACCA | 1836-1854 | 1836 |
| 378 | NGGUCUUGAAGGUGUUGAA | 867 | UUCAACACCUUCAAIACCN | 1836-1854 | 1836 |
| 379 | UGGUCUUGAAGGUGUUGAN | 868 | NUCAACACCUUCAAIACCA | 1836-1854 | 1836 |
| 380 | NGGUCUUGAAGGUGUUGAN | 869 | NUCAACACCUUCAAIACCN | 1836-1854 | 1836 |
| 381 | AAGCUGUUCCUGAUGUUGG | 870 | CCAACAUCAGGAACAGCUU | 1867-1885 | 1867 |
| 382 | AAGCUGUUCCUGAUGUUGN | 871 | NCAACAUCAGGAACAGCUU | 1867-1885 | 1867 |
| 383 | UAGCUGUUCCUGAUGUUGG | 872 | CCAACAUCAGGAACAGCUN | 1867-1885 | 1867 |
| 384 | UAGCUGUUCCUGAUGUUGN | 873 | NCAACAUCAGGAACAGCUU | 1867-1885 | 1867 |
| 385 | NAGCUGUUCCUGAUGUUGN | 874 | NCAACAUCAGGAACAGCUN | 1867-1885 | 1867 |
| 386 | AAGCUGUUCCUGAUGUUGG | 875 | CCAACAUCAGGAACAICUU | 1867-1885 | 1867 |
| 387 | AAGCUGUUCCUGAUGUUGN | 876 | NCAACAUCAGGAACAICUU | 1867-1885 | 1867 |
| 388 | UAGCUGUUCCUGAUGUUGG | 877 | CCAACAUCAGGAACAICUN | 1867-1885 | 1867 |
| 389 | UAGCUGUUCCUGAUGUUGN | 878 | NCAACAUCAGGAACAICUU | 1867-1885 | 1867 |
| 390 | NAGCUGUUCCUGAUGUUGN | 879 | NCAACAUCAGGAACAICUN | 1867-1885 | 1867 |
| 391 | ACAUGCAGUUCGAGUAGUA | 880 | UACUACUCGAACUGCAUGU | 2004-2022 | 2004 |
| 392 | ACAUGCAGUUCGAGUAGUN | 881 | NACUACUCGAACUGCAUGU | 2004-2022 | 2004 |
| 393 | UCAUGCAGUUCGAGUAGUA | 882 | UACUACUCGAACUGCAUGA | 2004-2022 | 2004 |
| 394 | UCAUGCAGUUCGAGUAGUN | 883 | NACUACUCGAACUGCAUGA | 2004-2022 | 2004 |
| 395 | NCAUGCAGUUCGAGUAGUN | 884 | NACUACUCGAACUGCAUGN | 2004-2022 | 2004 |
| 396 | GAAGCCAACACUGCAGGUG | 885 | CACCUGCAGUGUUGGCUUC | 2234-2252 | 2234 |
| 397 | UAAGCCAACACUGCAGGUG | 886 | CACCUGCAGUGUUGGCUUA | 2234-2252 | 2234 |
| 398 | NAAGCCAACACUGCAGGUG | 887 | CACCUGCAGUGUUGGCUUN | 2234-2252 | 2234 |
| 399 | UAAGCCAACACUGCAGGUN | 888 | NACCUGCAGUGUUGGCUUA | 2234-2252 | 2234 |
| 400 | NAAGCCAACACUGCAGGUN | 889 | NACCUGCAGUGUUGGCUUN | 2234-2252 | 2234 |
| 401 | GAAGCCAACACUGCAGGUG | 890 | CACCUGCAGUGUUGICUUC | 2234-2252 | 2234 |
| 402 | UAAGCCAACACUGCAGGUG | 891 | CACCUGCAGUGUUGICUUA | 2234-2252 | 2234 |
| 403 | NAAGCCAACACUGCAGGUG | 892 | CACCUGCAGUGUUGICUUN | 2234-2252 | 2234 |
| 404 | UAAGCCAACACUGCAGGUN | 893 | NACCUGCAGUGUUGICUUA | 2234-2252 | 2234 |
| 405 | NAAGCCAACACUGCAGGUN | 894 | NACCUGCAGUGUUGICUUN | 2234-2252 | 2234 |
| 406 | CUGUAACAGGUCAUGUCCA | 895 | UGGACAUGACCUGUUACAG | 2536-2554 | 2536 |
| 407 | UUGUAACAGGUCAUGUCCA | 896 | UGGACAUGACCUGUUACAA | 2536-2554 | 2536 |
| 408 | NUGUAACAGGUCAUGUCCA | 897 | UGGACAUGACCUGUUACAN | 2536-2554 | 2536 |
| 409 | UUGUAACAGGUCAUGUCCN | 898 | NGGACAUGACCUGUUACAA | 2536-2554 | 2536 |
| 410 | NUGUAACAGGUCAUGUCCN | 899 | NGGACAUGACCUGUUACAN | 2536-2554 | 2536 |
| 411 | CCGUUGAAGCUGUAGCUCU | 900 | AGAGCUACAGCUUCAACGG | 2797-2815 | 2797 |
| 412 | UCGUUGAAGCUGUAGCUCU | 901 | AGAGCUACAGCUUCAACGA | 2797-2815 | 2797 |
| 413 | NCGUUGAAGCUGUAGCUCU | 902 | AGAGCUACAGCUUCAACGN | 2797-2815 | 2797 |
| 414 | UCGUUGAAGCUGUAGCUCN | 903 | NGAGCUACAGCUUCAACGA | 2797-2815 | 2797 |
| 415 | NCGUUGAAGCUGUAGCUCN | 904 | NGAGCUACAGCUUCAACGN | 2797-2815 | 2797 |
| 416 | CCGUUGAAGCUGUAGCUCU | 905 | AGAGCUACAGCUUCAACIG | 2797-2815 | 2797 |
| 417 | UCGUUGAAGCUGUAGCUCU | 906 | AGAGCUACAGCUUCAACIA | 2797-2815 | 2797 |
| 418 | NCGUUGAAGCUGUAGCUCU | 907 | AGAGCUACAGCUUCAACIN | 2797-2815 | 2797 |
| 419 | UCGUUGAAGCUGUAGCUCN | 908 | NGAGCUACAGCUUCAACIA | 2797-2815 | 2797 |
| 420 | NCGUUGAAGCUGUAGCUCN | 909 | NGAGCUACAGCUUCAACIN | 2797-2815 | 2797 |
| 421 | CAGUACAGUGAAGGCACUG | 910 | CAGUGCCUUCACUGUACUG | 1445-1463 | 1445 |
| 422 | UAGUACAGUGAAGGCACUG | 911 | CAGUGCCUUCACUGUACUA | 1445-1463 | 1445 |
| 423 | UAGUACAGUGAAGGCACUN | 912 | NAGUGCCUUCACUGUACUA | 1445-1463 | 1445 |
| 424 | NAGUACAGUGAAGGCACUG | 913 | CAGUGCCUUCACUGUACUN | 1445-1463 | 1445 |
| 425 | NAGUACAGUGAAGGCACUN | 914 | NAGUGCCUUCACUGUACUN | 1445-1463 | 1445 |
| 426 | CUCGAAGCUGUUCCUGAUG | 915 | CAUCAGGAACAGCUUCGAG | 1871-1889 | 1871 |
| 427 | UUCGAAGCUGUUCCUGAUG | 916 | CAUCAGGAACAGCUUCGAA | 1871-1889 | 1871 |
| 428 | UUCGAAGCUGUUCCUGAUN | 917 | NAUCAGGAACAGCUUCGAA | 1871-1889 | 1871 |
| 429 | NUCGAAGCUGUUCCUGAUG | 918 | CAUCAGGAACAGCUUCGAN | 1871-1889 | 1871 |
| 430 | NUCGAAGCUGUUCCUGAUN | 919 | NAUCAGGAACAGCUUCGAN | 1871-1889 | 1871 |
| 431 | CUCGAAGCUGUUCCUGAUG | 920 | CAUCAGGAACAGCUUCIAG | 1871-1889 | 1871 |
| 432 | UUCGAAGCUGUUCCUGAUG | 921 | CAUCAGGAACAGCUUCIAA | 1871-1889 | 1871 |
| 433 | UUCGAAGCUGUUCCUGAUN | 922 | NAUCAGGAACAGCUUCIAA | 1871-1889 | 1871 |
| 434 | NUCGAAGCUGUUCCUGAUG | 923 | CAUCAGGAACAGCUUCIAN | 1871-1889 | 1871 |
| 435 | NUCGAAGCUGUUCCUGAUN | 924 | NAUCAGGAACAGCUUCIAN | 1871-1889 | 1871 |
| 436 | UCUUGUUCAGGCAAAUCAG | 925 | CUGAUUUGCCUGAACAAGA | 4992-5010 | 4992 |
| 437 | UCUUGUUCAGGCAAAUCAN | 926 | NUGAUUUGCCUGAACAAGA | 4992-5010 | 4992 |
| 438 | NCUUGUUCAGGCAAAUCAG | 927 | CUGAUUUGCCUGAACAAGN | 4992-5010 | 4992 |
| 439 | NCUUGUUCAGGCAAAUCAN | 928 | NUGAUUUGCCUGAACAAGN | 4992-5010 | 4992 |
| 440 | UCACCAAAGUGGUUGUCCU | 929 | AGGACAACCACUUUGGUGA | 6798-6816 | 6798 |
| 441 | UCACCAAAGUGGUUGUCCN | 930 | NGGACAACCACUUUGGUGA | 6798-6816 | 6798 |
| 442 | NCACCAAAGUGGUUGUCCU | 931 | AGGACAACCACUUUGGUGN | 6798-6816 | 6798 |
| 443 | NCACCAAAGUGGUUGUCCN | 932 | NGGACAACCACUUUGGUGN | 6798-6816 | 6798 |
| 444 | UCACCAAAGUGGUUGUCCU | 933 | AGGACAACCACUUUIGUGA | 6798-6816 | 6798 |
| 445 | UCACCAAAGUGGUUGUCCN | 934 | NGGACAACCACUUUIGUGA | 6798-6816 | 6798 |
| 446 | NCACCAAAGUGGUUGUCCU | 935 | AGGACAACCACUUUIGUGN | 6798-6816 | 6798 |
| 447 | NCACCAAAGUGGUUGUCCN | 936 | NGGACAACCACUUUIGUGN | 6798-6816 | 6798 |
| 448 | GAGCAGAGGUUGUUCUGGU | 937 | ACCAGAACAACCUCUGCUC | 8739-8757 | 8739 |
| 449 | UAGCAGAGGUUGUUCUGGU | 938 | ACCAGAACAACCUCUGCUA | 8739-8757 | 8739 |
| 450 | UAGCAGAGGUUGUUCUGGN | 939 | NCCAGAACAACCUCUGCUA | 8739-8757 | 8739 |
| 451 | NAGCAGAGGUUGUUCUGGU | 940 | ACCAGAACAACCUCUGCUN | 8739-8757 | 8739 |
| 452 | NAGCAGAGGUUGUUCUGGN | 941 | NCCAGAACAACCUCUGCUN | 8739-8757 | 8739 |
| 453 | GAGCAGAGGUUGUUCUGGU | 942 | ACCAGAACAACCUCUICUC | 8739-8757 | 8739 |
| 454 | UAGCAGAGGUUGUUCUGGU | 943 | ACCAGAACAACCUCUICUA | 8739-8757 | 8739 |
| 455 | UAGCAGAGGUUGUUCUGGN | 944 | NCCAGAACAACCUCUICUA | 8739-8757 | 8739 |
| 456 | NAGCAGAGGUUGUUCUGGU | 945 | ACCAGAACAACCUCUICUN | 8739-8757 | 8739 |
| 457 | NAGCAGAGGUUGUUCUGGN | 946 | NCCAGAACAACCUCUICUN | 8739-8757 | 8739 |
| 458 | UAGAUUGUGCUGGUUGUAG | 947 | CUACAACCAGCACAAUCUC | 9310-9328 | 9310 |
| 459 | UAGAUUGUGCUGGUUGUAG | 948 | CUACAACCAGCACAAUCUA | 9310-9328 | 9310 |
| 460 | NAGAUUGUGCUGGUUGUAG | 949 | CUACAACCAGCACAAUCUN | 9310-9328 | 9310 |
| 461 | UAGAUUGUGCUGGUUGUAN | 950 | NUACAACCAGCACAAUCUA | 9310-9328 | 9310 |
| 462 | NAGAUUGUGCUGGUUGUAN | 951 | NUACAACCAGCACAAUCUN | 9310-9328 | 9310 |
| 463 | CAGAAGUUGUGCUGGUUGU | 952 | ACAACCAGCACAACUUCUG | 10206-10224 | 10206 |
| 464 | UAGAAGUUGUGCUGGUUGU | 953 | ACAACCAGCACAACUUCUA | 10206-10224 | 10206 |
| 465 | NAGAAGUUGUGCUGGUUGU | 954 | ACAACCAGCACAACUUCUN | 10206-10224 | 10206 |
| 466 | UAGAAGUUGUGCUGGUUGN | 955 | NCAACCAGCACAACUUCUA | 10206-10224 | 10206 |
| 467 | NAGAAGUUGUGCUGGUUGN | 956 | NCAACCAGCACAACUUCUN | 10206-10224 | 10206 |
| 468 | CUUGUCACCAAAGUGGUUG | 957 | CAACCACUUUGGUGACAAG | 11014-11032 | 11014 |
| 469 | UUUGUCACCAAAGUGGUUG | 958 | CAACCACUUUGGUGACAAA | 11014-11032 | 11014 |
| 470 | NUUGUCACCAAAGUGGUUG | 959 | CAACCACUUUGGUGACAAN | 11014-11032 | 11014 |
| 471 | UUUGUCACCAAAGUGGUUN | 960 | NAACCACUUUGGUGACAAA | 11014-11032 | 11014 |
| 472 | NUUGUCACCAAAGUGGUUN | 961 | NAACCACUUUGGUGACAAN | 11014-11032 | 11014 |
| 473 | CAGAGGUUGUGUUGGUUGU | 962 | ACAACCAACACAACUUCUG | 11361-11379 | 11361 |
| 474 | UAGAGGUUGUGUUGGUUGU | 963 | ACAACCAACACAACUUCUA | 11361-11379 | 11361 |
| 475 | NAGAGGUUGUGUUGGUUGU | 964 | ACAACCAACACAACUUCUN | 11361-11379 | 11361 |
| 476 | UAGAGGUUGUGUUGGUUGN | 965 | NCAACCAACACAACUUCUA | 11361-11379 | 11361 |
| 477 | NAGAGGUUGUGUUGGUUGN | 966 | NCAACCAACACAACUUCUN | 11361-11379 | 11361 |
| 478 | UCUAGUUGUAGGAGCAGAG | 967 | CUCUGCUCCUACAACUAGA | 12965-12983 | 12965 |
| 479 | GCUAGUUGUAGGAGCAGAG | 968 | CUCUGCUCCUACAACUAGC | 12965-12983 | 12965 |
| 480 | NCUAGUUGUAGGAGCAGAG | 969 | CUCUGCUCCUACAACUAGN | 12965-12983 | 12965 |
| 481 | UCUAGUUGUAGGAGCAGAN | 970 | NUCUGCUCCUACAACUAGA | 12965-12983 | 12965 |
| 482 | NCUAGUUGUAGGAGCAGAN | 971 | NUCUGCUCCUACAACUAGN | 12965-12983 | 12965 |
| 483 | UGGUUCAGGAACACUUCCC | 972 | GGGAAGUGUUCCUGAACCA | 1567-1585 | 1567 |
| 484 | NGGUUCAGGAACACUUCCC | 973 | GGGAAGUGUUCCUGAACCN | 1567-1585 | 1567 |
| 485 | UGGUUCAGGAACACUUCCN | 974 | NGGAAGUGUUCCUGAACCA | 1567-1585 | 1567 |
| 486 | NGGUUCAGGAACACUUCCN | 975 | NGGAAGUGUUCCUGAACCN | 1567-1585 | 1567 |
| 487 | UGGUUCAGGAACACUUCCC | 976 | GGGAAGUGUUCCUIAACCA | 1567-1585 | 1567 |
| 488 | NGGUUCAGGAACACUUCCC | 977 | GGGAAGUGUUCCUIAACCN | 1567-1585 | 1567 |
| 489 | UGGUUCAGGAACACUUCCN | 978 | NGGAAGUGUUCCUIAACCA | 1567-1585 | 1567 |
| 490 | NGGUUCAGGAACACUUCCN | 979 | NGGAAGUGUUCCUIAACCN | 1567-1585 | 1567 |
| 491 | GAUGUCGUCGAAGUUCCCA | 980 | UGGGAACUUCGACGACAUC | 3155-3173 | 3155 |
| 492 | UAUGUCGUCGAAGUUCCCA | 981 | UGGGAACUUCGACGACAUA | 3155-3173 | 3155 |
| 493 | NAUGUCGUCGAAGUUCCCA | 982 | UGGGAACUUCGACGACAUN | 3155-3173 | 3155 |
| 494 | UAUGUCGUCGAAGUUCCCN | 983 | NGGGAACUUCGACGACAUA | 3155-3173 | 3155 |
| 495 | NAUGUCGUCGAAGUUCCCN | 984 | NGGGAACUUCGACGACAUN | 3155-3173 | 3155 |
| 496 | GAUGUCGUCGAAGUUCCCA | 985 | UGGGAACUUCGACIACAUC | 3155-3173 | 3155 |
| 497 | UAUGUCGUCGAAGUUCCCA | 986 | UGGGAACUUCGACIACAUA | 3155-3173 | 3155 |
| 498 | NAUGUCGUCGAAGUUCCCA | 987 | UGGGAACUUCGACIACAUN | 3155-3173 | 3155 |
| 499 | UAUGUCGUCGAAGUUCCCN | 988 | NGGGAACUUCGACIACAUA | 3155-3173 | 3155 |
| 500 | NAUGUCGUCGAAGUUCCCN | 989 | NGGGAACUUCGACIACAUN | 3155-3173 | 3155 |
| 501 | AUUUCUGCCAAGAGGAGGU | 990 | ACCUCCUCUUGGCAGAAAU | 6777-6795 | 6777 |
| 502 | AUUUCUGCCAAGAGGAGGN | 991 | NCCUCCUCUUGGCAGAAAU | 6777-6795 | 6777 |
| 503 | UUUUCUGCCAAGAGGAGGU | 992 | ACCUCCUCUUGGCAGAAAA | 6777-6795 | 6777 |
| 504 | UUUUCUGCCAAGAGGAGGN | 993 | NCCUCCUCUUGGCAGAAAA | 6777-6795 | 6777 |
| 505 | NUUUCUGCCAAGAGGAGGN | 994 | NCCUCCUCUUGGCAGAAAN | 6777-6795 | 6777 |
| 506 | AUUUCUGCCAAGAGGAGGU | 995 | ACCUCCUCUUGICAGAAAU | 6777-6795 | 6777 |
| 507 | AUUUCUGCCAAGAGGAGGN | 996 | NCCUCCUCUUGICAGAAAU | 6777-6795 | 6777 |
| 508 | UUUUCUGCCAAGAGGAGGU | 997 | ACCUCCUCUUGICAGAAAA | 6777-6795 | 6777 |
| 509 | UUUUCUGCCAAGAGGAGGN | 998 | NCCUCCUCUUGICAGAAAA | 6777-6795 | 6777 |
| 510 | NUUUCUGCCAAGAGGAGGN | 999 | NCCUCCUCUUGICAGAAAN | 6777-6795 | 6777 |
| 511 | UGUUGUUGAAGAUGAUCUC | 1000 | GAGAUCAUCUUCAACAACA | 15051-15069 | 15051 |
| 512 | UGUUGUUGAAGAUGAUCUN | 1001 | NAGAUCAUCUUCAACAACA | 15051-15069 | 15051 |
| 513 | NGUUGUUGAAGAUGAUCUC | 1002 | GAGAUCAUCUUCAACAACN | 15051-15069 | 15051 |
| 514 | NGUUGUUGAAGAUGAUCUN | 1003 | NAGAUCAUCUUCAACAACN | 15051-15069 | 15051 |
| 515 | UGUUGUUGUAGGUUUCCUU | 1004 | AAGGAAACCUACAACAACA | 5346-5364 | 5346 |
| 516 | NGUUGUUGUAGGUUUCCUU | 1005 | AAGGAAACCUACAACAACN | 5346-5364 | 5346 |
| 517 | UGUUGUUGUAGGUUUCCUN | 1006 | NAGGAAACCUACAACAACA | 5346-5364 | 5346 |
| 518 | NGUUGUUGUAGGUUUCCUN | 1007 | NAGGAAACCUACAACAACN | 5346-5364 | 5346 |
| 519 | AUCUUGUCCUCAUCAAAGA | 1008 | UCUUUGAUGAGGACAAGAU | 3694-3712 | 3694 |
| 520 | AUCUUGUCCUCAUCAAAGN | 1009 | NCUUUGAUGAGGACAAGAU | 3694-3712 | 3694 |
| 521 | NUCUUGUCCUCAUCAAAGN | 1010 | NCUUUGAUGAGGACAAGAN | 3694-3712 | 3694 |
| 522 | UUCUUGUCCUCAUCAAAGA | 1011 | UCUUUGAUGAGGACAAGAA | 3694-3712 | 3694 |
| 523 | UUCUUGUCCUCAUCAAAGN | 1012 | NCUUUGAUGAGGACAAGAA | 3694-3712 | 3694 |
| 524 | GCAUCUUGUCCUCAUCAAA | 1013 | UUUGAUGAGGACAAGAUGC | 3696-3714 | 3696 |
| 525 | UCAUCUUGUCCUCAUCAAA | 1014 | UUUGAUGAGGACAAGAUGA | 3696-3714 | 3696 |
| 526 | UCAUCUUGUCCUCAUCAAN | 1015 | NUUGAUGAGGACAAGAUGA | 3696-3714 | 3696 |
| 527 | NCAUCUUGUCCUCAUCAAA | 1016 | UUUGAUGAGGACAAGAUGN | 3696-3714 | 3696 |
| 528 | NCAUCUUGUCCUCAUCAAN | 1017 | NUUGAUGAGGACAAGAUGN | 3696-3714 | 3696 |
| 529 | GCUAGUUGUAGGAGCAGAG | 1018 | CUCUGCUCCUACAACUAGC | 6998-7016 | 6998 |
| 530 | UCUAGUUGUAGGAGCAGAG | 1019 | CUCUGCUCCUACAACUAGA | 6998-7016 | 6998 |
| 531 | NCUAGUUGUAGGAGCAGAG | 1020 | CUCUGCUCCUACAACUAGN | 6998-7016 | 6998 |
| 532 | UCUAGUUGUAGGAGCAGAN | 1021 | NUCUGCUCCUACAACUAGA | 6998-7016 | 6998 |
| 533 | NCUAGUUGUAGGAGCAGAN | 1022 | NUCUGCUCCUACAACUAGN | 6998-7016 | 6998 |
| 534 | UAGUUGUAGAAGCAGAGGU | 1023 | ACCUCUGCUUCUACAACUA | 7980-7998 | 7980 |
| 535 | NAGUUGUAGAAGCAGAGGU | 1024 | ACCUCUGCUUCUACAACUN | 7980-7998 | 7980 |
| 536 | UAGUUGUAGAAGCAGAGGN | 1025 | NCCUCUGCUUCUACAACUA | 7980-7998 | 7980 |
| 537 | NAGUUGUAGAAGCAGAGGN | 1026 | NCCUCUGCUUCUACAACUN | 7980-7998 | 7980 |
| 538 | CAGAAGUUGUGCUGGUUAU | 1027 | AUAACCAGCACAACUUCUG | 8448-8466 | 8448 |
| 539 | UAGAAGUUGUGCUGGUUAU | 1028 | AUAACCAGCACAACUUCUA | 8448-8466 | 8448 |
| 540 | NAGAAGUUGUGCUGGUUAU | 1029 | AUAACCAGCACAACUUCUN | 8448-8466 | 8448 |
| 541 | UAGAAGUUGUGCUGGUUAN | 1030 | NUAACCAGCACAACUUCUA | 8448-8466 | 8448 |
| 542 | NAGAAGUUGUGCUGGUUAN | 1031 | NUAACCAGCACAACUUCUN | 8448-8466 | 8448 |
| 543 | CCAACACUGCAGGUGAUGU | 1032 | ACAUCACCUGCAGUGUUGG | 2230-2248 | 2230 |
| 544 | UCAACACUGCAGGUGAUGU | 1033 | ACAUCACCUGCAGUGUUGA | 2230-2248 | 2230 |
| 545 | NCAACACUGCAGGUGAUGU | 1034 | ACAUCACCUGCAGUGUUGN | 2230-2248 | 2230 |
| 546 | UCAACACUGCAGGUGAUGN | 1035 | NCAUCACCUGCAGUGUUGA | 2230-2248 | 2230 |
| 547 | NCAACACUGCAGGUGAUGN | 1036 | NCAUCACCUGCAGUGUUGN | 2230-2248 | 2230 |
| 548 | CCAACACUGCAGGUGAUGU | 1037 | ACAUCACCUGCAGUIUUGG | 2230-2248 | 2230 |
| 549 | UCAACACUGCAGGUGAUGU | 1038 | ACAUCACCUGCAGUIUUGA | 2230-2248 | 2230 |
| 550 | NCAACACUGCAGGUGAUGU | 1039 | ACAUCACCUGCAGUIUUGN | 2230-2248 | 2230 |
| 551 | UCAACACUGCAGGUGAUGN | 1040 | NCAUCACCUGCAGUIUUGA | 2230-2248 | 2230 |
| 552 | NCAACACUGCAGGUGAUGN | 1041 | NCAUCACCUGCAGUIUUGN | 2230-2248 | 2230 |
| 553 | CAUCUUGUCCUCAUCAAAG | 1042 | CUUUGAUGAGGACAAGAUG | 3695-3713 | 3695 |
| 554 | UAUCUUGUCCUCAUCAAAG | 1043 | CUUUGAUGAGGACAAGAUA | 3695-3713 | 3695 |
| 555 | NAUCUUGUCCUCAUCAAAG | 1044 | CUUUGAUGAGGACAAGAUN | 3695-3713 | 3695 |
| 556 | UAUCUUGUCCUCAUCAAAN | 1045 | NUUUGAUGAGGACAAGAUA | 3695-3713 | 3695 |
| 557 | NAUCUUGUCCUCAUCAAAN | 1046 | NUUUGAUGAGGACAAGAUN | 3695-3713 | 3695 |
| 558 | GUCGUGUGGUAGAUGACGU | 1047 | ACGUCAUCUACCACACGAC | 3910-3928 | 3910 |
| 559 | UUCGUGUGGUAGAUGACGU | 1048 | ACGUCAUCUACCACACGAA | 3910-3928 | 3910 |
| 560 | NUCGUGUGGUAGAUGACGU | 1049 | ACGUCAUCUACCACACGAN | 3910-3928 | 3910 |
| 561 | UUCGUGUGGUAGAUGACGN | 1050 | NCGUCAUCUACCACACGAA | 3910-3928 | 3910 |
| 562 | NUCGUGUGGUAGAUGACGN | 1051 | NCGUCAUCUACCACACGAN | 3910-3928 | 3910 |
| 563 | GUCGUGUGGUAGAUGACGU | 1052 | ACGUCAUCUACCACACIAC | 3910-3928 | 3910 |
| 564 | UUCGUGUGGUAGAUGACGU | 1053 | ACGUCAUCUACCACACIAA | 3910-3928 | 3910 |
| 565 | NUCGUGUGGUAGAUGACGU | 1054 | ACGUCAUCUACCACACIAN | 3910-3928 | 3910 |
| 566 | UUCGUGUGGUAGAUGACGN | 1055 | NCGUCAUCUACCACACIAA | 3910-3928 | 3910 |
| 567 | NUCGUGUGGUAGAUGACGN | 1056 | NCGUCAUCUACCACACIAN | 3910-3928 | 3910 |

The MUC5AC RNAi agent sense strands and antisense strands that comprise or consist of the nucleotide sequences in Table 2 can be modified nucleotides or unmodified nucleotides. In some embodiments, the MUC5AC RNAi agents having the sense and antisense strand sequences that comprise or consist of any of the nucleotide sequences in Table 2 are all or substantially all modified nucleotides.

In some embodiments, the antisense strand of a MUC5AC RNAi agent disclosed herein comprises at least 15 contiguous nucleotides differing by 0, 1, 2, or 3 nucleotides from any of the antisense strand sequences in Table 2. In some embodiments, the sense strand of a MUC5AC RNAi agent disclosed herein comprises at least 15 contiguous nucleotides differing by 0, 1, 2, or 3 nucleotides from any of the sense strand sequences in Table 2.

As used herein, each N listed in a sequence disclosed in Table 2 may be independently selected from any and all nucleobases (including those found on both modified and unmodified nucleotides). In some embodiments, an N nucleotide listed in a sequence disclosed in Table 2 has a nucleobase that is complementary to the N nucleotide at the corresponding position on the other strand. In some embodiments, an N nucleotide listed in a sequence disclosed in Table 2 has a nucleobase that is not complementary to the N nucleotide at the corresponding position on the other strand. In some embodiments, an N nucleotide listed in a sequence disclosed in Table 2 has a nucleobase that is the same as the N nucleotide at the corresponding position on the other strand. In some embodiments, an N nucleotide listed in a sequence disclosed in Table 2 has a nucleobase that is different from the N nucleotide at the corresponding position on the other strand.

Certain modified MUC5AC RNAi agent sense and antisense strands are provided in Table 3, Table 4, Table 5, Table 6, Table 7, and Table 11. Certain modified MUC5AC RNAi agent antisense strands, as well as their underlying unmodified nucleobase sequences, are provided in Table 3. Certain modified MUC5AC RNAi agent sense strands, as well as their underlying unmodified nucleobase sequences, are provided in Tables 4, 5, and 6. In forming MUC5AC RNAi agents, each of the nucleotides in each of the underlying base sequences listed in Tables 3, 4, 5, 6, and 7, as well as in Table 2, above, can be a modified nucleotide.

The MUC5AC RNAi agents described herein are formed by annealing an antisense strand with a sense strand. A sense strand containing a sequence listed in Table 2, Table 4, Table 5, Table 6, Table 7, or Table 11 can be hybridized to any antisense strand containing a sequence listed in Table 2, Table 3, or Table 11, provided the two sequences have a region of at least 85% complementarity over a contiguous 16, 17, 18, 19, 20, or 21 nucleotide sequence.

In some embodiments, a MUC5AC RNAi agent antisense strand comprises a nucleotide sequence of any of the sequences in Table 2 or Table 3.

In some embodiments, a MUC5AC RNAi agent comprises or consists of a duplex having the nucleobase sequences of the sense strand and the antisense strand of any of the sequences in Table 2, Table 3, Table 4, Table 5, Table 6, Table 7, or Table 11.

Examples of antisense strands containing modified nucleotides are provided in Table 3. Examples of sense strands containing modified nucleotides are provided in Tables 4, 5 and 6.

As used in Tables 3, 4, 5, 6, 7, and 11, the following notations are used to indicate modified nucleotides, targeting groups, and linking groups:
A=adenosine-3′-phosphate
C=cytidine-3′-phosphate
G=guanosine-3′-phosphate
U=uridine-3′-phosphate
I=inosine-3′-phosphate
a=2′-O-methyladenosine-3′-phosphate
as=2′-O-methyladenosine-3′-phosphorothioate
c=2′-O-methylcytidine-3′-phosphate
cs=2′-O-methylcytidine-3′-phosphorothioate
g=2′-O-methylguanosine-3′-phosphate
gs=2′-O-methylguanosine-3′-phosphorothioate
i=2′-O-methylinosine-3′-phosphate
is=2′-O-methylinosine-3′-phosphorothioate
t=2′-O-methyl-5-methyluridine-3′-phosphate
ts=2′-O-methyl-5-methyluridine-3′-phosphorothioate
u=2′-O-methyluridine-3′-phosphate
us=2′-O-methyluridine-3′-phosphorothioate
Af=2′-fluoroadenosine-3′-phosphate
Afs=2′-fluoroadenosine-3′-phosporothioate
Cf=2′-fluorocytidine-3′-phosphate
Cfs=2′-fluorocytidine-3′-phosphorothioate
Gf=2′-fluoroguanosine-3′-phosphate
Gfs=2′-fluoroguanosine-3′-phosphorothioate
Tf=2′-fluoro-5′-methyluridine-3′-phosphate
Tfs=2′-fluoro-5′-methyluridine-3′-phosphorothioate
Uf=2′-fluorouridine-3′-phosphate
Ufs=2′-fluorouridine-3′-phosphorothioate
dT=2′-deoxythymidine-3′-phosphate
A_{UNA}=2′,3′-seco-adenosine-3′-phosphate
A_{UNAS}=2′,3′-seco-adenosine-3′-phosphorothioate
C_{UNA}=2′,3′-seco-cytidine-3′-phosphate
C_{UNAS}=2′,3′-seco-cytidine-3′-phosphorothioate
G_{UNA}=2′,3′-seco-guanosine-3′-phosphate
G_{UNAS}=2′,3′-seco-guanosine-3′-phosphorothioate
U_{UNA}=2′,3′-seco-uridine-3′-phosphate
U_{UNAS}=2′,3′-seco-uridine-3′-phosphorothioate
a_2N=see Table 12
a_2Ns=see Table 12
(invAb)=inverted abasic deoxyribonucleotide-5′-phosphate, see Table 12
(invAb)s=inverted abasic deoxyribonucleotide-5′-phosphorothioate, see Table 12
s=phosphorothioate linkage
p=terminal phosphate (as synthesized)
vpdN=vinyl phosphonate deoxyribonucleotide
cPrpa=5′-cyclopropyl phosphonate-2′-O-methyladenosine-3′-phosphate (see Table 12)
cPrpas=5′-cyclopropyl phosphonate-2′-O-methyladenosine-3′-phosphorothioate (see Table 12)
cPrpu=5′-cyclopropyl phosphonate-2′-O-methyluridine-3′-phosphate (see Table 12)
cPrpus=5′-cyclopropyl phosphonate-2′-O-methyluridine-3′-phosphorothioate (see Table 12)
(Alk-SS-C6)=see Table 12
(C6-SS-Alk)=see Table 12
(C6-SS-C6)=see Table 12
(6-SS-6)=see Table 12
(C6-SS-Alk-Me)=see Table 12
(NH2-C6)=see Table 12
(TriAlk14)=see Table 12
(TriAlk14)s=see Table 12
—C6-=see Table 12
—C6s-=see Table 12
-L6-C6-=see Table 12
-L6-C6s-=see Table 12
-Alk-cyHex-=see Table 12
-Alk-cyHexs-=see Table 12
(TA14)=see Table 12 (structure of (TriAlk14)s after conjugation)
(TA14)s=see Table 12 (structure of (TriAlk14)s after conjugation)

As the person of ordinary skill in the art would readily understand, unless otherwise indicated by the sequence (such as, for example, by a phosphorothioate linkage “s”), when present in an oligonucleotide, the nucleotide monomers are mutually linked by 5′-3′-phosphodiester bonds. As the person of ordinary skill in the art would clearly understand, the inclusion of a phosphorothioate linkage as shown in the modified nucleotide sequences disclosed herein replaces the phosphodiester linkage typically present in oligonucleotides. Further, the person of ordinary skill in the art would readily understand that the terminal nucleotide at the 3′ end of a given oligonucleotide sequence would typically have a hydroxyl (—OH) group at the respective 3′ position of the given monomer instead of a phosphate moiety ex vivo. Additionally, for the embodiments disclosed herein, when viewing the respective strand 5′→3′, the inverted abasic residues are inserted such that the 3′ position of the deoxyribose is linked at the 3′ end of the preceding monomer on the respective strand (see, e.g., Table 12). Moreover, as the person of ordinary skill would readily understand and appreciate, while the phosphorothioate chemical structures depicted herein typically show the anion on the sulfur atom, the inventions disclosed herein encompass all phosphorothioate tautomers (e.g., where the sulfur atom has a double-bond and the anion is on an oxygen atom). Unless expressly indicated otherwise herein, such understandings of the person of ordinary skill in the art are used when describing the MUC5AC RNAi agents and compositions of MUC5AC RNAi agents disclosed herein.

Certain examples of targeting groups and linking groups used with the MUC5AC RNAi agents disclosed herein are included in the chemical structures provided below in Table 12. Each sense strand and/or antisense strand disclosed herein can have any targeting groups or linking groups listed herein, as well as other targeting or linking groups, conjugated to the 5′ and/or 3′ end of the sequence.

TABLE 3
MUC5AC RNAi Agent Antisense Strand Sequences

| | | | Underlying Base | |
|---|---|---|---|---|
| | | SEQ | Sequence (5′→3′) | SEQ |
| AS | | ID | (Shown as an Unmodified | ID |
| Strand ID | Modified Antisense Strand (5′→3′) | NO. | Nucleotide Sequence) | NO. |
| AM10579-AS | usUfsusGfaUfgGfcCfuUfgGfaGfcAfgGfsu | 1057 | UUUGAUGGCCUUGGAGCAGGU | 1517 |
| AM10581-AS | asAfsusCfuUfgAfuGfgCfcUfuGfgAfgCfsa | 1058 | AAUCUUGAUGGCCUUGGAGCA | 1518 |
| AM10583-AS | usUfsusGfaAfcUfcGfgGfgCfuGfaGfgUfsu | 1059 | UUUGAACUCGGGGCUGAGGUU | 1519 |
| AM10585-AS | usGfsasUfgCfuGfcAfcUfgCfuUfcUfgGfsg | 1060 | UGAUGCUGCACUGCUUCUGGG | 1520 |
| AM10587-AS | usUfsasGfuCfgCfaGfaAfcAfgAfgGfgCfsa | 1061 | UUAGUCGCAGAACAGAGGGCA | 1521 |
| AM10589-AS | asGfsusAfgUfcGfcAfgAfaCfaGfaGfgGfsc | 1062 | AGUAGUCGCAGAACAGAGGGC | 1522 |
| AM10591-AS | usUfsasGfuAfgUfcGfcAfgAfaCfaGfaGfsg | 1063 | UUAGUAGUCGCAGAACAGAGG | 1523 |
| AM10593-AS | usGfsusAfgUfaGfuCfgCfaGfaAfcAfgAfsg | 1064 | UGUAGUAGUCGCAGAACAGAG | 1524 |
| AM10595-AS | usUfsgsUfaGfuAfgUfcGfcAfgAfaCfaGfsc | 1065 | UUGUAGUAGUCGCAGAACAGC | 1525 |
| AM10597-AS | asUfsasGfuUfgUfaGfcAfgAfuGfgGfuGfsg | 1066 | AUAGUUGUAGCAGAUGGGUGG | 1526 |
| AM10599-AS | usUfscsCfaCfgUfcGfaAfcCfaCfuUfuGfsc | 1067 | UUCCACGUCGAACCACUUUGC | 1527 |
| AM10601-AS | usAfsasGfuCfcAfcGfuCfgAfaCfcAfcUfsc | 1068 | UAAGUCCACGUCGAACCACUC | 1528 |
| AM10603-AS | usGfsgsAfaGfuCfcAfcGfuCfgAfaCfcAfsc | 1069 | UGGAAGUCCACGUCGAACCAC | 1529 |
| AM10605-AS | usGfsgsAfaGfU_{UNA}CfcAfcGfuCfgAfaCfcAfsc | 1070 | UGGAAGUCCACGUCGAACCAC | 1529 |
| AM10739-AS | cPrpasAfsgsGfuCfuUfgUfaGfuGfgAfaGfcUfsg | 1071 | AAGGUCUUGUAGUGGAAGCUG | 1530 |
| AM10741-AS | cPrpasAfsgsGfuCfU_{UNA}UfgUfaGfuGfgAfaGfcUfsg | 1072 | AAGGUCUUGUAGUGGAAGCUG | 1530 |
| AM10743-AS | cPrpusAfscsCfaGfuGfcUfgAfgCfaUfaCfuUfsc | 1073 | UACCAGUGCUGAGCAUACUUC | 1531 |
| AM10744-AS | cPrpusAfscsCfaGfU_{UNA}GfcUfgAfgCfaUfaCfuUfsc | 1074 | UACCAGUGCUGAGCAUACUUC | 1531 |
| AM10747-AS | cPrpusUfsusGfaAfgguguUfgAfaGfaAfgGfsc | 1075 | UUUGAAGGUGUUGAAGAAGGC | 1532 |
| AM10764-AS | asGfsasUfgCfuGfgUfcUfuCfuUfgUfcCfsc | 1076 | AGAUGCUGGUCUUCUUGUCCC | 1533 |
| AM10766-AS | usGfsusUfgAfuGfaAfgAfuGfcUfgGfuCfsc | 1077 | UGUUGAUGAAGAUGCUGGUCC | 1534 |
| AM10768-AS | usUfscsUfuGfuUfcAfgGfcAfaAfuCfaGfsc | 1078 | UUCUUGUUCAGGCAAAUCAGC | 1535 |
| AM10770-AS | asUfsgsUfuGfuUfgUfaGfgUfuUfcCfuUfsg | 1079 | AUGUUGUUGUAGGUUUCCUUG | 1536 |
| AM10772-AS | asUfsgsAfuGfuUfgUfuGfuAfgGfuUfuCfsc | 1080 | AUGAUGUUGUUGUAGGUUUCC | 1537 |
| AM10790-AS | usUfsgsAfuGfaAfgAfuGfcUfgGfuCfuUfsc | 1081 | UUGAUGAAGAUGCUGGUCUUC | 1538 |
| AM10792-AS | usGfsasUfcUfgGfuAfgUfuGfuAfgCfaGfsc | 1082 | UGAUCUGGUAGUUGUAGCAGC | 1539 |
| AM10794-AS | usCfscsUfgAfuCfuGfgUfaGfuUfgUfaGfsc | 1083 | UCCUGAUCUGGUAGUUGUAGC | 1540 |
| AM10796-AS | usAfsgsUfuGfuAfgCfaGfaUfgGfgUfgGfsg | 1084 | UAGUUGUAGCAGAUGGGUGGG | 1541 |
| AM10798-AS | usCfsasAfcAfcUfgGfaUfgCfgGfaUfcUfsc | 1085 | UCAACACUGGAUGCGGAUCUC | 1542 |
| AM10800-AS | usUfsgsUfuCfgAfuGfcUfcAfcCfuCfuGfsg | 1086 | UUGUUCGAUGCUCACCUCUGG | 1543 |
| AM10802-AS | usAfsusCfuUfgAfaGfgGfuCfcCfuGfcUfsg | 1087 | UAUCUUGAAGGGUCCCUGCUG | 1544 |
| AM10804-AS | usCfsgsUfaGfuUfgAfgGfcAfcAfuCfuUfsg | 1088 | UCGUAGUUGAGGCACAUCUUG | 1545 |
| AM10806-AS | usCfsusCfgUfaGfuUfgAfgGfcAfcAfuCfsc | 1089 | UCUCGUAGUUGAGGCACAUCC | 1546 |
| AM10808-AS | asAfsgsGfuCfuUfgUfaGfuGfgAfaGfcUfsg | 1090 | AAGGUCUUGUAGUGGAAGCUG | 1530 |
| AM10810-AS | usUfsusCfaGfgCfaGfgUfcUfcGfcUfgUfsc | 1091 | UUUCAGGCAGGUCUCGCUGUC | 1547 |
| AM10812-AS | usUfscsUfgAfaGfaUfgGfuGfaCfgUfuGfsg | 1092 | UUCUGAAGAUGGUGACGUUGG | 1548 |
| AM10814-AS | usGfsusCfuGfaAfgAfuGfgUfgAfcGfuUfsg | 1093 | UGUCUGAAGAUGGUGACGUUG | 1549 |
| AM10816-AS | usGfsgsAfaGfU_{UNA}CfaUfcGfgCfcUfgGfaUfsg | 1094 | UGGAAGUCAUCGGCCUGGAUG | 1550 |
| AM10818-AS | usUfsusGfaAfgguguUfgAfaGfaAfgGfsc | 1095 | UUUGAAGGUGUUGAAGAAGGC | 1532 |
| AM10821-AS | usGfscsAfgUfuCfgAfgUfaGfuAfgGfuUfsc | 1096 | UGCAGUUCGAGUAGUAGGUUC | 1551 |
| AM10823-AS | usUfsusGfgAfgCfaGfgUfgGfuCfcCfuGfsu | 1097 | UUUGGAGCAGGUGGUCCCUGU | 1552 |
| AM10825-AS | usCfsusUfgAfuGfgCfcUfuGfgAfgCfaGfsg | 1098 | UCUUGAUGGCCUUGGAGCAGG | 1553 |
| AM10827-AS | usUfsgsUfcAfuCfgUfgGfuUfcCfaCfaUfsg | 1099 | UUGUCAUCGUGGUUCCACAUG | 1554 |
| AM10829-AS | asCfsasGfaAfgCfaGfaGfgUfcUfuGfcCfsu | 1100 | ACAGAAGCAGAGGUCUUGCCU | 1555 |
| AM10831-AS | usCfsasGfuUfgGfuGfcAfgUfcUfgUfgGfsa | 1101 | UCAGUUGGUGCAGUCUGUGGA | 1556 |
| AM10833-AS | usCfsasGfuAfcAfgUfgAfaGfgCfaCfuGfsc | 1102 | UCAGUACAGUGAAGGCACUGC | 1557 |
| AM10835-AS | usGfscsUfgUfuGfaAfgUfuCfcCfaCfaGfsc | 1103 | UGCUGUUGAAGUUCCCACAGC | 1558 |
| AM10837-AS | usGfsasUfgCfuGfuUfgAfaGfuUfcCfcAfsc | 1104 | UGAUGCUGUUGAAGUUCCCAC | 1559 |
| AM10839-AS | usGfsgsUfcUfuGfaAfgGfuGfuUfgAfaGfsc | 1105 | UGGUCUUGAAGGUGUUGAAGC | 1560 |
| AM10841-AS | asAfsgsCfuGfuUfcCfuGfaUfgUfuGfgGfsg | 1106 | AAGCUGUUCCUGAUGUUGGGG | 1561 |
| AM10843-AS | asCfsasUfgCfaGfuUfcGfaGfuAfgUfaGfsg | 1107 | ACAUGCAGUUCGAGUAGUAGG | 1562 |
| AM10845-AS | usAfsasGfcCfaAfcAfcUfgCfaGfgUfgAfsc | 1108 | UAAGCCAACACUGCAGGUGAC | 1563 |
| AM10847-AS | usUfsgsUfaAfcAfgGfuCfaUfgUfcCfaGfsc | 1109 | UUGUAACAGGUCAUGUCCAGC | 1564 |
| AM10849-AS | usCfsgsUfuGfaAfgCfuGfuAfgCfuCfuGfsc | 1110 | UCGUUGAAGCUGUAGCUCUGC | 1565 |
| AM11065-AS | usAfscsCfaGfU_{UNA}GfcUfgAfgCfaUfaCfuUfsc | 1111 | UACCAGUGCUGAGCAUACUUC | 1531 |
| AM11264-AS | cPrpusGfsgsAfuCfuCfaUfaGfuUfgUfaGfcAfsg | 1112 | UGGAUCUCAUAGUUGUAGCAG | 1566 |
| AM11266-AS | cPrpusGfsgsAfuCfU_{UNA}CfaUfaGfuUfgUfaGfcAfsg | 1113 | UGGAUCUCAUAGUUGUAGCAG | 1566 |
| AM11268-AS | cPrpusGfsusCfaAfaCfcAfcUfuGfgUfcCfaGfsg | 1114 | UGUCAAACCACUUGGUCCAGG | 1567 |
| AM11271-AS | cPrpusUfsgsUfuGfuUfgAfaGfaUfgAfuCfuCfsg | 1115 | UUGUUGUUGAAGAUGAUCUCG | 1568 |
| AM11272-AS | cPrpusUfsgsUfuguugaaGfaUfgAfucucsg | 1116 | UUGUUGUUGAAGAUGAUCUCG | 1568 |
| AM11275-AS | usAfsgsUfaCfaGfuGfaAfgGfcAfcUfgCfsu | 1117 | UAGUACAGUGAAGGCACUGCU | 1569 |
| AM11277-AS | usUfscsGfaAfgCfuGfuUfcCfuGfaUfgUfsc | 1118 | UUCGAAGCUGUUCCUGAUGUC | 1570 |
| AM11279-AS | usCfsusUfgUfuCfaGfgCfaAfaUfcAfgCfsc | 1119 | UCUUGUUCAGGCAAAUCAGCC | 1571 |
| AM11281-AS | usCfsasCfcAfaAfgUfgGfuUfgUfcCfuGfsg | 1120 | UCACCAAAGUGGUUGUCCUGG | 1572 |
| AM11283-AS | usAfsgsCfaGfaGfgUfuGfuUfcUfgGfuUfsg | 1121 | UAGCAGAGGUUGUUCUGGUUG | 1573 |
| AM11285-AS | usAfsgsAfuUfgUfgCfuGfgUfuGfuAfgCfsg | 1122 | UAGAUUGUGCUGGUUGUAGCG | 1574 |
| AM11287-AS | usAfsgsAfaGfuUfgUfgCfuGfgUfuGfuGfsg | 1123 | UAGAAGUUGUGCUGGUUGUGG | 1575 |
| AM11289-AS | usUfsusGfuCfaCfcAfaAfgUfgGfuUfgUfsc | 1124 | UUUGUCACCAAAGUGGUUGUC | 1576 |
| AM11291-AS | usAfsgsAfgGfuUfgUfgUfuGfgUfuGfuAfsg | 1125 | UAGAGGUUGUGUUGGUUGUAG | 1577 |
| AM11293-AS | usCfsusAfgUfuGfuAfgGfaGfcAfgAfgGfsu | 1126 | UCUAGUUGUAGGAGCAGAGGU | 1578 |
| AM11401-AS | cPrpusUfsgsUfaGfuAfgUfcGfcAfgAfaCfaGfsc | 1127 | UUGUAGUAGUCGCAGAACAGC | 1525 |
| AM11403-AS | usUfsgsUfaGfuAfgUfcAfcAfgAfaCfaGfsc | 1128 | UUGUAGUAGUCACAGAACAGC | 1579 |
| AM11404-AS | cPrpusUfsgsUfaGfuAfgUfcAfcAfgAfaCfaGfsc | 1129 | UUGUAGUAGUCACAGAACAGC | 1579 |
| AM11405-AS | cPrpusUfsgsuaguagucAfcAfgAfacagsc | 1130 | UUGUAGUAGUCACAGAACAGC | 1579 |
| AM11462-AS | cPrpusCfsasuaguuguaGfcAfcAfugggsu | 1131 | UCAUAGUUGUAGCACAUGGGU | 1580 |
| AM11464-AS | cPrpusGfsusUfgUfuGfaAfgAfuGfaUfcUfgGfsu | 1132 | UGUUGUUGAAGAUGAUCUGGU | 1581 |
| AM11465-AS | cPrpusGfsusuguugaagAfuGfaUfcuggsu | 1133 | UGUUGUUGAAGAUGAUCUGGU | 1581 |
| AM11467-AS | cPrpusGfsusUfgAfaGfuUfaCfcAfcAfgAfgCfsc | 1134 | UGUUGAAGUUACCACAGAGCC | 1582 |
| AM11469-AS | cPrpusAfscsUfuUfuCfaUfuCfuCfcAfcGfcUfsc | 1135 | UACUUUUCAUUCUCCACGCUC | 1583 |
| AM11471-AS | cPrpusAfsgsCfaUfaCfuUfuUfcAfuUfcUfcCfsc | 1136 | UAGCAUACUUUUCAUUCUCCC | 1584 |
| AM11473-AS | cPrpusCfsasUfaCfaUfgCfaGfuUfcGfaGfaAfsg | 1137 | UCAUACAUGCAGUUCGAGAAG | 1585 |
| AM11475-AS | cPrpusGfsusCfaUfaCfaUfgCfaGfuUfcGfaGfsc | 1138 | UGUCAUACAUGCAGUUCGAGC | 1586 |
| AM11477-AS | cPrpusUfsgsUfcAfuAfcAfuGfcAfgUfuCfgAfsg | 1139 | UUGUCAUACAUGCAGUUCGAG | 1587 |
| AM11479-AS | cPrpusUfsasGfuAfgUfcAfcAfgAfaCfaGfuGfsg | 1140 | UUAGUAGUCACAGAACAGUGG | 1588 |
| AM11481-AS | cPrpusGfsusAfgUfaGfuCfaCfaGfaAfcAfgUfsg | 1141 | UGUAGUAGUCACAGAACAGUG | 1589 |
| AM11495-AS | cPrpusUfsgsUfaGfuAfgUfcicAfgAfaCfaGfsc | 1142 | UUGUAGUAGUCICAGAACAGC | 1590 |
| AM11496-AS | cPrpusUfsgsUfaGfuAfgUfcgcAfgAfaCfaGfsc | 1143 | UUGUAGUAGUCGCAGAACAGC | 1525 |
| AM11498-AS | asCfsasUfgCfaGfuUfcGfaGfaAfgAfaGfsg | 1144 | ACAUGCAGUUCGAGAAGAAGG | 1591 |
| AM11499-AS | cPrpasCfsasUfgCfaGfuUfcGfaGfaAfgAfaGfsg | 1145 | ACAUGCAGUUCGAGAAGAAGG | 1591 |
| AM11740-AS | cPrpasUfsasGfuUfgUfaGfcAfcAfuGfgGfuGfsg | 1146 | AUAGUUGUAGCACAUGGGUGG | 1592 |
| AM11741-AS | cPrpasUfsasguuguagcAfcAfuGfggugsg | 1147 | AUAGUUGUAGCACAUGGGUGG | 1592 |
| AM11742-AS | cPrpusGfsgsaucucauaGfuUfgUfagcasg | 1148 | UGGAUCUCAUAGUUGUAGCAG | 1566 |
| AM11745-AS | cPrpusUfsgsuuguugaaGfaUfgAfucucsg | 1149 | UUGUUGUUGAAGAUGAUCUCG | 1568 |
| AM11821-AS | cPrpusGfsusAfguagucaCfaGfaAfcagusg | 1150 | UGUAGUAGUCACAGAACAGUG | 1589 |
| AM11823-AS | cPrpusGfsusaguagucaCfaGfaAfcagusg | 1151 | UGUAGUAGUCACAGAACAGUG | 1589 |
| AM11825-AS | cPrpusGfsusaguagucaCfaGfaAfcagusc | 1152 | UGUAGUAGUCACAGAACAGUG | 1593 |
| AM11971-AS | usGfsgsUfuCfaGfgAfaCfaCfuUfcCfcCfsa | 1153 | UGGUUCAGGAACACUUCCCCA | 1594 |
| AM11973-AS | usCfsasAfcAfcUfgCfaGfgUfgAfuGfuCfsc | 1154 | UCAACACUGCAGGUGAUGUCC | 1595 |
| AM11975-AS | usAfsusGfuCfgUfcGfaAfgUfuCfcCfaCfsa | 1155 | UAUGUCGUCGAAGUUCCCACA | 1596 |
| AM11977-AS | usAfsusCfuUfgUfcCfuCfaUfcAfaAfgAfsc | 1156 | UAUCUUGUCCUCAUCAAAGAC | 1597 |
| AM11979-AS | usUfscsGfuGfuGfgUfaGfaUfgAfcGfuCfsc | 1157 | UUCGUGUGGUAGAUGACGUCC | 1598 |
| AM11982-AS | asUfsusUfcUfgCfcAfaGfaGfgAfgGfuGfsc | 1158 | AUUUCUGCCAAGAGGAGGUGC | 1599 |
| AM11984-AS | usGfsusUfgUfuGfaAfgAfuGfaUfcUfcGfsu | 1159 | UGUUGUUGAAGAUGAUCUCGU | 1600 |
| AM11986-AS | usUfsgsUfuguugaaGfaUfgAfucucsg | 1160 | UUGUUGUUGAAGAUGAUCUCG | 1568 |
| AM12158-AS | usGfsusUfgUfuGfuAfgGfuUfuCfcUfuGfsc | 1161 | UGUUGUUGUAGGUUUCCUUGC | 1601 |
| AM12159-AS | cPrpusGfsusUfgUfuGfuAfgGfuUfuCfcUfuGfsc | 1162 | UGUUGUUGUAGGUUUCCUUGC | 1601 |
| AM12161-AS | usGfsusuguuguagGfuUfuCfcuugsc | 1163 | UGUUGUUGUAGGUUUCCUUGC | 1601 |
| AM12162-AS | cPrpusGfsusuguuguagGfuUfuCfcuugsc | 1164 | UGUUGUUGUAGGUUUCCUUGC | 1601 |
| AM12163-AS | cPrpusUfscsUfuGfuUfcAfgGfcAfaAfuCfaGfsc | 1165 | UUCUUGUUCAGGCAAAUCAGC | 1535 |
| AM12165-AS | usUfscsuuguucagGfcAfaAfucagsc | 1166 | UUCUUGUUCAGGCAAAUCAGC | 1535 |
| AM12166-AS | cPrpusUfscsuuguucagGfcAfaAfucagsc | 1167 | UUCUUGUUCAGGCAAAUCAGC | 1535 |
| AM12167-AS | cPrpusUfscsuugU_{UNA}UcagGfcAfaAfucagsc | 1168 | UUCUUGUUCAGGCAAAUCAGC | 1535 |
| AM12169-AS | cPrpusCfsusUfgUfuCfaGfgCfaAfaUfcAfgCfsc | 1169 | UCUUGUUCAGGCAAAUCAGCC | 1571 |
| AM12171-AS | usCfsusuguucaggCfaAfaUfcagcsc | 1170 | UCUUGUUCAGGCAAAUCAGCC | 1571 |
| AM12172-AS | cPrpusCfsusuguucaggCfaAfaUfcagcsc | 1171 | UCUUGUUCAGGCAAAUCAGCC | 1571 |
| AM12173-AS | cPrpusGfsusUfgAfuGfaAfgAfuGfcUfgGfuCfsc | 1172 | UGUUGAUGAAGAUGCUGGUCC | 1534 |
| AM12175-AS | usGfsusugaugaagAfuGfcUfggucsc | 1173 | UGUUGAUGAAGAUGCUGGUCC | 1534 |
| AM12176-AS | cPrpusGfsusugaugaagAfuGfcUfggucsc | 1174 | UGUUGAUGAAGAUGCUGGUCC | 1534 |
| AM12177-AS | cPrpusGfsusugaU_{UNA}gaagAfuGfcUfggucsc | 1175 | UGUUGAUGAAGAUGCUGGUCC | 1534 |
| AM12178-AS | cPrpusUfsgsUfugU_{UNA}UgaaGfaUfgAfucucsg | 1176 | UUGUUGUUGAAGAUGAUCUCG | 1568 |
| AM12180-AS | cPrpusUfsgsUfuguU_{UNA}gaaGfaUfgAfucucsg | 1177 | UUGUUGUUGAAGAUGAUCUCG | 1568 |
| AM12181-AS | cPrpusUfsgsUfU_{UNA}guugaaGfaUfgAfucucsg | 1178 | UUGUUGUUGAAGAUGAUCUCG | 1568 |
| AM12182-AS | cPrpusUfsgsiuguugaaGfaUfgAfucucsg | 1179 | UUGIUGUUGAAGAUGAUCUCG | 1602 |
| AM12189-AS | asUfscsUfuGfuCfcUfcAfuCfaAfaGfaUfsg | 1180 | AUCUUGUCCUCAUCAAAGAUG | 1603 |
| AM12191-AS | usCfsasUfcUfuGfuCfcUfcAfuCfaAfaGfsc | 1181 | UCAUCUUGUCCUCAUCAAAGC | 1604 |
| AM12193-AS | usCfsusAfgUfuGfuAfgGfaGfcAfgAfgAfsc | 1182 | UCUAGUUGUAGGAGCAGAGAC | 1605 |
| AM12195-AS | usAfsgsUfuGfuAfgAfaGfcAfgAfgGfuUfsg | 1183 | UAGUUGUAGAAGCAGAGGUUG | 1606 |
| AM12197-AS | usAfsgsAfaGfuUfgUfgCfuGfgUfuAfuAfsg | 1184 | UAGAAGUUGUGCUGGUUAUAG | 1607 |
| AM12516-AS | usUfsgsuaguagucGfcAfgAfacagsc | 1185 | UUGUAGUAGUCGCAGAACAGC | 1525 |
| AM12519-AS | usUfsgsuagU_{UNA}agucGfcAfgAfacagsc | 1186 | UUGUAGUAGUCGCAGAACAGC | 1525 |
| AM12608-AS | usUfscsuuguucagGfcAfaAfucagsg | 1187 | UUCUUGUUCAGGCAAAUCAGG | 1608 |
| AM12609-AS | usUfscsuuGfuucagGfcAfaAfucagsc | 1188 | UUCUUGUUCAGGCAAAUCAGC | 1535 |
| AM12610-AS | usUfscsUfuguucagGfcAfaAfucagsc | 1189 | UUCUUGUUCAGGCAAAUCAGC | 1535 |
| AM12611-AS | usUfscsuuguU_{UNA}cagGfcAfaAfucagsc | 1190 | UUCUUGUUCAGGCAAAUCAGC | 1535 |
| AM12612-AS | cPrpuUfcuuguucagGfcAfaAfucagsc | 1191 | UUCUUGUUCAGGCAAAUCAGC | 1535 |
| AM08569-AS | usGfsgsAfuCfuCfaUfaGfuUfgUfaGfcAfsg | 1716 | UGGAUCUCAUAGUUGUAGCAG | 1566 |
| AM07104-AS | usUfsgsUfuGfuUfgAfaGfaUfgAfuCfuCfsg | 1717 | UUGUUGUUGAAGAUGAUCUCG | 1568 |

TABLE 4
MUC5AC Agent Sense Strand Sequences
(Shown Without Linkers, Conjugates, or Capping Moieties)

| | | | Underlying Base | |
|---|---|---|---|---|
| | | SEQ | Sequence (5′→3′) | SEQ |
| | Modified | ID | (Shown as an Unmodified | ID |
| Strand ID | Sense Strand (5′→3′) | NO. | Nucleotide Sequence) | NO. |
| AM10578-SS-NL | asccugcucCfAfAfgiccaucaaa | 1192 | ACCUGCUCCAAGICCAUCAAA | 1609 |
| AM10580-SS-NL | usgcuccaaGfGfCfcaucaagauu | 1193 | UGCUCCAAGGCCAUCAAGAUU | 1610 |
| AM10582-SS-NL | asaccucagCfUfCfcgaguucaaa | 1194 | AACCUCAGCUCCGAGUUCAAA | 1611 |
| AM10584-SS-NL | csccagaagCfAfGfugcaicauca | 1195 | CCCAGAAGCAGUGCAICAUCA | 1612 |
| AM10586-SS-NL | usgcccucuGfUfUfcugciacuaa | 1196 | UGCCCUCUGUUCUGCIACUAA | 1613 |
| AM10588-SS-NL | gscccucugUfUfCfuicgacuacu | 1197 | GCCCUCUGUUCUICGACUACU | 1614 |
| AM10590-SS-NL | cscucuguuCfUfGfcgacuacuaa | 1198 | CCUCUGUUCUGCGACUACUAA | 1615 |
| AM10592-SS-NL | csucuguucUfGfCfgacuacuaca | 1199 | CUCUGUUCUGCGACUACUACA | 1616 |
| AM10594-SS-NL | gscuguucuGfCfGfacuacuacaa | 1200 | GCUGUUCUGCGACUACUACAA | 1617 |
| AM10596-SS-NL | cscacccauCfUfGfcuacaacuau | 1201 | CCACCCAUCUGCUACAACUAU | 1618 |
| AM10598-SS-NL | gscaaagugGfUfUfcgaciuggaa | 1202 | GCAAAGUGGUUCGACIUGGAA | 1619 |
| AM10600-SS-NL | gsagugguuCfGfAfcgugiacuua | 1203 | GAGUGGUUCGACGUGIACUUA | 1620 |
| AM10602-SS-NL | gsugguucgAfCfGfugiacuucca | 1204 | GUGGUUCGACGUGIACUUCCA | 1621 |
| AM10604-SS-NL | gsugguucgAfCfGfuggacuucca | 1205 | GUGGUUCGACGUGGACUUCCA | 1622 |
| AM10738-SS-NL | csagcuuccAfCfUfacaaiaccuu | 1206 | CAGCUUCCACUACAAIACCUU | 1623 |
| AM10740-SS-NL | csagcuuccAfCfUfacaagaccuu | 1207 | CAGCUUCCACUACAAGACCUU | 1624 |
| AM10742-SS-NL | gsa_2NaguaugCfUfCfagcacugiua | 1208 | G(A^{2N})AGUAUGCUCAGCACUGIUA | 1625 |
| AM10745-SS-NL | gsa_2NaguaugCfUfCfaguacugiua | 1209 | G(A^{2N})AGUAUGCUCAGUACUGIUA | 1626 |
| AM10746-SS-NL | gsccuucuuCfAfAfcaccuucaaa | 1210 | GCCUUCUUCAACACCUUCAAA | 1627 |
| AM10748-SS-NL | gsccuucuuCfAfAfcaucuucaaa | 1211 | GCCUUCUUCAACAUCUUCAAA | 1628 |
| AM10749-SS-NL | gsccuucuuCfAfAfcacuuucaaa | 1212 | GCCUUCUUCAACACUUUCAAA | 1629 |
| AM10763-SS-NL | gsggacaagAfAfGfaccaicaucu | 1213 | GGGACAAGAAGACCAICAUCU | 1630 |
| AM10765-SS-NL | gsgaccagcAfUfCfuucaucaaca | 1214 | GGACCAGCAUCUUCAUCAACA | 1631 |
| AM10767-SS-NL | gscugauuuGfCfCfugaacaagaa | 1215 | GCUGAUUUGCCUGAACAAGAA | 1632 |
| AM10769-SS-NL | csa_2NaggaaaCfCfUfacaacaacau | 1216 | C(A^{2N})AGGAAACCUACAACAACAU | 1633 |
| AM10771-SS-NL | gsgaaaccuAfCfAfacaacaucau | 1217 | GGAAACCUACAACAACAUCAU | 1634 |
| AM10789-SS-NL | gsa_2NagaccaGfCfAfucuucaucaa | 1218 | G(A^{2N})AGACCAGCAUCUUCAUCAA | 1635 |
| AM10791-SS-NL | gscugcuacAfAfCfuaccaiauca | 1219 | GCUGCUACAACUACCAIAUCA | 1636 |
| AM10793-SS-NL | gscuacaacUfAfCfcagaucaiga | 1220 | GCUACAACUACCAGAUCAIGA | 1637 |
| AM10795-SS-NL | csccacccaUfCfUfgcuacaacua | 1221 | CCCACCCAUCUGCUACAACUA | 1638 |
| AM10797-SS-NL | gsagauccgCfAfUfccaguiuuga | 1222 | GAGAUCCGCAUCCAGUIUUGA | 1639 |
| AM10799-SS-NL | cscagagguGfAfGfcauciaacaa | 1223 | CCAGAGGUGAGCAUCIAACAA | 1640 |
| AM10801-SS-NL | csagcagggAfCfCfcuucaagaua | 1224 | CAGCAGGGACCCUUCAAGAUA | 1641 |
| AM10803-SS-NL | csa_2NagauguGfCfCfucaacuacia | 1225 | C(A^{2N})AGAUGUGCCUCAACUACIA | 1642 |
| AM10805-SS-NL | gsgaugugcCfUfCfaacuaciaga | 1226 | GGAUGUGCCUCAACUACIAGA | 1643 |
| AM10807-SS-NL | csagcuuccAfCfUfacaaiaccuu | 1227 | CAGCUUCCACUACAAIACCUU | 1623 |
| AM10809-SS-NL | gsacagcgaGfAfCfcugcuugaaa | 1228 | GACAGCGAGACCUGCUUGAAA | 1644 |
| AM10811-SS-NL | cscaacgucAfCfCfaucuucagaa | 1229 | CCAACGUCACCAUCUUCAGAA | 1645 |
| AM10813-SS-NL | csa_2NacgucaCfCfAfucuucaiaca | 1230 | C(A^{2N})ACGUCACCAUCUUCAIACA | 1646 |
| AM10815-SS-NL | csauccaggCfCfGfaugacuucca | 1231 | CAUCCAGGCCGAUGACUUCCA | 1647 |
| AM10817-SS-NL | gsccuucuuCfAfAfcaccuucaaa | 1232 | GCCUUCUUCAACACCUUCAAA | 1627 |
| AM10819-SS-NL | gsa_2NaguaugCfUfCfagcacugiua | 1233 | G(A^{2N})AGUAUGCUCAGCACUGIUA | 1625 |
| AM10820-SS-NL | gsa_2NaccuacUfAfCfucgaacuica | 1234 | G(A^{2N})ACCUACUACUCGAACUICA | 1648 |
| AM10822-SS-NL | ascagggacCfAfCfcugcuucaaa | 1235 | ACAGGGACCACCUGCUUCAAA | 1649 |
| AM10824-SS-NL | cscugcuccAfAfGfgcuaucaaga | 1236 | CCUGCUCCAAGGCUAUCAAGA | 1650 |
| AM10826-SS-NL | csa_2NuguggaAfCfCfacgauiacaa | 1237 | C(A^{2N})UGUGGAACCACGAUIACAA | 1651 |
| AM10828-SS-NL | asggcaagaCfCfUfcugcuucuiu | 1238 | AGGCAAGACCUCUGCUUCUIU | 1652 |
| AM10830-SS-NL | usccacagaCfUfGfcaccaacuia | 1239 | UCCACAGACUGCACCAACUIA | 1653 |
| AM10832-SS-NL | gscagugccUfUfCfacuguacuia | 1240 | GCAGUGCCUUCACUGUACUIA | 1654 |
| AM10834-SS-NL | gscugugggAfAfCfuucaacaica | 1241 | GCUGUGGGAACUUCAACAICA | 1655 |
| AM10836-SS-NL | gsugggaacUfUfCfaacaicauca | 1242 | GUGGGAACUUCAACAICAUCA | 1656 |
| AM10838-SS-NL | gscuucaacAfCfCfuucaaiacca | 1243 | GCUUCAACACCUUCAAIACCA | 1657 |
| AM10840-SS-NL | cscccaacaUfCfAfggaacaicuu | 1244 | CCCCAACAUCAGGAACAICUU | 1658 |
| AM10842-SS-NL | cscuacuacUfCfGfaacuicaugu | 1245 | CCUACUACUCGAACUICAUGU | 1659 |
| AM10844-SS-NL | gsucaccugCfAfGfuguugicuua | 1246 | GUCACCUGCAGUGUUGICUUA | 1660 |
| AM10846-SS-NL | gscuggacaUfGfAfccuguuacaa | 1247 | GCUGGACAUGACCUGUUACAA | 1661 |
| AM10848-SS-NL | gscagagcuAfCfAfgcuucaacia | 1248 | GCAGAGCUACAGCUUCAACIA | 1662 |
| AM11066-SS-NL | gsa_2NaguaugCfUfCfaguacugiua | 1249 | G(A^{2N})AGUAUGCUCAGUACUGIUA | 1626 |
| AM11263-SS-NL | csugcuacaAfCfUfaugagaucca | 1250 | CUGCUACAACUAUGAGAUCCA | 1663 |
| AM11265-SS-NL | csugcuacaAfCfUfaugaiaucca | 1251 | CUGCUACAACUAUGAIAUCCA | 1664 |
| AM11267-SS-NL | cscuggaccAfAfGfugguuugaca | 1252 | CCUGGACCAAGUGGUUUGACA | 1665 |
| AM11269-SS-NL | cscuggaccAfAfGfugguuuiaca | 1253 | CCUGGACCAAGUGGUUUIACA | 1666 |
| AM11270-SS-NL | csgagaucaUfCfUfucaacaacaa | 1254 | CGAGAUCAUCUUCAACAACAA | 1667 |
| AM11274-SS-NL | asgcagugcCfUfUfcacuguacua | 1255 | AGCAGUGCCUUCACUGUACUA | 1668 |
| AM11276-SS-NL | gsacaucagGfAfAfcagcuuciaa | 1256 | GACAUCAGGAACAGCUUCIAA | 1669 |
| AM11278-SS-NL | gsgcugauuUfGfCfcugaacaaga | 1257 | GGCUGAUUUGCCUGAACAAGA | 1670 |
| AM11280-SS-NL | cscaggacaAfCfCfacuuuiguga | 1258 | CCAGGACAACCACUUUIGUGA | 1671 |
| AM11282-SS-NL | csaaccagaAfCfAfaccucuicua | 1259 | CAACCAGAACAACCUCUICUA | 1672 |
| AM11284-SS-NL | csgcuacaaCfCfAfgcacaaucua | 1260 | CGCUACAACCAGCACAAUCUA | 1673 |
| AM11286-SS-NL | cscacaaccAfGfCfacaacuucua | 1261 | CCACAACCAGCACAACUUCUA | 1674 |
| AM11288-SS-NL | gsacaaccaCfUfUfuggugacaaa | 1262 | GACAACCACUUUGGUGACAAA | 1675 |
| AM11290-SS-NL | csuacaaccAfAfCfacaacuucua | 1263 | CUACAACCAACACAACUUCUA | 1676 |
| AM11292-SS-NL | asccucugcUfCfCfuacaacuaga | 1264 | ACCUCUGCUCCUACAACUAGA | 1677 |
| AM11400-SS-NL | gscuguucuGfCfGfacuacuacaa | 1265 | GCUGUUCUGCGACUACUACAA | 1617 |
| AM11402-SS-NL | gscuguucuGfUfGfacuacuacaa | 1266 | GCUGUUCUGUGACUACUACAA | 1678 |
| AM11463-SS-NL | asccagaucAfUfCfuucaacaaca | 1267 | ACCAGAUCAUCUUCAACAACA | 1679 |
| AM11466-SS-NL | gsgcucuguGfGfUfaacuucaaca | 1268 | GGCUCUGUGGUAACUUCAACA | 1680 |
| AM11468-SS-NL | gsagcguggAfGfAfaugaaaagua | 1269 | GAGCGUGGAGAAUGAAAAGUA | 1681 |
| AM11470-SS-NL | gsggagaauGfAfAfaaguaugcua | 1270 | GGGAGAAUGAAAAGUAUGCUA | 1682 |
| AM11472-SS-NL | csuucucgaAfCfUfgcauguauga | 1271 | CUUCUCGAACUGCAUGUAUGA | 1683 |
| AM11474-SS-NL | gscucgaacUfGfCfauguaugaca | 1272 | GCUCGAACUGCAUGUAUGACA | 1684 |
| AM11476-SS-NL | csucgaacuGfCfAfuguaugacaa | 1273 | CUCGAACUGCAUGUAUGACAA | 1685 |
| AM11478-SS-NL | cscacuguuCfUfGfugacuacuaa | 1274 | CCACUGUUCUGUGACUACUAA | 1686 |
| AM11480-SS-NL | csacuguucUfGfUfgacuacuaca | 1275 | CACUGUUCUGUGACUACUACA | 1687 |
| AM11497-SS-NL | cscuucuucUfCfGfaacuicaugu | 1276 | CCUUCUUCUCGAACUICAUGU | 1688 |
| AM11739-SS-NL | cscacccauGfUfGfcuacaacuau | 1277 | CCACCCAUGUGCUACAACUAU | 1689 |
| AM11743-SS-NL | csugcuaCfaAfcUfaugaiaucca | 1278 | CUGCUACAACUAUGAIAUCCA | 1664 |
| AM11744-SS-NL | csgagauCfaUfcUfucaacaacaa | 1279 | CGAGAUCAUCUUCAACAACAA | 1667 |
| AM11822-SS-NL | csacuguUfcUfgUfgacuacuaca | 1280 | CACUGUUCUGUGACUACUACA | 1687 |
| AM11824-SS-NL | gsacuguUfcUfgUfgacuacuaca | 1281 | GACUGUUCUGUGACUACUACA | 1690 |
| AM11970-SS-NL | usggggaagUfGfUfuccuiaacca | 1282 | UGGGGAAGUGUUCCUIAACCA | 1691 |
| AM11972-SS-NL | gsgacaucaCfCfUfgcaguiuuga | 1283 | GGACAUCACCUGCAGUIUUGA | 1692 |
| AM11974-SS-NL | usgugggaaCfUfUfcgaciacaua | 1284 | UGUGGGAACUUCGACIACAUA | 1693 |
| AM11976-SS-NL | gsucuuugaUfGfAfggacaagaua | 1285 | GUCUUUGAUGAGGACAAGAUA | 1694 |
| AM11978-SS-NL | gsgacgucaUfCfUfaccacaciaa | 1286 | GGACGUCAUCUACCACACIAA | 1695 |
| AM11980-SS-NL | csugcuacaAfCfUfaugaiaucca | 1287 | CUGCUACAACUAUGAIAUCCA | 1664 |
| AM11981-SS-NL | gscaccuccUfCfUfugicagaaau | 1288 | GCACCUCCUCUUGICAGAAAU | 1696 |
| AM11983-SS-NL | ascgagaucAfUfCfuucaacaaca | 1289 | ACGAGAUCAUCUUCAACAACA | 1697 |
| AM11985-SS-NL | csgagaucaUfCfUfucaacaacaa | 1290 | CGAGAUCAUCUUCAACAACAA | 1667 |
| AM12157-SS-NL | gscaaggaaAfCfCfuacaacaaca | 1291 | GCAAGGAAACCUACAACAACA | 1698 |
| AM12160-SS-NL | gscaaggAfaAfcCfuacaacaaca | 1292 | GCAAGGAAACCUACAACAACA | 1698 |
| AM12164-SS-NL | gscugauUfuGfcCfugaacaagaa | 1293 | GCUGAUUUGCCUGAACAAGAA | 1632 |
| AM12168-SS-NL | gscugauUfuGfcCfuga_2Nacaagaa | 1294 | GCUGAUUUGCCUG(A^{2N})ACAAGAA | 1699 |
| AM12170-SS-NL | gsgcugaUfuUfgCfcugaacaaga | 1295 | GGCUGAUUUGCCUGAACAAGA | 1670 |
| AM12174-SS-NL | gsgaccaGfcAfuCfuucaucaaca | 1296 | GGACCAGCAUCUUCAUCAACA | 1631 |
| AM12179-SS-NL | csgagaucaUfCfUfuca_2Nacaacaa | 1297 | CGAGAUCAUCUUC(A^{2N})ACAACAA | 1700 |
| AM12188-SS-NL | csaucuuugAfUfGfaggacaagau | 1298 | CAUCUUUGAUGAGGACAAGAU | 1701 |
| AM12190-SS-NL | gscuuugauGfAfGfgacaagauga | 1299 | GCUUUGAUGAGGACAAGAUGA | 1702 |
| AM12192-SS-NL | gsucucugcUfCfCfuacaacuaga | 1300 | GUCUCUGCUCCUACAACUAGA | 1703 |
| AM12194-SS-NL | csaaccucuGfCfUfucuacaacua | 1301 | CAACCUCUGCUUCUACAACUA | 1704 |
| AM12196-SS-NL | csua_2NuaaccAfGfCfacaacuucua | 1302 | CU(A^{2N})UAACCAGCACAACUUCUA | 1705 |
| AM12198-SS-NL | csuauaaccAfGfCfacaacuucua | 1303 | CUAUAACCAGCACAACUUCUA | 1706 |
| AM12515-SS-NL | gscuguucuGfcGfaCfuacuacaa | 1304 | GCUGUUCUGCGACUACUACAA | 1617 |
| AM12517-SS-NL | gscuguuCfuGfcGfacuacuacaa | 1305 | GCUGUUCUGCGACUACUACAA | 1617 |
| AM12518-SS-NL | gscuguucuGfcGfacuacuacaa | 1306 | GCUGUUCUGCGACUACUACAA | 1617 |
| AM12520-SS-NL | gscuguuuuGfcGfacuacuacaa | 1307 | GCUGUUUUGCGACUACUACAA | 1707 |
| AM12521-SS-NL | gscuguucuGfcGfauuacuacaa | 1308 | GCUGUUCUGCGAUUACUACAA | 1708 |
| AM12522-SS-NL | gscuguucuGfcGfacuauuacaa | 1309 | GCUGUUCUGCGACUAUUACAA | 1709 |
| AM12523-SS-NL | gscuguucuGfcGfAfcuacuacaa | 1310 | GCUGUUCUGCGACUACUACAA | 1617 |
| AM12605-SS-NL | gscugauuuGfcCfugaacaagaa | 1311 | GCUGAUUUGCCUGAACAAGAA | 1632 |
| AM12606-SS-NL | gscugauuuGfcCfuGfaacaagaa | 1312 | GCUGAUUUGCCUGAACAAGAA | 1632 |
| AM12607-SS-NL | cscugauUfuGfcCfugaacaagaa | 1313 | CCUGAUUUGCCUGAACAAGAA | 1710 |
| AM12715-SS-NL | csgagaucaUfCfUfucaacaacaa | 1314 | CGAGAUCAUCUUCAACAACAA | 1667 |
| AM13074-SS-NL | gscugauUfuGfcCfugaacaagaa | 1315 | GCUGAUUUGCCUGAACAAGAA | 1632 |
| AM14080-SS-NL | gscuguucuGfCfGfacuacuacaa | 1316 | GCUGUUCUGCGACUACUACAA | 1617 |
| AM14081-SS-NL | gscugguucuGfCfGfacuacuacaa | 1317 | GCUGGUUCUGCGACUACUACAA | 1711 |
| AM14084-SS-NL | gscguucuGfCfGfacuacuacaa | 1318 | GCGUUCUGCGACUACUACAA | 1712 |

(A^{2N}) = 2-aminoadenine-containing nucleotide; I = hypoxanthine (inosine) nucleotide
** For the constructs in Table 4 above, a capping moiety, such as for example, (InvAb) or s(InvAb), or a conjugate is typically located at the 3′ end of the modified sense strand sequence shown (see, e.g., Table 5, below).

TABLE 5
MUC5AC Agent Sense Strand Sequences (Shown With TriAlk14 Linker
(see Table 12 for structure information)).

| | | | Underlying Base | |
|---|---|---|---|---|
| | | | Sequence (5′→3′) | |
| | | SEQ | (Shown as | SEQ |
| | | ID | an Unmodified | ID |
| Strand ID | Modified Sense Strand (5′→3′) | NO. | Nucleotide Sequence) | NO. |
| AM10578-SS | (TriAlk14)accugcucCfAfAfgiccaucaaas(invAb) | 1319 | ACCUGCUCCAAGICCAUCAAA | 1609 |
| AM10580-SS | (TriAlk14)ugcuccaaGfGfCfcaucaagauus(invAb) | 1320 | UGCUCCAAGGCCAUCAAGAUU | 1610 |
| AM10582-SS | (TriAlk14)aaccucagCfUfCfcgaguucaaas(invAb) | 1321 | AACCUCAGCUCCGAGUUCAAA | 1611 |
| AM10584-SS | (TriAlk14)cccagaagCfAfGfugcaicaucas(invAb) | 1322 | CCCAGAAGCAGUGCAICAUCA | 1612 |
| AM10586-SS | (TriAlk14)ugcccucuGfUfUfcugciacuaas(invAb) | 1323 | UGCCCUCUGUUCUGCIACUAA | 1613 |
| AM10588-SS | (TriAlk14)gcccucugUfUfCfuicgacuacus(invAb) | 1324 | GCCCUCUGUUCUICGACUACU | 1614 |
| AM10590-SS | (TriAlk14)ccucuguuCfUfGfcgacuacuaas(invAb) | 1325 | CCUCUGUUCUGCGACUACUAA | 1615 |
| AM10592-SS | (TriAlk14)cucuguucUfGfCfgacuacuacas(invAb) | 1326 | CUCUGUUCUGCGACUACUACA | 1616 |
| AM10594-SS | (TriAlk14)gcuguucuGfCfGfacuacuacaas(invAb) | 1327 | GCUGUUCUGCGACUACUACAA | 1617 |
| AM10596-SS | (TriAlk14)ccacccauCfUfGfcuacaacuaus(invAb) | 1328 | CCACCCAUCUGCUACAACUAU | 1618 |
| AM10598-SS | (TriAlk14)gcaaagugGfUfUfcgaciuggaas(invAb) | 1329 | GCAAAGUGGUUCGACIUGGAA | 1619 |
| AM10600-SS | (TriAlk14)gagugguuCfGfAfcgugiacuuas(invAb) | 1330 | GAGUGGUUCGACGUGIACUUA | 1620 |
| AM10602-SS | (TriAlk14)gugguucgAfCfGfugiacuuccas(invAb) | 1331 | GUGGUUCGACGUGIACUUCCA | 1621 |
| AM10604-SS | (TriAlk14)gugguucgAfCfGfuggacuuccas(invAb) | 1332 | GUGGUUCGACGUGGACUUCCA | 1622 |
| AM10738-SS | (TriAlk14)csagcuuccAfCfUfacaaiaccuus(invAb) | 1333 | CAGCUUCCACUACAAIACCUU | 1623 |
| AM10740-SS | (TriAlk14)csagcuuccAfCfUfacaagaccuus(invAb) | 1334 | CAGCUUCCACUACAAGACCUU | 1624 |
| AM10742-SS | (TriAlk14)gsa_2NaguaugCfUfCfagcacugiuas(invAb) | 1335 | G(A^{2N})AGUAUGCUCAGCACUGIUA | 1625 |
| AM10745-SS | (TriAlk14)gsa_2NaguaugCfUfCfaguacugiuas(invAb) | 1336 | G(A^{2N})AGUAUGCUCAGUACUGIUA | 1626 |
| AM10746-SS | (TriAlk14)gsccuucuuCfAfAfcaccuucaaas(invAb) | 1337 | GCCUUCUUCAACACCUUCAAA | 1627 |
| AM10748-SS | (TriAlk14)gsccuucuuCfAfAfcaucuucaaas(invAb) | 1338 | GCCUUCUUCAACAUCUUCAAA | 1628 |
| AM10749-SS | (TriAlk14)gsccuucuuCfAfAfcacuuucaaas(invAb) | 1339 | GCCUUCUUCAACACUUUCAAA | 1629 |
| AM10763-SS | (TriAlk14)gggacaagAfAfGfaccaicaucus(invAb) | 1340 | GGGACAAGAAGACCAICAUCU | 1630 |
| AM10765-SS | (TriAlk14)ggaccagcAfUfCfuucaucaacas(invAb) | 1341 | GGACCAGCAUCUUCAUCAACA | 1631 |
| AM10767-SS | (TriAlk14)gcugauuuGfCfCfugaacaagaas(invAb) | 1342 | GCUGAUUUGCCUGAACAAGAA | 1632 |
| AM10769-SS | (TriAlk14)ca_2NaggaaaCfCfUfacaacaacaus(invAb) | 1343 | C(A^{2N})AGGAAACCUACAACAACAU | 1633 |
| AM10771-SS | (TriAlk14)ggaaaccuAfCfAfacaacaucaus(invAb) | 1344 | GGAAACCUACAACAACAUCAU | 1634 |
| AM10789-SS | (TriAlk14)ga_2NagaccaGfCfAfucuucaucaas(invAb) | 1345 | G(A^{2N})AGACCAGCAUCUUCAUCAA | 1635 |
| AM10791-SS | (TriAlk14)gcugcuacAfAfCfuaccaiaucas(invAb) | 1346 | GCUGCUACAACUACCAIAUCA | 1636 |
| AM10793-SS | (TriAlk14)gcuacaacUfAfCfcagaucaigas(invAb) | 1347 | GCUACAACUACCAGAUCAIGA | 1637 |
| AM10795-SS | (TriAlk14)cccacccaUfCfUfgcuacaacuas(invAb) | 1348 | CCCACCCAUCUGCUACAACUA | 1638 |
| AM10797-SS | (TriAlk14)gagauccgCfAfUfccaguiuugas(invAb) | 1349 | GAGAUCCGCAUCCAGUIUUGA | 1639 |
| AM10799-SS | (TriAlk14)ccagagguGfAfGfcauciaacaas(invAb) | 1350 | CCAGAGGUGAGCAUCIAACAA | 1640 |
| AM10801-SS | (TriAlk14)cagcagggAfCfCfcuucaagauas(invAb) | 1351 | CAGCAGGGACCCUUCAAGAUA | 1641 |
| AM10803-SS | (TriAlk14)ca_2NagauguGfCfCfucaacuacias(invAb) | 1352 | C(A^{2N})AGAUGUGCCUCAACUACIA | 1642 |
| AM10805-SS | (TriAlk14)ggaugugcCfUfCfaacuaciagas(invAb) | 1353 | GGAUGUGCCUCAACUACIAGA | 1643 |
| AM10807-SS | (TriAlk14)cagcuuccAfCfUfacaaiaccuus(invAb) | 1354 | CAGCUUCCACUACAAIACCUU | 1623 |
| AM10809-SS | (TriAlk14)gacagcgaGfAfCfcugcuugaaas(invAb) | 1355 | GACAGCGAGACCUGCUUGAAA | 1644 |
| AM10811-SS | (TriAlk14)ccaacgucAfCfCfaucuucagaas(invAb) | 1356 | CCAACGUCACCAUCUUCAGAA | 1645 |
| AM10813-SS | (TriAlk14)ca_2NacgucaCfCfAfucuucaiacas(invAb) | 1357 | C(A^{2N})ACGUCACCAUCUUCAIACA | 1646 |
| AM10815-SS | (TriAlk14)cauccaggCfCfGfaugacuuccas(invAb) | 1358 | CAUCCAGGCCGAUGACUUCCA | 1647 |
| AM10817-SS | (TriAlk14)gccuucuuCfAfAfcaccuucaaas(invAb) | 1359 | GCCUUCUUCAACACCUUCAAA | 1627 |
| AM10819-SS | (TriAlk14)ga_2NaguaugCfUfCfagcacugiuas(invAb) | 1360 | G(A^{2N})AGUAUGCUCAGCACUGIUA | 1625 |
| AM10820-SS | (TriAlk14)ga_2NaccuacUfAfCfucgaacuicas(invAb) | 1361 | G(A^{2N})ACCUACUACUCGAACUICA | 1648 |
| AM10822-SS | (TriAlk14)acagggacCfAfCfcugcuucaaas(invAb) | 1362 | ACAGGGACCACCUGCUUCAAA | 1649 |
| AM10824-SS | (TriAlk14)ccugcuccAfAfGfgcuaucaagas(invAb) | 1363 | CCUGCUCCAAGGCUAUCAAGA | 1650 |
| AM10826-SS | (TriAlk14)ca_2NuguggaAfCfCfacgauiacaas(invAb) | 1364 | C(A^{2N})UGUGGAACCACGAUIACAA | 1651 |
| AM10828-SS | (TriAlk14)aggcaagaCfCfUfcugcuucuius(invAb) | 1365 | AGGCAAGACCUCUGCUUCUIU | 1652 |
| AM10830-SS | (TriAlk14)uccacagaCfUfGfcaccaacuias(invAb) | 1366 | UCCACAGACUGCACCAACUIA | 1653 |
| AM10832-SS | (TriAlk14)gcagugccUfUfCfacuguacuias(invAb) | 1367 | GCAGUGCCUUCACUGUACUIA | 1654 |
| AM10834-SS | (TriAlk14)gcugugggAfAfCfuucaacaicas(invAb) | 1368 | GCUGUGGGAACUUCAACAICA | 1655 |
| AM10836-SS | (TriAlk14)gugggaacUfUfCfaacaicaucas(invAb) | 1369 | GUGGGAACUUCAACAICAUCA | 1656 |
| AM10838-SS | (TriAlk14)gcuucaacAfCfCfuucaaiaccas(invAb) | 1370 | GCUUCAACACCUUCAAIACCA | 1657 |
| AM10840-SS | (TriAlk14)ccccaacaUfCfAfggaacaicuus(invAb) | 1371 | CCCCAACAUCAGGAACAICUU | 1658 |
| AM10842-SS | (TriAlk14)ccuacuacUfCfGfaacuicaugus(invAb) | 1372 | CCUACUACUCGAACUICAUGU | 1659 |
| AM10844-SS | (TriAlk14)gucaccugCfAfGfuguugicuuas(invAb) | 1373 | GUCACCUGCAGUGUUGICUUA | 1660 |
| AM10846-SS | (TriAlk14)gcuggacaUfGfAfccuguuacaas(invAb) | 1374 | GCUGGACAUGACCUGUUACAA | 1661 |
| AM10848-SS | (TriAlk14)gcagagcuAfCfAfgcuucaacias(invAb) | 1375 | GCAGAGCUACAGCUUCAACIA | 1662 |
| AM11066-SS | (TriAlk14)ga_2NaguaugCfUfCfaguacugiuas(invAb) | 1376 | G(A^{2N})AGUAUGCUCAGUACUGIUA | 1626 |
| AM11263-SS | (TriAlk14)csugcuacaAfCfUfaugagauccas(invAb) | 1377 | CUGCUACAACUAUGAGAUCCA | 1663 |
| AM11265-SS | (TriAlk14)csugcuacaAfCfUfaugaiauccas(invAb) | 1378 | CUGCUACAACUAUGAIAUCCA | 1664 |
| AM11267-SS | (TriAlk14)cscuggaccAfAfGfugguuugacas(invAb) | 1379 | CCUGGACCAAGUGGUUUGACA | 1665 |
| AM11269-SS | (TriAlk14)cscuggaccAfAfGfugguuuiacas(invAb) | 1380 | CCUGGACCAAGUGGUUUIACA | 1666 |
| AM11270-SS | (TriAlk14)csgagaucaUfCfUfucaacaacaas(invAb) | 1381 | CGAGAUCAUCUUCAACAACAA | 1667 |
| AM11274-SS | (TriAlk14)agcagugcCfUfUfcacuguacuas(invAb) | 1382 | AGCAGUGCCUUCACUGUACUA | 1668 |
| AM11276-SS | (TriAlk14)gacaucagGfAfAfcagcuuciaas(invAb) | 1383 | GACAUCAGGAACAGCUUCIAA | 1669 |
| AM11278-SS | (TriAlk14)ggcugauuUfGfCfcugaacaagas(invAb) | 1384 | GGCUGAUUUGCCUGAACAAGA | 1670 |
| AM11280-SS | (TriAlk14)ccaggacaAfCfCfacuuuigugas(invAb) | 1385 | CCAGGACAACCACUUUIGUGA | 1671 |
| AM11282-SS | (TriAlk14)caaccagaAfCfAfaccucuicuas(invAb) | 1386 | CAACCAGAACAACCUCUICUA | 1672 |
| AM11284-SS | (TriAlk14)cgcuacaaCfCfAfgcacaaucuas(invAb) | 1387 | CGCUACAACCAGCACAAUCUA | 1673 |
| AM11286-SS | (TriAlk14)ccacaaccAfGfCfacaacuucuas(invAb) | 1388 | CCACAACCAGCACAACUUCUA | 1674 |
| AM11288-SS | (TriAlk14)gacaaccaCfUfUfuggugacaaas(invAb) | 1389 | GACAACCACUUUGGUGACAAA | 1675 |
| AM11290-SS | (TriAlk14)cuacaaccAfAfCfacaacuucuas(invAb) | 1390 | CUACAACCAACACAACUUCUA | 1676 |
| AM11292-SS | (TriAlk14)accucugcUfCfCfuacaacuagas(invAb) | 1391 | ACCUCUGCUCCUACAACUAGA | 1677 |
| AM11400-SS | (TriAlk14)gscuguucuGfCfGfacuacuacaas(invAb) | 1392 | GCUGUUCUGCGACUACUACAA | 1617 |
| AM11402-SS | (TriAlk14)gscuguucuGfUfGfacuacuacaas(invAb) | 1393 | GCUGUUCUGUGACUACUACAA | 1678 |
| AM11463-SS | (TriAlk14)asccagaucAfUfCfuucaacaacas(invAb) | 1394 | ACCAGAUCAUCUUCAACAACA | 1679 |
| AM11466-SS | (TriAlk14)gsgcucuguGfGfUfaacuucaacas(invAb) | 1395 | GGCUCUGUGGUAACUUCAACA | 1680 |
| AM11468-SS | (TriAlk14)gsagcguggAfGfAfaugaaaaguas(invAb) | 1396 | GAGCGUGGAGAAUGAAAAGUA | 1681 |
| AM11470-SS | (TriAlk14)gsggagaauGfAfAfaaguaugcuas(invAb) | 1397 | GGGAGAAUGAAAAGUAUGCUA | 1682 |
| AM11472-SS | (TriAlk14)csuucucgaAfCfUfgcauguaugas(invAb) | 1398 | CUUCUCGAACUGCAUGUAUGA | 1683 |
| AM11474-SS | (TriAlk14)gscucgaacUfGfCfauguaugacas(invAb) | 1399 | GCUCGAACUGCAUGUAUGACA | 1684 |
| AM11476-SS | (TriAlk14)csucgaacuGfCfAfuguaugacaas(invAb) | 1400 | CUCGAACUGCAUGUAUGACAA | 1685 |
| AM11478-SS | (TriAlk14)cscacuguuCfUfGfugacuacuaas(invAb) | 1401 | CCACUGUUCUGUGACUACUAA | 1686 |
| AM11480-SS | (TriAlk14)csacuguucUfGfUfgacuacuacas(invAb) | 1402 | CACUGUUCUGUGACUACUACA | 1687 |
| AM11497-SS | (TriAlk14)cscuucuucUfCfGfaacuicaugus(invAb) | 1403 | CCUUCUUCUCGAACUICAUGU | 1688 |
| AM11739-SS | (TriAlk14)cscacccauGfUfGfcuacaacuaus(invAb) | 1404 | CCACCCAUGUGCUACAACUAU | 1689 |
| AM11743-SS | (TriAlk14)csugcuaCfaAfcUfaugaiauccas(invAb) | 1405 | CUGCUACAACUAUGAIAUCCA | 1664 |
| AM11744-SS | (TriAlk14)csgagauCfaUfcUfucaacaacaas(invAb) | 1406 | CGAGAUCAUCUUCAACAACAA | 1667 |
| AM11822-SS | (TriAlk14)csacuguUfcUfgUfgacuacuacas(invAb) | 1407 | CACUGUUCUGUGACUACUACA | 1687 |
| AM11824-SS | (TriAlk14)gsacuguUfcUfgUfgacuacuacas(invAb) | 1408 | GACUGUUCUGUGACUACUACA | 1690 |
| AM11970-SS | (TriAlk14)uggggaagUfGfUfuccuiaaccas(invAb) | 1409 | UGGGGAAGUGUUCCUIAACCA | 1691 |
| AM11972-SS | (TriAlk14)ggacaucaCfCfUfgcaguiuugas(invAb) | 1410 | GGACAUCACCUGCAGUIUUGA | 1692 |
| AM11974-SS | (TriAlk14)ugugggaaCfUfUfcgaciacauas(invAb) | 1411 | UGUGGGAACUUCGACIACAUA | 1693 |
| AM11976-SS | (TriAlk14)gucuuugaUfGfAfggacaagauas(invAb) | 1412 | GUCUUUGAUGAGGACAAGAUA | 1694 |
| AM11978-SS | (TriAlk14)ggacgucaUfCfUfaccacaciaas(invAb) | 1413 | GGACGUCAUCUACCACACIAA | 1695 |
| AM11980-SS | (TriAlk14)cugcuacaAfCfUfaugaiauccas(invAb) | 1414 | CUGCUACAACUAUGAIAUCCA | 1664 |
| AM11981-SS | (TriAlk14)gcaccuccUfCfUfugicagaaaus(invAb) | 1415 | GCACCUCCUCUUGICAGAAAU | 1696 |
| AM11983-SS | (TriAlk14)acgagaucAfUfCfuucaacaacas(invAb) | 1416 | ACGAGAUCAUCUUCAACAACA | 1697 |
| AM11985-SS | (TriAlk14)cgagaucaUfCfUfucaacaacaas(invAb) | 1417 | CGAGAUCAUCUUCAACAACAA | 1667 |
| AM12157-SS | (TriAlk14)gcaaggaaAfCfCfuacaacaacas(invAb) | 1418 | GCAAGGAAACCUACAACAACA | 1698 |
| AM12160-SS | (TriAlk14)gcaaggAfaAfcCfuacaacaacas(invAb) | 1419 | GCAAGGAAACCUACAACAACA | 1698 |
| AM12164-SS | (TriAlk14)gcugauUfuGfcCfugaacaagaas(invAb) | 1420 | GCUGAUUUGCCUGAACAAGAA | 1632 |
| AM12168-SS | (TriAlk14)gcugauUfuGfcCfuga_2Nacaagaas(invAb) | 1421 | GCUGAUUUGCCUG(A^{2N})ACAAGAA | 1699 |
| AM12170-SS | (TriAlk14)ggcugaUfuUfgCfcugaacaagas(invAb) | 1422 | GGCUGAUUUGCCUGAACAAGA | 1670 |
| AM12174-SS | (TriAlk14)ggaccaGfcAfuCfuucaucaacas(invAb) | 1423 | GGACCAGCAUCUUCAUCAACA | 1631 |
| AM12179-SS | (TriAlk14)csgagaucaUfCfUfuca_2Nacaacaas(invAb) | 1424 | CGAGAUCAUCUUC(A^{2N})ACAACAA | 1700 |
| AM12188-SS | (TriAlk14)caucuuugAfUfGfaggacaagaus(invAb) | 1425 | CAUCUUUGAUGAGGACAAGAU | 1701 |
| AM12190-SS | (TriAlk14)gcuuugauGfAfGfgacaagaugas(invAb) | 1426 | GCUUUGAUGAGGACAAGAUGA | 1702 |
| AM12192-SS | (TriAlk14)gucucugcUfCfCfuacaacuagas(invAb) | 1427 | GUCUCUGCUCCUACAACUAGA | 1703 |
| AM12194-SS | (TriAlk14)caaccucuGfCfUfucuacaacuas(invAb) | 1428 | CAACCUCUGCUUCUACAACUA | 1704 |
| AM12196-SS | (TriAlk14)cua_2NuaaccAfGfCfacaacuucuas(invAb) | 1429 | CU(A^{2N})UAACCAGCACAACUUCUA | 1705 |
| AM12198-SS | (TriAlk14)cuauaaccAfGfCfacaacuucuas(invAb) | 1430 | CUAUAACCAGCACAACUUCUA | 1706 |
| AM12515-SS | (TriAlk14)gcuguucuGfcGfaCfuacuacaas(invAb) | 1431 | GCUGUUCUGCGACUACUACAA | 1617 |
| AM12517-SS | (TriAlk14)gcuguuCfuGfcGfacuacuacaas(invAb) | 1432 | GCUGUUCUGCGACUACUACAA | 1617 |
| AM12518-SS | (TriAlk14)gcuguucuGfcGfacuacuacaas(invAb) | 1433 | GCUGUUCUGCGACUACUACAA | 1617 |
| AM12520-SS | (TriAlk14)gcuguuuuGfcGfacuacuacaas(invAb) | 1434 | GCUGUUUUGCGACUACUACAA | 1707 |
| AM12521-SS | (TriAlk14)gcuguucuGfcGfauuacuacaas(invAb) | 1435 | GCUGUUCUGCGAUUACUACAA | 1708 |
| AM12522-SS | (TriAlk14)gcuguucuGfcGfacuauuacaas(invAb) | 1436 | GCUGUUCUGCGACUAUUACAA | 1709 |
| AM12523-SS | (TriAlk14)gcuguucuGfcGfAfcuacuacaas(invAb) | 1437 | GCUGUUCUGCGACUACUACAA | 1617 |
| AM12605-SS | (TriAlk14)gcugauuuGfcCfugaacaagaas(invAb) | 1438 | GCUGAUUUGCCUGAACAAGAA | 1632 |
| AM12606-SS | (TriAlk14)gcugauuuGfcCfuGfaacaagaas(invAb) | 1439 | GCUGAUUUGCCUGAACAAGAA | 1632 |
| AM12607-SS | (TriAlk14)ccugauUfuGfcCfugaacaagaas(invAb) | 1440 | CCUGAUUUGCCUGAACAAGAA | 1710 |
| AM13074-SS | (TriAlk14)gscugauUfuGfcCfugaacaagaas(invAb) | 1441 | GCUGAUUUGCCUGAACAAGAA | 1632 |
| AM14080-SS | (TriAlk14)gscuguucuGfCfGfacuacuacaa(invAb) | 1442 | GCUGUUCUGCGACUACUACAA | 1617 |
| AM14081-SS | (TriAlk14)gscugguucuGfCfGfacuacuacaas(invAb) | 1443 | GCUGGUUCUGCGACUACUACAA | 1711 |
| AM14084-SS | (TriAlk14)gscguucuGfCfGfacuacuacaas(invAb) | 1444 | GCGUUCUGCGACUACUACAA | 1712 |

(A^{2N}) = 2-aminoadenine-containing nucleotide; I = hypoxanthine (inosine) nucleotide

TABLE 6
Nucleotide Sequences With End Caps Shown For Certain
MUC5AC RNAi Agents Tested In Vitro.

| | | | Underlying Base | |
|---|---|---|---|---|
| | | | Sequence (5′→3′) | |
| | | SEQ | (Shown as | SEQ |
| | | ID | an Unmodified | ID |
| Strand ID | Modified Sense Strand (5′ → 3′) | NO. | Nucleotide Sequence) | NO. |
| AM10594-SS-S | (invAb)sgcuguucuGfCfGfacuacuacaas(invAb) | 1445 | GCUGUUCUGCGACUACUACAA | 1617 |
| AM10600-SS-S | (invAb)sgagugguuCfGfAfcgugiacuuas(invAb) | 1446 | GAGUGGUUCGACGUGIACUUA | 1620 |
| AM10765-SS-S | (invAb)sggaccagcAfUfCfuucaucaacas(invAb) | 1447 | GGACCAGCAUCUUCAUCAACA | 1631 |
| AM10767-SS-S | (invAb)sgcugauuuGfCfCfugaacaagaas(invAb) | 1448 | GCUGAUUUGCCUGAACAAGAA | 1632 |
| AM10769-SS-S | (invAb)sca_2NaggaaaCfCfUfacaacaacaus(invAb) | 1449 | C(A^{2N})AGGAAACCUACAACAACAU | 1633 |
| AM10771-SS-S | (invAb)sggaaaccuAfCfAfacaacaucaus(invAb) | 1450 | GGAAACCUACAACAACAUCAU | 1634 |
| AM10791-SS-S | (invAb)sgcugcuacAfAfCfuaccaiaucas(invAb) | 1451 | GCUGCUACAACUACCAIAUCA | 1636 |
| AM10793-SS-S | (invAb)sgcuacaacUfAfCfcagaucaigas(invAb) | 1452 | GCUACAACUACCAGAUCAIGA | 1637 |
| AM10795-SS-S | (invAb)scccacccaUfCfUfgcuacaacuas(invAb) | 1453 | CCCACCCAUCUGCUACAACUA | 1638 |
| AM10797-SS-S | (invAb)sgagauccgCfAfUfccaguiuugas(invAb) | 1454 | GAGAUCCGCAUCCAGUIUUGA | 1639 |
| AM10799-SS-S | (invAb)sccagagguGfAfGfcauciaacaas(invAb) | 1455 | CCAGAGGUGAGCAUCIAACAA | 1640 |
| AM10801-SS-S | (invAb)scagcagggAfCfCfcuucaagauas(invAb) | 1456 | CAGCAGGGACCCUUCAAGAUA | 1641 |
| AM10803-SS-S | (invAb)sca_2NagauguGfCfCfucaacuacias(invAb) | 1457 | C(A^{2N})AGAUGUGCCUCAACUACIA | 1642 |
| AM10813-SS-S | (invAb)sca_2NacgucaCfCfAfucuucaiacas(invAb) | 1458 | C(A^{2N})ACGUCACCAUCUUCAIACA | 1646 |
| AM10820-SS-S | (invAb)sga_2NaccuacUfAfCfucgaacuicas(invAb) | 1459 | G(A^{2N})ACCUACUACUCGAACUICA | 1648 |
| AM10826-SS-S | (invAb)sca_2NuguggaAfCfCfacgauiacaas(invAb) | 1460 | C(A^{2N})UGUGGAACCACGAUIACAA | 1651 |
| AM10828-SS-S | (invAb)saggcaagaCfCfUfcugcuucuius(invAb) | 1461 | AGGCAAGACCUCUGCUUCUIU | 1652 |
| AM10832-SS-S | (invAb)sgcagugccUfUfCfacuguacuias(invAb) | 1462 | GCAGUGCCUUCACUGUACUIA | 1654 |
| AM10836-SS-S | (invAb)sgugggaacUfUfCfaacaicaucas(invAb) | 1463 | GUGGGAACUUCAACAICAUCA | 1656 |
| AM10840-SS-S | (invAb)sccccaacaUfCfAfggaacaicuus(invAb) | 1464 | CCCCAACAUCAGGAACAICUU | 1658 |
| AM10842-SS-S | (invAb)sccuacuacUfCfGfaacuicaugus(invAb) | 1465 | CCUACUACUCGAACUICAUGU | 1659 |
| AM10844-SS-S | (invAb)sgucaccugCfAfGfuguugicuuas(invAb) | 1466 | GUCACCUGCAGUGUUGICUUA | 1660 |
| AM10846-SS-S | (invAb)sgcuggacaUfGfAfccuguuacaas(invAb) | 1467 | GCUGGACAUGACCUGUUACAA | 1661 |
| AM10848-SS-S | (invAb)sgcagagcuAfCfAfgcuucaacias(invAb) | 1468 | GCAGAGCUACAGCUUCAACIA | 1662 |
| AM11274-SS-S | (invAb)sagcagugcCfUfUfcacuguacuas(invAb) | 1469 | AGCAGUGCCUUCACUGUACUA | 1668 |
| AM11276-SS-S | (invAb)sgacaucagGfAfAfcagcuuciaas(invAb) | 1470 | GACAUCAGGAACAGCUUCIAA | 1669 |
| AM11278-SS-S | (invAb)sggcugauuUfGfCfcugaacaagas(invAb) | 1471 | GGCUGAUUUGCCUGAACAAGA | 1670 |
| AM11280-SS-S | (invAb)sccaggacaAfCfCfacuuuigugas(invAb) | 1472 | CCAGGACAACCACUUUIGUGA | 1671 |
| AM11286-SS-S | (invAb)sccacaaccAfGfCfacaacuucuas(invAb) | 1473 | CCACAACCAGCACAACUUCUA | 1674 |
| AM11288-SS-S | (invAb)sgacaaccaCfUfUfuggugacaaas(invAb) | 1474 | GACAACCACUUUGGUGACAAA | 1675 |
| AM11292-SS-S | (invAb)saccucugcUfCfCfuacaacuagas(invAb) | 1475 | ACCUCUGCUCCUACAACUAGA | 1677 |
| AM11978-SS-S | (invAb)sggacgucaUfCfUfaccacaciaas(invAb) | 1476 | GGACGUCAUCUACCACACIAA | 1695 |
| AM11980-SS-S | (invAb)scugcuacaAfCfUfaugaiauccas(invAb) | 1477 | CUGCUACAACUAUGAIAUCCA | 1664 |
| AM11983-SS-S | (invAb)sacgagaucAfUfCfuucaacaacas(invAb) | 1478 | ACGAGAUCAUCUUCAACAACA | 1697 |
| AM11985-SS-S | (invAb)scgagaucaUfCfUfucaacaacaas(invAb) | 1479 | CGAGAUCAUCUUCAACAACAA | 1667 |
| AM12164-SS-S | (invAb)sgcugauUfuGfcCfugaacaagaas(invAb) | 1480 | GCUGAUUUGCCUGAACAAGAA | 1632 |
| AM12168-SS-S | (invAb)sgcugauUfuGfcCfuga_2Nacaagaas(invAb) | 1481 | GCUGAUUUGCCUG(A^{2N})ACAAGAA | 1699 |
| AM12170-SS-S | (invAb)sggcugaUfuUfgCfcugaacaagas(invAb) | 1482 | GGCUGAUUUGCCUGAACAAGA | 1670 |

(A^{2N}) = 2-aminoadenine-containing nucleotide; I = hypoxanthine (inosine) nucleotide

TABLE 7
MUC5AC Agent Sense Strand Sequences (Shown with Targeting Ligand Conjugate.
The structure of αvβ6-SM6.1 is shown in Table 12, and the structure of
Tri-SM6.1- αvβ6-(TA14) is shown in FIG. 1.)

| | | | Corresponding |
|---|---|---|---|
| | | | Sense Strand |
| | | | AM Number |
| | | | Without |
| | | SEQ | Linker or |
| Strand | | ID | Conjugate |
| ID | Modified Sense Strand (5′→3′) | NO. | (See Table 4) |
| CS000387 | Tri-SM6.1-avb6-(TA14)gsa_2NaguaugCfUfCfaguacugiuas(invAb) | 1483 | AM10745-SS |
| CS000517 | Tri-SM6.1-avb6-(TA14)ascccauguGfCfUfacaacuaugas(invAb) | 1484 | AM09492-SS |
| CS000519 | Tri-SM6.1-avb6-(TA14)cscauacagCfAfGfuacaguuacas(invAb) | 1485 | AM09657-SS |
| CS000521 | Tri-SM6.1-avb6-(TA14)csugcuacaAfCfUfaugagauccas(invAb) | 1486 | AM11263-SS |
| CS000523 | Tri-SM6.1-avb6-(TA14)csugcuacaAfCfUfaugaiauccas(invAb) | 1487 | AM11265-SS |
| CS000525 | Tri-SM6.1-avb6-(TA14)cscuggaccAfAfGfugguuugacas(invAb) | 1488 | AM11267-SS |
| CS000527 | Tri-SM6.1-avb6-(TA14)cscuggaccAfAfGfugguuuiacas(invAb) | 1489 | AM11269-SS |
| CS000528 | Tri-SM6.1-avb6-(TA14)csgagaucaUfCfUfucaacaacaas(invAb) | 1490 | AM11270-SS |
| CS000578 | Tri-SM6.1-avb6-(TA14)gscuguucuGfCfGfacuacuacaas(invAb) | 1491 | AM11400-SS |
| CS000583 | Tri-SM6.1-avb6-(TA14)gscuguucuGfUfGfacuacuacaas(invAb) | 1492 | AM11402-SS |
| CS000608 | Tri-SM6.1-avb6-(TA14)asccagaucAfUfCfuucaacaacas(invAb) | 1493 | AM11463-SS |
| CS000612 | Tri-SM6.1-avb6-(TA14)gsgcucuguGfGfUfaacuucaacas(invAb) | 1494 | AM11466-SS |
| CS000614 | Tri-SM6.1-avb6-(TA14)gsagcguggAfGfAfaugaaaaguas(invAb) | 1495 | AM11468-SS |
| CS000616 | Tri-SM6.1-avb6-(TA14)gsggagaauGfAfAfaaguaugcuas(invAb) | 1496 | AM11470-SS |
| CS000618 | Tri-SM6.1-avb6-(TA14)csuucucgaAfCfUfgcauguaugas(invAb) | 1497 | AM11472-SS |
| CS000620 | Tri-SM6.1-avb6-(TA14)gscucgaacUfGfCfauguaugacas(invAb) | 1498 | AM11474-SS |
| CS000622 | Tri-SM6.1-avb6-(TA14)csucgaacuGfCfAfuguaugacaas(invAb) | 1499 | AM11476-SS |
| CS000624 | Tri-SM6.1-avb6-(TA14)cscacuguuCfUfGfugacuacuaas(invAb) | 1500 | AM11478-SS |
| CS000626 | Tri-SM6.1-avb6-(TA14)csacuguucUfGfUfgacuacuacas(invAb) | 1501 | AM11480-SS |
| CS000665 | Tri-SM6.1-avb6-(TA14)cscuucuucUfCfGfaacuicaugus(invAb) | 1502 | AM11497-SS |
| CS001001 | Tri-SM6.1-avb6-(TA14)csagcuuccAfCfUfacaaiaccuus(invAb) | 1503 | AM10738-SS |
| CS001003 | Tri-SM6.1-avb6-(TA14)csagcuuccAfCfUfacaagaccuus(invAb) | 1504 | AM10740-SS |
| CS001005 | Tri-SM6.1-avb6-(TA14)gsa_2NaguaugCfUfCfagcacugiuas(invAb) | 1505 | AM10742-SS |
| CS001007 | Tri-SM6.1-avb6-(TA14)gsccuucuuCfAfAfcaccuucaaas(invAb) | 1506 | AM10746-SS |
| CS001009 | Tri-SM6.1-avb6-(TA14)gsccuucuuCfAfAfcaucuucaaas(invAb) | 1507 | AM10748-SS |
| CSOO1O1O | Tri-SM6.1-avb6-(TA14)gsccuucuuCfAfAfcacuuucaaas(invAb) | 1508 | AM10749-SS |
| CS001036 | Tri-SM6.1-avb6-(TA14)cscacccauGfUfGfcuacaacuaus(invAb) | 1509 | AM11739-SS |
| CS001040 | Tri-SM6.1-avb6-(TA14)csugcuaCfaAfcUfaugaiauccas(invAb) | 1510 | AM11743-SS |
| CS001041 | Tri-SM6.1-avb6-(TA14)csgagauCfaUfcUfucaacaacaas(invAb) | 1511 | AM11744-SS |
| CS001401 | Tri-SM6.1-avb6-(TA14)csgagaucaUfCfUfuca_2Nacaacaas(invAb) | 1512 | AM12179-SS |
| CS001644 | Tri-SM6.1-avb6-(TA14)gscugauUfuGfcCfugaacaagaas(invAb) | 1513 | AM13074-SS |
| CS002194 | Tri-SM6.1-avb6-(TA14)gscuguucuGfCfGfacuacuacaa(invAb) | 1514 | AM14080-SS |
| CS002195 | Tri-SM6.1-avb6-(TA14)gscugguucuGfCfGfacuacuacaas(invAb) | 1515 | AM14081-SS |
| CS002196 | Tri-SM6.1-avb6-(TA14)gscguucuGfCfGfacuacuacaas(invAb) | 1516 | AM14084-SS |

The MUC5AC RNAi agents disclosed herein are formed by annealing an antisense strand with a sense strand. A sense strand containing a sequence listed in Table 2, Table 4, Table 5, Table 6, or Table 7 can be hybridized to any antisense strand containing a sequence listed in Table 2 or Table 3, provided the two sequences have a region of at least 85% complementarity over a contiguous 16, 17, 18, 19, 20, or 21 nucleotide sequence.

As shown in Table 5 above, certain of the example MUC5AC RNAi agent nucleotide sequences are shown to further include reactive linking groups at one or both of the 5′ terminal end and the 3′ terminal end of the sense strand. For example, many of the MUC5AC RNAi agent sense strand sequences shown in Table 5 above have a (TriAlk14) linking group at the 5′ end of the nucleotide sequence. Other linking groups, such as an (NH2-C6) linking group or a (6-SS-6) or (C6-SS-C6) linking group, may be present as well or alternatively in certain embodiments. Such reactive linking groups are positioned to facilitate the linking of targeting ligands, targeting groups, and/or PK/PD modulators to the MUC5AC RNAi agents disclosed herein. Linking or conjugation reactions are well known in the art and provide for formation of covalent linkages between two molecules or reactants. Suitable conjugation reactions for use in the scope of the inventions herein include, but are not limited to, amide coupling reaction, Michael addition reaction, hydrazone formation reaction, inverse-demand Diels-Alder cycloaddition reaction, oxime ligation, and Copper (I)-catalyzed or strain-promoted azide-alkyne cycloaddition reaction cycloaddition reaction.

In some embodiments, targeting ligands, such as the integrin targeting ligands shown in the examples and figures disclosed herein, can be synthesized as activated esters, such as tetrafluorophenyl (TFP) esters, which can be displaced by a reactive amino group (e.g., NH₂—C₆) to attach the targeting ligand to the MUC5AC RNAi agents disclosed herein. In some embodiments, targeting ligands are synthesized as azides, which can be conjugated to a propargyl (e.g., TriAlk14) or DBCO group, for example, via Copper (I)-catalyzed or strain-promoted azide-alkyne cycloaddition reaction.

Additionally, the nucleotide sequences can be synthesized with a dT nucleotide at the 3′ terminal end of the sense strand, followed by (3′→5′) a linker (e.g., C6-SS-C6). The linker can, in some embodiments, facilitate the linkage to additional components, such as, for example, a PK/PD modulator or one or more targeting ligands. The disulfide bond of C6-SS-C6 can then be reduced, removing the dT from the molecule, which can then facilitate the conjugation of the desired PK/PD modulator. The terminal dT nucleotide would therefore not be a part of the fully conjugated construct.

In some embodiments, the antisense strand of a MUC5AC RNAi agent disclosed herein differs by 0, 1, 2, or 3 nucleotides from any of the antisense strand sequences in Table 3 or Table 11. In some embodiments, the sense strand of a MUC5AC RNAi agent disclosed herein differs by 0, 1, 2, or 3 nucleotides from any of the sense strand sequences in Table 4, Table 5, Table 6, Table 7, or Table 11.

In some embodiments, a MUC5AC RNAi agent antisense strand comprises a nucleotide sequence of any of the sequences in Table 2 or Table 3. In some embodiments, a MUC5AC RNAi agent antisense strand comprises the sequence of nucleotides (from 5′ end→3′ end) 1-17, 2-17, 1-18, 2-18, 1-19, 2-19, 1-20, 2-20, 1-21, 2-21, 1-22, 2-22, 1-23, 2-23, 1-24, or 2-24 of any of the sequences in Table 2, Table 3, or Table 11. In certain embodiments, a MUC5AC RNAi agent antisense strand comprises or consists of a modified sequence of any one of the modified sequences in Table 3 or Table 11.

In some embodiments, a MUC5AC RNAi agent sense strand comprises the nucleotide sequence of any of the sequences in Table 2 or Table 4. In some embodiments, a MUC5AC RNAi agent sense strand comprises the sequence of nucleotides (from 5′ end→3′ end) 1-17, 2-17, 3-17, 4-17, 1-18, 2-18, 3-18, 4-18, 1-19, 2-19, 3-19, 4-19, 1-20, 2-20, 3-20, 4-20, 1-21, 2-21, 3-21, 4-21, 1-22, 2-22, 3-22, 4-22, 1-23, 2-23, 3-23, 4-23, 1-24, 2-24, 3-24, or 4-24, of any of the sequences in Table 2, Table 4, Table 5, Table 6, Table 7, or Table 11. In certain embodiments, a MUC5AC RNAi agent sense strand comprises or consists of a modified sequence of any one of the modified sequences in Table 3 or Table 11.

For the RNAi agents disclosed herein, the nucleotide at position 1 of the antisense strand (from 5′ end→3′ end) can be perfectly complementary to a MUC5AC gene, or can be non-complementary to a MUC5AC gene. In some embodiments, the nucleotide at position 1 of the antisense strand (from 5′ end→3′ end) is a U, A, or dT (or a modified version of U, A or dT). In some embodiments, the nucleotide at position 1 of the antisense strand (from 5′ end→3′ end) forms an A:U or U:A base pair with the sense strand.

In some embodiments, a MUC5AC RNAi agent antisense strand comprises the sequence of nucleotides (from 5′ end→3′ end) 2-18 or 2-19 of any of the antisense strand sequences in Table 2, Table 3, or Table 11. In some embodiments, a MUC5AC RNAi sense strand comprises the sequence of nucleotides (from 5′ end→3′ end) 1-17 or 1-18 of any of the sense strand sequences in Table 2, Table 4, Table 5, Table 6, Table 7, or Table 11.

In some embodiments, a MUC5AC RNAi agent includes (i) an antisense strand comprising the sequence of nucleotides (from 5′ end→3′ end) 2-18 or 2-19 of any of the antisense strand sequences in Table 2, Table 3, or Table 11, and (ii) a sense strand comprising the sequence of nucleotides (from 5′ end→3′ end) 1-17 or 1-18 of any of the sense strand sequences in Table 2, Table 4, Table 5, Table 6, Table 7, or Table 11.

A sense strand containing a sequence listed in Table 2 or Table 4 can be hybridized to any antisense strand containing a sequence listed in Table 2 or Table 3 provided the two sequences have a region of at least 85% complementarity over a contiguous 16, 17, 18, 19, 20, or 21 nucleotide sequence. In some embodiments, the MUC5AC RNAi agent has a sense strand consisting of the modified sequence of any of the modified sequences in Table 4, Table 5, Table 6, Table 7, or Table 11, and an antisense strand consisting of the modified sequence of any of the modified sequences in Table 3 or Table 11. Certain representative sequence pairings are exemplified by the Duplex ID Nos. shown in Tables 8A, 8B, 8C, 9, 10A and 10B.

In some embodiments, a MUC5AC RNAi agent comprises, consists of, or consists essentially of a duplex represented by any one of the Duplex ID Nos. presented herein. In some embodiments, a MUC5AC RNAi agent consists of any of the Duplex ID Nos. presented herein. In some embodiments, a MUC5AC RNAi agent comprises the sense strand and antisense strand nucleotide sequences of any of the Duplex ID Nos. presented herein. In some embodiments, a MUC5AC RNAi agent comprises the sense strand and antisense strand nucleotide sequences of any of the Duplex ID Nos. presented herein and a targeting group, linking group, and/or other non-nucleotide group wherein the targeting group, linking group, and/or other non-nucleotide group is covalently linked (i.e., conjugated) to the sense strand or the antisense strand. In some embodiments, a MUC5AC RNAi agent includes the sense strand and antisense strand modified nucleotide sequences of any of the Duplex ID Nos. presented herein. In some embodiments, a MUC5AC RNAi agent comprises the sense strand and antisense strand modified nucleotide sequences of any of the Duplex ID Nos. presented herein and a targeting group, linking group, and/or other non-nucleotide group, wherein the targeting group, linking group, and/or other non-nucleotide group is covalently linked to the sense strand or the antisense strand.

In some embodiments, a MUC5AC RNAi agent comprises an antisense strand and a sense strand having the nucleotide sequences of any of the antisense strand/sense strand duplexes of Tables 2, 8A, 8B, 8C, 9, 10A, 10B, or 11, and comprises a targeting group. In some embodiments, a MUC5AC RNAi agent comprises an antisense strand and a sense strand having the nucleotide sequences of any of the antisense strand/sense strand duplexes of Tables 2, 8A, 8B, 8C, 9, 10A, 10B, or 11, and comprises one or more αvβ6 integrin targeting ligands.

In some embodiments, a MUC5AC RNAi agent comprises an antisense strand and a sense strand having the nucleotide sequences of any of the antisense strand/sense strand duplexes of Tables 2, 8A, 8B, 8C, 9, 10A, 10B, or 11, and comprises a targeting group that is an integrin targeting ligand. In some embodiments, a MUC5AC RNAi agent comprises an antisense strand and a sense strand having the nucleotide sequences of any of the antisense strand/sense strand duplexes of Tables 2, 8A, 8B, 8C, 9, 10A, 10B, or 11, and comprises one or more αvβ6 integrin targeting ligands or clusters of αvβ6 integrin targeting ligands (e.g., a tridentate αvβ6 integrin targeting ligand).

In some embodiments, a MUC5AC RNAi agent comprises an antisense strand and a sense strand having the modified nucleotide sequences of any of the antisense strand/sense strand duplexes of Tables 8A, 8B, 8C, 9, 10A, 10B, and 11.

In some embodiments, a MUC5AC RNAi agent comprises an antisense strand and a sense strand having the modified nucleotide sequences of any of the antisense strand/sense strand duplexes of Tables 8A, 8B, 8C, 9, 10A, 10B, and 11, and comprises an integrin targeting ligand.

In some embodiments, a MUC5AC RNAi agent comprises, consists of, or consists essentially of any of the duplexes of Tables 8A, 8B, 8C, 9, 10A, 10B, and 11.

TABLE 8A
MUC5AC RNAi Agent Duplexes with Corresponding Sense and Antisense Strand ID Numbers
and Sequence ID numbers for the modified and unmodified nucleotide sequences.
(Shown without Linking Agents or Conjugates)

| | | AS | AS | | SS | SS |
|---|---|---|---|---|---|---|
| | | modified | unmodified | | modified | unmodified |
| Duplex | AS ID | SEQ ID NO: | SEQ ID NO: | SS ID | SEQ ID NO: | SEQ ID NO: |
| AD07626 | AM10579-AS | 1057 | 1517 | AM10578-SS-NL | 1192 | 1609 |
| AD07627 | AM10581-AS | 1058 | 1518 | AM10580-SS-NL | 1193 | 1610 |
| AD07628 | AM10583-AS | 1059 | 1519 | AM10582-SS-NL | 1194 | 1611 |
| AD07629 | AM10585-AS | 1060 | 1520 | AM10584-SS-NL | 1195 | 1612 |
| AD07630 | AM10587-AS | 1061 | 1521 | AM10586-SS-NL | 1196 | 1613 |
| AD07631 | AM10589-AS | 1062 | 1522 | AM10588-SS-NL | 1197 | 1614 |
| AD07632 | AM10591-AS | 1063 | 1523 | AM10590-SS-NL | 1198 | 1615 |
| AD07633 | AM10593-AS | 1064 | 1524 | AM10592-SS-NL | 1199 | 1616 |
| AD07634 | AM10595-AS | 1065 | 1525 | AM10594-SS-NL | 1200 | 1617 |
| AD07635 | AM10597-AS | 1066 | 1526 | AM10596-SS-NL | 1201 | 1618 |
| AD07636 | AM10599-AS | 1067 | 1527 | AM10598-SS-NL | 1202 | 1619 |
| AD07637 | AM10601-AS | 1068 | 1528 | AM10600-SS-NL | 1203 | 1620 |
| AD07638 | AM10603-AS | 1069 | 1529 | AM10602-SS-NL | 1204 | 1621 |
| AD07639 | AM10605-AS | 1070 | 1529 | AM10604-SS-NL | 1205 | 1622 |
| AD07716 | AM10739-AS | 1071 | 1530 | AM10738-SS-NL | 1206 | 1623 |
| AD07717 | AM10741-AS | 1072 | 1530 | AM10740-SS-NL | 1207 | 1624 |
| AD07718 | AM10743-AS | 1073 | 1531 | AM10742-SS-NL | 1208 | 1625 |
| AD07719 | AM10744-AS | 1074 | 1531 | AM10742-SS-NL | 1208 | 1625 |
| AD07720 | AM10743-AS | 1073 | 1531 | AM10745-SS-NL | 1209 | 1626 |
| AD07721 | AM10747-AS | 1075 | 1532 | AM10746-SS-NL | 1210 | 1627 |
| AD07722 | AM10747-AS | 1075 | 1532 | AM10748-SS-NL | 1211 | 1628 |
| AD07723 | AM10747-AS | 1075 | 1532 | AM10749-SS-NL | 1212 | 1629 |
| AD07731 | AM10764-AS | 1076 | 1533 | AM10763-SS-NL | 1213 | 1630 |
| AD07732 | AM10766-AS | 1077 | 1534 | AM10765-SS-NL | 1214 | 1631 |
| AD07733 | AM10768-AS | 1078 | 1535 | AM10767-SS-NL | 1215 | 1632 |
| AD07734 | AM10770-AS | 1079 | 1536 | AM10769-SS-NL | 1216 | 1633 |
| AD07735 | AM10772-AS | 1080 | 1537 | AM10771-SS-NL | 1217 | 1634 |
| AD07744 | AM10790-AS | 1081 | 1538 | AM10789-SS-NL | 1218 | 1635 |
| AD07745 | AM10792-AS | 1082 | 1539 | AM10791-SS-NL | 1219 | 1636 |
| AD07746 | AM10794-AS | 1083 | 1540 | AM10793-SS-NL | 1220 | 1637 |
| AD07747 | AM10796-AS | 1084 | 1541 | AM10795-SS-NL | 1221 | 1638 |
| AD07748 | AM10798-AS | 1085 | 1542 | AM10797-SS-NL | 1222 | 1639 |
| AD07749 | AM10800-AS | 1086 | 1543 | AM10799-SS-NL | 1223 | 1640 |
| AD07750 | AM10802-AS | 1087 | 1544 | AM10801-SS-NL | 1224 | 1641 |
| AD07751 | AM10804-AS | 1088 | 1545 | AM10803-SS-NL | 1225 | 1642 |
| AD07752 | AM10806-AS | 1089 | 1546 | AM10805-SS-NL | 1226 | 1643 |
| AD07753 | AM10808-AS | 1090 | 1530 | AM10807-SS-NL | 1227 | 1623 |
| AD07754 | AM10810-AS | 1091 | 1547 | AM10809-SS-NL | 1228 | 1644 |
| AD07755 | AM10812-AS | 1092 | 1548 | AM10811-SS-NL | 1229 | 1645 |
| AD07756 | AM10814-AS | 1093 | 1549 | AM10813-SS-NL | 1230 | 1646 |
| AD07757 | AM10816-AS | 1094 | 1550 | AM10815-SS-NL | 1231 | 1647 |
| AD07758 | AM10818-AS | 1095 | 1532 | AM10817-SS-NL | 1232 | 1627 |
| AD07760 | AM10821-AS | 1096 | 1551 | AM10820-SS-NL | 1234 | 1648 |
| AD07761 | AM10823-AS | 1097 | 1552 | AM10822-SS-NL | 1235 | 1649 |
| AD07762 | AM10825-AS | 1098 | 1553 | AM10824-SS-NL | 1236 | 1650 |
| AD07763 | AM10827-AS | 1099 | 1554 | AM10826-SS-NL | 1237 | 1651 |
| AD07764 | AM10829-AS | 1100 | 1555 | AM10828-SS-NL | 1238 | 1652 |
| AD07765 | AM10831-AS | 1101 | 1556 | AM10830-SS-NL | 1239 | 1653 |
| AD07766 | AM10833-AS | 1102 | 1557 | AM10832-SS-NL | 1240 | 1654 |
| AD07767 | AM10835-AS | 1103 | 1558 | AM10834-SS-NL | 1241 | 1655 |
| AD07768 | AM10837-AS | 1104 | 1559 | AM10836-SS-NL | 1242 | 1656 |
| AD07769 | AM10839-AS | 1105 | 1560 | AM10838-SS-NL | 1243 | 1657 |
| AD07770 | AM10841-AS | 1106 | 1561 | AM10840-SS-NL | 1244 | 1658 |
| AD07771 | AM10843-AS | 1107 | 1562 | AM10842-SS-NL | 1245 | 1659 |
| AD07772 | AM10845-AS | 1108 | 1563 | AM10844-SS-NL | 1246 | 1660 |
| AD07773 | AM10847-AS | 1109 | 1564 | AM10846-SS-NL | 1247 | 1661 |
| AD07774 | AM10849-AS | 1110 | 1565 | AM10848-SS-NL | 1248 | 1662 |
| AD07941 | AM11065-AS | 1111 | 1531 | AM10819-SS-NL | 1233 | 1625 |
| AD08083 | AM11264-AS | 1112 | 1566 | AM11263-SS-NL | 1250 | 1663 |
| AD08084 | AM11264-AS | 1112 | 1566 | AM11265-SS-NL | 1251 | 1664 |
| AD08085 | AM11266-AS | 1113 | 1566 | AM11263-SS-NL | 1250 | 1663 |
| AD08086 | AM11268-AS | 1114 | 1567 | AM11267-SS-NL | 1252 | 1665 |
| AD08087 | AM11268-AS | 1114 | 1567 | AM11269-SS-NL | 1253 | 1666 |
| AD08088 | AM11271-AS | 1115 | 1568 | AM11270-SS-NL | 1254 | 1667 |
| AD08089 | AM11272-AS | 1116 | 1568 | AM11270-SS-NL | 1254 | 1667 |
| AD08094 | AM11275-AS | 1117 | 1569 | AM11274-SS-NL | 1255 | 1668 |
| AD08095 | AM11277-AS | 1118 | 1570 | AM11276-SS-NL | 1256 | 1669 |
| AD08096 | AM11279-AS | 1119 | 1571 | AM11278-SS-NL | 1257 | 1670 |
| AD08097 | AM11281-AS | 1120 | 1572 | AM11280-SS-NL | 1258 | 1671 |
| AD08098 | AM11283-AS | 1121 | 1573 | AM11282-SS-NL | 1259 | 1672 |
| AD08099 | AM11285-AS | 1122 | 1574 | AM11284-SS-NL | 1260 | 1673 |
| AD08100 | AM11287-AS | 1123 | 1575 | AM11286-SS-NL | 1261 | 1674 |
| AD08101 | AM11289-AS | 1124 | 1576 | AM11288-SS-NL | 1262 | 1675 |
| AD08102 | AM11291-AS | 1125 | 1577 | AM11290-SS-NL | 1263 | 1676 |
| AD08103 | AM11293-AS | 1126 | 1578 | AM11292-SS-NL | 1264 | 1677 |
| AD08173 | AM10595-AS | 1065 | 1525 | AM11400-SS-NL | 1265 | 1617 |
| AD08174 | AM11401-AS | 1127 | 1525 | AM11400-SS-NL | 1265 | 1617 |
| AD08175 | AM11403-AS | 1128 | 1579 | AM11402-SS-NL | 1266 | 1678 |
| AD08176 | AM11404-AS | 1129 | 1579 | AM11402-SS-NL | 1266 | 1678 |
| AD08177 | AM11405-AS | 1130 | 1579 | AM11402-SS-NL | 1266 | 1678 |
| AD08224 | AM11464-AS | 1132 | 1581 | AM11463-SS-NL | 1267 | 1679 |
| AD08225 | AM11465-AS | 1133 | 1581 | AM11463-SS-NL | 1267 | 1679 |
| AD08226 | AM11467-AS | 1134 | 1582 | AM11466-SS-NL | 1268 | 1680 |
| AD08227 | AM11469-AS | 1135 | 1583 | AM11468-SS-NL | 1269 | 1681 |
| AD08228 | AM11471-AS | 1136 | 1584 | AM11470-SS-NL | 1270 | 1682 |
| AD08229 | AM11473-AS | 1137 | 1585 | AM11472-SS-NL | 1271 | 1683 |
| AD08230 | AM11475-AS | 1138 | 1586 | AM11474-SS-NL | 1272 | 1684 |
| AD08231 | AM11477-AS | 1139 | 1587 | AM11476-SS-NL | 1273 | 1685 |
| AD08232 | AM11479-AS | 1140 | 1588 | AM11478-SS-NL | 1274 | 1686 |
| AD08233 | AM11481-AS | 1141 | 1589 | AM11480-SS-NL | 1275 | 1687 |
| AD08243 | AM11495-AS | 1142 | 1590 | AM11400-SS-NL | 1265 | 1617 |
| AD08244 | AM11496-AS | 1143 | 1525 | AM11400-SS-NL | 1265 | 1617 |
| AD08245 | AM11498-AS | 1144 | 1591 | AM11497-SS-NL | 1276 | 1688 |
| AD08246 | AM11499-AS | 1145 | 1591 | AM11497-SS-NL | 1276 | 1688 |
| AD08420 | AM11742-AS | 1148 | 1566 | AM11265-SS-NL | 1251 | 1664 |
| AD08421 | AM11742-AS | 1148 | 1566 | AM11263-SS-NL | 1250 | 1663 |
| AD08422 | AM11742-AS | 1148 | 1566 | AM11743-SS-NL | 1278 | 1664 |
| AD08423 | AM11272-AS | 1116 | 1568 | AM11744-SS-NL | 1279 | 1667 |
| AD08424 | AM11745-AS | 1149 | 1568 | AM11744-SS-NL | 1279 | 1667 |
| AD08468 | AM11821-AS | 1150 | 1589 | AM11480-SS-NL | 1275 | 1687 |
| AD08469 | AM11823-AS | 1151 | 1589 | AM11822-SS-NL | 1280 | 1687 |
| AD08470 | AM11825-AS | 1152 | 1593 | AM11824-SS-NL | 1281 | 1690 |
| AD08564 | AM11971-AS | 1153 | 1594 | AM11970-SS-NL | 1282 | 1691 |
| AD08565 | AM11973-AS | 1154 | 1595 | AM11972-SS-NL | 1283 | 1692 |
| AD08566 | AM11975-AS | 1155 | 1596 | AM11974-SS-NL | 1284 | 1693 |
| AD08567 | AM11977-AS | 1156 | 1597 | AM11976-SS-NL | 1285 | 1694 |
| AD08568 | AM11979-AS | 1157 | 1598 | AM11978-SS-NL | 1286 | 1695 |
| AD08569 | AM07100-AS | 1716 | 1566 | AM11980-SS-NL | 1287 | 1664 |
| AD08570 | AM11982-AS | 1158 | 1599 | AM11981-SS-NL | 1288 | 1696 |
| AD08571 | AM11984-AS | 1159 | 1600 | AM11983-SS-NL | 1289 | 1697 |
| AD08572 | AM07104-AS | 1717 | 1568 | AM11985-SS-NL | 1290 | 1667 |
| AD08573 | AM11986-AS | 1160 | 1568 | AM11985-SS-NL | 1290 | 1667 |
| AD08662 | AM12158-AS | 1161 | 1601 | AM12157-SS-NL | 1291 | 1698 |
| AD08663 | AM12159-AS | 1162 | 1601 | AM12157-SS-NL | 1291 | 1698 |
| AD08664 | AM12161-AS | 1163 | 1601 | AM12160-SS-NL | 1292 | 1698 |
| AD08665 | AM12162-AS | 1164 | 1601 | AM12160-SS-NL | 1292 | 1698 |
| AD08666 | AM12163-AS | 1165 | 1535 | AM10767-SS-NL | 1215 | 1632 |
| AD08667 | AM12165-AS | 1166 | 1535 | AM12164-SS-NL | 1293 | 1632 |
| AD08668 | AM12166-AS | 1167 | 1535 | AM12164-SS-NL | 1293 | 1632 |
| AD08669 | AM12167-AS | 1168 | 1535 | AM12164-SS-NL | 1293 | 1632 |
| AD08670 | AM12167-AS | 1168 | 1535 | AM12168-SS-NL | 1294 | 1699 |
| AD08671 | AM12169-AS | 1169 | 1571 | AM11278-SS-NL | 1257 | 1670 |
| AD08672 | AM12171-AS | 1170 | 1571 | AM12170-SS-NL | 1295 | 1670 |
| AD08673 | AM12172-AS | 1171 | 1571 | AM12170-SS-NL | 1295 | 1670 |
| AD08674 | AM12173-AS | 1172 | 1534 | AM10765-SS-NL | 1214 | 1631 |
| AD08675 | AM12175-AS | 1173 | 1534 | AM12174-SS-NL | 1296 | 1631 |
| AD08676 | AM12176-AS | 1174 | 1534 | AM12174-SS-NL | 1296 | 1631 |
| AD08677 | AM12177-AS | 1175 | 1534 | AM12174-SS-NL | 1296 | 1631 |
| AD08678 | AM12178-AS | 1176 | 1568 | AM11270-SS-NL | 1254 | 1667 |
| AD08679 | AM12178-AS | 1176 | 1568 | AM12179-SS-NL | 1297 | 1700 |
| AD08680 | AM12180-AS | 1177 | 1568 | AM11270-SS-NL | 1254 | 1667 |
| AD08681 | AM12181-AS | 1178 | 1568 | AM11270-SS-NL | 1254 | 1667 |
| AD08682 | AM12182-AS | 1179 | 1602 | AM11270-SS-NL | 1254 | 1667 |
| AD08687 | AM12189-AS | 1180 | 1603 | AM12188-SS-NL | 1298 | 1701 |
| AD08688 | AM12191-AS | 1181 | 1604 | AM12190-SS-NL | 1299 | 1702 |
| AD08689 | AM12193-AS | 1182 | 1605 | AM12192-SS-NL | 1300 | 1703 |
| AD08690 | AM12195-AS | 1183 | 1606 | AM12194-SS-NL | 1301 | 1704 |
| AD08691 | AM12197-AS | 1184 | 1607 | AM12196-SS-NL | 1302 | 1705 |
| AD08692 | AM12197-AS | 1184 | 1607 | AM12198-SS-NL | 1303 | 1706 |
| AD08889 | AM11401-AS | 1127 | 1525 | AM10594-SS-NL | 1200 | 1617 |
| AD08890 | AM12516-AS | 1185 | 1525 | AM12515-SS-NL | 1304 | 1617 |
| AD08891 | AM12516-AS | 1185 | 1525 | AM12517-SS-NL | 1305 | 1617 |
| AD08892 | AM12516-AS | 1185 | 1525 | AM12518-SS-NL | 1306 | 1617 |
| AD08893 | AM12519-AS | 1186 | 1525 | AM12518-SS-NL | 1306 | 1617 |
| AD08894 | AM12516-AS | 1185 | 1525 | AM12520-SS-NL | 1307 | 1707 |
| AD08895 | AM12516-AS | 1185 | 1525 | AM12521-SS-NL | 1308 | 1708 |
| AD08896 | AM12516-AS | 1185 | 1525 | AM12522-SS-NL | 1309 | 1709 |
| AD08897 | AM12516-AS | 1185 | 1525 | AM12523-SS-NL | 1310 | 1617 |
| AD08951 | AM12165-AS | 1166 | 1535 | AM12605-SS-NL | 1311 | 1632 |
| AD08952 | AM12165-AS | 1166 | 1535 | AM12606-SS-NL | 1312 | 1632 |
| AD08953 | AM12608-AS | 1187 | 1608 | AM12607-SS-NL | 1313 | 1710 |
| AD08954 | AM12609-AS | 1188 | 1535 | AM12164-SS-NL | 1293 | 1632 |
| AD08955 | AM12610-AS | 1189 | 1535 | AM12164-SS-NL | 1293 | 1632 |
| AD08956 | AM12611-AS | 1190 | 1535 | AM12164-SS-NL | 1293 | 1632 |
| AD08957 | AM12612-AS | 1191 | 1535 | AM12164-SS-NL | 1293 | 1632 |
| AD09240 | AM12165-AS | 1166 | 1535 | AM13074-SS-NL | 1315 | 1632 |
| AD09241 | AM12612-AS | 1191 | 1535 | AM13074-SS-NL | 1315 | 1632 |
| AD09863 | AM11401-AS | 1127 | 1525 | AM14080-SS-NL | 1316 | 1617 |
| AD09864 | AM11401-AS | 1127 | 1525 | AM14081-SS-NL | 1317 | 1711 |
| AD09865 | AM11401-AS | 1127 | 1525 | AM14084-SS-NL | 1318 | 1712 |

TABLE 8B
MUC5AC RNAi Agent Duplexes with Corresponding Sense and Antisense Strand ID Numbers
and Sequence ID numbers for the modified and unmodified nucleotide sequences.)

| | | AS | AS | | SS | SS |
|---|---|---|---|---|---|---|
| | | modified | unmodified | | modified | unmodified |
| Duplex | AS ID | SEQ ID NO: | SEQ ID NO: | SS ID | SEQ ID NO: | SEQ ID NO: |
| AD07626 | AM10579-AS | 1057 | 1517 | AM10578-SS | 1319 | 1609 |
| AD07627 | AM10581-AS | 1058 | 1518 | AM10580-SS | 1320 | 1610 |
| AD07628 | AM10583-AS | 1059 | 1519 | AM10582-SS | 1321 | 1611 |
| AD07629 | AM10585-AS | 1060 | 1520 | AM10584-SS | 1322 | 1612 |
| AD07630 | AM10587-AS | 1061 | 1521 | AM10586-SS | 1323 | 1613 |
| AD07631 | AM10589-AS | 1062 | 1522 | AM10588-SS | 1324 | 1614 |
| AD07632 | AM10591-AS | 1063 | 1523 | AM10590-SS | 1325 | 1615 |
| AD07633 | AM10593-AS | 1064 | 1524 | AM10592-SS | 1326 | 1616 |
| AD07634 | AM10595-AS | 1065 | 1525 | AM10594-SS | 1327 | 1617 |
| AD07635 | AM10597-AS | 1066 | 1526 | AM10596-SS | 1328 | 1618 |
| AD07636 | AM10599-AS | 1067 | 1527 | AM10598-SS | 1329 | 1619 |
| AD07637 | AM10601-AS | 1068 | 1528 | AM10600-SS | 1330 | 1620 |
| AD07638 | AM10603-AS | 1069 | 1529 | AM10602-SS | 1331 | 1621 |
| AD07639 | AM10605-AS | 1070 | 1529 | AM10604-SS | 1332 | 1622 |
| AD07716 | AM10739-AS | 1071 | 1530 | AM10738-SS | 1333 | 1623 |
| AD07717 | AM10741-AS | 1072 | 1530 | AM10740-SS | 1334 | 1624 |
| AD07718 | AM10743-AS | 1073 | 1531 | AM10742-SS | 1335 | 1625 |
| AD07719 | AM10744-AS | 1074 | 1531 | AM10742-SS | 1335 | 1625 |
| AD07720 | AM10743-AS | 1073 | 1531 | AM10745-SS | 1336 | 1626 |
| AD07721 | AM10747-AS | 1075 | 1532 | AM10746-SS | 1337 | 1627 |
| AD07722 | AM10747-AS | 1075 | 1532 | AM10748-SS | 1338 | 1628 |
| AD07723 | AM10747-AS | 1075 | 1532 | AM10749-SS | 1339 | 1629 |
| AD07731 | AM10764-AS | 1076 | 1533 | AM10763-SS | 1340 | 1630 |
| AD07732 | AM10766-AS | 1077 | 1534 | AM10765-SS | 1341 | 1631 |
| AD07733 | AM10768-AS | 1078 | 1535 | AM10767-SS | 1342 | 1632 |
| AD07734 | AM10770-AS | 1079 | 1536 | AM10769-SS | 1343 | 1633 |
| AD07735 | AM10772-AS | 1080 | 1537 | AM10771-SS | 1344 | 1634 |
| AD07744 | AM10790-AS | 1081 | 1538 | AM10789-SS | 1345 | 1635 |
| AD07745 | AM10792-AS | 1082 | 1539 | AM10791-SS | 1346 | 1636 |
| AD07746 | AM10794-AS | 1083 | 1540 | AM10793-SS | 1347 | 1637 |
| AD07747 | AM10796-AS | 1084 | 1541 | AM10795-SS | 1348 | 1638 |
| AD07748 | AM10798-AS | 1085 | 1542 | AM10797-SS | 1349 | 1639 |
| AD07749 | AM10800-AS | 1086 | 1543 | AM10799-SS | 1350 | 1640 |
| AD07750 | AM10802-AS | 1087 | 1544 | AM10801-SS | 1351 | 1641 |
| AD07751 | AM10804-AS | 1088 | 1545 | AM10803-SS | 1352 | 1642 |
| AD07752 | AM10806-AS | 1089 | 1546 | AM10805-SS | 1353 | 1643 |
| AD07753 | AM10808-AS | 1090 | 1530 | AM10807-SS | 1354 | 1623 |
| AD07754 | AM10810-AS | 1091 | 1547 | AM10809-SS | 1355 | 1644 |
| AD07755 | AM10812-AS | 1092 | 1548 | AM10811-SS | 1356 | 1645 |
| AD07756 | AM10814-AS | 1093 | 1549 | AM10813-SS | 1357 | 1646 |
| AD07757 | AM10816-AS | 1094 | 1550 | AM10815-SS | 1358 | 1647 |
| AD07758 | AM10818-AS | 1095 | 1532 | AM10817-SS | 1359 | 1627 |
| AD07760 | AM10821-AS | 1096 | 1551 | AM10820-SS | 1361 | 1648 |
| AD07761 | AM10823-AS | 1097 | 1552 | AM10822-SS | 1362 | 1649 |
| AD07762 | AM10825-AS | 1098 | 1553 | AM10824-SS | 1363 | 1650 |
| AD07763 | AM10827-AS | 1099 | 1554 | AM10826-SS | 1364 | 1651 |
| AD07764 | AM10829-AS | 1100 | 1555 | AM10828-SS | 1365 | 1652 |
| AD07765 | AM10831-AS | 1101 | 1556 | AM10830-SS | 1366 | 1653 |
| AD07766 | AM10833-AS | 1102 | 1557 | AM10832-SS | 1367 | 1654 |
| AD07767 | AM10835-AS | 1103 | 1558 | AM10834-SS | 1368 | 1655 |
| AD07768 | AM10837-AS | 1104 | 1559 | AM10836-SS | 1369 | 1656 |
| AD07769 | AM10839-AS | 1105 | 1560 | AM10838-SS | 1370 | 1657 |
| AD07770 | AM10841-AS | 1106 | 1561 | AM10840-SS | 1371 | 1658 |
| AD07771 | AM10843-AS | 1107 | 1562 | AM10842-SS | 1372 | 1659 |
| AD07772 | AM10845-AS | 1108 | 1563 | AM10844-SS | 1373 | 1660 |
| AD07773 | AM10847-AS | 1109 | 1564 | AM10846-SS | 1374 | 1661 |
| AD07774 | AM10849-AS | 1110 | 1565 | AM10848-SS | 1375 | 1662 |
| AD07941 | AM11065-AS | 1111 | 1531 | AM10819-SS | 1360 | 1625 |
| AD08083 | AM11264-AS | 1112 | 1566 | AM11263-SS | 1377 | 1663 |
| AD08084 | AM11264-AS | 1112 | 1566 | AM11265-SS | 1378 | 1664 |
| AD08085 | AM11266-AS | 1113 | 1566 | AM11263-SS | 1377 | 1663 |
| AD08086 | AM11268-AS | 1114 | 1567 | AM11267-SS | 1379 | 1665 |
| AD08087 | AM11268-AS | 1114 | 1567 | AM11269-SS | 1380 | 1666 |
| AD08088 | AM11271-AS | 1115 | 1568 | AM11270-SS | 1381 | 1667 |
| AD08089 | AM11272-AS | 1116 | 1568 | AM11270-SS | 1381 | 1667 |
| AD08094 | AM11275-AS | 1117 | 1569 | AM11274-SS | 1382 | 1668 |
| AD08095 | AM11277-AS | 1118 | 1570 | AM11276-SS | 1383 | 1669 |
| AD08096 | AM11279-AS | 1119 | 1571 | AM11278-SS | 1384 | 1670 |
| AD08097 | AM11281-AS | 1120 | 1572 | AM11280-SS | 1385 | 1671 |
| AD08098 | AM11283-AS | 1121 | 1573 | AM11282-SS | 1386 | 1672 |
| AD08099 | AM11285-AS | 1122 | 1574 | AM11284-SS | 1387 | 1673 |
| AD08100 | AM11287-AS | 1123 | 1575 | AM11286-SS | 1388 | 1674 |
| AD08101 | AM11289-AS | 1124 | 1576 | AM11288-SS | 1389 | 1675 |
| AD08102 | AM11291-AS | 1125 | 1577 | AM11290-SS | 1390 | 1676 |
| AD08103 | AM11293-AS | 1126 | 1578 | AM11292-SS | 1391 | 1677 |
| AD08173 | AM10595-AS | 1065 | 1525 | AM11400-SS | 1392 | 1617 |
| AD08174 | AM11401-AS | 1127 | 1525 | AM11400-SS | 1392 | 1617 |
| AD08175 | AM11403-AS | 1128 | 1579 | AM11402-SS | 1393 | 1678 |
| AD08176 | AM11404-AS | 1129 | 1579 | AM11402-SS | 1393 | 1678 |
| AD08177 | AM11405-AS | 1130 | 1579 | AM11402-SS | 1393 | 1678 |
| AD08224 | AM11464-AS | 1132 | 1581 | AM11463-SS | 1394 | 1679 |
| AD08225 | AM11465-AS | 1133 | 1581 | AM11463-SS | 1394 | 1679 |
| AD08226 | AM11467-AS | 1134 | 1582 | AM11466-SS | 1395 | 1680 |
| AD08227 | AM11469-AS | 1135 | 1583 | AM11468-SS | 1396 | 1681 |
| AD08228 | AM11471-AS | 1136 | 1584 | AM11470-SS | 1397 | 1682 |
| AD08229 | AM11473-AS | 1137 | 1585 | AM11472-SS | 1398 | 1683 |
| AD08230 | AM11475-AS | 1138 | 1586 | AM11474-SS | 1399 | 1684 |
| AD08231 | AM11477-AS | 1139 | 1587 | AM11476-SS | 1400 | 1685 |
| AD08232 | AM11479-AS | 1140 | 1588 | AM11478-SS | 1401 | 1686 |
| AD08233 | AM11481-AS | 1141 | 1589 | AM11480-SS | 1402 | 1687 |
| AD08243 | AM11495-AS | 1142 | 1590 | AM11400-SS | 1392 | 1617 |
| AD08244 | AM11496-AS | 1143 | 1525 | AM11400-SS | 1392 | 1617 |
| AD08245 | AM11498-AS | 1144 | 1591 | AM11497-SS | 1403 | 1688 |
| AD08246 | AM11499-AS | 1145 | 1591 | AM11497-SS | 1403 | 1688 |
| AD08420 | AM11742-AS | 1148 | 1566 | AM11265-SS | 1378 | 1664 |
| AD08421 | AM11742-AS | 1148 | 1566 | AM11263-SS | 1377 | 1663 |
| AD08422 | AM11742-AS | 1148 | 1566 | AM11743-SS | 1405 | 1664 |
| AD08423 | AM11272-AS | 1116 | 1568 | AM11744-SS | 1406 | 1667 |
| AD08424 | AM11745-AS | 1149 | 1568 | AM11744-SS | 1406 | 1667 |
| AD08468 | AM11821-AS | 1150 | 1589 | AM11480-SS | 1402 | 1687 |
| AD08469 | AM11823-AS | 1151 | 1589 | AM11822-SS | 1407 | 1687 |
| AD08470 | AM11825-AS | 1152 | 1593 | AM11824-SS | 1408 | 1690 |
| AD08564 | AM11971-AS | 1153 | 1594 | AM11970-SS | 1409 | 1691 |
| AD08565 | AM11973-AS | 1154 | 1595 | AM11972-SS | 1410 | 1692 |
| AD08566 | AM11975-AS | 1155 | 1596 | AM11974-SS | 1411 | 1693 |
| AD08567 | AM11977-AS | 1156 | 1597 | AM11976-SS | 1412 | 1694 |
| AD08568 | AM11979-AS | 1157 | 1598 | AM11978-SS | 1413 | 1695 |
| AD08569 | AM07100-AS | 1716 | 1566 | AM11980-SS | 1414 | 1664 |
| AD08570 | AM11982-AS | 1158 | 1599 | AM11981-SS | 1415 | 1696 |
| AD08571 | AM11984-AS | 1159 | 1600 | AM11983-SS | 1416 | 1697 |
| AD08572 | AM07104-AS | 1717 | 1568 | AM11985-SS | 1417 | 1667 |
| AD08573 | AM11986-AS | 1160 | 1568 | AM11985-SS | 1417 | 1667 |
| AD08662 | AM12158-AS | 1161 | 1601 | AM12157-SS | 1418 | 1698 |
| AD08663 | AM12159-AS | 1162 | 1601 | AM12157-SS | 1418 | 1698 |
| AD08664 | AM12161-AS | 1163 | 1601 | AM12160-SS | 1419 | 1698 |
| AD08665 | AM12162-AS | 1164 | 1601 | AM12160-SS | 1419 | 1698 |
| AD08666 | AM12163-AS | 1165 | 1535 | AM10767-SS | 1342 | 1632 |
| AD08667 | AM12165-AS | 1166 | 1535 | AM12164-SS | 1420 | 1632 |
| AD08668 | AM12166-AS | 1167 | 1535 | AM12164-SS | 1420 | 1632 |
| AD08669 | AM12167-AS | 1168 | 1535 | AM12164-SS | 1420 | 1632 |
| AD08670 | AM12167-AS | 1168 | 1535 | AM12168-SS | 1421 | 1699 |
| AD08671 | AM12169-AS | 1169 | 1571 | AM11278-SS | 1384 | 1670 |
| AD08672 | AM12171-AS | 1170 | 1571 | AM12170-SS | 1422 | 1670 |
| AD08673 | AM12172-AS | 1171 | 1571 | AM12170-SS | 1422 | 1670 |
| AD08674 | AM12173-AS | 1172 | 1534 | AM10765-SS | 1341 | 1631 |
| AD08675 | AM12175-AS | 1173 | 1534 | AM12174-SS | 1423 | 1631 |
| AD08676 | AM12176-AS | 1174 | 1534 | AM12174-SS | 1423 | 1631 |
| AD08677 | AM12177-AS | 1175 | 1534 | AM12174-SS | 1423 | 1631 |
| AD08678 | AM12178-AS | 1176 | 1568 | AM11270-SS | 1381 | 1667 |
| AD08679 | AM12178-AS | 1176 | 1568 | AM12179-SS | 1424 | 1700 |
| AD08680 | AM12180-AS | 1177 | 1568 | AM11270-SS | 1381 | 1667 |
| AD08681 | AM12181-AS | 1178 | 1568 | AM11270-SS | 1381 | 1667 |
| AD08682 | AM12182-AS | 1179 | 1602 | AM11270-SS | 1381 | 1667 |
| AD08687 | AM12189-AS | 1180 | 1603 | AM12188-SS | 1425 | 1701 |
| AD08688 | AM12191-AS | 1181 | 1604 | AM12190-SS | 1426 | 1702 |
| AD08689 | AM12193-AS | 1182 | 1605 | AM12192-SS | 1427 | 1703 |
| AD08690 | AM12195-AS | 1183 | 1606 | AM12194-SS | 1428 | 1704 |
| AD08691 | AM12197-AS | 1184 | 1607 | AM12196-SS | 1429 | 1705 |
| AD08692 | AM12197-AS | 1184 | 1607 | AM12198-SS | 1430 | 1706 |
| AD08889 | AM11401-AS | 1127 | 1525 | AM10594-SS | 1327 | 1617 |
| AD08890 | AM12516-AS | 1185 | 1525 | AM12515-SS | 1431 | 1617 |
| AD08891 | AM12516-AS | 1185 | 1525 | AM12517-SS | 1432 | 1617 |
| AD08892 | AM12516-AS | 1185 | 1525 | AM12518-SS | 1433 | 1617 |
| AD08893 | AM12519-AS | 1186 | 1525 | AM12518-SS | 1433 | 1617 |
| AD08894 | AM12516-AS | 1185 | 1525 | AM12520-SS | 1434 | 1707 |
| AD08895 | AM12516-AS | 1185 | 1525 | AM12521-SS | 1435 | 1708 |
| AD08896 | AM12516-AS | 1185 | 1525 | AM12522-SS | 1436 | 1709 |
| AD08897 | AM12516-AS | 1185 | 1525 | AM12523-SS | 1437 | 1617 |
| AD08951 | AM12165-AS | 1166 | 1535 | AM12605-SS | 1438 | 1632 |
| AD08952 | AM12165-AS | 1166 | 1535 | AM12606-SS | 1439 | 1632 |
| AD08953 | AM12608-AS | 1187 | 1608 | AM12607-SS | 1440 | 1710 |
| AD08954 | AM12609-AS | 1188 | 1535 | AM12164-SS | 1420 | 1632 |
| AD08955 | AM12610-AS | 1189 | 1535 | AM12164-SS | 1420 | 1632 |
| AD08956 | AM12611-AS | 1190 | 1535 | AM12164-SS | 1420 | 1632 |
| AD08957 | AM12612-AS | 1191 | 1535 | AM12164-SS | 1420 | 1632 |
| AD09240 | AM12165-AS | 1166 | 1535 | AM13074-SS | 1441 | 1632 |
| AD09241 | AM12612-AS | 1191 | 1535 | AM13074-SS | 1441 | 1632 |
| AD09863 | AM11401-AS | 1127 | 1525 | AM14080-SS | 1442 | 1617 |
| AD09864 | AM11401-AS | 1127 | 1525 | AM14081-SS | 1443 | 1711 |
| AD09865 | AM11401-AS | 1127 | 1525 | AM14084-SS | 1444 | 1712 |

TABLE 8C
MUC5AC RNAi Agent Duplexes with Corresponding Sense and Antisense
Strand ID Numbers and Sequence ID numbers for certain modified
and unmodified nucleotide sequences tested in vitro.

| | | AS | AS | | SS | SS |
|---|---|---|---|---|---|---|
| | | modified | unmodified | | modified | unmodified |
| Duplex | AS ID | SEQ ID NO: | SEQ ID NO: | SS ID | SEQ ID NO: | SEQ ID NO: |
| AD07634 | AM10595-AS | 1065 | 1525 | AM10594-SS-S | 1445 | 1617 |
| AD07637 | AM10601-AS | 1068 | 1528 | AM10600-SS-S | 1446 | 1620 |
| AD07732 | AM10766-AS | 1077 | 1534 | AM10765-SS-S | 1447 | 1631 |
| AD07733 | AM10768-AS | 1078 | 1535 | AM10767-SS-S | 1448 | 1632 |
| AD07734 | AM10770-AS | 1079 | 1536 | AM10769-SS-S | 1449 | 1633 |
| AD07735 | AM10772-AS | 1080 | 1537 | AM10771-SS-S | 1450 | 1634 |
| AD07745 | AM10792-AS | 1082 | 1539 | AM10791-SS-S | 1451 | 1636 |
| AD07746 | AM10794-AS | 1083 | 1540 | AM10793-SS-S | 1452 | 1637 |
| AD07747 | AM10796-AS | 1084 | 1541 | AM10795-SS-S | 1453 | 1638 |
| AD07748 | AM10798-AS | 1085 | 1542 | AM10797-SS-S | 1454 | 1639 |
| AD07749 | AM10800-AS | 1086 | 1543 | AM10799-SS-S | 1455 | 1640 |
| AD07750 | AM10802-AS | 1087 | 1544 | AM10801-SS-S | 1456 | 1641 |
| AD07751 | AM10804-AS | 1088 | 1545 | AM10803-SS-S | 1457 | 1642 |
| AD07756 | AM10814-AS | 1093 | 1549 | AM10813-SS-S | 1458 | 1646 |
| AD07760 | AM10821-AS | 1096 | 1551 | AM10820-SS-S | 1459 | 1648 |
| AD07763 | AM10827-AS | 1099 | 1554 | AM10826-SS-S | 1460 | 1651 |
| AD07764 | AM10829-AS | 1100 | 1555 | AM10828-SS-S | 1461 | 1652 |
| AD07766 | AM10833-AS | 1102 | 1557 | AM10832-SS-S | 1462 | 1654 |
| AD07768 | AM10837-AS | 1104 | 1559 | AM10836-SS-S | 1463 | 1656 |
| AD07770 | AM10841-AS | 1106 | 1561 | AM10840-SS-S | 1464 | 1658 |
| AD07771 | AM10843-AS | 1107 | 1562 | AM10842-SS-S | 1465 | 1659 |
| AD07772 | AM10845-AS | 1108 | 1563 | AM10844-SS-S | 1466 | 1660 |
| AD07773 | AM10847-AS | 1109 | 1564 | AM10846-SS-S | 1467 | 1661 |
| AD07774 | AM10849-AS | 1110 | 1565 | AM10848-SS-S | 1468 | 1662 |
| AD08094 | AM11275-AS | 1117 | 1569 | AM11274-SS-S | 1469 | 1668 |
| AD08095 | AM11277-AS | 1118 | 1570 | AM11276-SS-S | 1470 | 1669 |
| AD08096 | AM11279-AS | 1119 | 1571 | AM11278-SS-S | 1471 | 1670 |
| AD08097 | AM11281-AS | 1120 | 1572 | AM11280-SS-S | 1472 | 1671 |
| AD08100 | AM11287-AS | 1123 | 1575 | AM11286-SS-S | 1473 | 1674 |
| AD08101 | AM11289-AS | 1124 | 1576 | AM11288-SS-S | 1474 | 1675 |
| AD08103 | AM11293-AS | 1126 | 1578 | AM11292-SS-S | 1475 | 1677 |
| AD08568 | AM11979-AS | 1157 | 1598 | AM11978-SS-S | 1476 | 1695 |
| AD08569 | AM07100-AS | 1716 | 1566 | AM11980-SS-S | 1477 | 1664 |
| AD08571 | AM11984-AS | 1159 | 1600 | AM11983-SS-S | 1478 | 1697 |
| AD08572 | AM07104-AS | 1717 | 1568 | AM11985-SS-S | 1479 | 1667 |
| AD08573 | AM11986-AS | 1160 | 1568 | AM11985-SS-S | 1479 | 1667 |
| AD08666 | AM12163-AS | 1165 | 1535 | AM10767-SS-S | 1448 | 1632 |
| AD08667 | AM12165-AS | 1166 | 1535 | AM12164-SS-S | 1480 | 1632 |
| AD08668 | AM12166-AS | 1167 | 1535 | AM12164-SS-S | 1480 | 1632 |
| AD08669 | AM12167-AS | 1168 | 1535 | AM12164-SS-S | 1480 | 1632 |
| AD08670 | AM12167-AS | 1168 | 1535 | AM12168-SS-S | 1481 | 1699 |
| AD08671 | AM12169-AS | 1169 | 1571 | AM11278-SS-S | 1471 | 1670 |
| AD08672 | AM12171-AS | 1170 | 1571 | AM12170-SS-S | 1482 | 1670 |
| AD08673 | AM12172-AS | 1171 | 1571 | AM12170-SS-S | 1482 | 1670 |

TABLE 9
MUC5AC RNAi Agent Conjugated Duplexes with Corresponding Sense and Antisense Strand ID
Numbers and Sequence ID numbers for the modified and unmodified nucleotide sequences.
(Shown with Targeting Ligand Conjugates)

| | | AS | AS | | SS | SS |
|---|---|---|---|---|---|---|
| | | modified | unmodified | | modified | unmodified |
| Duplex | AS ID | SEQ ID NO: | SEQ ID NO: | SS ID | SEQ ID NO: | SEQ ID NO: |
| AC000313 | AM10743-AS | 1073 | 1531 | CS000387 | 1483 | 1626 |
| AC000431 | AM11264-AS | 1112 | 1566 | CS000521 | 1486 | 1663 |
| AC000432 | AM11264-AS | 1112 | 1566 | CS000523 | 1487 | 1664 |
| AC000433 | AM11266-AS | 1113 | 1566 | CS000521 | 1486 | 1663 |
| AC000434 | AM11268-AS | 1114 | 1567 | CS000525 | 1488 | 1665 |
| AC000435 | AM11268-AS | 1114 | 1567 | CS000527 | 1489 | 1666 |
| AC000436 | AM11271-AS | 1115 | 1568 | CS000528 | 1490 | 1667 |
| AC000437 | AM11272-AS | 1116 | 1568 | CS000528 | 1490 | 1667 |
| AC000480 | AM11401-AS | 1127 | 1525 | CS000578 | 1491 | 1617 |
| AC000482 | AM10595-AS | 1065 | 1525 | CS000578 | 1491 | 1617 |
| AC000483 | AM11495-AS | 1142 | 1590 | CS000578 | 1491 | 1617 |
| AC000484 | AM11496-AS | 1143 | 1525 | CS000578 | 1491 | 1617 |
| AC000485 | AM11403-AS | 1128 | 1579 | CS000583 | 1492 | 1678 |
| AC000486 | AM11404-AS | 1129 | 1579 | CS000583 | 1492 | 1678 |
| AC000487 | AM11405-AS | 1130 | 1579 | CS000583 | 1492 | 1678 |
| AC000502 | AM11462-AS | 1131 | 1580 | CS000517 | 1484 | 1718 |
| AC000504 | AM11464-AS | 1132 | 1581 | CS000608 | 1493 | 1679 |
| AC000505 | AM11465-AS | 1133 | 1581 | CS000608 | 1493 | 1679 |
| AC000506 | AM11467-AS | 1134 | 1582 | CS000612 | 1494 | 1680 |
| AC000507 | AM11469-AS | 1135 | 1583 | CS000614 | 1495 | 1681 |
| AC000508 | AM11471-AS | 1136 | 1584 | CS000616 | 1496 | 1682 |
| AC000509 | AM11473-AS | 1137 | 1585 | CS000618 | 1497 | 1683 |
| AC000510 | AM11475-AS | 1138 | 1586 | CS000620 | 1498 | 1684 |
| AC000511 | AM11477-AS | 1139 | 1587 | CS000622 | 1499 | 1685 |
| AC000512 | AM11479-AS | 1140 | 1588 | CS000624 | 1500 | 1686 |
| AC000513 | AM11481-AS | 1141 | 1589 | CS000626 | 1501 | 1687 |
| AC000805 | AM10739-AS | 1071 | 1530 | CS001001 | 1503 | 1623 |
| AC000806 | AM10741-AS | 1072 | 1530 | CS001003 | 1504 | 1624 |
| AC000807 | AM10743-AS | 1073 | 1531 | CS001005 | 1505 | 1625 |
| AC000808 | AM10744-AS | 1074 | 1531 | CS001005 | 1505 | 1625 |
| AC000809 | AM10747-AS | 1075 | 1532 | CS001007 | 1506 | 1627 |
| AC000810 | AM10747-AS | 1075 | 1532 | CS001009 | 1507 | 1628 |
| AC000811 | AM10747-AS | 1075 | 1532 | CS001010 | 1508 | 1629 |
| AC001128 | AM12178-AS | 1176 | 1568 | CS000528 | 1490 | 1667 |
| AC001129 | AM12178-AS | 1176 | 1568 | CS001401 | 1512 | 1700 |
| AC001130 | AM12180-AS | 1177 | 1568 | CS000528 | 1490 | 1667 |
| AC001131 | AM12181-AS | 1178 | 1568 | CS000528 | 1490 | 1667 |
| AC000832 | AM11742-AS | 1148 | 1566 | CS000523 | 1487 | 1664 |
| AC000833 | AM11742-AS | 1148 | 1566 | CS000521 | 1486 | 1663 |
| AC000834 | AM11742-AS | 1148 | 1566 | CS001040 | 1510 | 1664 |
| AC000835 | AM11272-AS | 1116 | 1568 | CS001041 | 1511 | 1667 |
| AC000836 | AM11745-AS | 1149 | 1568 | CS001041 | 1511 | 1667 |
| AC001305 | AM12165-AS | 1166 | 1535 | CS001644 | 1513 | 1632 |
| AC001306 | AM12612-AS | 1191 | 1535 | CS001644 | 1513 | 1632 |
| AC001708 | AM11401-AS | 1127 | 1525 | CS002194 | 1514 | 1617 |
| AC001709 | AM11401-AS | 1127 | 1525 | CS002195 | 1515 | 1711 |
| AC001710 | AM11401-AS | 1127 | 1525 | CS002196 | 1516 | 1712 |

TABLE 10A
Conjugate Duplex ID Numbers Referencing
Position Targeted On MUC5AC (MUC5AC) Gene

| | | | Targeted MUC5AC |
|---|---|---|---|
| | | | Gene Position |
| Duplex | AS ID | SS ID | (Of SEQ ID NO: 1) |
| AC000313 | AM10743-AS | CS000387 | 1921 |
| AC000431 | AM11264-AS | CS000521 | 5029 |
| AC000432 | AM11264-AS | CS000523 | 5029 |
| AC000433 | AM11266-AS | CS000521 | 5029 |
| AC000434 | AM11268-AS | CS000525 | 9729 |
| AC000435 | AM11268-AS | CS000527 | 9729 |
| AC000436 | AM11271-AS | CS000528 | 15052 |
| AC000437 | AM11272-AS | CS000528 | 15052 |
| AC000480 | AM11401-AS | CS000578 | 3535 |
| AC000482 | AM10595-AS | CS000578 | 3535 |
| AC000483 | AM11495-AS | CS000578 | 3535 |
| AC000484 | AM11496-AS | CS000578 | 3535 |
| AC000485 | AM11403-AS | CS000583 | 3535 |
| AC000486 | AM11404-AS | CS000583 | 3535 |
| AC000487 | AM11405-AS | CS000583 | 3535 |
| AC000502 | AM11462-AS | CS000517 | N/A (murine-specific) |
| AC000504 | AM11464-AS | CS000608 | N/A (murine-specific) |
| AC000505 | AM11465-AS | CS000608 | N/A (murine-specific) |
| AC000506 | AM11467-AS | CS000612 | N/A (murine-specific) |
| AC000507 | AM11469-AS | CS000614 | N/A (murine-specific) |
| AC000508 | AM11471-AS | CS000616 | N/A (murine-specific) |
| AC000509 | AM11473-AS | CS000618 | N/A (murine-specific) |
| AC000510 | AM11475-AS | CS000620 | N/A (murine-specific) |
| AC000511 | AM11477-AS | CS000622 | N/A (murine-specific) |
| AC000512 | AM11479-AS | CS000624 | N/A (murine-specific) |
| AC000513 | AM11481-AS | CS000626 | N/A (murine-specific) |
| AC000805 | AM10739-AS | CS001001 | 304 |
| AC000806 | AM10741-AS | CS001003 | 304 |
| AC000807 | AM10743-AS | CS001005 | 1921 |
| AC000808 | AM10744-AS | CS001005 | 1921 |
| AC000809 | AM10747-AS | CS001007 | 1832 |
| AC000810 | AM10747-AS | CS001009 | 1832 |
| AC000811 | AM10747-AS | CS001010 | 1832 |
| AC001128 | AM12178-AS | CS000528 | 15052 |
| AC001129 | AM12178-AS | CS001401 | 15052 |
| AC001130 | AM12180-AS | CS000528 | 15052 |
| AC001131 | AM12181-AS | CS000528 | 15052 |
| AC000832 | AM11742-AS | CS000523 | 5029 |
| AC000833 | AM11742-AS | CS000521 | 5029 |
| AC000834 | AM11742-AS | CS001040 | 5029 |
| AC000835 | AM11272-AS | CS001041 | 15052 |
| AC000836 | AM11745-AS | CS001041 | 15052 |
| AC001305 | AM12165-AS | CS001644 | 4993 |
| AC001306 | AM12612-AS | CS001644 | 4993 |
| AC001708 | AM11401-AS | CS002194 | 3535 |
| AC001709 | AM11401-AS | CS002195 | 3535 |
| AC001710 | AM11401-AS | CS002196 | 3535 |

TABLE 10B
Conjugate ID Numbers and Corresponding AD Duplex Numbers,
Referencing Position Targeted On MUC5AC (MUC5AC) Gene

| | Corresponding | Targeted MUC5AC |
|---|---|---|
| AC Duplex | AD Duplex | Gene Position |
| Number | Number | (Of SEQ ID NO: 1) |
| AC000313 | AD07720 | 1921 |
| AC000431 | AD08083 | 5029 |
| AC000432 | AD08084 | 5029 |
| AC000433 | AD08085 | 5029 |
| AC000434 | AD08086 | 9729 |
| AC000435 | AD08087 | 9729 |
| AC000436 | AD08088 | 15052 |
| AC000437 | AD08089 | 15052 |
| AC000480 | AD08174 | 3535 |
| AC000482 | AD08173 | 3535 |
| AC000483 | AD08243 | 3535 |
| AC000484 | AD08244 | 3535 |
| AC000485 | AD08175 | 3535 |
| AC000486 | AD08176 | 3535 |
| AC000487 | AD08177 | 3535 |
| AC000502 | AD08222 | N/A (murine-specific) |
| AC000503 | AD08223 | N/A (murine-specific) |
| AC000504 | AD08224 | N/A (murine-specific) |
| AC000505 | AD08225 | N/A (murine-specific) |
| AC000506 | AD08226 | N/A (murine-specific) |
| AC000507 | AD08227 | N/A (murine-specific) |
| AC000508 | AD08228 | N/A (murine-specific) |
| AC000509 | AD08229 | N/A (murine-specific) |
| AC000510 | AD08230 | N/A (murine-specific) |
| AC000511 | AD08231 | N/A (murine-specific) |
| AC000512 | AD08232 | N/A (murine-specific) |
| AC000513 | AD08233 | N/A (murine-specific) |
| AC000805 | AD07716 | 304 |
| AC000806 | AD07717 | 304 |
| AC000807 | AD07718 | 1921 |
| AC000808 | AD07719 | 1921 |
| AC000809 | AD07721 | 1832 |
| AC000810 | AD07722 | 1832 |
| AC000811 | AD07723 | 1832 |
| AC001128 | AD08678 | 15052 |
| AC001129 | AD08679 | 15052 |
| AC001130 | AD08680 | 15052 |
| AC001131 | AD08681 | 15052 |
| AC000832 | AD08420 | 5029 |
| AC000833 | AD08421 | 5029 |
| AC000834 | AD08422 | 5029 |
| AC000835 | AD08423 | 15052 |
| AC000836 | AD08424 | 15052 |
| AC001305 | AD09240 | 4993 |
| AC001306 | AD09241 | 4993 |
| AC001708 | AD09863 | 3535 |
| AC001709 | AD09864 | 3535 |
| AC001710 | AD09865 | 3535 |

TABLE 11
Conjugate ID Numbers With Chemically Modified Antisense and Sense Strands (including Linkers and Conjugates)

| | Sense Strand (Fully Modified with Conjugated Targeting | | | |
|---|---|---|---|---|
| AC ID Number | Ligand) (5′→3′) | SEQ ID NO. | Antisense Strand (5′→3′) | SEQ ID NO. |
| AC000313 | Tri-SM6.1-avb6-(TA14)gsa_2NaguaugCfUfCfaguacugiuas(invAb) | 1483 | cPrpusAfscsCfaGfuGfcUfgAfgCfaUfaCfuUfsc | 1073 |
| AC000431 | Tri-SM6.1-avb6-(TA14)csugcuacaAfCfUfaugagauccas(invAb) | 1486 | cPrpusGfsgsAfuCfuCfaUfaGfuUfgUfaGfcAfsg | 1112 |
| AC000432 | Tri-SM6.1-avb6-(TA14)csugcuacaAfCfUfaugaiauccas(invAb) | 1487 | cPrpusGfsgsAfuCfuCfaUfaGfuUfgUfaGfcAfsg | 1112 |
| AC000433 | Tri-SM6.1-avb6-(TA14)csugcuacaAfCfUfaugagauccas(invAb) | 1486 | cPrpusGfsgsAfuCfUuNACfaUfaGfuUfgUfaGfcAfsg | 1113 |
| AC000434 | Tri-SM6.1-avb6-(TA14)cscuggaccAfAfGfugguuugacas(invAb) | 1488 | cPrpusGfsusCfaAfaCfcAfcUfuGfgUfcCfaGfsg | 1114 |
| AC000435 | Tri-SM6.1-avb6-(TA14)cscuggaccAfAfGfugguuuiacas(invAb) | 1489 | cPrpusGfsusCfaAfaCfcAfcUfuGfgUfcCfaGfsg | 1114 |
| AC000436 | Tri-SM6.1-avb6-(TA14)csgagaucaUfCfUfucaacaacaas(invAb) | 1490 | cPrpusUfsgsUfuGfuUfgAfaGfaUfgAfuCfuCfsg | 1115 |
| AC000437 | Tri-SM6.1-avb6-(TA14)csgagaucaUfCfUfucaacaacaas(invAb) | 1490 | cPrpusUfsgsUfuguugaaGfaUfgAfucucsg | 1116 |
| AC000480 | Tri-SM6.1-avb6-(TA14)gscuguucuGfCfGfacuacuacaas(invAb) | 1491 | cPrpusUfsgsUfaGfuAfgUfcGfcAfgAfaCfaGfsc | 1127 |
| AC000482 | Tri-SM6.1-avb6-(TA14)gscuguucuGfCfGfacuacuacaas(invAb) | 1491 | usUfsgsUfaGfuAfgUfcGfcAfgAfaCfaGfsc | 1065 |
| AC000483 | Tri-SM6.1-avb6-(TA14)gscuguucuGfCfGfacuacuacaas(invAb) | 1491 | cPrpusUfsgsUfaGfuAfgUfcicAfgAfaCfaGfsc | 1142 |
| AC000484 | Tri-SM6.1-avb6-(TA14)gscuguucuGfCfGfacuacuacaas(invAb) | 1491 | cPrpusUfsgsUfaGfuAfgUfcgcAfgAfaCfaGfsc | 1143 |
| AC000485 | Tri-SM6.1-avb6-(TA14)gscuguucuGfUfGfacuacuacaas(invAb) | 1492 | usUfsgsUfaGfuAfgUfcAfcAfgAfaCfaGfsc | 1128 |
| AC000486 | Tri-SM6.1-avb6-(TA14)gscuguucuGfUfGfacuacuacaas(invAb) | 1492 | cPrpusUfsgsUfaGfuAfgUfcAfcAfgAfaCfaGfsc | 1129 |
| AC000487 | Tri-SM6.1-avb6-(TA14)gscuguucuGfUfGfacuacuacaas(invAb) | 1492 | cPrpusUfsgsuaguagucAfcAfgAfacagsc | 1130 |
| AC000502 | Tri-SM6.1-avb6-(TA14)ascccauguGfCfUfacaacuaugas(invAb) | 1484 | cPrpusCfsasuaguuguaGfcAfcAfugggsu | 1131 |
| AC000503 | Tri-SM6.1-avb6-(TA14)asccagaucAfUfCfuucaacaacas(invAb) | 1493 | usGfsusUfgUfuGfaAfgAfuGfaUfcUfgGfsu | 1715 |
| AC000504 | Tri-SM6.1-avb6-(TA14)asccagaucAfUfCfuucaacaacas(invAb) | 1493 | cPrpusGfsusUfgUfuGfaAfgAfuGfaUfcUfgGfsu | 1132 |
| AC000505 | Tri-SM6.1-avb6-(TA14)asccagaucAfUfCfuucaacaacas(invAb) | 1493 | cPrpusGfsusuguugaagAfuGfaUfcuggsu | 1133 |
| AC000506 | Tri-SM6.1-avb6-(TA14)gsgcucuguGfGfUfaacuucaacas(invAb) | 1494 | cPrpusGfsusUfgAfaGfuUfaCfcAfcAfgAfgCfsc | 1134 |
| AC000507 | Tri-SM6.1-avb6-(TA14)gsagcguggAfGfAfaugaaaaguas(invAb) | 1495 | cPrpusAfscsUfuUfuCfaUfuCfuCfcAfcGfcUfsc | 1135 |
| AC000508 | Tri-SM6.1-avb6-(TA14)gsggagaauGfAfAfaaguaugcuas(invAb) | 1496 | cPrpusAfsgsCfaUfaCfuUfuUfcAfuUfcUfcCfsc | 1136 |
| AC000509 | Tri-SM6.1-avb6-(TA14)csuucucgaAfCfUfgcauguaugas(invAb) | 1497 | cPrpusCfsasUfaCfaUfgCfaGfuUfcGfaGfaAfsg | 1137 |
| AC000510 | Tri-SM6.1-avb6-(TA14)gscucgaacUfGfCfauguaugacas(invAb) | 1498 | cPrpusGfsusCfaUfaCfaUfgCfaGfuUfcGfaGfsc | 1138 |
| AC000511 | Tri-SM6.1-avb6-(TA14)csucgaacuGfCfAfuguaugacaas(invAb) | 1499 | cPrpusUfsgsUfcAfuAfcAfuGfcAfgUfuCfgAfsg | 1139 |
| AC000512 | Tri-SM6.1-avb6-(TA14)cscacuguuCfUfGfugacuacuaas(invAb) | 1500 | cPrpusUfsasGfuAfgUfcAfcAfgAfaCfaGfuGfsg | 1140 |
| AC000513 | Tri-SM6.1-avb6-(TA14)csacuguucUfGfUfgacuacuacas(invAb) | 1501 | cPrpusGfsusAfgUfaGfuCfaCfaGfaAfcAfgUfsg | 1141 |
| AC000805 | Tri-SM6.1-avb6-(TA14)csagcuuccAfCfUfacaaiaccuus(invAb) | 1503 | cPrpasAfsgsGfuCfuUfgUfaGfuGfgAfaGfcUfsg | 1071 |
| AC000806 | Tri-SM6.1-avb6-(TA14)csagcuuccAfCfUfacaagaccuus(invAb) | 1504 | cPrpasAfsgsGfuCfU_{UNA}UfgUfaGfuGfgAfaGfcUfsg | 1072 |
| AC000807 | Tri-SM6.1-avb6-(TA14)gsa_2NaguaugCfUfCfagcacugiuas(invAb) | 1505 | cPrpusAfscsCfaGfuGfcUfgAfgCfaUfaCfuUfsc | 1073 |
| AC000808 | Tri-SM6.1-avb6-(TA14)gsa_2NaguaugCfUfCfagcacugiuas(invAb) | 1505 | cPrpusAfscsCfaGfU_{UNA}GfcUfgAfgCfaUfaCfuUfsc | 1074 |
| AC000809 | Tri-SM6.1-avb6-(TA14)gsccuucuuCfAfAfcaccuucaaas(invAb) | 1506 | cPrpusUfsusGfaAfgguguUfgAfaGfaAfgGfsc | 1075 |
| AC000810 | Tri-SM6.1-avb6-(TA14)gsccuucuuCfAfAfcaucuucaaas(invAb) | 1507 | cPrpusUfsusGfaAfgguguUfgAfaGfaAfgGfsc | 1075 |
| AC000811 | Tri-SM6.1-avb6-(TA14)gsccuucuuCfAfAfcacuuucaaas(invAb) | 1508 | cPrpusUfsusGfaAfgguguUfgAfaGfaAfgGfsc | 1075 |
| AC000832 | Tri-SM6.1-avb6-(TA14)csugcuacaAfCfUfaugaiauccas(invAb) | 1487 | cPrpusGfsgsaucucauaGfuUfgUfagcasg | 1148 |
| AC000833 | Tri-SM6.1-avb6-(TA14)csugcuacaAfCfUfaugagauccas(invAb) | 1486 | cPrpusGfsgsaucucauaGfuUfgUfagcasg | 1148 |
| AC000834 | Tri-SM6.1-avb6-(TA14)csugcuaCfaAfcUfaugaiauccas(invAb) | 1510 | cPrpusGfsgsaucucauaGfuUfgUfagcasg | 1148 |
| AC000835 | Tri-SM6.1-avb6-(TA14)csgagauCfaUfcUfucaacaacaas(invAb) | 1511 | cPrpusUfsgsUfuguugaaGfaUfgAfucucsg | 1116 |
| AC000836 | Tri-SM6.1-avb6-(TA14)csgagauCfaUfcUfucaacaacaas(invAb) | 1511 | cPrpusUfsgsuuguugaaGfaUfgAfucucsg | 1149 |
| AC001128 | Tri-SM6.1-avb6-(TA14)csgagaucaUfCfUfucaacaacaas(invAb) | 1490 | cPrpusUfsgsUfugUu_{UNA}UgaaGfaUfgAfucucsg | 1176 |
| AC001129 | Tri-SM6.1-avb6-(TA14)csgagaucaUfCfUfuca_2Nacaacaas(invAb) | 1512 | cPrpusUfsgsUfugU_{UNA}UgaaGfaUfgAfucucsg | 1176 |
| AC001130 | Tri-SM6.1-avb6-(TA14)csgagaucaUfCfUfucaacaacaas(invAb) | 1490 | cPrpusUfsgsUfuguU_{UNA}gaaGfaUfgAfucucsg | 1177 |
| AC001131 | Tri-SM6.1-avb6-(TA14)csgagaucaUfCfUfucaacaacaas(invAb) | 1490 | cPrpusUfsgsUfU_{UNA}guugaaGfaUfgAfucucsg | 1178 |
| AC001305 | Tri-SM6.1-avb6-(TA14)gscugauUfuGfcCfugaacaagaas(invAb) | 1513 | usUfscsuuguucagGfcAfaAfucagsc | 1166 |
| AC001306 | Tri-SM6.1-avb6-(TA14)gscugauUfuGfcCfugaacaagaas(invAb) | 1513 | cPrpuUfcuuguucagGfcAfaAfucagsc | 1191 |
| AC001708 | Tri-SM6.1-avb6-(TA14)gscuguucuGfCfGfacuacuacaa(invAb) | 1514 | cPrpusUfsgsUfaGfuAfgUfcGfcAfgAfaCfaGfsc | 1127 |
| AC001709 | Tri-SM6.1-avb6-(TA14)gscugguucuGfCfGfacuacuacaas(invAb) | 1515 | cPrpusUfsgsUfaGfuAfgUfcGfcAfgAfaCfaGfsc | 1127 |
| AC001710 | Tri-SM6.1-avb6-(TA14)gscguucuGfCfGfacuacuacaas(invAb) | 1516 | cPrpusUfsgsUfaGfuAfgUfcGfcAfgAfaCfaGfsc | 1127 |

In some embodiments, a MUC5AC RNAi agent is prepared or provided as a salt, mixed salt, or a free-acid. In some embodiments, a MUC5AC RNAi agent is prepared or provided as a pharmaceutically acceptable salt. In some embodiments, a MUC5AC RNAi agent is prepared or provided as a pharmaceutically acceptable sodium or potassium salt The RNAi agents described herein, upon delivery to a cell expressing an MUC5AC gene, inhibit or knockdown expression of one or more MUC5AC genes in vivo and/or in vitro.

### Targeting Groups, Linking Groups, Pharmacokinetic/Pharmacodynamic (PK/PD) Modulators, and Delivery Vehicles

In some embodiments, a MUC5AC RNAi agent contains or is conjugated to one or more non-nucleotide groups including, but not limited to, a targeting group, a linking group, a pharmacokinetic/pharmacodynamic (PK/PD) modulator, a delivery polymer, or a delivery vehicle. The non-nucleotide group can enhance targeting, delivery, or attachment of the RNAi agent. The non-nucleotide group can be covalently linked to the 3′ and/or 5′ end of either the sense strand and/or the antisense strand. In some embodiments, a MUC5AC RNAi agent contains a non-nucleotide group linked to the 3′ and/or 5′ end of the sense strand. In some embodiments, a non-nucleotide group is linked to the 5′ end of a MUC5AC RNAi agent sense strand. A non-nucleotide group can be linked directly or indirectly to the RNAi agent via a linker/linking group. In some embodiments, a non-nucleotide group is linked to the RNAi agent via a labile, cleavable, or reversible bond or linker.

In some embodiments, a non-nucleotide group enhances the pharmacokinetic or biodistribution properties of an RNAi agent or conjugate to which it is attached to improve cell- or tissue-specific distribution and cell-specific uptake of the conjugate. In some embodiments, a non-nucleotide group enhances endocytosis of the RNAi agent.

Targeting groups or targeting moieties enhance the pharmacokinetic or biodistribution properties of a conjugate or RNAi agent to which they are attached to improve cell-specific (including, in some cases, organ specific) distribution and cell-specific (or organ specific) uptake of the conjugate or RNAi agent. A targeting group can be monovalent, divalent, trivalent, tetravalent, or have higher valency for the target to which it is directed. Representative targeting groups include, without limitation, compounds with affinity to cell surface molecule, cell receptor ligands, hapten, antibodies, monoclonal antibodies, antibody fragments, and antibody mimics with affinity to cell surface molecules. In some embodiments, a targeting group is linked to an RNAi agent using a linker, such as a PEG linker or one, two, or three abasic and/or ribitol (abasic ribose) residues, which in some instances can serve as linkers.

A targeting group, with or without a linker, can be attached to the 5′ or 3′ end of any of the sense and/or antisense strands disclosed in Tables 2, 3, 4, 5, 6, 7, and 11. A linker, with or without a targeting group, can be attached to the 5′ or 3′ end of any of the sense and/or antisense strands disclosed in Tables 2, 3, 4, 5, 6, 7, and 11.

The MUC5AC RNAi agents described herein can be synthesized having a reactive group, such as an amino group (also referred to herein as an amine), at the 5′-terminus and/or the 3′-terminus. The reactive group can be used subsequently to attach a targeting moiety using methods typical in the art.

For example, in some embodiments, the MUC5AC RNAi agents disclosed herein can be synthesized having an NH₂-C₆ group at the 5′-terminus of the sense strand of the RNAi agent. The terminal amino group subsequently can be reacted to form a conjugate with, for example, a group that includes an αvβ6 integrin targeting ligand. In some embodiments, the MUC5AC RNAi agents disclosed herein are synthesized having one or more alkyne groups at the 5′-terminus of the sense strand of the RNAi agent. The terminal alkyne group(s) can subsequently be reacted to form a conjugate with, for example, a group that includes an αvβ6 integrin targeting ligand.

In some embodiments, a targeting group comprises an integrin targeting ligand. In some embodiments, an integrin targeting ligand is an αvβ6 integrin targeting ligand. The use of an αvβ6 integrin targeting ligand facilitates cell-specific targeting to cells having αvβ6 on its respective surface, and binding of the integrin targeting ligand can facilitate entry of the therapeutic agent, such as an RNAi agent, to which it is linked, into cells such as epithelial cells, including pulmonary epithelial cells and renal epithelial cells. Integrin targeting ligands can be monomeric or monovalent (e.g., having a single integrin targeting moiety) or multimeric or multivalent (e.g., having multiple integrin targeting moieties). The targeting group can be attached to the 3′ and/or 5′ end of the RNAi oligonucleotide using methods known in the art. The preparation of targeting groups, such as αvβ6 integrin targeting ligands, is described, for example, in International Patent Application Publication No. WO 2018/085415 and in International Patent Application Publication No. WO 2019/089765, the contents of each of which are incorporated herein in its entirety.

In some embodiments, targeting groups are linked to the MUC5AC RNAi agents without the use of an additional linker. In some embodiments, the targeting group is designed having a linker readily present to facilitate the linkage to a MUC5AC RNAi agent. In some embodiments, when two or more RNAi agents are included in a composition, the two or more RNAi agents can be linked to their respective targeting groups using the same linkers. In some embodiments, when two or more RNAi agents are included in a composition, the two or more RNAi agents are linked to their respective targeting groups using different linkers.

In some embodiments, a linking group is conjugated to the RNAi agent. The linking group facilitates covalent linkage of the agent to a targeting group, pharmacokinetic modulator, delivery polymer, or delivery vehicle. The linking group can be linked to the 3′ and/or the 5′ end of the RNAi agent sense strand or antisense strand. In some embodiments, the linking group is linked to the RNAi agent sense strand. In some embodiments, the linking group is conjugated to the 5′ or 3′ end of an RNAi agent sense strand. In some embodiments, a linking group is conjugated to the 5′ end of an RNAi agent sense strand. Examples of linking groups, include but are not limited to: C6-SS-C6, 6-SS-6, reactive groups such a primary amines (e.g., NH2-C6) and alkynes, alkyl groups, abasic residues/nucleotides, amino acids, tri-alkyne functionalized groups, ribitol, and/or PEG groups. Examples of certain linking groups are provided in Table 12.

A linker or linking group is a connection between two atoms that links one chemical group (such as an RNAi agent) or segment of interest to another chemical group (such as a targeting group, pharmacokinetic modulator, or delivery polymer) or segment of interest via one or more covalent bonds. A labile linkage contains a labile bond. A linkage can optionally include a spacer that increases the distance between the two joined atoms. A spacer may further add flexibility and/or length to the linkage. Spacers include, but are not be limited to, alkyl groups, alkenyl groups, alkynyl groups, aryl groups, aralkyl groups, aralkenyl groups, and aralkynyl groups; each of which can contain one or more heteroatoms, heterocycles, amino acids, nucleotides, and saccharides. Spacer groups are well known in the art and the preceding list is not meant to limit the scope of the description. In some embodiments, a MUC5AC RNAi agent is conjugated to a polyethylene glycol (PEG) moiety, or to a hydrophobic group having 12 or more carbon atoms, such as a cholesterol or palmitoyl group.

In some embodiments, a MUC5AC RNAi agent is linked to one or more pharmacokinetic/pharmacodynamic (PK/PD) modulators. PK/PD modulators can increase circulation time of the conjugated drug and/or increase the activity of the RNAi agent through improved cell receptor binding, improved cellular uptake, and/or other means. Various PK/PD modulators suitable for use with RNAi agents are known in the art. In some embodiments, the PK/PD modulatory can be cholesterol or cholesteryl derivatives, or in some circumstances a PK/PD modulator can be comprised of alkyl groups, alkenyl groups, alkynyl groups, aryl groups, aralkyl groups, aralkenyl groups, or aralkynyl groups, each of which may be linear, branched, cyclic, and/or substituted or unsubstituted. In some embodiments, the location of attachment for these moieties is at the 5′ or 3′ end of the sense strand, at the 2′ position of the ribose ring of any given nucleotide of the sense strand, and/or attached to the phosphate or phosphorothioate backbone at any position of the sense strand.

Any of the MUC5AC RNAi agent nucleotide sequences listed in Tables 2, 3, 4, 5, 6, 7, and 11, whether modified or unmodified, can contain 3′ and/or 5′ targeting group(s), linking group(s), and/or PK/PD modulator(s). Any of the MUC5AC RNAi agent sequences listed in Tables 3, 4, 5, 6, 7, and 11, or are otherwise described herein, which contain a 3′ or 5′ targeting group, linking group, and/or PK/PD modulator can alternatively contain no 3′ or 5′ targeting group, linking group, or PK/PD modulator, or can contain a different 3′ or 5′ targeting group, linking group, or pharmacokinetic modulator including, but not limited to, those depicted in Table 12. Any of the MUC5AC RNAi agent duplexes listed in Tables 8A, 8B, 8C, 9, 10A, 10B, and 11, whether modified or unmodified, can further comprise a targeting group or linking group, including, but not limited to, those depicted in Table 11, and the targeting group or linking group can be attached to the 3′ or 5′ terminus of either the sense strand or the antisense strand of the MUC5AC RNAi agent duplex.

Examples of certain modified nucleotides, capping moieties, and linking groups are provided in Table 12.

TABLE 12
Structures Representing Various Modified Nucleotides, Capping Moieties, and
Linking Groups (wherein [symbol] indicates the point of connection)

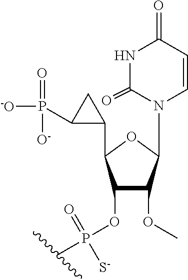

cPrpus

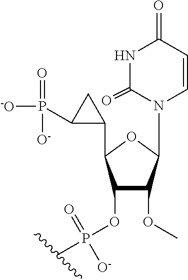

cPrpu

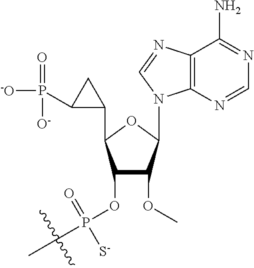

cPrpas

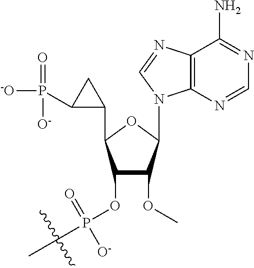

cPrpa

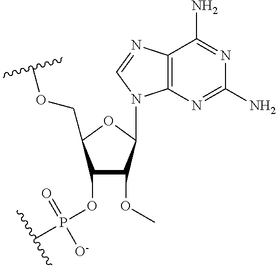

a_2N

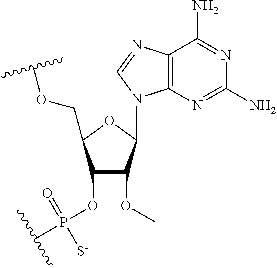

a_2Ns
When positioned internally:

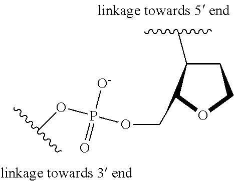

(invAb)
When positioned internally:

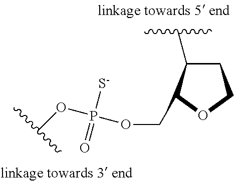

(invAb)s
When positioned at the 3′ terminal end:

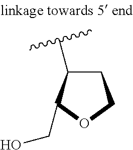

(invAb)
When positioned at the 3′ terminal end:

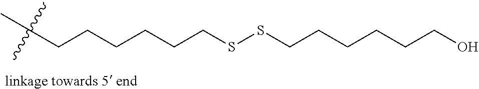 [SMILES: *CCCCCCSSCCCCCCO]

(C6-SS-C6)
When positioned internally:

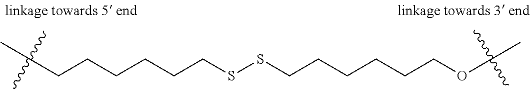 [SMILES: *CCCCCCSSCCCCCCO*]

(C6-SS-C6)
When positioned at the 3′ terminal end:

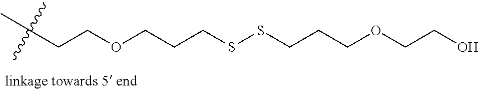 [SMILES: *CCOCCCSSCCCOCCO]

(6-SS-6)
When positioned internally:

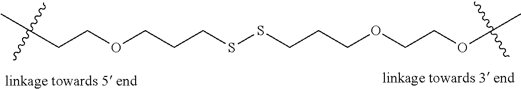 [SMILES: *CCOCCCSSCCCOCCO*]

(6-SS-6)

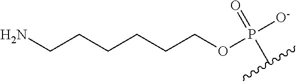 [SMILES: *P(=O)([O-])OCCCCCCN]

(NH2-C6)

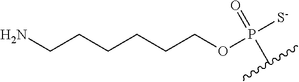 [SMILES: *P(=O)([S-])OCCCCCCN]

(NH2-C6)s

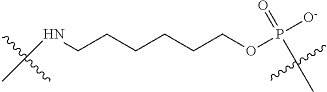 [SMILES: *NCCCCCCOP(*)(=O)[O-]]

-C6-

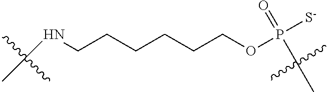 [SMILES: *NCCCCCCOP(*)(=O)[S-]]

-C6s-

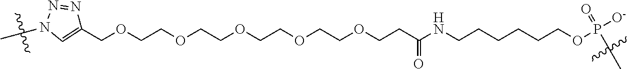 [SMILES: *n1cc(COCCOCCOCCOCCOCCC(=O)NCCCCCCOP(*)(=O)[O-])nn1]

-L6-C6-

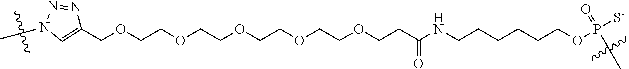 [SMILES: *n1cc(COCCOCCOCCOCCOCCC(=O)NCCCCCCOP(*)(=O)[S-])nn1]

-L6-C6s-

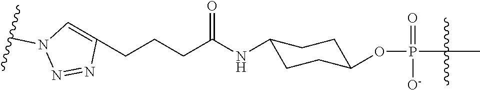 [SMILES: *n1cc(CCCC(=O)NC2CCC(OP(*)(=O)[O-])CC2)nn1]

-Alk-cyHex-

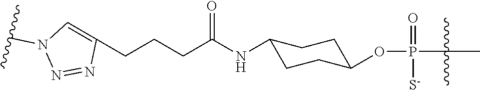 [SMILES: *n1cc(CCCC(=O)NC2CCC(OP(*)(=O)[S-])CC2)nn1]

-Alk-cyHexs-

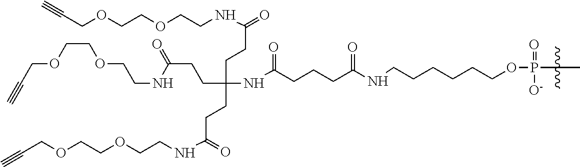 [SMILES: *P(=O)([O-])OCCCCCCNC(=O)CCCC(=O)NC(CCC(=O)NCCOCCOCC#C)(CCC(=O)NCCOCCOCC#C)CCC(=O)NCCOCCOCC#C]

(TriAlk14)

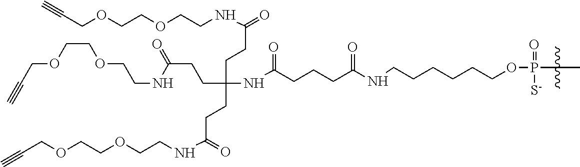 [SMILES: *P(=O)([S-])OCCCCCCNC(=O)CCCC(=O)NC(CCC(=O)NCCOCCOCC#C)(CCC(=O)NCCOCCOCC#C)CCC(=O)NCCOCCOCC#C]

(TriAlk14)s

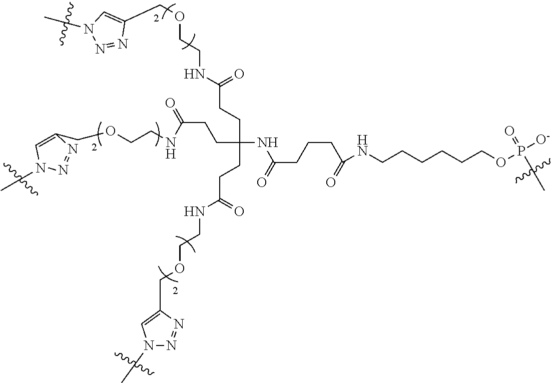

(TA14)

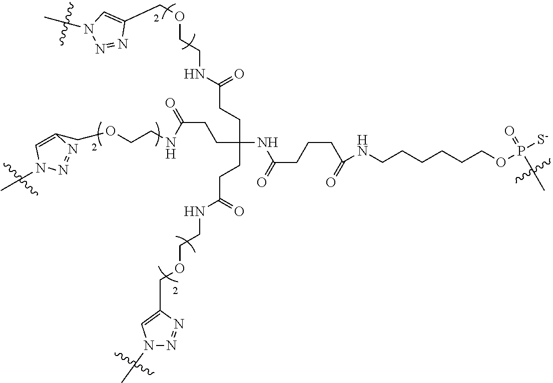

(TA14)s

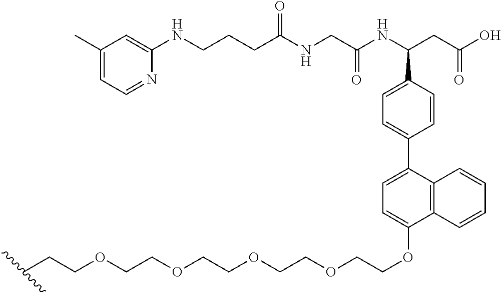 [SMILES: *CCOCCOCCOCCOCCOc1ccc(-c2ccc([C@H](CC(=O)O)NC(=O)CNC(=O)CCCNc3cc(C)ccn3)cc2)c2ccccc12]

SM6.1-αvβ6

Alternatively, other linking groups known in the art may be used. In many instances, linking groups can be commercially acquired or alternatively, are incorporated into commercially available nucleotide phosphoramidites. (See, e.g., International Patent Application Publication No. WO 2019/161213, which is incorporated herein by reference in its entirety).

In some embodiments, a MUC5AC RNAi agent is delivered without being conjugated to a targeting ligand or pharmacokinetic/pharmacodynamic (PK/PD) modulator (referred to as being “naked” or a “naked RNAi agent”).

In some embodiments, a MUC5AC RNAi agent is conjugated to a targeting group, a linking group, a PK modulator, and/or another non-nucleotide group to facilitate delivery of the MUC5AC RNAi agent to the cell or tissue of choice, for example, to an epithelial cell in vivo. In some embodiments, a MUC5AC RNAi agent is conjugated to a targeting group wherein the targeting group includes an integrin targeting ligand. In some embodiments, the integrin targeting ligand is an αvβ6 integrin targeting ligand. In some embodiments, a targeting group includes one or more αvβ6 integrin targeting ligands.

In some embodiments, a delivery vehicle may be used to deliver an RNAi agent to a cell or tissue. A delivery vehicle is a compound that improves delivery of the RNAi agent to a cell or tissue. A delivery vehicle can include, or consist of, but is not limited to: a polymer, such as an amphipathic polymer, a membrane active polymer, a peptide, a melittin peptide, a melittin-like peptide (MLP), a lipid, a reversibly modified polymer or peptide, or a reversibly modified membrane active polyamine.

In some embodiments, the RNAi agents can be combined with lipids, nanoparticles, polymers, liposomes, micelles, DPCs or other delivery systems available in the art for nucleic acid delivery. The RNAi agents can also be chemically conjugated to targeting groups, lipids (including, but not limited to cholesteryl and cholesteryl derivatives), encapsulating in nanoparticles, liposomes, micelles, conjugating to polymers or DPCs (see, for example WO 2000/053722, WO 2008/022309, WO 2011/104169, and WO 2012/083185, WO 2013/032829, WO 2013/158141, each of which is incorporated herein by reference), by iontophoresis, or by incorporation into other delivery vehicles or systems available in the art such as hydrogels, cyclodextrins, biodegradable nanocapsules, bioadhesive microspheres, or proteinaceous vectors. In some embodiments the RNAi agents can be conjugated to antibodies having affinity for pulmonary epithelial cells. In some embodiments, the RNAi agents can be linked to targeting ligands that have affinity for pulmonary epithelial cells or receptors present on pulmonary epithelial cells.

### Pharmaceutical Compositions and Formulations

The MUC5AC RNAi agents disclosed herein can be prepared as pharmaceutical compositions or formulations (also referred to herein as “medicaments”). In some embodiments, pharmaceutical compositions include at least one MUC5AC RNAi agent. These pharmaceutical compositions are particularly useful in the inhibition of the expression of MUC5AC mRNA in a target cell, a group of cells, a tissue, or an organism. The pharmaceutical compositions can be used to treat a subject having a disease, disorder, or condition that would benefit from reduction in the level of the target mRNA, or inhibition in expression of the target gene. The pharmaceutical compositions can be used to treat a subject at risk of developing a disease or disorder that would benefit from reduction of the level of the target mRNA or an inhibition in expression the target gene. In one embodiment, the method includes administering a MUC5AC RNAi agent linked to a targeting ligand as described herein, to a subject to be treated. In some embodiments, one or more pharmaceutically acceptable excipients (including vehicles, carriers, diluents, and/or delivery polymers) are added to the pharmaceutical compositions that include a MUC5AC RNAi agent, thereby forming a pharmaceutical formulation or medicament suitable for in vivo delivery to a subject, including a human.

The pharmaceutical compositions that include a MUC5AC RNAi agent and methods disclosed herein decrease the level of the target mRNA in a cell, group of cells, group of cells, tissue, organ, or subject, including by administering to the subject a therapeutically effective amount of a herein described MUC5AC RNAi agent, thereby inhibiting the expression of MUC5AC mRNA in the subject. In some embodiments, the subject has been previously identified or diagnosed as having a disease or disorder that can be mediated at least in part by a reduction in MUC5AC expression. In some embodiments, the subject has been previously diagnosed with having one or more mucoobstructive lung diseases, such as asthma, CF, COPD, NCFB, PCD. In some embodiments the mucoobstructive lung disease is severe asthma.

In some embodiments the subject has been previously diagnosed with having interstitial lung diseases, cancer (such as lung adenocarcinomas, pancreatic cancer, salivary gland carcinoma, breast cancer, cholangiocarcinoma, ovarian cancer, and other tumors), respiratory infections (such as respiratory syncytial virus, influenza, rhinovirus), otitis media, inflammatory bowel disease, gallstone disease, allergic rhinitis, chronic rhinosinusitis or nasal polyposis.

Embodiments of the present disclosure include pharmaceutical compositions for delivering a MUC5AC RNAi agent to a pulmonary epithelial cell in vivo. Such pharmaceutical compositions can include, for example, a MUC5AC RNAi agent conjugated to a targeting group that comprises an integrin targeting ligand. In some embodiments, the integrin targeting ligand is comprised of an αvβ6 integrin ligand.

In some embodiments, the described pharmaceutical compositions including a MUC5AC RNAi agent are used for treating or managing clinical presentations in a subject that would benefit from the inhibition of expression of MUC5AC. In some embodiments, a therapeutically or prophylactically effective amount of one or more of pharmaceutical compositions is administered to a subject in need of such treatment. In some embodiments, administration of any of the disclosed MUC5AC RNAi agents can be used to decrease the number, severity, and/or frequency of symptoms of a disease in a subject.

In some embodiments, the described MUC5AC RNAi agents are optionally combined with one or more additional (i.e., second, third, etc.) therapeutics. A second therapeutic can be another MUC5AC RNAi agent (e.g., a MUC5AC RNAi agent that targets a different sequence within a MUC5AC gene). In some embodiments, a second therapeutic can be an RNAi agent that targets the MUC5AC gene. An additional therapeutic can also be a small molecule drug, antibody, antibody fragment, and/or aptamer. The MUC5AC RNAi agents, with or without the one or more additional therapeutics, can be combined with one or more excipients to form pharmaceutical compositions.

The described pharmaceutical compositions that include a MUC5AC RNAi agent can be used to treat at least one symptom in a subject having a disease or disorder that would benefit from reduction or inhibition in expression of MUC5AC mRNA. In some embodiments, the subject is administered a therapeutically effective amount of one or more pharmaceutical compositions that include a MUC5AC RNAi agent thereby treating the symptom. In other embodiments, the subject is administered a prophylactically effective amount of one or more MUC5AC RNAi agents, thereby preventing or inhibiting the at least one symptom.

In some embodiments, one or more of the described MUC5AC RNAi agents are administered to a mammal in a pharmaceutically acceptable carrier or diluent. In some embodiments, the mammal is a human.

The route of administration is the path by which a MUC5AC RNAi agent is brought into contact with the body. In general, methods of administering drugs, oligonucleotides, and nucleic acids, for treatment of a mammal are well known in the art and can be applied to administration of the compositions described herein. The MUC5AC RNAi agents disclosed herein can be administered via any suitable route in a preparation appropriately tailored to the particular route. Thus, in some embodiments, the herein described pharmaceutical compositions are administered via inhalation, intranasal administration, intratracheal administration, or oropharyngeal aspiration administration. In some embodiments, the pharmaceutical compositions can be administered by injection, for example, intravenously, intramuscularly, intracutaneously, subcutaneously, intraarticularly, intraocularly, or intraperitoneally, or topically.

The pharmaceutical compositions including a MUC5AC RNAi agent described herein can be delivered to a cell, group of cells, tissue, or subject using oligonucleotide delivery technologies known in the art. In general, any suitable method recognized in the art for delivering a nucleic acid molecule (in vitro or in vivo) can be adapted for use with the compositions described herein. For example, delivery can be by local administration, (e.g., direct injection, implantation, or topical administering), systemic administration, or subcutaneous, intravenous, intraperitoneal, or parenteral routes, including intracranial (e.g., intraventricular, intraparenchymal and intrathecal), intramuscular, transdermal, airway (aerosol), nasal, oral, rectal, or topical (including buccal and sublingual) administration. In some embodiments, the compositions are administered via inhalation, intranasal administration, oropharyngeal aspiration administration, or intratracheal administration. For example, in some embodiments, it is desired that the MUC5AC RNAi agents described herein inhibit the expression of an MUC5AC gene in the pulmonary epithelium, for which administration via inhalation (e.g., by an inhaler device, such as a metered-dose inhaler, or a nebulizer such as a jet or vibrating mesh nebulizer, or a soft mist inhaler) is particularly suitable and advantageous.

In some embodiments, the pharmaceutical compositions described herein comprise one or more pharmaceutically acceptable excipients. The pharmaceutical compositions described herein are formulated for administration to a subject.

As used herein, a pharmaceutical composition or medicament includes a pharmacologically effective amount of at least one of the described therapeutic compounds and one or more pharmaceutically acceptable excipients. Pharmaceutically acceptable excipients (excipients) are substances other than the Active Pharmaceutical Ingredient (API, therapeutic product, e.g., MUC5AC RNAi agent) that are intentionally included in the drug delivery system. Excipients do not exert or are not intended to exert a therapeutic effect at the intended dosage. Excipients can act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, detergents, dextran, dextrose, diluents, disintegrants, emulsifiers, extenders, fillers, flavors, glidants, humectants, lubricants, oils, polymers, preservatives, saline, salts, solvents, sugars, surfactants, suspending agents, sustained release matrices, sweeteners, thickening agents, tonicity agents, vehicles, water-repelling agents, and wetting agents.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor® ELTM (BASF, Parsippany, NJ) or phosphate buffered saline (PBS). It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of the drug that can be in microcrystalline form, for example, in the form of an aqueous microcrystalline suspension. Liposomal formulations or biodegradable polymer systems can also be used to present the drug for both intra-articular and ophthalmic administration.

Formulations suitable for inhalation administration can be prepared by incorporating the active compound in the desired amount in an appropriate solvent, followed by sterile filtration. In general, formulations for inhalation administration are sterile solutions at physiological pH and have low viscosity (<5 cP). Salts may be added to the formulation to balance tonicity. In some cases, surfactants or co-solvents can be added to increase active compound solubility and improve aerosol characteristics. In some cases, excipients can be added to control viscosity in order to ensure size and distribution of nebulized droplets.

In some embodiments, pharmaceutical formulations that include the MUC5AC RNAi agents disclosed herein suitable for inhalation administration can be prepared in water for injection (sterile water), isotonic saline (0.9% saline), or an aqueous sodium phosphate buffer (for example, the MUC5AC RNAi agent formulated in 0.5 mM sodium phosphate monobasic, 0.5 mM sodium phosphate dibasic, in water).

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

The MUC5AC RNAi agents can be formulated in compositions in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the therapeutic effect to be achieved, and the limitations inherent in the art of compounding such an active compound for the treatment of individuals.

A pharmaceutical composition can contain other additional components commonly found in pharmaceutical compositions. Such additional components include, but are not limited to: anti-pruritics, astringents, local anesthetics, or anti-inflammatory agents (e.g., antihistamine, diphenhydramine, etc.). It is also envisioned that cells, tissues, or isolated organs that express or comprise the herein defined RNAi agents may be used as “pharmaceutical compositions.” As used herein, “pharmacologically effective amount,” “therapeutically effective amount,” or simply “effective amount” refers to that amount of an RNAi agent to produce a pharmacological, therapeutic, or preventive result.

In some embodiments, the methods disclosed herein further comprise the step of administering a second therapeutic or treatment in addition to administering an RNAi agent disclosed herein. In some embodiments, the second therapeutic is another MUC5AC RNAi agent (e.g., a MUC5AC RNAi agent that targets a different sequence within the MUC5AC target). In other embodiments, the second therapeutic can be a small molecule drug, an antibody, an antibody fragment, and/or an aptamer.

In some embodiments, described herein are compositions that include a combination or cocktail of at least two MUC5AC RNAi agents having different sequences. In some embodiments, the two or more MUC5AC RNAi agents are each separately and independently linked to targeting groups. In some embodiments, the two or more MUC5AC RNAi agents are each linked to targeting groups that include or consist of integrin targeting ligands. In some embodiments, the two or more MUC5AC RNAi agents are each linked to targeting groups that include or consist of αvβ6 integrin targeting ligands.

Described herein are compositions for delivery of MUC5AC RNAi agents to pulmonary epithelial cells. Furthermore, compositions for delivery of MUC5AC RNAi agents to cells, including renal epithelial cells and/or epithelial cells in the GI or reproductive tract and/or and ocular surface epithelial cells in the eye, in vivo, are generally described herein.

Generally, an effective amount of a MUC5AC RNAi agent disclosed herein will be in the range of from about 0.0001 to about 20 mg/kg of body weight/pulmonary deposited dose (PDD), e.g., from about 0.001 to about 5 mg/kg of body weight/pulmonary deposited dose. In some embodiments, an effective amount of a MUC5AC RNAi agent will be in the range of from about 0.01 mg/kg to about 3.0 mg/kg of body weight per pulmonary deposited dose. In some embodiments, an effective amount of a MUC5AC RNAi agent will be in the range of from about 0.03 mg/kg to about 2.0 mg/kg of body weight per pulmonary deposited dose. In some embodiments, an effective amount of a MUC5AC RNAi agent will be in the range of from about 0.01 to about 1.0 mg/kg of pulmonary deposited dose per body weight. In some embodiments, an effective amount of a MUC5AC RNAi agent will be in the range of from about 0.25 to about 1.0 mg/kg of pulmonary deposited dose per body weight. In some embodiments, an effective amount of a MUC5AC RNAi agent will be in the range of from about 0.25 mg/kg of pulmonary deposited dose per body weight. In some embodiments, an effective amount of a MUC5AC RNAi agent will be in the range of from about 0.50 mg/kg of pulmonary deposited dose per body weight. In some embodiments, an effective amount of a MUC5AC RNAi agent will be in the range of from about 1.0 mg/kg of pulmonary deposited dose per body weight. Calculating the pulmonary deposited dose (PDD) is done in accordance with methods known in the art. (See Wolff R. K., Dorato M. A., Toxicologic Testing of Inhaled Pharmaceutical Aerosols, Crit Rev Toxicol., 1993; 23(4):343-369; Tepper et al., International J. Toxicology, 2016, vol. 35(4):376-392). A comparable and alternatively acceptable method of calculating dose that is well known in the art, especially for human subjects, is determining the respirable delivered dose (RDD). RDD refers to the amount of drug contained in droplets of a size suitable for penetration into the lungs. Generally, an effective amount of a MUC5AC RNAi agent disclosed herein will be in the range of from about 0.001 to about 5 mg respirable delivered dose (RDD)/kg body weight.

For clinical applications, the amount of MUC5AC RNAi agent needed to be loaded into the delivery device of choice (e.g., a nebulizer) that is required to produce such RDDs in human subjects will depend upon the delivery device used (see, for example, Hatley RHM, Byrne SM, Variability in delivered dose and respirable delivered dose from nebulizers: are current regulatory testing guidelines sufficient to produce meaningful information?, Med Devices, 2017, 10:17-28). Some lower efficient nebulizers, for example, RDD is approximately 15%-25% of the dose loaded into the nebulizer. For other more efficient devices, for example, RDD is approximately 50%, approximately 60%, or even higher than 60% of the dose loaded into the nebulizer. In some embodiments, a fixed dose of, for example, approximately 5 mg, approximately 10 gm, approximately 20 mg, approximately 25 mg, approximately 50 mg, approximately 75 mg, approximately 100 mg, approximately 150 mg, approximately 200 mg, approximately 250 mg, or approximately 300 mg of MUC5AC RNAi agent may be loaded into the respective device of choice, which will produce an RDD from about 0.001 to about 5 mg/kg of body weight per dose. The amount desired or required to be administered will also likely depend on such variables as the overall health status of the patient, the relative biological efficacy of the compound delivered, the formulation of the drug, the presence and types of excipient in the formulation, and the route of administration. Also, it is to be understood that the initial dosage administered can be increased beyond the above upper level to rapidly achieve the desired blood-level or tissue-level, or the initial dosage can be smaller than the optimum. In various embodiments, a dose may be administered daily, weekly, bi-weekly, tri-weekly, once monthly, once quarterly (i.e. once every three months), or once every six months. In various embodiments, a dose may be administered at other intervals contained within the range provided above.

For treatment of disease or for formation of a medicament or composition for treatment of a disease, the pharmaceutical compositions described herein including a MUC5AC RNAi agent can be combined with an excipient or with a second therapeutic agent or treatment including, but not limited to: a second or other RNAi agent, a small molecule drug, an antibody, an antibody fragment, peptide, and/or an aptamer.

The described MUC5AC RNAi agents, when added to pharmaceutically acceptable excipients or adjuvants, can be packaged into kits, containers, packs, or dispensers. The pharmaceutical compositions described herein can be packaged in dry powder or aerosol inhalers, other metered-dose inhalers, nebulizers, pre-filled syringes, or vials.

### Methods of Treatment and Inhibition of MUC5AC Expression

The MUC5AC RNAi agents disclosed herein can be used to treat a subject (e.g., a human or other mammal) having a disease or disorder that would benefit from administration of the RNAi agent. In some embodiments, the RNAi agents disclosed herein can be used to treat a subject (e.g., a human) that would benefit from a reduction and/or inhibition in expression of MUC5AC mRNA and/or a reduction in MUC5AC receptor levels.

In some embodiments, the RNAi agents disclosed herein can be used to treat a subject (e.g., a human) having a disease or disorder for which the subject would benefit from reduction in MUC5AC receptors, including but not limited to, mucoobstructive lung diseases (such as asthma, CF, COPD, NCFB, PCD), allergic bronchopulmonary aspergillosis, interstitial lung diseases, cancer (such as lung adenocarcinomas, pancreatic cancer, salivary gland carcinoma, breast cancer, cholangiocarcinoma, ovarian cancer, and other tumors), respiratory infections (such as respiratory syncytial virus, influenza, rhinovirus), otitis media, inflammatory bowel disease, gallstone disease, allergic rhinitis, chronic rhinosinusitis and nasal polyposis. In some embodiments the pulmonary diseases is severe asthma. Treatment of a subject can include therapeutic and/or prophylactic treatment. The subject is administered a therapeutically effective amount of any one or more MUC5AC RNAi agents described herein. The subject can be a human, patient, or human patient. The subject may be an adult, adolescent, child, or infant. Administration of a pharmaceutical composition described herein can be to a human being or animal.

Increased membrane MUC5AC activity is known to promote mucoobstruction tissues. In some embodiments, the described MUC5AC RNAi agents are used to treat at least one symptom mediated at least in part by a reduction in MUC5AC levels, in a subject. The subject is administered a therapeutically effective amount of any one or more of the described MUC5AC RNAi agents. In some embodiments, the subject is administered a prophylactically effective amount of any one or more of the described RNAi agents, thereby treating the subject by preventing or inhibiting the at least one symptom.

In certain embodiments, the present disclosure provides methods for treatment of diseases, disorders, conditions, or pathological states mediated at least in part by MUC5AC gene expression, in a patient in need thereof, wherein the methods include administering to the patient any of the MUC5AC RNAi agents described herein.

In some embodiments, the MUC5AC RNAi agents are used to treat or manage a clinical presentation or pathological state in a subject, wherein the clinical presentation or pathological state is mediated at least in part by a reduction in MUC5AC expression. The subject is administered a therapeutically effective amount of one or more of the MUC5AC RNAi agents or MUC5AC RNAi agent-containing compositions described herein. In some embodiments, the method comprises administering a composition comprising a MUC5AC RNAi agent described herein to a subject to be treated.

In a further aspect, the disclosure features methods of treatment (including prophylactic or preventative treatment) of diseases or symptoms that may be addressed by a reduction in MUC5AC receptor levels, the methods comprising administering to a subject in need thereof a MUC5AC RNAi agent that includes an antisense strand comprising the sequence of any of the sequences in Table 2, Table 3, or Table 11. Also described herein are compositions for use in such methods.

The described MUC5AC RNAi agents and/or compositions that include MUC5AC RNAi agents can be used in methods for therapeutic treatment of disease or conditions caused by enhanced or elevated MUC5AC protein or MUC5AC gene expression. Such methods include administration of a MUC5AC RNAi agent as described herein to a subject, e.g., a human or animal subject.

In another aspect, the disclosure provides methods for the treatment (including prophylactic treatment) of a pathological state (such as a condition or disease) mediated at least in part by MUC5AC expression, wherein the methods include administering to a subject a therapeutically effective amount of an RNAi agent that includes an antisense strand comprising the sequence of any of the sequences in Table 2, Table 3, or Table 11.

In some embodiments, methods for inhibiting expression of an MUC5AC gene are disclosed herein, wherein the methods include administering to a cell an RNAi agent that includes an antisense strand comprising the sequence of any of the sequences in Table 2, Table 3, or Table 11.

In some embodiments, methods for the treatment (including prophylactic treatment) of a pathological state mediated at least in part by MUC5AC expression are disclosed herein, wherein the methods include administering to a subject a therapeutically effective amount of an RNAi agent that includes a sense strand comprising the sequence of any of the sequences in Table 2, Table 4, Table 5, Table 6, Table 7, or Table 11.

In some embodiments, methods for inhibiting expression of an MUC5AC gene are disclosed herein, wherein the methods comprise administering to a cell an RNAi agent that includes a sense strand comprising the sequence of any of the sequences in Table 2, Table 4, Table 5, Table 6, Table 7, or Table 11.

In some embodiments, methods for the treatment (including prophylactic treatment) of a pathological state mediated at least in part by MUC5AC expression are disclosed herein, wherein the methods include administering to a subject a therapeutically effective amount of an RNAi agent that includes a sense strand comprising the sequence of any of the sequences in Table 4, Table 5, Table 6, Table 7, or Table 11, and an antisense strand comprising the sequence of any of the sequences in Table 3 or Table 11.

In some embodiments, methods for inhibiting expression of a MUC5AC gene are disclosed herein, wherein the methods include administering to a cell an RNAi agent that includes a sense strand comprising the sequence of any of the sequences in Table 4, Table 5, Table 6, Table 7, or Table 11. and an antisense strand comprising the sequence of any of the sequences in Table 3 or Table 11.

In some embodiments, methods of inhibiting expression of a MUC5AC gene are disclosed herein, wherein the methods include administering to a subject a MUC5AC RNAi agent that includes a sense strand consisting of the nucleobase sequence of any of the sequences in Table 4, Table 5, Table 6, Table 7, or Table 11, and the antisense strand consisting of the nucleobase sequence of any of the sequences in Table 3 or Table 11. In other embodiments, disclosed herein are methods of inhibiting expression of a MUC5AC gene, wherein the methods include administering to a subject a MUC5AC RNAi agent that includes a sense strand consisting of the modified sequence of any of the modified sequences in Table 4, Table 5, Table 6, Table 7, or Table 11, and the antisense strand consisting of the modified sequence of any of the modified sequences in Table 3 or Table 11.

In some embodiments, methods for inhibiting expression of an MUC5AC gene in a cell are disclosed herein, wherein the methods include administering one or more MUC5AC RNAi agents comprising a duplex structure of one of the duplexes set forth in Tables 8A, 8B, 8C, 9, 10A, 10B, and 11.

In some embodiments, the quantity or amount of MUC5AC protein and/or MUC5AC mRNA in certain pulmonary epithelial cells of subject to whom a described MUC5AC RNAi agent is administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99%, relative to the subject prior to being administered the MUC5AC RNAi agent or to a subject not receiving the MUC5AC RNAi agent. In some embodiments, MUC5AC protein levels in certain epithelial cells of a subject to whom a described MUC5AC RNAi agent is administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99%, relative to the subject prior to being administered the MUC5AC RNAi agent or to a subject not receiving the MUC5AC RNAi agent. The gene expression level, protein level, and/or mRNA level in the subject may be reduced in a cell, group of cells, and/or tissue of the subject. In some embodiments, the MUC5AC mRNA levels in certain epithelial cells subject to whom a described MUC5AC RNAi agent has been administered is reduced by at least about 30%, 35%, 40%, 45%, 50%, 55%, 60%, 65%, 70%, 75%, 80%, 85%, 90%, 95%, or 98% relative to the subject prior to being administered the MUC5AC RNAi agent or to a subject not receiving the MUC5AC RNAi agent.

A reduction in MUC5AC mRNA and MUC5AC protein levels can be assessed by any methods known in the art. Reduction or decrease in MUC5AC mRNA and/or MUC5AC protein levels are collectively referred to herein as a decrease in, reduction of, or inhibition of MUC5AC gene expression. The Examples set forth herein illustrate known methods for assessing inhibition of MUC5AC.

### Cells, Tissues, Organs, and Non-Human Organisms

Cells, tissues, organs, and non-human organisms that include at least one of the MUC5AC RNAi agents described herein are contemplated. The cell, tissue, organ, or non-human organism is made by delivering the RNAi agent to the cell, tissue, organ, or non-human organism.

### Additional Illustrative Embodiments

Provided here are certain additional illustrative embodiments of the disclosed technology. These embodiments are illustrative only and do not limit the scope of the present disclosure or of the claims attached hereto.

Embodiment 1. An RNAi agent for inhibiting expression of a Mucin 5AC gene, comprising: an antisense strand comprising at least 17 contiguous nucleotides differing by 0 or 1 nucleotides from any one of the sequences provided in Table 2 or Table 3; and a sense strand comprising a nucleotide sequence that is at least partially complementary to the antisense strand

Embodiment 2. The RNAi agent of Embodiment 1, wherein the antisense strand comprises nucleotides 2-18 of any one of the sequences provided in Table 2 or Table 3.

Embodiment 3. The RNAi agent of Embodiment 1 or Embodiment 2, wherein the sense strand comprises a nucleotide sequence of at least 17 contiguous nucleotides differing by 0 or 1 nucleotides from any one of the sequences provided in Table 2 or Table 4, and wherein the sense strand has a region of at least 85% complementarity over the 17 contiguous nucleotides to the antisense strand.

Embodiment 4. The RNAi agent of any one of Embodiments 1-3, wherein at least one nucleotide of the RNAi agent is a modified nucleotide or includes a modified internucleoside linkage.

Embodiment 5. The RNAi agent of any one of Embodiments 1-4, wherein all or substantially all of the nucleotides are modified nucleotides.

Embodiment 6. The RNAi agent of any one of Embodiments 4-5, wherein the modified nucleotide is selected from the group consisting of: 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide.

Embodiment 7. The RNAi agent of Embodiment 5, wherein all or substantially all of the nucleotides are modified with 2′-O-methyl nucleotides, 2′-fluoro nucleotides, or combinations thereof.

Embodiment 8. The RNAi agent of any one of Embodiments 1-7, wherein the antisense strand comprises the nucleotide sequence of any one of the modified antisense strand sequences provided in Table 3 or Table 11.

Embodiment 9. The RNAi agent of any one of Embodiments 1-8, wherein the sense strand comprises the nucleotide sequence of any one of the modified sense strand sequences provided in Table 4 or Table 11.

Embodiment 10. The RNAi agent of Embodiment 1, wherein the antisense strand comprises the nucleotide sequence of any one of the modified antisense strand sequences provided in Table 3 or Table 11, and the sense strand comprises the nucleotide sequence of any one of the modified sense strand sequences provided in Table 4 or Table 11.

Embodiment 11. The RNAi agent of any one of Embodiments 1-10, wherein the sense strand is between 18 and 30 nucleotides in length, and the antisense strand is between 18 and 30 nucleotides in length.

Embodiment 12. The RNAi agent of Embodiment 11, wherein the sense strand and the antisense strand are each between 18 and 27 nucleotides in length.

Embodiment 13. The RNAi agent of Embodiment 12, wherein the sense strand and the antisense strand are each between 18 and 24 nucleotides in length.

Embodiment 14. The RNAi agent of Embodiment 13, wherein the sense strand and the antisense strand are each 21 nucleotides in length.

Embodiment 15. The RNAi agent of Embodiment 14, wherein the RNAi agent has two blunt ends.

Embodiment 16. The RNAi agent of any one of Embodiments 1-15, wherein the sense strand comprises one or two terminal caps.

Embodiment 17. The RNAi agent of any one of Embodiments 1-16, wherein the sense strand comprises one or two inverted abasic residues.

Embodiment 18. The RNAi agent of Embodiment 1, wherein the RNAi agent is comprised of a sense strand and an antisense strand that form a duplex having the structure of any one of the duplexes in Table 8A, Table 8B, Table 8C, Table 9, Table 10A, or Table 10B.

Embodiment 19. The RNAi agent of Embodiment 18, wherein all or substantially all of the nucleotides are modified nucleotides.

Embodiment 20. The RNAi agent of Embodiment 1, wherein the antisense strand consists of, consists essentially of, or comprises a nucleotide sequence that differs by 0 or 1 nucleotides from one of the following nucleotide sequences (5′→3′):

(SEQ ID NO: 79)
UUGUAGUAGUCGCAGAACA;
or
(SEQ ID NO: 83)
UUCUUGUUCAGGCAAAUCA.

Embodiment 21. The RNAi agent of Embodiment 1, wherein the antisense strand consists of, consists essentially of, or comprises a nucleotide sequence that differs by 0 or 1 nucleotides from one of the following nucleotide sequences (5′→3′):

(SEQ ID NO: 1525)
UUGUAGUAGUCGCAGAACAGC;
or
(SEQ ID NO: 1535)
UUCUUGUUCAGGCAAAUCAGC.

Embodiment 22. The RNAi agent of Embodiment 1, wherein the sense strand consists of, consists essentially of, or comprises a nucleotide sequence that differs by 0 or 1 nucleotides from one of the following nucleotide sequences (5′→3′):

(SEQ ID NO: 568)
UGUUCUGCGACUACUACAA;
or
(SEQ ID NO: 572)
UGAUUUGCCUGAACAAGAA.

Embodiment 23. The RNAi agent of Embodiment 20, 21, or 22, wherein all or substantially all of the nucleotides are modified nucleotides

Embodiment 24. The RNAi agent of Embodiment 1, wherein the antisense strand comprises, consists of, or consists essentially of a modified nucleotide sequence that differs by 0 or 1 nucleotides from one of the following nucleotide sequences (5′→3′):

(SEQ ID NO: 1127)
cPrpusUfsgsUfaGfuAfgUfcGfcAfgAfaCfaGfsc;
(SEQ ID NO: 1065)
usUfsgsUfaGfuAfgUfcGfcAfgAfaCfaGfsc;
(SEQ ID NO: 1166)
usUfscsuuguucagGfcAfaAfucagsc;
or
(SEQ ID NO: 1191)
cPrpuUfcuuguucagGfcAfaAfucagsc;

wherein a, c, g, and u represent 2′-O-methyl adenosine, 2′-O-methyl cytidine, 2′-O-methyl guanosine, and 2′-O-methyl uridine, respectively; Af, Cf, Gf, and Uf represent 2′-fluoro adenosine, 2′-fluoro cytidine, 2′-fluoro guanosine, and 2′-fluoro uridine, respectively; cPrpu represents a 5′-cyclopropyl phosphonate-2′-O-methyl uridine; s represents a phosphorothioate linkage; and wherein all or substantially all of the nucleotides on the sense strand are modified nucleotides.

Embodiment 25. The RNAi agent of Embodiment 1, wherein the sense strand comprises, consists of, or consists essentially of a modified nucleotide sequence that differs by 0 or 1 nucleotides from one of the following nucleotide sequences (5′→3′):

(SEQ ID NO: 1265)
gscuguucuGfCfGfacuacuacaa;
or
(SEQ ID NO: 1315)
gscugauUfuGfcCfugaacaagaa;

wherein a, c, g, and u represent 2′-O-methyl adenosine, 2′-O-methyl cytidine, 2′-O-methyl guanosine, and 2′-O-methyl uridine, respectively; Af, Cf, Gf, and Uf represent 2′-fluoro adenosine, 2′-fluoro cytidine, 2′-fluoro guanosine, and 2′-fluoro uridine, respectively; and s represents a phosphorothioate linkage; and wherein all or substantially all of the nucleotides on the antisense strand are modified nucleotides.

Embodiment 26. The RNAi agent of any one of Embodiments 20-25, wherein the sense strand further includes inverted abasic residues at the 3′ terminal end of the nucleotide sequence, at the 5′ end of the nucleotide sequence, or at both.

Embodiment 27. The RNAi agent of any one of Embodiments 1-26, wherein the RNAi agent is linked to a targeting ligand.

Embodiment 28. The RNAi agent of Embodiment 27, wherein the targeting ligand has affinity for a cell receptor expressed on an epithelial cell.

Embodiment 29. The RNAi agent of Embodiment 28, wherein the targeting ligand comprises an integrin targeting ligand.

Embodiment 30. The RNAi agent of Embodiment 29, wherein the integrin targeting ligand is an αvβ6 integrin targeting ligand.

Embodiment 31. The RNAi agent of Embodiment 30, wherein the targeting ligand comprises the structure:

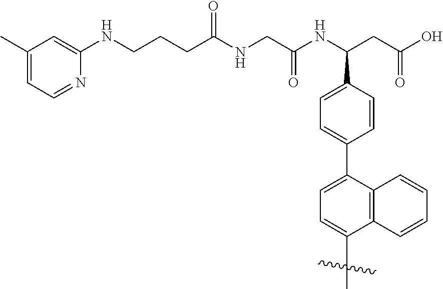 [SMILES: *c1ccc(-c2ccc([C@H](CC(=O)O)NC(=O)CNC(=O)CCCNc3cc(C)ccn3)cc2)c2ccccc12]

or a pharmaceutically acceptable salt thereof, or

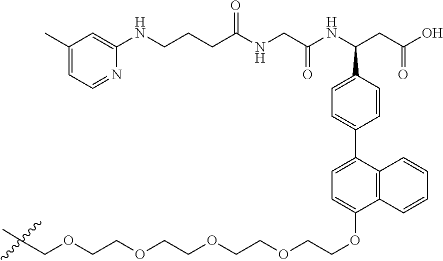 [SMILES: *COCCOCCOCCOCCOc1ccc(-c2ccc([C@H](CC(=O)O)NC(=O)CNC(=O)CCCNc3cc(C)ccn3)cc2)c2ccccc12]

or a pharmaceutically acceptable salt thereof, wherein [symbol] indicates the point of connection to the RNAi agent.

Embodiment 32. The RNAi agent of any one of Embodiments 27-30, wherein RNAi agent is conjugated to a targeting ligand having the following structure:

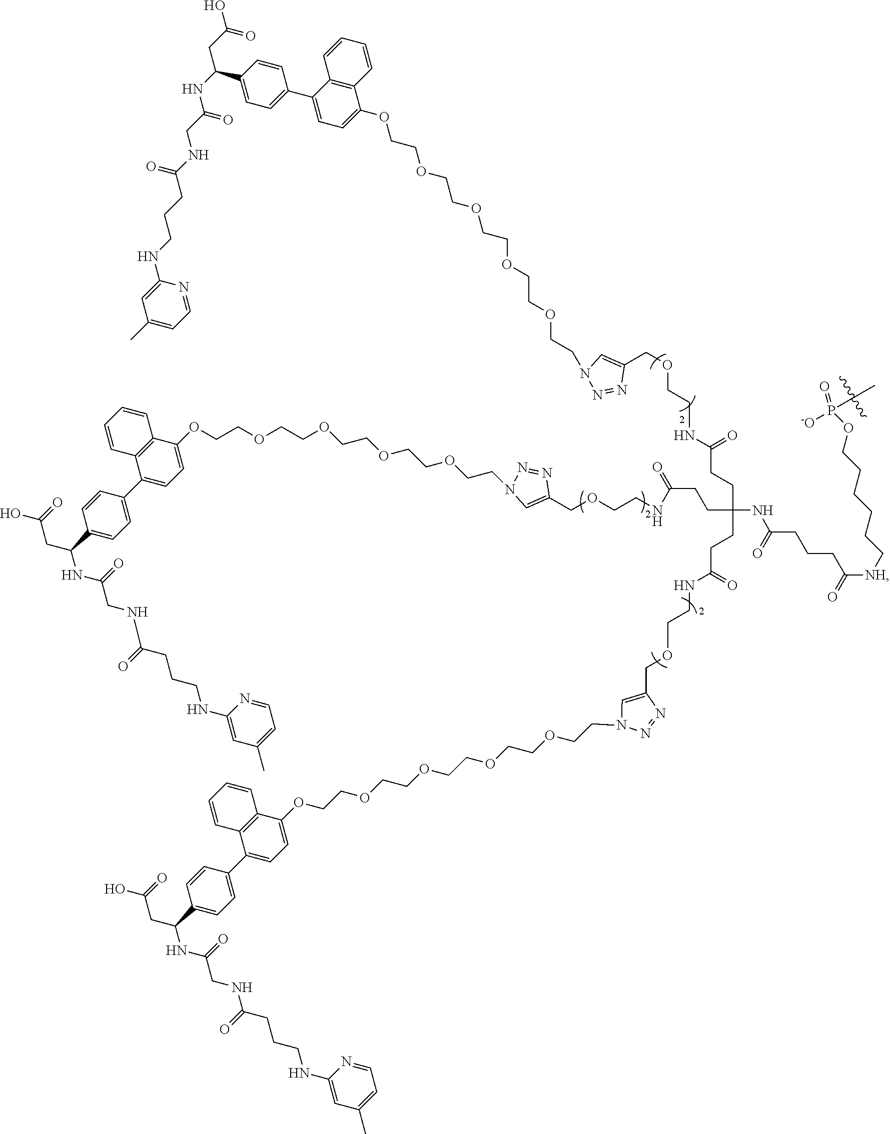

pharmaceutically acceptable salt thereof, wherein [symbol] indicates the point of connection to the RNAi agent.

Embodiment 33. The RNAi agent of any one of Embodiments 27-30, wherein the targeting ligand has the following structure:

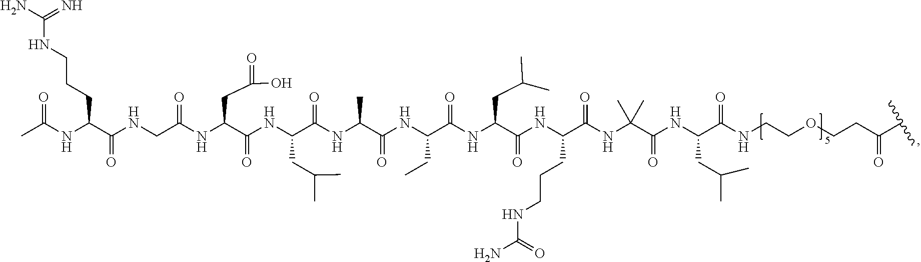

pharmaceutically acceptable salt thereof, wherein [symbol] indicates the point of connection to the RNAi agent.

Embodiment 34. The RNAi agent of any one of Embodiments 27-33, wherein the targeting ligand is conjugated to the sense strand.

Embodiment 35. The RNAi agent of Embodiment 34, wherein the targeting ligand is conjugated to the 5′ terminal end of the sense strand.

Embodiment 36. A composition comprising the RNAi agent of any one of Embodiments 1-35, wherein the composition further comprises a pharmaceutically acceptable excipient.

Embodiment 37. The composition of Embodiment 36, further comprising a second RNAi agent capable of inhibiting the expression of Mucin 5AC gene expression.

Embodiment 38. The composition of any one of Embodiments 36-37, further comprising one or more additional therapeutics.

Embodiment 39. The composition of any one of Embodiments 36-38, wherein the composition is formulated for administration by inhalation.

Embodiment 40. The composition of Embodiment 39, wherein the composition is delivered by a metered-dose inhaler, jet nebulizer, vibrating mesh nebulizer, or soft mist inhaler.

Embodiment 41. The composition of any of Embodiments 36-40, wherein the RNAi agent is a sodium salt.

Embodiment 42. The composition of any of Embodiments 36-41, wherein the pharmaceutically acceptable excipient is water for injection.

Embodiment 43. The composition of any of Embodiments 36-42, wherein the pharmaceutically acceptable excipient is isotonic saline.

Embodiment 44. A method for inhibiting expression of a MUC5AC gene in a cell, the method comprising introducing into a cell an effective amount of an RNAi agent of any one of Embodiments 1-35 or the composition of any one of Embodiments 36-43.

Embodiment 45. The method of Embodiment 44, wherein the cell is within a subject.

Embodiment 46. The method of Embodiment 45, wherein the subject is a human subject.

Embodiment 47. The method of any one of claims 44-46, wherein following the administration of the RNAi agent the Mucin 5AC gene expression is inhibited by at least about 30%.

Embodiment 48. A method of treating one or more symptoms or diseases associated with MUC5AC protein levels, the method comprising administering to a human subject in need thereof a therapeutically effective amount of the composition of any one of Embodiments 36-43.

Embodiment 49. The method of Embodiment 48, wherein the disease is a mucoobstructive lung disease.

Embodiment 50. The method of Embodiment 49, wherein the mucoobstructive lung disease is asthma (including severe asthma), cystic fibrosis (CF), bronchiectasis (NCFB), or chronic obstructive pulmonary disease (COPD).

Embodiment 51. The method of Embodiment 50, wherein the disease is asthma (including severe asthma).

Embodiment 52. The method of Embodiment 48, wherein the disease is cancer.

Embodiment 53. The method of Embodiment 52, wherein the cancer is lung adenocarcinoma, pancreatic cancer, salivary gland carcinoma, breast cancer, cholangiocarcinoma, or ovarian cancer.

Embodiment 54. The method of any one of Embodiments 44-53, wherein the RNAi agent is administered at a pulmonary deposited dose (PDD) of about 0.01 mg/kg to about 5.0 mg/kg of body weight of the subject.

Embodiment 55. The method of any one of Embodiments 44-53, wherein the RNAi agent is administered at a pulmonary deposited dose (PDD) of about 0.1 mg/kg to about 2.0 mg/kg of body weight of the subject.

Embodiment 56. The method of any one of Embodiments 44-53, wherein the RNAi agent is administered at a respirable delivered dose (RDD) of about 0.01 mg/kg to about 5.0 mg/kg of body weight of the subject.

Embodiment 57. The method of any one of Embodiments 44-53, wherein the RNAi agent is administered at a respirable delivered dose (RDD) of about 0.1 mg/kg to about 2.0 mg/kg of body weight of the subject.

Embodiment 58. The method of any of Embodiments 44-57, wherein the RNAi agent is administered in two or more doses.

Embodiment 59. Use of the RNAi agent of any one of Embodiments 1-35, for the treatment of a disease, disorder, or symptom that is mediated at least in part by Mucin 5AC protein levels.

Embodiment 60. Use of the composition according to any one of Embodiments 36-43, for the treatment of a disease, disorder, or symptom that is mediated at least in part by Mucin 5AC gene expression.

Embodiment 61. Use of the composition according to any one of Embodiments 36-43, for the manufacture of a medicament for treatment of a disease, disorder, or symptom that is mediated at least in part by Mucin 5AC gene expression.

Embodiment 62. The use of any one of Embodiments 59-61, wherein the disease is asthma (including severe asthma).

Embodiment 63. A method of making an RNAi agent of any one of Embodiments 1-35, comprising annealing a sense strand and an antisense strand to form a double-stranded ribonucleic acid molecule.

Embodiment 64. The method of Embodiment 63, wherein the sense strand comprises a targeting ligand.

Embodiment 65. The method of Embodiment 64, comprising conjugating a targeting ligand to the sense strand.

Embodiment 66. An RNAi agent for inhibiting expression of a Mucin 5AC gene, comprising:
an antisense strand comprising at least 15 contiguous nucleotides differing by 0, 1, 2, or 3 nucleotides from any one of the sequences provided in Table 2, Table 3, or Table 11; and a sense strand comprising a nucleotide sequence that is at least partially complementary to the antisense strand.

Embodiment 67. An RNAi agent for inhibiting expression of a Mucin 5AC (MUC5AC) gene, comprising:
an antisense strand comprising at least 15 contiguous nucleotides differing by 0, 1, 2, or 3 nucleotides from any one of the sequences disclosed in Table 2 or Table 3; and a sense strand comprising a nucleotide sequence that is at least partially complementary to the antisense strand.

Embodiment 68. An RNAi agent for inhibiting expression of a Mucin 5AC (MUC5AC) gene, comprising:
a sense strand comprising at least 15 contiguous nucleotides differing by 0, 1, 2, or 3 nucleotides from a stretch of the same length of nucleotides of SEQ ID NO:1; and an antisense strand comprising a nucleotide sequence that is at least partially complementary to the sense strand.

Embodiment 69. An inhibitor of a MUC5AC gene comprising an antisense nucleotide sequence having at least 15 contiguous nucleotides differing by 0, 1, 2, or 3 nucleotides that are complementary to any of the target nucleotide sequences in Table 1.

Embodiment 70. An RNAi agent comprising (i) an antisense strand comprising a nucleotide sequence having at least 15 contiguous nucleotides differing by 0, 1, 2, or 3 nucleotides from any of the nucleotide sequences in Table 2, Table 3 or Table 11, and (ii) a sense strand at least partially complementary to the antisense strand.

Embodiment 71. An RNAi agent comprising (i) an antisense strand comprising, consisting of, or consisting essentially of a nucleotide sequence from any of the antisense strand nucleotide sequences in Table 2, Table 3 or Table 11, and (ii) a sense strand comprising, consisting of, or consisting essentially of a nucleotide sequence from any of the sense strand nucleotide sequences in Table 2, Table 4, Table 5, Table 6, Table 7, or Table 11.

Embodiment 72. An RNAi agent comprising an antisense strand and sense strand annealed to form a duplex, wherein the duplex has the structure of any of the duplexes set forth in Table 8A, Table 8B, Table 8C, Table 9, Table 10, or Table 11.

Embodiment 73. An RNAi agent for inhibiting expression of a Mucin 5AC gene, comprising:
an antisense strand comprising at least 17 contiguous nucleotides differing by 0 or 1 nucleotides from any one of the sequences provided in Table 2 or Table 3; and
a sense strand comprising a nucleotide sequence that is at least partially complementary to the antisense strand,
   wherein optionally all or substantially all of the nucleotides of the sense strand and the antisense strand modified nucleotides, and wherein the sense strand is optionally linked to a targeting ligand.

The above provided embodiments and items are now illustrated with the following, non-limiting examples.

### EXAMPLES

### Example 1. Synthesis of MUC5AC RNAi Agents

MUC5AC RNAi agent duplexes disclosed herein were synthesized in accordance with the following:

### A. Synthesis.

The sense and antisense strands of the MUC5AC RNAi agents were synthesized according to phosphoramidite technology on solid phase used in oligonucleotide synthesis. Depending on the scale, a MerMade96E® (Bioautomation), a MerMadel2® (Bioautomation), or an OP Pilot 100 (GE Healthcare) was used. Syntheses were performed on a solid support made of controlled pore glass (CPG, 500 Å or 600 Å, obtained from Prime Synthesis, Aston, PA, USA). All RNA and 2′-modified RNA phosphoramidites were purchased from Thermo Fisher Scientific (Milwaukee, WI, USA). Specifically, the 2′-O-methyl phosphoramidites that were used included the following: (5′-O-dimethoxytrityl-N⁶-(benzoyl)-2′-O-methyl-adenosine-3′-O-(2-cyanoethyl-N,N-diisopropylamino) phosphoramidite, 5′-O-dimethoxy-trityl-N⁴-(acetyl)-2′-O-methyl-cytidine-3′-O-(2-cyanoethyl-N,N-diisopropyl-amino) phosphoramidite, (5′-O-dimethoxytrityl-N²-(isobutyryl)-2′-O-methyl-guanosine-3′-O-(2-cyanoethyl-N,N-diisopropylamino) phosphoramidite, and 5′-O-dimethoxytrityl-2′-O-methyl-uridine-3′-O-(2-cyanoethyl-N,N-diisopropylamino) phosphoramidite. The 2′-deoxy-2′-fluoro-phosphoramidites carried the same protecting groups as the 2′-O-methyl RNA amidites. 5′-dimethoxytrityl-2′-O-methyl-inosine-3′-O-(2-cyanoethyl-N,N-diisopropylamino) phosphoramidites were purchased from Glen Research (Virginia). The inverted abasic (3′-O-dimethoxytrityl-2′-deoxyribose-5′-O-(2-cyanoethyl-N,N-diisopropylamino) phosphoramidites were purchased from ChemGenes (Wilmington, MA, USA). The following UNA phosphoramidites were used: 5′-(4,4′-Dimethoxytrityl)-N6-(benzoyl)-2′,3′-seco-adenosine, 2′-benzoyl-3′-[(2-cyanoethyl)-(N,N-diisopropyl)]-phosphoramidite, 5′-(4,4′-Dimethoxytrityl)-N-acetyl-2′,3′-seco-cytosine, 2′-benzoyl-3′-[(2-cyanoethyl)-(N,N-diisopropyl)]-phosphoramidite, 5′-(4,4′-Dimethoxytrityl)-N-isobutyryl-2′,3′-seco-guanosine, 2′-benzoyl-3′-[(2-cyanoethyl)-(N,N-diisopropyl)]-phosphoramidite, and 5′-(4,4′-Dimethoxy-trityl)-2′,3′-seco-uridine, 2′-benzoyl-3′-[(2-cyanoethyl)-(N,N-diisopropyl)]-phosphoramidite. TFA aminolink phosphoramidites were also commercially purchased (ThermoFisher). Linker L6 was purchased as propargyl-PEG5-NHS from BroadPharm (catalog #BP-20907) and coupled to the NH₂—C₆ group from an aminolink phosphoramidite to form -L6-C6-, using standard coupling conditions. The linker Alk-cyHex was similarly commercially purchased from Lumiprobe (alkyne phosphoramidite, 5′-terminal) as a propargyl-containing compound phosphoramidite compound to form the linker -Alk-cyHex-. In each case, phosphorothioate linkages were introduced as specified using the conditions set forth herein. The cyclopropyl phosphonate phosphoramidites were synthesized in accordance with International Patent Application Publication No. WO 2017/214112 (see also Altenhofer et. al., Chem. Communications (Royal Soc. Chem.), 57(55):6808-6811 (2021)).

Tri-alkyne-containing phosphoramidites were dissolved in anhydrous dichloromethane or anhydrous acetonitrile (50 mM), while all other amidites were dissolved in anhydrous acetonitrile (50 mM) and molecular sieves (3 Å) were added. 5-Benzylthio-1H-tetrazole (BTT, 250 mM in acetonitrile) or 5-Ethylthio-1H-tetrazole (ETT, 250 mM in acetonitrile) was used as activator solution. Coupling times were 10 minutes (RNA), 90 seconds (2′ O-Me), and 60 seconds (2′ F). In order to introduce phosphorothioate linkages, a 100 mM solution of 3-phenyl 1,2,4-dithiazoline-5-one (POS, obtained from PolyOrg, Inc., Leominster, MA, USA) in anhydrous acetonitrile was employed.

Alternatively, tri-alkyne moieties were introduced post-synthetically (see section E, below). For this route, the sense strand was functionalized with a 5′ and/or 3′ terminal nucleotide containing a primary amine. TFA aminolink phosphoramidite was dissolved in anhydrous acetonitrile (50 mM) and molecular sieves (3 Å) were added. 5-Benzylthio-1H-tetrazole (BTT, 250 mM in acetonitrile) or 5-Ethylthio-1H-tetrazole (ETT, 250 mM in acetonitrile) was used as activator solution. Coupling times were 10 minutes (RNA), 90 seconds (2′ O-Me), and 60 seconds (2′ F). In order to introduce phosphorothioate linkages, a 100 mM solution of 3-phenyl 1,2,4-dithiazoline-5-one (POS, obtained from PolyOrg, Inc., Leominster, MA, USA) in anhydrous acetonitrile was employed.

### B. Cleavage and Deprotection of Support Bound Oligomer.

After finalization of the solid phase synthesis, the dried solid support was treated with a 1:1 volume solution of 40 wt. % methylamine in water and 28% to 31% ammonium hydroxide solution (Aldrich) for 1.5 hours at 30° C. The solution was evaporated and the solid residue was reconstituted in water (see below).

### C. Purification.

Crude oligomers were purified by anionic exchange HPLC using a TSKgel SuperQ-5PW 13 μm column and Shimadzu LC-8 system. Buffer A was 20 mM Tris, 5 mM EDTA, pH 9.0 and contained 20% Acetonitrile and buffer B was the same as buffer A with the addition of 1.5 M sodium chloride. UV traces at 260 nm were recorded. Appropriate fractions were pooled then run on size exclusion HPLC using a GE Healthcare XK 16/40 column packed with Sephadex G-25 fine with a running buffer of 100 mM ammonium bicarbonate, pH 6.7 and 20% Acetonitrile or filtered water. Alternatively, pooled fractions were desalted and exchanged into an appropriate buffer or solvent system via tangential flow filtration.

### D. Annealing.

Complementary strands were mixed by combining equimolar RNA solutions (sense and antisense) in 1×PBS (Phosphate-Buffered Saline, 1×, Corning, Cellgro) to form the RNAi agents. Some RNAi agents were lyophilized and stored at −15 to −25° C. Duplex concentration was determined by measuring the solution absorbance on a UV-Vis spectrometer in 1×PBS. The solution absorbance at 260 nm was then multiplied by a conversion factor (0.050 mg/(mL-cm)) and the dilution factor to determine the duplex concentration.

### E. Conjugation of Tri-Alkyne Linker.

In some embodiments a tri-alkyne linker is conjugated to the sense strand of the RNAi agent on resin as a phosphoramidite (see Example 1G for the synthesis of an example tri-alkyne linker phosphoramidite and Example TA for the conjugation of the phosphoramidite.). In other embodiments, a tri-alkyne linker may be conjugated to the sense strand following cleavage from the resin, described as follows: either prior to or after annealing, in some embodiments, the 5′ or 3′ amine functionalized sense strand is conjugated to a tri-alkyne linker. An example tri-alkyne linker structure that can be used in forming the constructs disclosed herein is as follows:

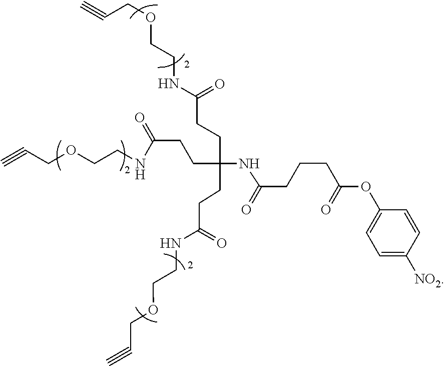

To conjugate the tri-alkyne linker to the annealed duplex, amine-functionalized duplex was dissolved in 90% DMSO/10% H₂O, at ˜50-70 mg/mL. 40 equivalents triethylamine was added, followed by 3 equivalents tri-alkyne-PNP. Once complete, the conjugate was precipitated twice in a solvent system of 1× phosphate buffered saline/acetonitrile (1:14 ratio), and dried.

### F. Synthesis of Targeting Ligand SM6.1

### ((S)-3-(4-(4-((14-azido-3,6,9,12-tetraoxatetradecyl)oxy)naphthalen-1-yl)phenyl)-3-(2-(4-((4-methylpyridin-2-yl)amino)butanamido)acetamido)propanoic acid)

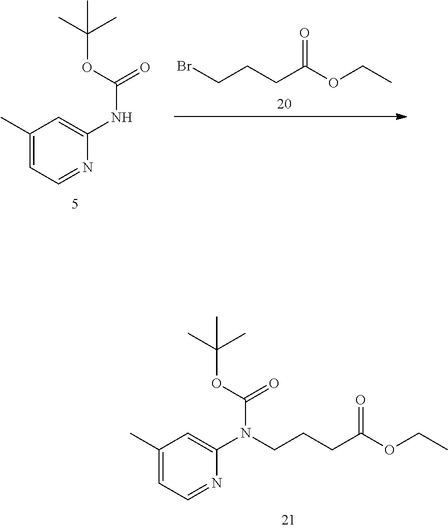

Compound 5 (tert-Butyl(4-methylpyridin-2-yl)carbamate) (0.501 g, 2.406 mmol, 1 equiv.) was dissolved in DMF (17 mL). To the mixture was added NaH (0.116 mg, 3.01 mmol, 1.25 eq, 60% dispersion in oil) The mixture stirred for 10 min before adding Compound 20 (Ethyl 4-Bromobutyrate (0.745 g, 3.82 mmol, 0.547 mL)) (Sigma 167118). After 3 hours the reaction was quenched with ethanol (18 mL) and concentrated. The concentrate was dissolved in DCM (50 mL) and washed with saturated aq. NaCl solution (1×50 mL), dried over Na₂SO₄, filtered and concentrated. The product was purified on silica column, gradient 0-5% Methanol in DCM.

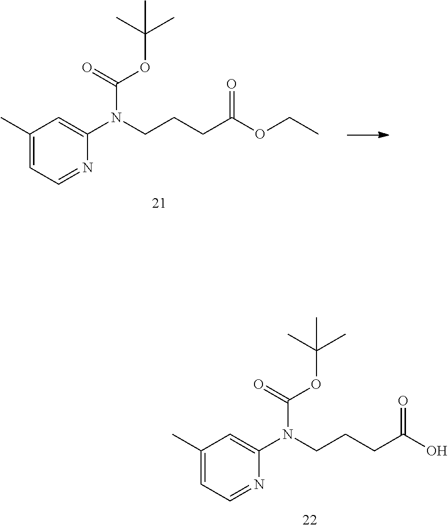

Compound 21 was dissolved (0.80 g, 2.378 mmol) in 100 mL of Acetone: 0.1 M NaOH [1:1]. The reaction was monitored by TLC (5% ethyl acetate in hexane). The organics were concentrated away, and the residue was acidified to pH 3-4 with 0.3 M Citric Acid (40 mL). The product was extracted with DCM (3×75 mL). The organics were pooled, dried over Na₂SO₄, filtered and concentrated. The product was used without further purification.

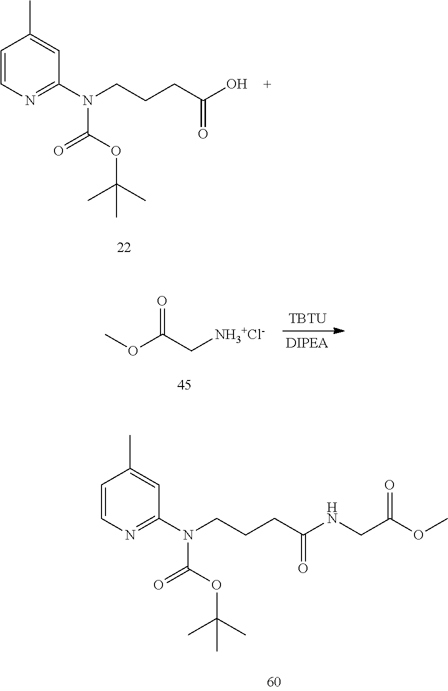

To a solution of Compound 22 (1.1 g, 3.95 mmol, 1 equiv.), Compound 45 (595 mg, 4.74 mmol, 1.2 equiv.), and TBTU (1.52 g, 4.74 mmol, 1.2 equiv.) in anhydrous DMF (10 mL) was added diisopropylethylamine (2.06 mL, 11.85 mmol, 3 equiv.) at 0° C. The reaction mixture was warmed to room temperature and stirred 3 hours. The reaction was quenched by saturated NaHCO₃ solution (10 mL). The aqueous phase was extracted with ethyl acetate (3×10 mL) and the organic phase was combined, dried over anhydrous Na₂SO₄, and concentrated. The product was separated by CombiFlash® using silica gel as the stationary phase. LC-MS: calculated [M+H]+ 366.20, found 367.

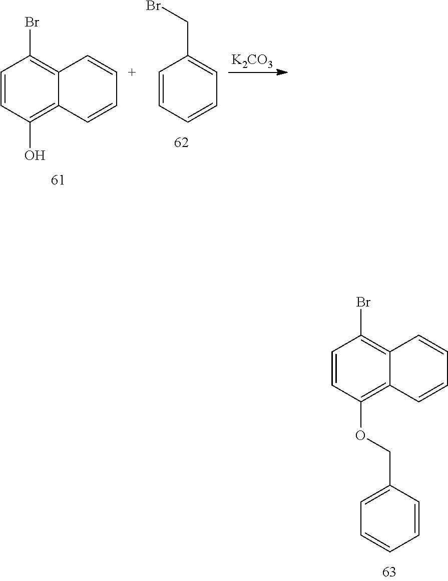

To a solution of compound 61 (2 g, 8.96 mmol, 1 equiv.), and compound 62 (2.13 mL, 17.93 mmol, 2 equiv.) in anhydrous DMF (10 mL) was added K₂CO₃ (2.48 g, 17.93 mmol, 2 equiv.) at 0° C. The reaction mixture was warmed to room temperature and stirred overnight. The reaction was quenched by water (10 mL). The aqueous phase was extracted with ethyl acetate (3×10 mL) and the organic phase was combined, dried over anhydrous Na₂SO₄, and concentrated. The product was separated by CombiFlash® using silica gel as the stationary phase.

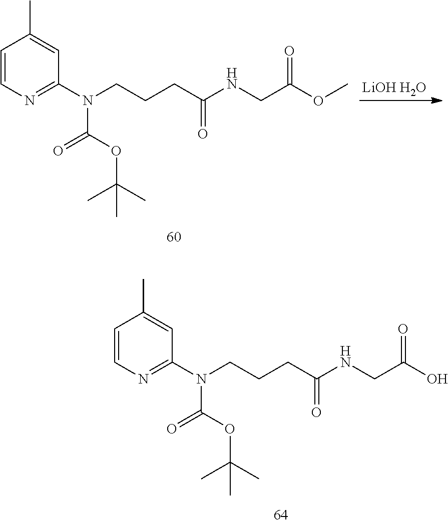

To a solution of compound 60 (1.77 g, 4.84 mmol, 1 equiv.) in THF (5 mL) and H₂O (5 mL) was added lithium hydroxide monohydrate (0.61 g, 14.53 mmol, 3 equiv.) portion-wise at 0° C. The reaction mixture was warmed to room temperature. After stirring at room temperature for 3 hours, the reaction mixture was acidified by HCl (6 N) to pH 3.0. The aqueous phase was extracted with ethyl acetate (3×20 mL) and the organic layer was combined, dried over Na₂SO₄, and concentrated. LC-MS: calculated [M+H]+ 352.18, found 352.

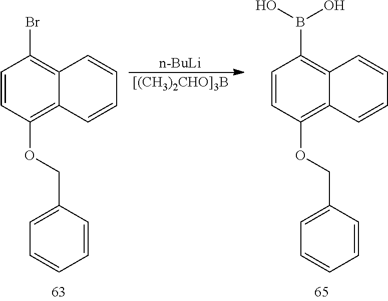

To a solution of compound 63 (1.88 g, 6.0 mmol, 1.0 equiv.) in anhydrous THF (20 mL) was added n-BuLi in hexane (3.6 mL, 9.0 mmol, 1.5 equiv.) drop-wise at −78° C. The reaction was kept at −78° C. for another 1 hour. Triisopropylborate (2.08 mL, 9.0 mmol, 1.5 equiv.) was then added into the mixture at −78° C. The reaction was then warmed up to room temperature and stirred for another 1 hour. The reaction was quenched by saturated NH4Cl solution (20 mL) and the pH was adjusted to 3. The aqueous phase was extracted with EtOAc (3×20 mL) and the organic phase was combined, dried over Na₂SO₄, and concentrated.

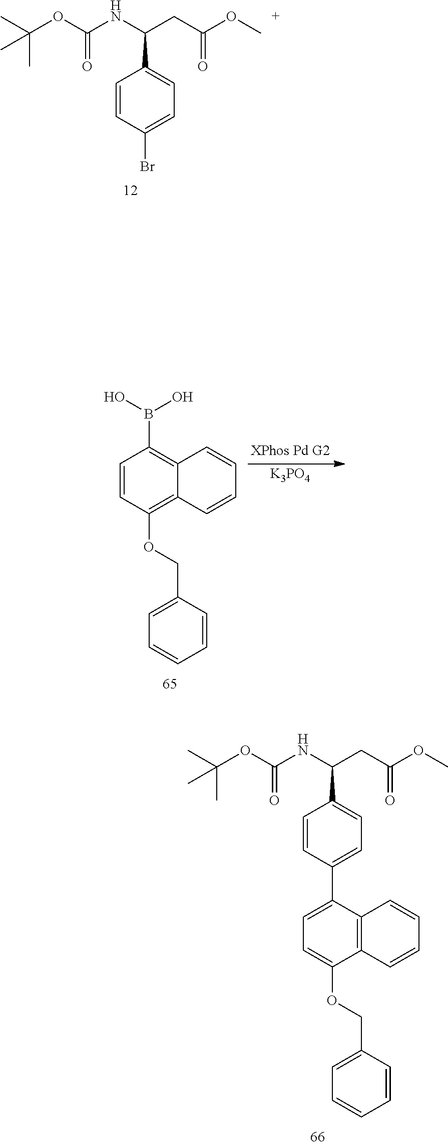

Compound 12 (300 mg, 0.837 mmol, 1.0 equiv.), Compound 65 (349 mg, 1.256 mmol, 1.5 equiv.), XPhos Pd G2 (13 mg, 0.0167 mmol, 0.02 equiv.), and K₃PO₄ (355 mg, 1.675 mmol, 2.0 equiv.) were mixed in a round-bottom flask. The flask was sealed with a screw-cap septum, and then evacuated and backfilled with nitrogen (this process was repeated a total of 3 times). Then, THF (8 mL) and water (2 mL) were added via syringe. The mixture was bubbled with nitrogen for 20 min and the reaction was kept at room temperature for overnight. The reaction was quenched with water (10 mL), and the aqueous phase was extracted with ethyl acetate (3×10 mL). The organic phase was dried over Na₂SO₄, concentrated, and purified via CombiFlash® using silica gel as the stationary phase and was eluted with 15% EtOAc in hexane. LC-MS: calculated [M+H]+ 512.24, found 512.56.

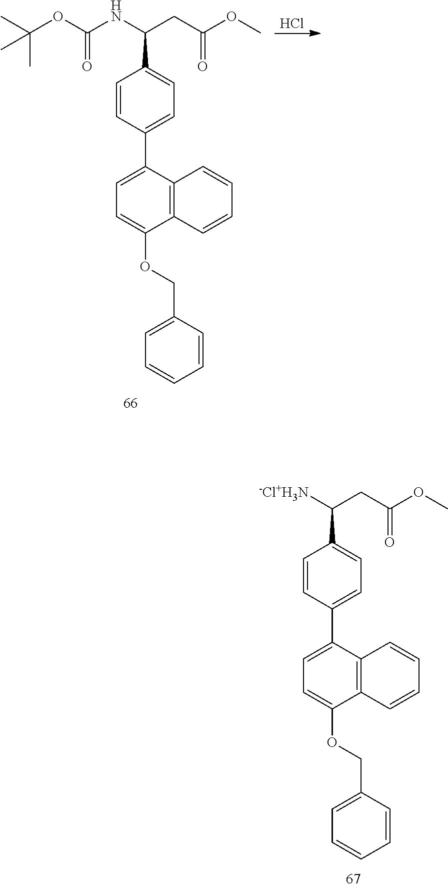

Compound 66 (858 mg, 1.677 mmol, 1.0 equiv.) was cooled by ice bath. HCl in dioxane (8.4 mL, 33.54 mmol, 20 equiv.) was added into the flask. The reaction was warmed to room temperature and stirred for another 1 hr. The solvent was removed by rotary evaporator and the product was directly used without further purification. LC-MS: calculated [M+H]+ 412.18, found 412.46.

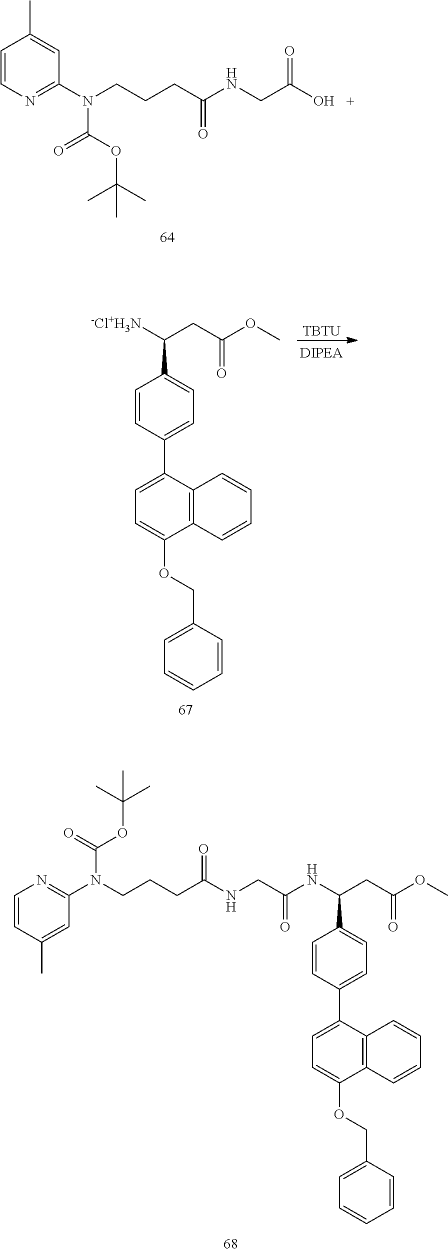

To a solution of compound 64 (500 mg, 1.423 mmol, 1 equiv.), compound 67 (669 mg, 1.494 mmol, 1.05 equiv.), and TBTU (548 mg, 0.492 mmol, 1.2 equiv.) in anhydrous DMF (15 mL) was added diisopropylethylamine (0.744 mL, 4.268 mmol, 3 equiv.) at 0° C. The reaction mixture was warmed to room temperature and stirred for another 1 hr. The reaction was quenched by saturated NaHCO₃ aqueous solution (10 mL) and the product was extracted with ethyl acetate (3×20 mL). The organic phase was combined, dried over Na₂SO₄, and concentrated. The product was purified by CombiFlash® using silica gel as the stationary phase and was eluted with 3-4% methanol in DCM. The yield was 96.23%. LC-MS: calculated [M+H]+ 745.35, found 746.08.

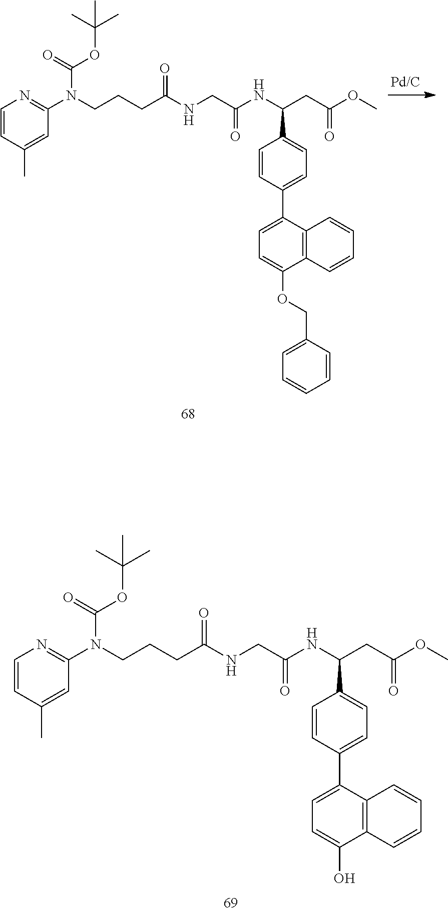 [SMILES: COC(=O)C[C@H](NC(=O)CNC(=O)CCCN(C(=O)OC(C)(C)C)c1cc(C)ccn1)c1ccc(-c2ccc(OCc3ccccc3)c3ccccc23)cc1 ; COC(=O)C[C@H](NC(=O)CNC(=O)CCCN(C(=O)OC(C)(C)C)c1cc(C)ccn1)c1ccc(-c2ccc(O)c3ccccc23)cc1]

To a solution of compound 68 (1.02 g, 1.369 mmol, 1 equiv.) in ethyl acetate (10 mL) was added 10% Pd/C (0.15 g, 50% H₂O) at room temperature. The reaction mixture was warmed to room temperature and the reaction was monitored by LC-MS. The reaction was kept at room temperature overnight. The solids were filtered through Celite® and the solvent was removed by rotary evaporator. The product was directly used without further purification. LC-MS: [M+H]+ 655.31, found 655.87.

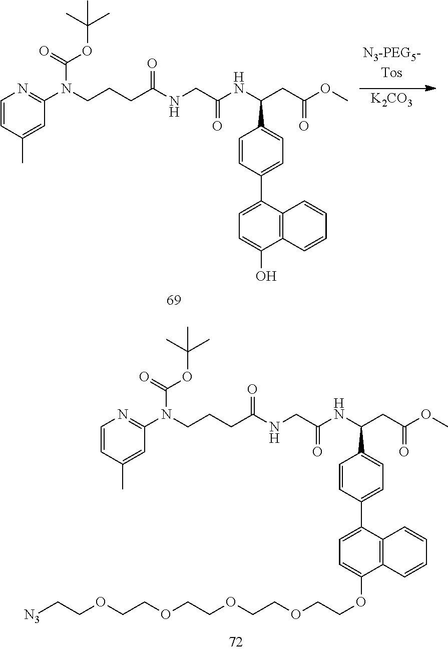

To a solution of compound 69 (100 mg, 0.152 mmol, 1 equiv.) and azido-PEG₅-OTs (128 mg, 0.305 mmol, 2 equiv.) in anhydrous DMF (2 mL) was added K₂CO₃ (42 mg, 0.305 mmol, 2 equiv.) at 0° C. The reaction mixture was stirred for 6 hours at 80° C. The reaction was quenched by saturated NaHCO₃ solution and the aqueous layer was extracted with ethyl acetate (3×10 mL). The organic phase was combined, dried over Na₂SO₄, and concentrated. LC-MS: calculated [M+H]+ 900.40, found 901.46.

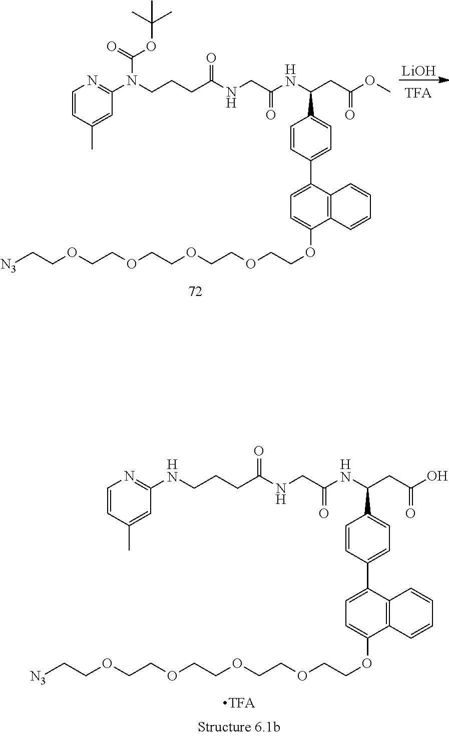

To a solution of compound 72 (59 mg, 0.0656 mmol, 1.0 equiv.) in THF (2 mL) and water (2 mL) was added lithium hydroxide (5 mg, 0.197 mmol, 3.0 equiv.) at room temperature. The mixture was stirred at room temperature for another 1 hr. The pH was adjusted to 3.0 by HCl (6N) and the aqueous phase was extracted with EtOAc (3×10 mL). The organic phase was combined, dried over Na₂SO₄, and concentrated. TFA (0.5 mL) and DCM (0.5 mL) was added into the residue and the mixture was stirred at room temperature for another 3 hr. The solvent was removed by rotary evaporator. LC-MS: calculated [M+H]+ 786.37, found 786.95.

### G. Synthesis of TriAlk14

TriAlk14 and (TriAlk14)s as shown in Table 12, above, may be synthesized using the synthetic route shown below. Compound 14 may be added to the sense strand as a phosphoramidite using standard oligonucleotide synthesis techniques, or compound 22 may be conjugated to the sense strand comprising an amine in an amide coupling reaction.

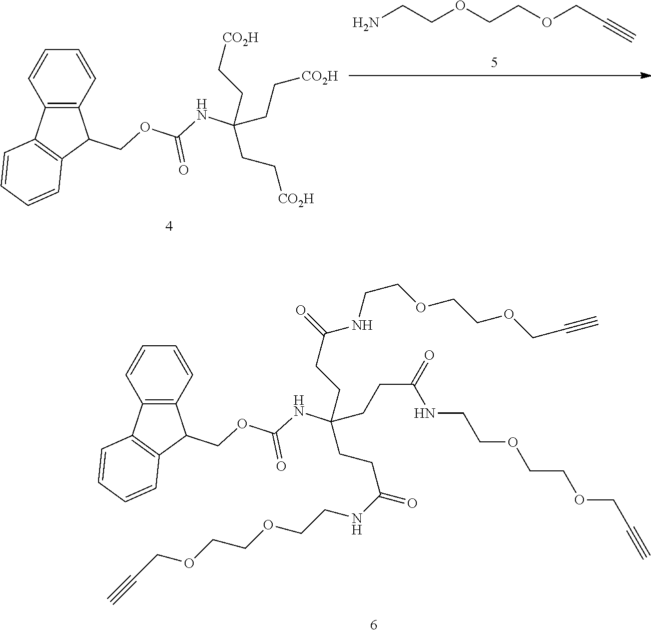

To a 3-L jacketed reactor was added 500 mL DCM and 4 (75.0 g, 0.16 mol). The internal temperature of the reaction was cooled to 0° C. and TBTU (170.0 g, 0.53 mol) was added. The suspension was then treated with the amine 5 (75.5 g, 0.53 mol) dropwise keeping the internal temperature less than 5° C. The reaction was then treated with DIPEA (72.3 g, 0.56 mol) slowly, keeping the internal temperature less than 5° C. After the addition was complete, the reaction was warmed up to 23° C. over 1 hour, and allowed to stir for 3 hours. A 10% kicker charge of all three reagents were added and allowed to stir an additional 3 hours. The reaction was deemed complete when <1% of 4 remained. The reaction mixture was washed with saturated ammonium chloride solution (2×500 mL) and once with saturated sodium bicarbonate solution (500 mL). The organic layer was then dried over sodium sulfate and concentrated to an oil. The mass of the crude oil was 188 g which contained 72% 6 by QNMR. The crude oil was carried to the next step. Calculated mass for C₄₆H₆₀N₄O₁₁=845.0 m/z. Found [M+H]=846.0.

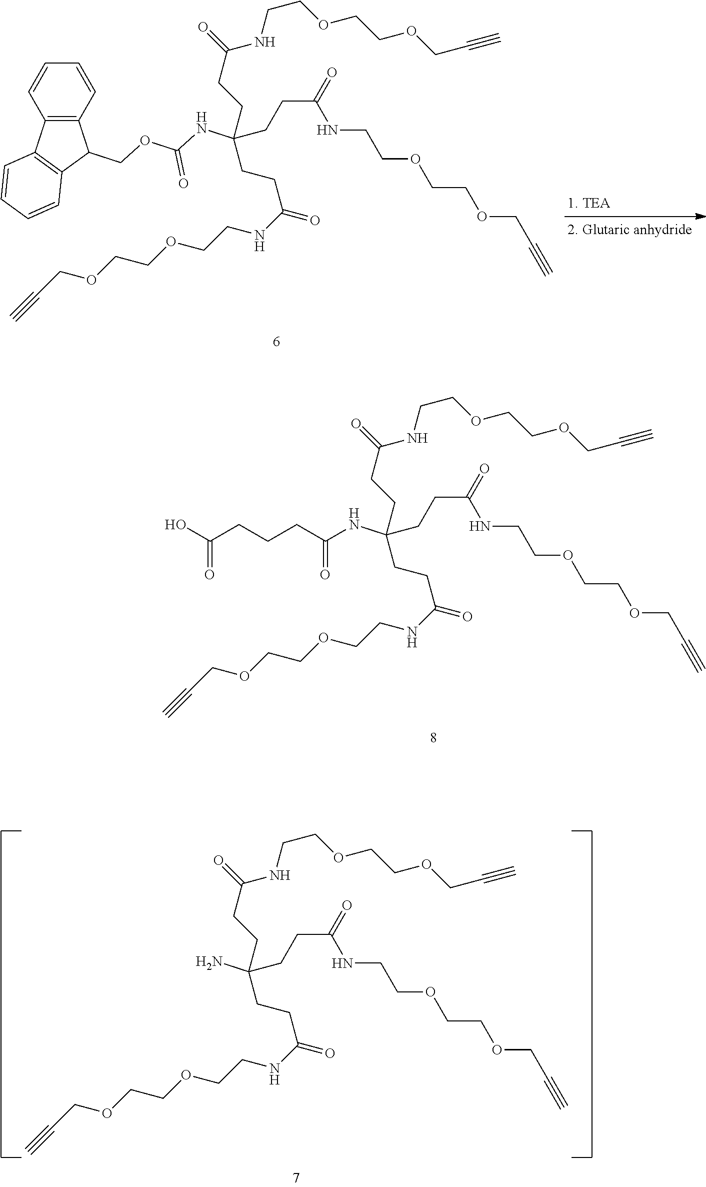

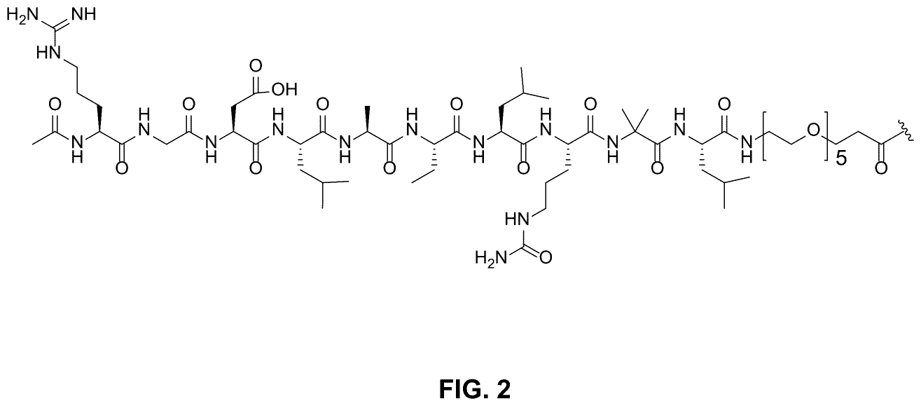

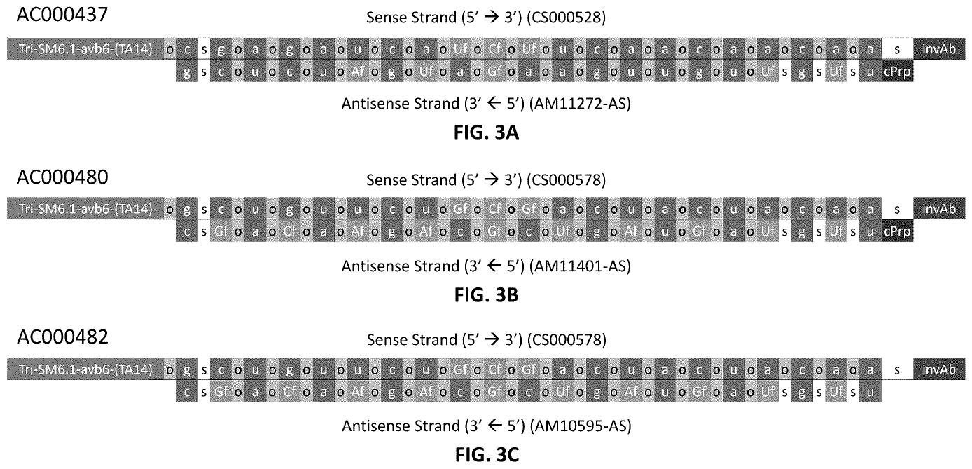

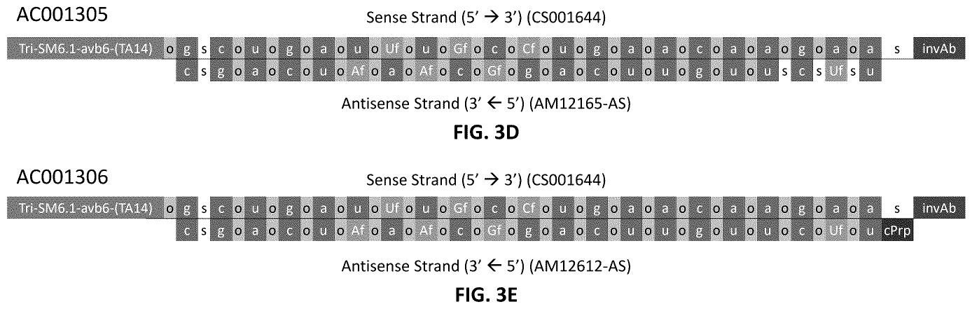

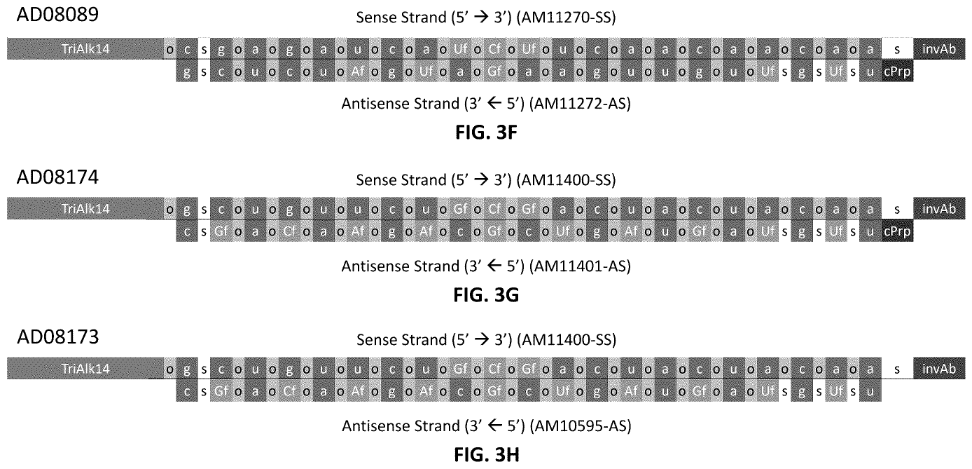

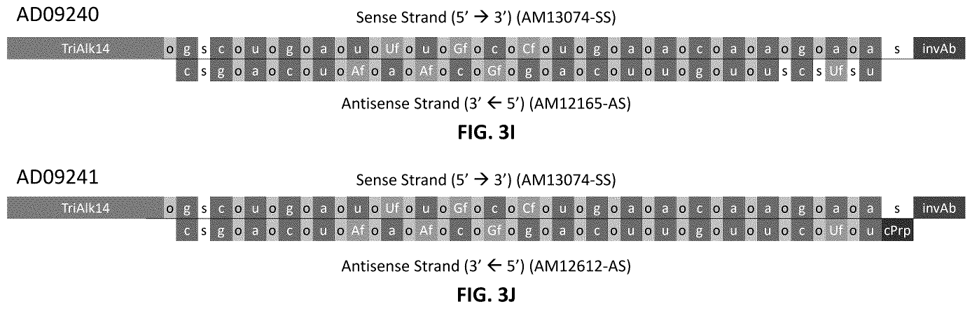

The 121.2 g of crude oil containing 72 wt % compound 6 (86.0 g, 0.10 mol) was dissolved in DMF (344 mL) and treated with TEA (86 mL, 20 v/v %), keeping the internal temperature below 23° C. The formation of dibenzofulvene (DBF) relative to the consumption of Fmoc-amine 6 was monitored via HPLC method 1 (FIG. 2) and the reaction was complete within 10 hours. To the solution was added glutaric anhydride (12.8 g, 0.11 mol) and the intermediate amine 7 was converted to compound 8 within 2 hours. Upon completion, the DMF and TEA were removed at 30° C. under reduced pressure resulting in 100 g of a crude oil. Due to the high solubility of compound 7 in water, an aqueous workup could not be used, and chromatography is the only way to remove DBF, TMU, and glutaric anhydride. The crude oil (75 g) was purified on a Teledyne ISCO Combi-Flash® purification system in three portions. The crude oil (25 g) was loaded onto a 330 g silica column and eluted from 0-20% methanol/DCM over 30 minutes resulting in 42 g of compound 8 (54% yield over 3 steps). Calculated mass for C₃₆H₅₅N₄O₁₂=736.4 m/z. Found [M+H]=737.0.

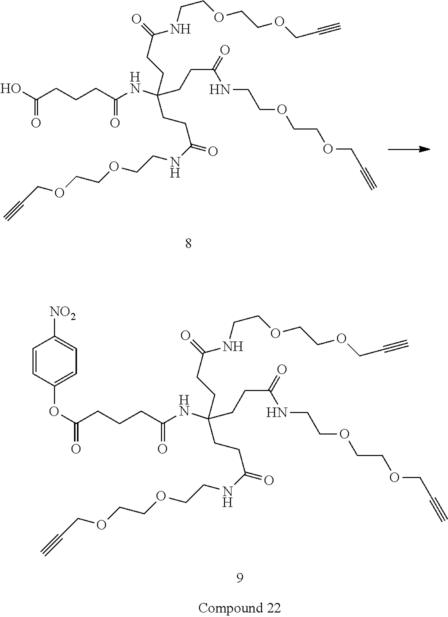 [SMILES: C#CCOCCOCCNC(=O)CCC(CCC(=O)NCCOCCOCC#C)(CCC(=O)NCCOCCOCC#C)NC(=O)CCCC(=O)O ; C#CCOCCOCCNC(=O)CCC(CCC(=O)NCCOCCOCC#C)(CCC(=O)NCCOCCOCC#C)NC(=O)CCCC(=O)Oc1ccc([N+](=O)[O-])cc1]

Compound 8 (42.0 g, 0.057 mol) was co-stripped with 10 volumes of acetonitrile prior to use to remove any residual methanol from chromatography solvents. The oil was redissolved in DMF (210 mL) and cooled to 0° C. The solution was treated with 4-nitrophenol (8.7 g, 0.063 moL) followed by EDC-hydrochloride (12.0 g, 0.063 mol) and found to reach completion within 10 hours. The solution was cooled to 0° C. and 10 volumes ethyl acetate was added followed by 10 volumes saturated ammonium chloride solution, keeping the internal temperature below 15° C. The layers were allowed to separate and the ethyl acetate layer was washed with brine. The combined aqueous layers were extracted twice with 5 volumes ethyl acetate. The combined organic layers were dried over sodium sulfate and concentrated to an oil. The crude oil (55 g) was purified on a Teledyne ISCO Combi-Flash® purification system in three portions. The crude oil (25 g) was loaded onto a 330 g silica column and eluted from 0-10% methanol/DCM over 30 minutes resulting in 22 g of pure 9 (Compound 22) (50% yield). Calculated mass for C₄₂H₅₉N₅O₁₄=857.4 m/z. Found [M+H]=858.0.

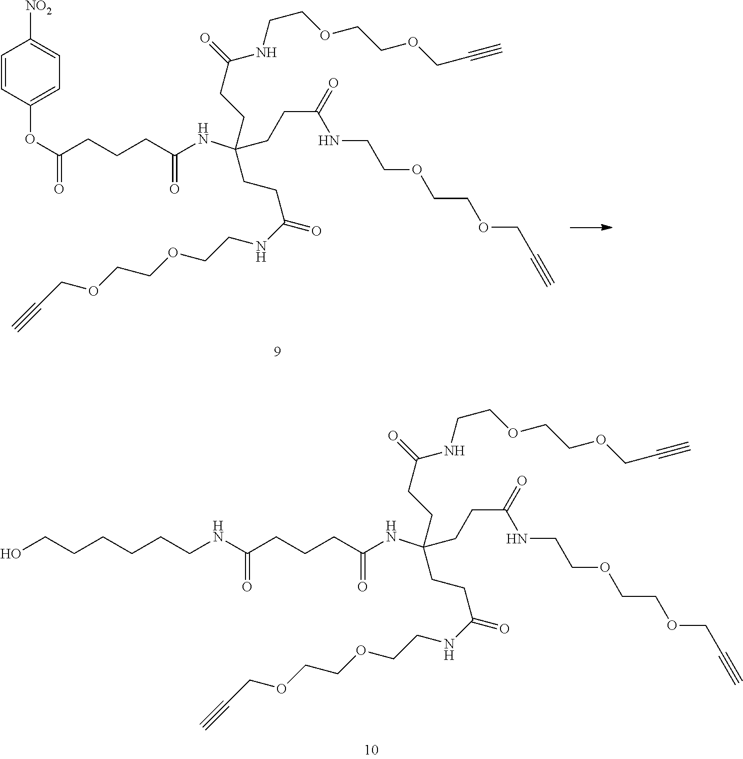 [SMILES: C#CCOCCOCCNC(=O)CCC(CCC(=O)NCCOCCOCC#C)(CCC(=O)NCCOCCOCC#C)NC(=O)CCCC(=O)Oc1ccc([N+](=O)[O-])cc1 ; C#CCOCCOCCNC(=O)CCC(CCC(=O)NCCOCCOCC#C)(CCC(=O)NCCOCCOCC#C)NC(=O)CCCC(=O)NCCCCCCO]

A solution of ester 9 (49.0 g, 57.1 mmol) and 6-amino-1-hexanol (7.36 g, 6.28 mmol) in dichloromethane (3 volumes) was treated with triethylamine (11.56 g, 111.4 mmol) dropwise. The reaction was monitored by observing the disappearance of compound 9 on HPLC Method 1 and was found to be complete in 10 minutes. The crude reaction mixture was diluted with 5 volumes dichloromethane and washed with saturated ammonium chloride (5 volumes) and brine (5 volumes). The organic layer was dried over sodium sulfate and concentrated to an oil. The crude oil was purified on a Teledyne ISCO Combi-Flash® purification system using a 330 g silica column. The 4-nitrophenol was eluted with 100% ethyl acetate and 10 was flushed from the column using 20% methanol/DCM resulting in a colorless oil (39 g, 81% yield). Calculated mass for C₄₂H₆₉N₅O₁₂=836.0 m/z. Found [M+H]=837.0.

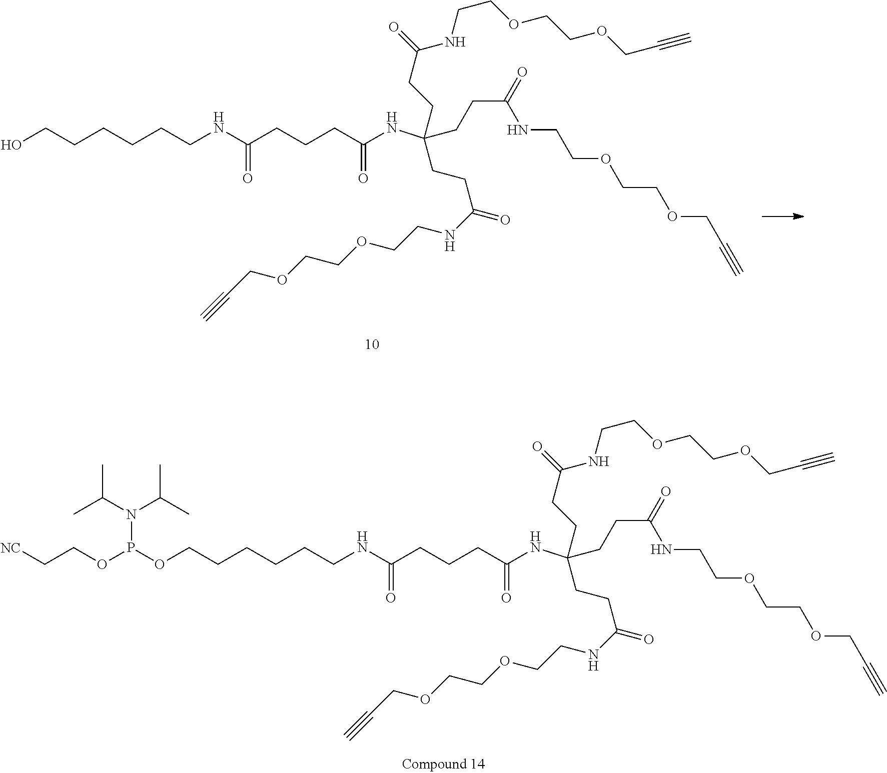 [SMILES: C#CCOCCOCCNC(=O)CCC(CCC(=O)NCCOCCOCC#C)(CCC(=O)NCCOCCOCC#C)NC(=O)CCCC(=O)NCCCCCCO ; C#CCOCCOCCNC(=O)CCC(CCC(=O)NCCOCCOCC#C)(CCC(=O)NCCOCCOCC#C)NC(=O)CCCC(=O)NCCCCCCOP(OCCC#N)N(C(C)C)C(C)C]

Alcohol 10 was co-stripped twice with 10 volumes of acetonitrile to remove any residual methanol from chromatography solvents and once more with dry dichloromethane (KF<60 ppm) to remove trace water. The alcohol 10 (2.30 g, 2.8 mmol) was dissolved in 5 volumes dry dichloromethane (KF<50 ppm) and treated with diisopropylammonium tetrazolide (188 mg, 1.1 mmol). The solution was cooled to 0° C. and treated with 2-cyanoethyl N,N,N′,N′-tetraisopropylphosphoramidite (1.00 g, 3.3 mmol) dropwise. The solution was removed from ice-bath and stirred at 20° C. The reaction was found to be complete within 3-6 hours. The reaction mixture was cooled to 0° C. and treated with 10 volumes of a 1:1 solution of saturated ammonium bicarbonate/brine and then warmed to ambient over 1 minute and allowed to stir an additional 3 minutes at 20° C. The biphasic mixture was transferred to a separatory funnel and 10 volumes of dichloromethane was added. The organic layer was separated and washed with 10 volumes of saturated sodium bicarbonate solution to hydrolyze unreacted bis-phosphorous reagent. The organic layer was dried over sodium sulfate and concentrated to an oil resulting in 3.08 g of 94 wt % Compound 14. Calculated mass for C₅₁H₈₆N₇O₁₃P=1035.6 m/z. Found [M+H]=1036.

### H. Conjugation of Targeting Ligands.

Either prior to or after annealing, the 5′ or 3′ tridentate alkyne functionalized sense strand is conjugated to targeting ligands. The following example describes the conjugation of targeting ligands to the annealed duplex: Stock solutions of 0.5M Tris(3-hydroxypropyltriazolylmethyl)amine (THPTA), 0.5M of Cu(II) sulfate pentahydrate (Cu(II)SO₄·5H₂O) and 2M solution of sodium ascorbate were prepared in deionized water. A 75 mg/mL solution in DMSO of targeting ligand was made. In a 1.5 mL centrifuge tube containing tri-alkyne functionalized duplex (3 mg, 75 μL, 40 mg/mL in deionized water, ˜15,000 g/mol), 25 μL of 1M Hepes pH 8.5 buffer is added. After vortexing, 35 μL of DMSO was added and the solution is vortexed. Targeting ligand was added to the reaction (6 equivalents/duplex, 2 equivalents/alkyne, ˜15 μL) and the solution is vortexed. Using pH paper, pH was checked and confirmed to be pH˜8. In a separate 1.5 mL centrifuge tube, 50 μL of 0.5M THPTA was mixed with 10 μL of 0.5M Cu(II)SO₄·5H₂O, vortexed, and incubated at room temp for 5 min. After 5 min, THPTA/Cu solution (7.2 μL, 6 equivalents 5:1 THPTA:Cu) was added to the reaction vial, and vortexed. Immediately afterwards, 2M ascorbate (5 μL, 50 equivalents per duplex, 16.7 per alkyne) was added to the reaction vial and vortexed. Once the reaction was complete (typically complete in 0.5-1 h), the reaction was immediately purified by non-denaturing anion exchange chromatography.

### Example 2. In Vitro Testing of MUC5AC RNAi Agents

Certain chemically modified candidate sequence duplexes shown Table 8C above (with the antisense strand sequence set forth in Table 3 and the nucleotide and end cap portion of the sense strand found in Table 6), were tested in vitro. The MUC5AC RNAi agents were prepared in accordance with the procedures set forth in Example 1.

Evaluation of MUC5AC RNAi agents in vitro was performed by transfection of A549 cells, a human lung epithelial cell line. Cells were plated at ˜7,500 cells per well in 96-well format, and each of the RNAi agent duplexes shown in Table 12 was transfected at three concentrations (10 nM, 1 nM, and 0.1 nM), using LipoFectamine RNAiMax (Thermo Fisher) transfection reagent. Relative expression of each of the MUC5AC RNAi agents was determined by qRT-PCR by comparing the expression levels of MUC5AC mRNA to an endogenous control, and normalized to untreated A549 cells (ΔΔCT analysis), as shown in Table 12.

Table 12, below, lists the AD duplex number for the sequence being examined, as well as in parenthesis the gene position being targeted by that particular RNAi agent. Thus, for example, for Duplex ID AD08101, average relative expression at 1 nM of 0.377 shows MUC5AC gene knockdown of 62.3%, and average relative expression at 0.1 nM shows inhibition of 53.0% (0.470) normalized to untreated wells (mock control).

TABLE 13
In Vitro Testing of MUC5AC RNAi Agents.

| Duplex | Avg. Rel. | High | Low | Avg. Rel. | High | Low | Avg. Rel. | High | Low |
|---|---|---|---|---|---|---|---|---|---|
| ID No. | Exp. 10 nM | (error) | (error) | Exp. 1 nM | (error) | (error) | Exp. 0.1 nM | (error) | (error) |
| AD08101 | 0.230 | 0.042 | 0.051 | 0.377 | 0.049 | 0.056 | 0.470 | 0.056 | 0.064 |
| (11014_1) | | | | | | | | | |
| AD08666 | 0.258 | 0.047 | 0.058 | 0.394 | 0.068 | 0.082 | 0.474 | 0.102 | 0.131 |
| (4993_2) | | | | | | | | | |
| AD08668 | 0.228 | 0.052 | 0.068 | 0.401 | 0.065 | 0.078 | 0.554 | 0.181 | 0.270 |
| (4993_4) | | | | | | | | | |
| AD07732 | 0.240 | 0.057 | 0.075 | 0.427 | 0.083 | 0.103 | 0.527 | 0.087 | 0.105 |
| (3099_1) | | | | | | | | | |
| AD08100 | 0.286 | 0.031 | 0.035 | 0.382 | 0.048 | 0.054 | 0.550 | 0.081 | 0.096 |
| (10206_1) | | | | | | | | | |
| AD08103 | 0.198 | 0.063 | 0.092 | 0.439 | 0.079 | 0.097 | 0.656 | 0.086 | 0.099 |
| (12965_1) | | | | | | | | | |
| AD07734 | 0.186 | 0.047 | 0.062 | 0.515 | 0.089 | 0.107 | 0.614 | 0.085 | 0.099 |
| (5347_1) | | | | | | | | | |
| AD07634 | 0.161 | 0.025 | 0.030 | 0.423 | 0.046 | 0.051 | 0.769 | 0.129 | 0.154 |
| (3535_1) | | | | | | | | | |
| AD08671 | 0.353 | 0.047 | 0.054 | 0.416 | 0.078 | 0.096 | 0.588 | 0.068 | 0.077 |
| (4992_2) | | | | | | | | | |
| AD07763 | 0.138 | 0.034 | 0.046 | 0.432 | 0.065 | 0.077 | 0.826 | 0.149 | 0.182 |
| (610_1) | | | | | | | | | |
| AD08667 | 0.293 | 0.056 | 0.069 | 0.427 | 0.051 | 0.057 | 0.677 | 0.151 | 0.195 |
| (4993_3) | | | | | | | | | |
| AD07733 | 0.253 | 0.031 | 0.036 | 0.501 | 0.084 | 0.102 | 0.655 | 0.123 | 0.152 |
| (4993_1) | | | | | | | | | |
| AD08669 | 0.302 | 0.037 | 0.042 | 0.461 | 0.057 | 0.065 | 0.658 | 0.083 | 0.094 |
| (4993_5) | | | | | | | | | |
| AD08673 | 0.369 | 0.061 | 0.073 | 0.468 | 0.060 | 0.069 | 0.624 | 0.070 | 0.079 |
| (4992_4) | | | | | | | | | |
| AD07774 | 0.182 | 0.053 | 0.074 | 0.565 | 0.088 | 0.104 | 0.860 | 0.099 | 0.112 |
| (2797_1) | | | | | | | | | |
| AD07735 | 0.288 | 0.037 | 0.043 | 0.565 | 0.097 | 0.117 | 0.774 | 0.071 | 0.078 |
| (5350_1) | | | | | | | | | |
| AD08670 | 0.360 | 0.033 | 0.036 | 0.513 | 0.072 | 0.084 | 0.814 | 0.198 | 0.261 |
| (4993_6) | | | | | | | | | |
| AD07637 | 0.539 | 0.061 | 0.069 | 0.606 | 0.057 | 0.063 | 0.674 | 0.051 | 0.055 |
| (5300_1) | | | | | | | | | |
| AD08571 | 0.250 | 0.039 | 0.047 | 0.612 | 0.046 | 0.050 | 0.978 | 0.089 | 0.099 |
| (15051_1) | | | | | | | | | |
| AD08572 | 0.389 | 0.052 | 0.059 | 0.688 | 0.080 | 0.091 | 0.819 | 0.098 | 0.112 |
| (15052_7) | | | | | | | | | |
| AD08568 | 0.448 | 0.069 | 0.082 | 0.661 | 0.080 | 0.091 | 0.789 | 0.082 | 0.092 |
| (3910_1) | | | | | | | | | |
| AD08569 | 0.282 | 0.034 | 0.038 | 0.666 | 0.096 | 0.113 | 0.978 | 0.046 | 0.048 |
| (5029_9) | | | | | | | | | |
| AD08573 | 0.398 | 0.027 | 0.029 | 0.736 | 0.069 | 0.076 | 0.814 | 0.118 | 0.138 |
| (15052_8) | | | | | | | | | |
| AD08096 | 0.359 | 0.040 | 0.045 | 0.621 | 0.076 | 0.086 | 1.008 | 0.080 | 0.087 |
| (4992_1) | | | | | | | | | |
| AD08672 | 0.432 | 0.052 | 0.059 | 0.669 | 0.057 | 0.063 | 0.925 | 0.040 | 0.042 |
| (4992_3) | | | | | | | | | |
| AD07756 | 0.372 | 0.064 | 0.078 | 0.693 | 0.053 | 0.058 | 1.082 | 0.146 | 0.169 |
| (1618_1) | | | | | | | | | |
| AD07773 | 0.446 | 0.042 | 0.046 | 0.837 | 0.117 | 0.136 | 0.935 | 0.153 | 0.183 |
| (2536_1) | | | | | | | | | |
| AD07760 | 0.379 | 0.059 | 0.070 | 0.806 | 0.141 | 0.171 | 1.096 | 0.151 | 0.175 |
| (2001_1) | | | | | | | | | |
| AD07771 | 0.389 | 0.032 | 0.035 | 0.872 | 0.093 | 0.104 | 1.205 | 0.161 | 0.185 |
| (2004_1) | | | | | | | | | |
| Mock | 1.000 | 0.164 | 0.197 | 1.000 | 0.164 | 0.197 | 1.000 | 0.164 | 0.197 |
| Control | | | | | | | | | |

### Example 3. In Vitro Testing of MUC5AC RNAi Agents

Certain chemically modified candidate sequence duplexes shown Table 8C above (with the antisense strand sequence set forth in Table 3 and the nucleotide and end cap portion of the sense strand found in Table 6), were tested in vitro. The MUC5AC RNAi agents were prepared in accordance with the procedures set forth in Example 1.

Evaluation of MUC5AC RNAi agents in vitro was performed by transfection of A549 cells, a human lung epithelial cell line. Cells were plated at ˜7,500 cells per well in 96-well format, and each of the RNAi agent duplexes shown in Table 12 was transfected at three concentrations (10 nM, 1 nM, and 0.1 nM), using LipoFectamine RNAiMax (Thermo Fisher) transfection reagent. Relative expression of each of the MUC5AC RNAi agents was determined by qRT-PCR by comparing the expression levels of MUC5AC mRNA to an endogenous control, and normalized to untreated A549 cells (ΔΔCT analysis), as shown in Table 12.

Table 12, below, lists the AD duplex number for the sequence being examined, as well as in parenthesis the gene position being targeted by that particular RNAi agent. Thus, for example, for Duplex ID AD08101, average relative expression at 1 nM of 0.377 shows MUC5AC gene knockdown of 62.3%, and average relative expression at 0.1 nM shows inhibition of 53.0% (0.470) normalized to untreated wells (mock control).

TABLE 14
In Vitro Testing of MUC5AC RNAi Agents.

| Duplex | Avg. Rel. | High | Low | Avg. Rel. | High | Low | Avg. Rel. | High | Low |
|---|---|---|---|---|---|---|---|---|---|
| ID No. | Exp. 10 nM | (error) | (error) | Exp. 1 nM | (error) | (error) | Exp. 0.1 nM | (error) | (error) |
| AD07733 | 0.156 | 0.047 | 0.067 | 0.192 | 0.030 | 0.035 | 0.336 | 0.024 | 0.026 |
| (4993_1) | | | | | | | | | |
| AD08096 | 0.171 | 0.022 | 0.026 | 0.275 | 0.035 | 0.040 | 0.406 | 0.027 | 0.028 |
| (4992_1) | | | | | | | | | |
| AD07767 | 0.140 | 0.019 | 0.022 | 0.195 | 0.097 | 0.193 | 0.536 | 0.226 | 0.392 |
| (1758_1) | | | | | | | | | |
| AD08103 | 0.197 | 0.057 | 0.080 | 0.276 | 0.042 | 0.050 | 0.409 | 0.046 | 0.052 |
| (12965_1) | | | | | | | | | |
| AD08098 | 0.172 | 0.032 | 0.039 | 0.259 | 0.076 | 0.109 | 0.466 | 0.042 | 0.046 |
| (8739_1) | | | | | | | | | |
| AD07763 | 0.094 | 0.024 | 0.032 | 0.293 | 0.033 | 0.037 | 0.514 | 0.026 | 0.027 |
| (610_1) | | | | | | | | | |
| AD08100 | 0.242 | 0.046 | 0.056 | 0.260 | 0.051 | 0.064 | 0.417 | 0.041 | 0.045 |
| (10206_1) | | | | | | | | | |
| AD08101 | 0.236 | 0.054 | 0.071 | 0.322 | 0.044 | 0.052 | 0.364 | 0.039 | 0.043 |
| (11014_1) | | | | | | | | | |
| AD07751 | 0.161 | 0.058 | 0.091 | 0.335 | 0.051 | 0.061 | 0.536 | 0.035 | 0.037 |
| (5533_1) | | | | | | | | | |
| AD07634 | 0.159 | 0.034 | 0.043 | 0.385 | 0.035 | 0.039 | 0.507 | 0.061 | 0.069 |
| (3535_1) | | | | | | | | | |
| AD07770 | 0.114 | 0.022 | 0.028 | 0.377 | 0.041 | 0.046 | 0.599 | 0.059 | 0.066 |
| (1867_1) | | | | | | | | | |
| AD07747 | 0.347 | 0.057 | 0.069 | 0.330 | 0.042 | 0.049 | 0.413 | 0.033 | 0.036 |
| (5020_1) | | | | | | | | | |
| AD07749 | 0.365 | 0.061 | 0.073 | 0.337 | 0.017 | 0.018 | 0.464 | 0.060 | 0.068 |
| (5441_1) | | | | | | | | | |
| AD08095 | 0.204 | 0.024 | 0.027 | 0.380 | 0.064 | 0.077 | 0.624 | 0.046 | 0.049 |
| (1871_1) | | | | | | | | | |
| AD08097 | 0.364 | 0.056 | 0.066 | 0.384 | 0.068 | 0.083 | 0.511 | 0.061 | 0.069 |
| (6798_1) | | | | | | | | | |
| AD07750 | 0.432 | 0.063 | 0.074 | 0.240 | 0.073 | 0.105 | 0.589 | 0.056 | 0.062 |
| (5519_1) | | | | | | | | | |
| AD07774 | 0.168 | 0.031 | 0.038 | 0.432 | 0.072 | 0.087 | 0.679 | 0.040 | 0.042 |
| (2797_1) | | | | | | | | | |
| AD07732 | 0.352 | 0.056 | 0.067 | 0.402 | 0.147 | 0.231 | 0.535 | 0.154 | 0.217 |
| (3099_1) | | | | | | | | | |
| AD07764 | 0.424 | 0.080 | 0.099 | 0.431 | 0.051 | 0.057 | 0.505 | 0.048 | 0.052 |
| (923_1) | | | | | | | | | |
| AD07771 | 0.367 | 0.064 | 0.077 | 0.493 | 0.090 | 0.109 | 0.597 | 0.078 | 0.090 |
| (2004_1) | | | | | | | | | |
| AD07748 | 0.518 | 0.131 | 0.176 | 0.413 | 0.078 | 0.095 | 0.559 | 0.068 | 0.077 |
| (5042_1) | | | | | | | | | |
| AD07768 | 0.278 | 0.039 | 0.046 | 0.561 | 0.084 | 0.099 | 0.728 | 0.041 | 0.043 |
| (1761_1) | | | | | | | | | |
| AD07756 | 0.522 | 0.081 | 0.096 | 0.464 | 0.081 | 0.099 | 0.708 | 0.052 | 0.056 |
| (1618_1) | | | | | | | | | |
| AD07772 | 0.476 | 0.055 | 0.062 | 0.588 | 0.057 | 0.063 | 0.654 | 0.070 | 0.078 |
| (2234_1) | | | | | | | | | |
| AD07773 | 0.498 | 0.039 | 0.043 | 0.596 | 0.060 | 0.067 | 0.672 | 0.055 | 0.060 |
| (2536_1) | | | | | | | | | |
| AD07746 | 0.578 | 0.058 | 0.065 | 0.671 | 0.086 | 0.099 | 0.740 | 0.052 | 0.056 |
| (4446_1) | | | | | | | | | |
| AD08094 | 0.646 | 0.056 | 0.061 | 0.721 | 0.071 | 0.079 | 0.762 | 0.076 | 0.084 |
| (1445_1) | | | | | | | | | |
| AD07766 | 0.760 | 0.103 | 0.119 | 0.806 | 0.067 | 0.072 | 0.793 | 0.082 | 0.092 |
| (1446_1) | | | | | | | | | |
| AD07745 | 0.902 | 0.194 | 0.247 | 0.840 | 0.088 | 0.098 | 0.821 | 0.049 | 0.052 |
| (4443_1) | | | | | | | | | |
| Mock | 1.000 | 0.109 | 0.122 | 1.000 | 0.109 | 0.122 | 1.000 | 0.109 | 0.122 |
| Control | | | | | | | | | |

### Example 4. House Dust Mite (HDM) Induced Allergic Asthma Model

To study the properties of certain MUC5AC RNAi agents in vivo, the house dust mite (HDM) induced allergic asthma mouse model was used. To induce mouse Muc5ac expression, female Balb/c mice (6-8 weeks in age) were administered 50 pg house dust mite protein acquired commercially in 25 μL of isotonic saline intranasally using a pipette for 5 consecutive days. 72 hours after the fifth daily dose, mice were euthanized and whole lungs were harvested for mRNA expression analysis. Compared to unchallenged, naïve mice, relative expression of mouse Muc5ac mRNA in HDM challenged mice is shown to increase approximately 100 fold.

### Example 5. In Vivo Intratracheal Administration of MUC5AC RNAi Agents in the HDM Model

The HDM induced allergic asthma mouse model described in Example 4, above, was used. The following Table 15 sets forth the dosing Groups:

TABLE 15
MUC5AC RNAi Agent and Dosing for Example 5.

| | | Study Days IT | IN Dose | Study Days IN | Animals |
|---|---|---|---|---|---|
| GROUP | IT Dose Administered | Administered | Administered | Dose Administered | Per Group |
| 1 | No treatment | N/A | No treatment | N/A | 6 |
| 2 | No treatment | N/A | Saline | Days 8-12 | 6 |
| 3 | Saline | 1, 3, 5, and 8 | Saline | Days 8-12 | 3 |
| 4 | No treatment | N/A | HDM | Days 8-12 | 6 |
| 5 | Saline | 1, 3, 5, and 8 | HDM | Days 8-12 | 4 |
| 6 | 5.0 mg/kg Tri-SM6.1-αvβ6-AD07022 | 1, 3, 5, and 8 | HDM | Days 8-12 | 4 |
| 7 | 5.0 mg/kg Tri-SM6.1-αvβ6-AD07720 | 1, 3, 5, and 8 | HDM | Days 8-12 | 5 |
| 8 | 5.0 mg/kg Tri-SM6.1-αvβ6-AD07719 | 1, 3, 5, and 8 | HDM | Days 8-12 | 5 |

As noted in Table 15 above, the mice in Group 1 received no treatment throughout. For the mice in Groups 3, 5, 6, 7 and 8, on study days 1, 3, 5, and 8, female Balb/c mice were administered a single dose of 50 microliters via a microsprayer device (Penn Century, Philadelphia, PA) suitable for intratracheal (IT) administration of isotonic saline or 5.0 mg/kg the respective MUC5AC RNAi agent formulated in isotonic saline as noted in Table 15.

As shown in Table 15, each of the MUC5AC RNAi agents (Groups 6, 7 and 8) were conjugated to a tridentate small molecule αvβ6 epithelial cell targeting ligand (Tri-SM6.1, see FIG. 1) at the 5′ terminal end of the sense strand.

The chemically modified sequences for MUC5AC RNAi agents AD07720 and AD07719 (Groups 7 and 8) are shown in Table 7B (showing duplex), Table 3 (showing respective antisense strand), and Table 5 (showing respective sense strand with linker but without tridentate small molecule αvβ6 epithelial cell targeting ligand (Tri-SM6.1).

AD07022 has mouse-specific sequences that do not have homology with the human MUC5AC gene, and were chemically modified as follows:

Tri-SM6.1-αvβ6-AD07022
Modified Sense Strand (5′→3′):
(SEQ ID NO: 1714)
Tri-SM6.1-αvβ6-(TA14)cscauacagCfAfGfuacaguuacas
(invAb)
Modified Antisense Strand (5′→3′):
(SEQ ID NO: 1713)
cPrpusGfsusAfaCfuGfuAfcUfgCfuGfuAfuGfsg

On each of Days 8 through 12, the mice in Groups 2 through 8 were administered a single dose intranasally (IN) using a pipette with 25 microliters of isotonic saline (Groups 2 and 3) or 50 micrograms of house dust mite formulated in isotonic saline (referred to in Table 15 as HDM).

Mice were sacrificed on study day 15, and total RNA was isolated from both lungs following collection and homogenization. Mouse Muc5ac mRNA expression was quantitated by probe-based quantitative PCR, normalized to mouse beta-actin expression, and expressed as fraction of vehicle control group (geometric mean, +/−95% confidence interval).

TABLE 16
Average Relative Mouse MUC5AC mRNA
at Sacrifice (Day 15) in Example 5

| | Average Relative | Low | High |
|---|---|---|---|
| Group ID | mMuc5AC mRNA | (error) | (error) |
| Group 1 (No Treatment) | 1.000 | 0.213 | 0.270 |
| Group 2 (IN Saline) | 1.941 | 0.638 | 0.951 |
| Group 3 (IT Saline & IN Saline) | 1.706 | 0.532 | 0.774 |
| Group 4 (IN HDM) | 117.876 | 26.269 | 33.801 |
| Group 5 (IT Saline & IN HDM) | 95.585 | 21.822 | 28.277 |
| Group 6 (IT 5 mg/kg Tri-SM6.1- | 13.444 | 3.410 | 4.569 |
| αvβ6-AD07022 & IN HDM) | | | |
| Group 7 (IT 5 mg/kg Tri-SM6.1- | 71.812 | 16.633 | 21.647 |
| αvβ6-AD07720 & IN HDM) | | | |
| Group 8 (IT 5 mg/kg Tri-SM6.1- | 90.537 | 27.214 | 38.910 |
| αvβ6-AD07719 & IN HDM) | | | |

The data were normalized to the non-treatment group (Group 1). As shown in the data in Table 16 above, the HDM mouse model performed as expected with respect to promoting an increase in MUC5AC expression after exposure to HDM. The data show that Groups 7 and 8, which each had nucleotide sequences targeting position 1921 of the MUC5AC gene and has homology to both the human and mouse gene transcript, provided only a very minimal reduction in MUC5AC protein compared to the HDM model mice of Groups 4 and 5 with no RNAi agent, indicating only a minimal amount of inhibition for these specific RNAi agents. Alternatively, the mouse-specific RNAi agent of AD07022 (Group 6) showed a substantial reduction in Muc5ac mouse mRNA levels (only 13.444) compared to the groups where HDM was administered without a MUC5AC RNAi agent.

### Example 6. In Vivo Intratracheal Administration of MUC5AC RNAi Agents in the HDM Model

The HDM induced allergic asthma mouse model described in Example 4, above, was used. The following Table 17 sets forth the dosing Groups:

TABLE 17
MUC5AC RNAi Agent and Dosing for Example 6.

| | | | | Study Days | Animals | Targeted |
|---|---|---|---|---|---|---|
| | | Study Days IT | IN Dose | IN Dose | Per | Gene |
| GROUP | IT Dose Administered | Administered | Administered | Administered | Group | Position |
| 1 | Saline | 1, 3, 5, and 8 | Saline | Days 8-12 | 6 | N/A |
| 2 | Saline | 1, 3, 5, and 8 | HDM | Days 8-12 | 5 | N/A |
| 3 | 5.0 mg/kg Tri-SM6.1-αvβ6-AD07022 | 1, 3, 5, and 8 | HDM | Days 8-12 | 6 | Mouse- |
| | | | | | | specific |
| 4 | 5.0 mg/kg Tri-SM6.1-αvβ6-AD08083 | 1, 3, 5, and 8 | HDM | Days 8-12 | 5 | 5029 |
| 5 | 5.0 mg/kg Tri-SM6.1-αvβ6-AD08084 | 1, 3, 5, and 8 | HDM | Days 8-12 | 5 | 5029 |
| 6 | 5.0 mg/kg Tri-SM6.1-αvβ6-AD08085 | 1, 3, 5, and 8 | HDM | Days 8-12 | 4 | 5029 |
| 7 | 5.0 mg/kg Tri-SM6.1-αvβ6-AD08086 | 1, 3, 5, and 8 | HDM | Days 8-12 | 5 | 9729 |
| 8 | 5.0 mg/kg Tri-SM6.1-αvβ6-AD08087 | 1, 3, 5, and 8 | HDM | Days 8-12 | 6 | 9729 |
| 9 | 5.0 mg/kg Tri-SM6.1-αvβ6-AD08088 | 1, 3, 5, and 8 | HDM | Days 8-12 | 5 | 15052 |
| 10 | 5.0 mg/kg Tri-SM6.1-αvβ6-AD08089 | 1, 3, 5, and 8 | HDM | Days 8-12 | 5 | 15052 |
| 11 | 5.0 mg/kg Tri-SM6.1-αvβ6-AD07022 | 1 and 8 | HDM | Days 8-12 | 5 | Mouse- |
| | | | | | | specific |

For the mice in Groups 1-10, on study days 1, 3, 5, and 8, female Balb/c mice were administered a single dose of 50 microliters via a microsprayer device (Penn Century, Philadelphia, PA) suitable for intratracheal (IT) administration of isotonic saline or 5.0 mg/kg of the respective MUC5AC RNAi agent formulated in isotonic saline as noted in Table 17. For the mice in Group 11, the MUC5AC RNAi agent was administered only on days 1 and 8.

As shown in Table 17, each of the MUC5AC RNAi agents (Groups 3-11) were conjugated to a tridentate small molecule αvβ6 epithelial cell targeting ligand (Tri-SM6.1, see FIG. 1) at the 5′ terminal end of the sense strand. The chemically modified sequences for MUC5AC RNAi agents AD08083, AD08084, AD08085, AD08086, AD08087, AD08088, and AD08089 (Groups 4 through 10) are shown in Table 7B (showing duplex), Table 3 (showing respective antisense strand), and Table 5 (showing respective sense strand with linker but without tridentate small molecule αvβ6 epithelial cell targeting ligand (Tri-SM6.1)).

AD07022 has mouse-specific sequences that do not have homology with the human MUC5AC gene, and were chemically modified as shown above in Example 5.

On each of Days 8 through 12, the mice were administered a single dose intranasally (IN) using a pipette with 25 microliters of isotonic saline (Group 2) or 50 micrograms of house dust mite formulated in isotonic saline (referred to in Table 17 as HDM).

Mice were sacrificed on study day 15, and total RNA was isolated from both lungs following collection and homogenization. Mouse Muc5ac mRNA expression was quantitated by probe-based quantitative PCR, normalized to mouse beta-actin expression, and expressed as fraction of vehicle control group (geometric mean, +/−95% confidence interval).

TABLE 18
Average Relative Mouse MUC5AC mRNA
at Sacrifice (Day 15) in Example 6

| | Average Relative | Low | High |
|---|---|---|---|
| Group ID | mMuc5ac mRNA | (error) | (error) |
| Group 1 (IT Saline & IN Saline) | 1.000 | 0.305 | 0.440 |
| Group 2 (IT Saline & IN HDM) | 115.127 | 17.128 | 20.122 |
| Group 3 (IT 5 mg/kg Tri-SM6.1- | 19.053 | 6.287 | 9.383 |
| αvβ6-AD07022 & IN HDM) | | | |
| Group 4 (IT 5 mg/kg Tri-SM6.1- | 35.333 | 13.193 | 21.054 |
| αvβ6-AD08083 & IN HDM) | | | |
| Group 5 (IT 5 mg/kg Tri-SM6.1- | 26.634 | 12.943 | 25.180 |
| αvβ6-AD08084 & IN HDM) | | | |
| Group 6 (IT 5 mg/kg Tri-SM6.1- | 34.602 | 3.503 | 3.897 |
| αvβ6-AD08085 & IN HDM) | | | |
| Group 7 (IT 5 mg/kg Tri-SM6.1- | 55.475 | 15.377 | 21.273 |
| αvβ6-AD08086 & IN HDM) | | | |
| Group 8 (IT 5 mg/kg Tri-SM6.1- | 66.631 | 19.703 | 27.976 |
| αvβ6-AD08087 & IN HDM) | | | |
| Group 9 (IT 5 mg/kg Tri-SM6.1- | 26.879 | 4.505 | 5.412 |
| αvβ6-AD08088 & IN HDM) | | | |
| Group 10 (IT 5 mg/kg Tri-SM6.1- | 14.903 | 2.441 | 2.919 |
| αvβ6-AD08089 & IN HDM) | | | |
| Group 11 (IT 5 mg/kg Tri-SM6.1- | 14.457 | 6.005 | 10.271 |
| αvβ6-AD07022 & IN HDM) | | | |

The data were normalized to the IT and IN saline-only dosed group (Group 1). As shown in the data in Table 18 above, the HDM mouse model performed as expected with respect to promoting an increase in MUC5AC expression after exposure to HDM. The data show that Groups 7 and 8, which both had nucleotide sequences targeting position 9729 of the MUC5AC gene and has homology to both the human and mouse gene transcript, provided only a moderate reduction in MUC5AC protein compared to the HDM model mice of Group 2 with no RNAi agent, indicating only a moderate amount of inhibition for these specific RNAi agents. Alternatively, the remaining MUC5AC RNAi agents tested (targeting gene position 5029 in Groups 4-6 and gene position 15052 in Groups 9 and 10) each showed substantial inhibition compared to Group 2, as did the mouse-specific MUC5AC RNAi agent of AD07022 (Group 6).

### Example 7. In Vivo Intratracheal Administration of MUC5AC RNAi Agents in Rats

The HDM induced allergic asthma mouse model described in Example 4, above, was used. The following Table 19 sets forth certain dosing Groups included in the study:

TABLE 19
MUC5AC RNAi Agent and Dosing for Example 7.

| | | | | Study Days | Animals | Targeted |
|---|---|---|---|---|---|---|
| | | Study Days IT | IN Dose | IN Dose | Per | Gene |
| GROUP | IT Dose Administered | Administered | Administered | Administered | Group | Position |
| 1 | Saline | 1, 3, 5, and 8 | Saline | Days 8-12 | 6 | N/A |
| 2 | Saline | 1, 3, 5, and 8 | HDM | Days 8-12 | 6 | N/A |
| 3 | 5.0 mg/kg Tri-SM6.1-αvβ6-AD07022 | 1, 3, 5, and 8 | HDM | Days 8-12 | 5 | Mouse- |
| | | | | | | Specific |
| 4 | 5.0 mg/kg Tri-SM6.1-αvβ6-AD08173 | 1, 3, 5, and 8 | HDM | Days 8-12 | 6 | 3535 |
| 5 | 5.0 mg/kg Tri-SM6.1-αvβ6-AD08174 | 1, 3, 5, and 8 | HDM | Days 8-12 | 6 | 3535 |
| 6 | 5.0 mg/kg Tri-SM6.1-αvβ6-AD08243 | 1, 3, 5, and 8 | HDM | Days 8-12 | 6 | 3535 |
| 7 | 5.0 mg/kg Tri-SM6.1-αvβ6-AD08244 | 1, 3, 5, and 8 | HDM | Days 8-12 | 6 | 3535 |
| 8 | 5.0 mg/kg Tri-SM6.1-αvβ6-AD08175 | 1, 3, 5, and 8 | HDM | Days 8-12 | 6 | 3535 |
| 9 | 5.0 mg/kg Tri-SM6.1-αvβ6-AD08176 | 1, 3, 5, and 8 | HDM | Days 8-12 | 6 | 3535 |
| 10 | 5.0 mg/kg Tri-SM6.1-αvβ6-AD08177 | 1, 3, 5, and 8 | HDM | Days 8-12 | 6 | 3535 |

For the mice in Groups 1-5, on study days 1, 3, 5, and 8, female Balb/c mice were administered a single dose of 50 microliters via a microsprayer device (Penn Century, Philadelphia, PA) suitable for intratracheal (IT) administration of isotonic saline or 5.0 mg/kg the respective MUC5AC RNAi agent formulated in isotonic saline as noted in Table 19.

As shown in Table 19, each of the MUC5AC RNAi agents (Groups 3-5) were conjugated to a tridentate small molecule αvβ6 epithelial cell targeting ligand (Tri-SM6.1, see FIG. 1) at the 5′ terminal end of the sense strand. The chemically modified sequences for MUC5AC RNAi agents AD08173 and AD08174 (Groups 4 and 5) are shown in Table 7B (showing duplex), Table 3 (showing respective antisense strand), and Table 5 (showing respective sense strand with linker but without tridentate small molecule αvβ6 epithelial cell targeting ligand (Tri-SM6.1)). Each of the MUC5AC RNAi agents with sequences targeting position 3535 have a mismatch in what is understood to be an important location from the mouse gene, and therefore it is expected that the MUC5AC RNAi agents would show little to no inhibitory activity in view of the mismatch.

AD07022 has mouse-specific sequences that do not have homology with the human MUC5AC gene, and were chemically modified as shown above in Example 5.

On each of Days 8 through 12, the mice were administered a single dose intranasally (IN) using a pipette with 25 microliters of isotonic saline (Group 1 only) or 50 micrograms of house dust mite formulated in isotonic saline (referred to in Table 19 as HDM).

Mice were sacrificed on study day 15, and total RNA was isolated from both lungs following collection and homogenization. Mouse Muc5ac mRNA expression was quantitated by probe-based quantitative PCR, normalized to mouse beta-actin expression, and expressed as fraction of vehicle control group (geometric mean, +/−95% confidence interval).

TABLE 20
Average Relative Mouse MUC5AC mRNA
at Sacrifice (Day 15) in Example 7

| | Average Relative | Low | High |
|---|---|---|---|
| Group ID | mMuc5ac mRNA | (error) | (error) |
| Group 1 (IT Saline & IN Saline) | 1.000 | 0.197 | 0.245 |
| Group 2 (IT Saline & IN HDM) | 132.247 | 31.248 | 40.917 |
| Group 3 (IT 5 mg/kg Tri-SM6.1- | 13.139 | 2.426 | 2.975 |
| αvβ6-AD07022 & IN HDM) | | | |
| Group 4 (IT 5 mg/kg Tri-SM6.1- | 96.522 | 13.056 | 15.098 |
| αvβ6-AD08173 & IN HDM) | | | |
| Group 5 (IT 5 mg/kg Tri-SM6.1- | 57.983 | 15.132 | 20.476 |
| αvβ6-AD08174 & IN HDM) | | | |
| Group 6 (IT 5 mg/kg Tri-SM6.1- | 55.592 | 8.761 | 10.400 |
| αvβ6-AD08243 & IN HDM) | | | |
| Group 7 (IT 5 mg/kg Tri-SM6.1- | 75.149 | 17.661 | 23.087 |
| αvβ6-AD08244 & IN HDM) | | | |
| Group 8 (IT 5 mg/kg Tri-SM6.1- | 75.420 | 10.876 | 12.708 |
| αvβ6-AD08175 & IN HDM) | | | |
| Group 9 (IT 5 mg/kg Tri-SM6.1- | 72.203 | 12.062 | 14.482 |
| αvβ6-AD08176 & IN HDM) | | | |
| Group 10 (IT 5 mg/kg Tri-SM6.1- | 67.222 | 12.063 | 14.701 |
| αvβ6-AD08177 & IN HDM) | | | |

The data were normalized to the IT and IN saline-only dosed group (Group 1). As noted above, given the nature of the mismatch to the mouse gene for the MUC5AC RNAi agents in Groups 4 and 5 (targeting position 3535 of the human gene), minimal inhibition is expected. As shown in the data in Table 20 above, the HDM mouse model performed as expected with respect to promoting an increase in MUC5AC expression after exposure to HDM, as shown in Groups 1 and 2. Unexpectedly, the MUC5AC RNAi agents targeting position 3535 still showed moderate levels of inhibition despite the mismatch to the mouse gene, indicating that MUC5AC RNAi agents targeting this position may be viable as human therapeutic candidates.

### Example 8. In Vivo Intratracheal Administration of MUC5AC RNAi Agents in the HDM Model

The HDM induced allergic asthma mouse model described in Example 4, above, was used. The following Table 17 sets forth the dosing Groups:

TABLE 21
MUC5AC RNAi Agent and Dosing for Example 8.

| | | | | Study Days | Animals | Targeted |
|---|---|---|---|---|---|---|
| | | Study Days IT | IN Dose | IN Dose | Per | Gene |
| GROUP | IT Dose Administered | Administered | Administered | Administered | Group | Position |
| 1 | Saline | 1 and 7 | Saline | Days 7-11 | 6 | N/A |
| 2 | Saline | 1 and 7 | HDM | Days 7-11 | 6 | N/A |
| 3 | 5.0 mg/kg Tri-SM6.1-αvβ6-AD07022 | 1, 2, 4 and 7 | HDM | Days 7-11 | 6 | Mouse- |
| | | | | | | specific |
| 4 | 5.0 mg/kg Tri-SM6.1-αvβ6-AD07022 | 1 and 7 | HDM | Days 7-11 | 6 | Mouse- |
| | | | | | | specific |
| 5 | 2.5 mg/kg Tri-SM6.1-αvβ6-AD07022 | 1 and 7 | HDM | Days 7-11 | 6 | Mouse- |
| | | | | | | specific |
| 6 | 1.0 mg/kg Tri-SM6.1-αvβ6-AD07022 | 1 and 7 | HDM | Days 7-11 | 6 | Mouse- |
| | | | | | | specific |
| 7 | 5.0 mg/kg Tri-SM6.1-αvβ6-AD08089 | 1, 2, 4 and 7 | HDM | Days 7-11 | 6 | 15052 |
| 8 | 5.0 mg/kg Tri-SM6.1-αvβ6-AD08089 | 1 and 7 | HDM | Days 7-11 | 6 | 15052 |
| 9 | 2.5 mg/kg Tri-SM6.1-αvβ6-AD08089 | 1 and 7 | HDM | Days 7-11 | 6 | 15052 |
| 10 | 1.0 mg/kg Tri-SM6.1-αvβ6-AD08089 | 1 and 7 | HDM | Days 7-11 | 6 | 15052 |
| 11 | 1.0 mg/kg Tri-SM6.1-αvβ6-AD08089 | 1 | HDM | Days 7-11 | 6 | 15052 |

Female Balb/c mice were administered a single dose of 50 microliters via a microsprayer device (Penn Century, Philadelphia, PA) suitable for intratracheal (IT) administration of isotonic saline or an MUC5AC RNAi agent formulated in isotonic saline, on the dates and at the concentrations set forth in Table 21 above.

As shown in Table 21, each of the MUC5AC RNAi agents (Groups 3-11) were conjugated to a tridentate small molecule αvβ6 epithelial cell targeting ligand (see FIG. 1) at the 5′ terminal end of the sense strand. The chemically modified sequences for MUC5AC RNAi agents AD08089 is shown in Table 7B (showing duplex), Table 3 (showing respective antisense strand), and Table 5 (showing respective sense strand with linker but without tridentate small molecule αvβ6 epithelial cell targeting ligand).

AD07022 has mouse-specific sequences that do not have homology with the human MUC5AC gene, and were chemically modified as shown above in Example 5.

On each of Days 7 through 11, the mice were administered a single dose intranasally (IN) using a pipette with 25 microliters of isotonic saline (Group 1) or 50 micrograms of house dust mite formulated in isotonic saline (referred to in Table 21 as HDM).

Mice were sacrificed on study day 14, and total RNA was isolated from both lungs following collection and homogenization. Mouse Muc5ac mRNA expression was quantitated by probe-based quantitative PCR, normalized to mouse beta-actin expression, and expressed as fraction of vehicle control group (geometric mean, +/−95% confidence interval).

TABLE 22
Average Relative Mouse MUC5AC mRNA at Sacrifice (Day 14) in Example 8

| | Average Relative | Low | High |
|---|---|---|---|
| Group ID | mMuc5ac mRNA | (error) | (error) |
| Group 1 (IT Saline & IN Saline) | 1.000 | 0.315 | 0.459 |
| Group 2 (IT Saline & IN HDM) | 112.848 | 44.187 | 72.623 |
| Group 3 (IT 5.0 mg/kg Tri-SM6.1-αvβ6- | 12.455 | 3.896 | 5.669 |
| AD07022 (days 1, 2, 4, & 7) & IN HDM) | | | |
| Group 4 (IT 5.0 mg/kg Tri-SM6.1-αvβ6- | 16.521 | 4.908 | 6.982 |
| AD07022 (days 1 & 7) & IN HDM) | | | |
| Group 5 (IT 2.5 mg/kg Tri-SM6.1-αvβ6- | 26.846 | 5.096 | 6.290 |
| AD07022 & IN HDM) | | | |
| Group 6 (IT 1.0 mg/kg Tri-SM6.1-αvβ6- | 26.521 | 9.295 | 14.311 |
| AD07022 & IN HDM) | | | |
| Group 7 (IT 5.0 mg/kg Tri-SM6.1-αvβ6- | 10.978 | 3.101 | 4.322 |
| AD08089 (days 1, 2, 4, & 7) & IN HDM) | | | |
| Group 8 (IT 5.0 mg/kg Tri-SM6.1-αvβ6- | 17.629 | 6.752 | 10.944 |
| AD08089 (days 1 & 7) & IN HDM) | | | |
| Group 9 (IT 2.5 mg/kg Tri-SM6.1-αvβ6- | 17.746 | 4.647 | 6.296 |
| AD08089 & IN HDM) | | | |
| Group 10 (IT 1.0 mg/kg Tri-SM6.1-αvβ6- | 21.106 | 4.109 | 5.103 |
| AD08089 & IN HDM) | | | |
| Group 11 (IT 1.0 mg/kg Tri-SM6.1-αvβ6- | 42.413 | 14.428 | 21.868 |
| AD08089 (day 1 only) & IN HDM) | | | |

The data were normalized to the IT and IN saline-only dosed group (Group 1). As shown in the data in Table 22 above, the HDM mouse model performed as expected with respect to promoting an increase in MUC5AC expression after exposure to HDM. The data show that AD08089, which has nucleotide sequences targeting position 15052 of the MUC5AC gene and has homology to both the human and mouse gene transcript, provided substantial inhibition of MUC5AC and was generally comparable to the highly active mouse-specific MUC5AC RNAi agent of AD07022.

### Example 9. In Vivo Inhaled Aerosolized Administration of MUC5AC RNAi Agents in Cynomolgus Monkeys

On study day 1, male cynomolgus monkeys were administered a single dose on each of days 1, 8, and 15 at 1 mg/kg pulmonary deposited dose (PDD) of the MUC5AC RNAi agent AC001305 or AC001306. Using a vibrating mesh nebulizer (Aeroneb Solo), aerosol was delivered to restrained, anesthetized monkeys intubated intratracheally. Intubated animals were connected to a ventilator, which was used to control respiratory minute volume. Test article aerosol was generated via an Aeroneb Solo mesh nebulizer connected in-line with the exposure system. Exposures times were determined from aerosol trials in which the efficiency of the system was determined by placing a filter at the end of the endotracheal tube, collecting the aerosol during the course of the exposure. The MUC5AC RNAi agent was conjugated to a tridentate small molecule αvβ6 epithelial cell targeting ligand (see FIG. 1) at the 5′ terminal end of the sense strand, formulated in isotonic saline. The chemically modified sequences for MUC5AC RNAi agents AC001305 and AC001306 are shown in Table 11. The antisense strand sequence of AC001305 is also shown as AM12165 in Table 3, and the antisense strand sequence of AC001306 is also shown as AM12166 in Table 3, both of which target position 4993 of the MUC5AC gene.

The dosing groups were as described in the following Table 23:

TABLE 23
MUC5AC RNAi Agent and Dosing for Example 9
Group ID

Group 1 (isotonic saline on Days 1, 8, 15
Group 2 (1.0 mg/kg pulmonary deposited
dose AC001305 on Days 1, 8, 15
Group 3 (1.0 mg/kg pulmonary deposited
dose AC001306 on Days 1, 8, 15

Two (2) monkeys were dosed per group. Monkeys were sacrificed on study day 22, and total RNA was isolated from lung samples following collection and homogenization. The data in Table 24, below, shows mRNA expression sampled from the distal left caudal lobe. Cynomolgus monkey MUC5AC mRNA expression was quantitated by probe-based quantitative PCR, normalized to Cynomolgus monkey beta-actin expression, and expressed as fraction of vehicle control group (geometric mean, +/−95% confidence interval).

TABLE 24A
Cynomolgus Monkey Mucosal Tissue Muc5ac mRNA
Relative Expression at Sacrifice in Example 9

| | Relative | | |
|---|---|---|---|
| | cMuc5ac mRNA | | |
| | Expression | Low | High |
| Group ID | (n = 2) | (error) | (error) |
| Group 1 (isotonic saline) | 1.000 | 0.386 | 0.628 |
| Group 2 (1.0 mg/kg deposited | 0.034 | 0.010 | 0.015 |
| dose AC001305 on Days 1, 8, 15) | | | |
| Group 3 (1.0 mg/kg deposited | 0.171 | 0.085 | 0.169 |
| dose AC001306 on Days 1, 8, 15 | | | |

TABLE 24B
Cynomolgus Monkey Right Cranial Hilar Muc5ac mRNA
Relative Expression at Sacrifice in Example 9

| | Relative | | |
|---|---|---|---|
| | cMuc5ac mRNA | | |
| | Expression | Low | High |
| Group ID | (n = 2) | (error) | (error) |
| Group 1 (isotonic saline) | 1.000 | 0.708 | 2.421 |
| Group 2 (1.0 mg/kg deposited | 0.034 | 0.010 | 0.015 |
| dose AC001305 on Days 1, 8, 15) | | | |
| Group 3 (1.0 mg/kg deposited | 0.180 | 0.089 | 0.174 |
| dose AC001306 on Days 1, 8, 15 | | | |

TABLE 24C
Cynomolgus Monkey Right Cranial Mid Airway Muc5ac mRNA
Relative Expression at Sacrifice in Example 9

| | Relative | | |
|---|---|---|---|
| | cMuc5ac mRNA | | |
| | Expression | Low | High |
| Group ID | (n = 2) | (error) | (error) |
| Group 1 (isotonic saline) | 1.000 | 0.489 | 0.956 |
| Group 2 (1.0 mg/kg deposited | 0.162 | 0.099 | 0.256 |
| dose AC001305 on Days 1, 8, 15) | | | |
| Group 3 (1.0 mg/kg deposited | 0.077 | 0.041 | 0.086 |
| dose AC001306 on Days 1, 8, 15 | | | |

As reported in Tables 24A, 24B, and 24C above, the MUC5AC RNAi agents

### Example 10. Aerosolized Administration of MUC5AC RNAi Agents in Sheep

Sheep exposed to inhaled ascaris antigen exhibit responses typical of allergic asthma, including an acute phase response (AR), late phase response (LR), and airway hyperreactivity (AHR) as shown by Abraham et. al. (Am Rev Respir Dis., 1983), and the model has been shown to respond well to standard of care therapies (Caniga, et. al., J Inflamm., 2013). Accordingly, the model may be used to determine the impact of sheep Muc5ac (sMuc5ac) mRNA silencing on airway mechanics and AHR upon treatment with MUC5AC RNAi agents. Test article delivery to intubated sheep, airway mechanics assessments detecting changes in pulmonary resistance (RL) following challenge with Ascaris suum antigen, and AHR assessments by performing cumulative concentration response curves to inhaled carbachol were performed according to published procedures (Abraham et. al., J Clin Invest., 1994).

Two (2) ascaris-sensitive sheep with previously established responses to Ascaris suum challenge were administered 1 mg/kg pulmonary deposited dose levels of AC000480 on days 1, 8 and 15. The chemical structure of AC000480 is shown, for example, in Table 11 and is designed to target position 3535 on the MUC5AC gene. On day 21, AHR was assessed by determining the cumulative carbachol concentration (in breath units, BU) that increased RL to 400% over the post-1×PBS value (PC₄₀₀). On day 22, sheep were challenged with Ascaris suum extract, and R_{L} was monitored out to 8 hours post-challenge. On day 23, AHR was again assessed in the same manner as on day 21. To monitor duration of effect, sheep were again challenged with Ascaris suum extract on day 51, bracketed on day 50 and day 52 with assessments of AHR.

TABLE 25
Airway Mechanics Results

| | | | Control Trial (no treatment) | | | | Drug Trial: Day 22 | | | | Drug Trial: Day 51 | | | |
|---|---|---|---|---|---|---|---|---|---|---|---|---|---|---|
| | | | Animal # | | | | Animal # | | | | Animal # | | | |
| Timepoint | | | 2489 | 2497 | Mean | S.D. | 2489 | 2497 | Mean | S.D. | 2489 | 2497 | Mean | S.D. |
| Baseline | | RL | 0.99 | 0.99 | 0.99 | 0.00 | 1.00 | 1.00 | 1.00 | 0.00 | 1.00 | 1.00 | 1.00 | 0.00 |
| Post- | | RL | 6.66 | 6.62 | 6.64 | 0.03 | 4.28 | 5.21 | 4.75 | 0.66 | 6.35 | 6.38 | 6.37 | 0.02 |
| Ascaris | | % | 573% | 569% | 571% | 3% | 328% | 421% | 375% | 66% | 535% | 538% | 537% | 2% |
| 1 | h | RL | 4.51 | 4.40 | 4.46 | 0.08 | 2.35 | 3.59 | 2.97 | 0.88 | 4.60 | 4.61 | 4.61 | 0.01 |
| | | % | 356% | 344% | 350% | 8% | 135% | 259% | 197% | 88% | 360% | 361% | 361% | 1% |
| 2 | h | RL | 2.53 | 2.57 | 2.55 | 0.03 | 1.81 | 1.66 | 1.74 | 0.11 | 2.42 | 2.64 | 2.53 | 0.16 |
| | | % | 156% | 160% | 158% | 3% | 81% | 66% | 74% | 11% | 142% | 164% | 153% | 16% |
| 3 | h | RL | 1.55 | 1.53 | 1.54 | 0.01 | 1.37 | 1.36 | 1.37 | 0.01 | 1.68 | 1.62 | 1.65 | 0.04 |
| | | % | 57% | 55% | 56% | 1% | 37% | 36% | 37% | 1% | 68% | 62% | 65% | 4% |
| 4 | h | RL | 1.06 | 1.21 | 1.14 | 0.11 | 1.05 | 1.09 | 1.07 | 0.03 | 1.05 | 1.07 | 1.06 | 0.01 |
| | | % | 7% | 22% | 15% | 11% | 5% | 9% | 7% | 3% | 5% | 7% | 6% | 1% |
| 5 | h | RL | 1.52 | 1.66 | 1.59 | 0.10 | 1.14 | 1.19 | 1.17 | 0.04 | 1.64 | 1.55 | 1.60 | 0.06 |
| | | % | 54% | 68% | 61% | 10% | 14% | 19% | 17% | 4% | 64% | 55% | 60% | 6% |
| 6 | h | RL | 2.13 | 2.09 | 2.11 | 0.03 | 1.28 | 1.34 | 1.31 | 0.04 | 2.07 | 2.18 | 2.13 | 0.08 |
| | | % | 115% | 111% | 113% | 3% | 28% | 34% | 31% | 4% | 107% | 118% | 113% | 8% |
| 6.5 | h | RL | 2.26 | 2.21 | 2.24 | 0.04 | 1.51 | 1.49 | 1.50 | 0.01 | 2.22 | 2.26 | 2.24 | 0.03 |
| | | % | 128% | 123% | 126% | 4% | 51% | 49% | 50% | 1% | 122% | 126% | 124% | 3% |
| 7 | h | RL | 2.32 | 2.26 | 2.29 | 0.04 | 1.39 | 1.40 | 1.40 | 0.01 | 2.37 | 2.29 | 2.33 | 0.06 |
| | | % | 134% | 128% | 131% | 4% | 39% | 40% | 40% | 1% | 137% | 129% | 133% | 6% |
| 7.5 | h | RL | 2.12 | 2.16 | 2.14 | 0.03 | 1.27 | 1.35 | 1.31 | 0.06 | 2.32 | 2.05 | 2.19 | 0.19 |
| | | % | 114% | 118% | 116% | 3% | 27% | 35% | 31% | 6% | 132% | 105% | 119% | 19% |
| 8 | h | RL | 2.08 | 2.03 | 2.06 | 0.04 | 1.16 | 1.28 | 1.22 | 0.08 | 2.12 | 2.13 | 2.13 | 0.01 |
| | | % | 110% | 105% | 108% | 4% | 16% | 28% | 22% | 8% | 112% | 113% | 113% | 1% |

TABLE 26
AHR Results

| | BU Carbachol to Produce PC400 | |
|---|---|---|
| Sheep # | 24 h pre-ascaris | 24 h post Ascaris |

Control Trial

| | | |
|---|---|---|
| 2489 | 24 | 13 |
| 2497 | 31 | 13 |

Drug Trial: Day 22

| | | |
|---|---|---|
| 2489 | 25 | 26 |
| 2497 | 26 | 25 |

Drug Trial: Day 51

| | | |
|---|---|---|
| 2489 | 26 | 13 |
| 2497 | 29 | 12 |

As shown in Table 25, treatment with AC000480 resulted in attenuation of AR as well as LR upon challenge on day 22. For example, untreated sheep display a mean LR increase of 126% in R_{L} at 6.5 h compared to baseline, where AC000480 treated sheep on day 22 challenge show a more attenuated LR increase of 50% in R_{L} at 6.5 h compared to baseline. In addition, 24 h after the day 22 ascaris challenge both AC000480 treated sheep showed no signs of ascaris induced airway hyperresponsiveness, as shown by equivalent number of carbachol breath units required to produce PC₄₀₀. In contrast, in the control trial without AC000480 treatment, sheep required approximately half the amount of carbachol breath units to induce PC₄₀₀ post-ascaris challenge, signifying airway hyperresponsiveness.

With no additional dosing after day 15, sheep returned to baseline airway mechanics and AHR upon the day 51 ascaris challenge.

### Example 11. Aerosolized Administration of MUC5AC RNAi Agents in Sheep

The sheep model of allergic asthma airway inflammation described in example 10, above, was used. Three (3) ascaris sensitive sheep with previously established responses to Ascaris suum challenge were administered 1 mg/kg pulmonary deposited dose levels of AC000482 on days 1, 8 and 15. The chemical structure of AC000482 is shown, for example, in Table 11 and is designed to target position 3535 on the MUC5AC gene. On day 21, AHR was assessed by determining the cumulative carbachol concentration (in breath units, BU) that increased R_{L} to 400% over the post-1×PBS value (PC₄₀₀). On day 22, sheep were challenged with Ascaris suum extract, and R_{L} was monitored out to 8 h post-challenge. On day 23, AHR was again assessed as on day 21.

TABLE 27
Airway Mechanics Results

| | | | Control (no treatment) | | | | | Drug Trial: Day 22 | | | | |
|---|---|---|---|---|---|---|---|---|---|---|---|---|
| | | | Animal # | | | Mean | | Animal # | | | Mean | |
| Timepoint | | | 2485 | 2515 | 2535 | Mean | S.D. | 2485 | 2515 | 2535 | Mean | S.D. |
| Baseline | | RL | 0.99 | 1.00 | 1.00 | 1.00 | 0.01 | 1.00 | 1.00 | 1.00 | 1.00 | 0.00 |
| Post- | | RL | 6.43 | 6.94 | 6.89 | 6.75 | 0.28 | 5.49 | 6.44 | 6.09 | 6.01 | 0.48 |
| Ascaris | | % | 549% | 594% | 589% | 577% | 24% | 449% | 544% | 509% | 501% | 48% |
| 1 | h | RL | 4.10 | 4.67 | 4.40 | 4.39 | 0.29 | 4.68 | 4.27 | 4.10 | 4.35 | 0.30 |
| | | % | 314% | 367% | 340% | 340% | 26% | 368% | 327% | 310% | 335% | 30% |
| 2 | h | RL | 2.62 | 2.51 | 2.64 | 2.59 | 0.07 | 2.81 | 2.10 | 2.05 | 2.32 | 0.43 |
| | | % | 165% | 151% | 164% | 160% | 8% | 181% | 110% | 105% | 132% | 43% |
| 3 | h | RL | 1.65 | 1.46 | 1.58 | 1.56 | 0.10 | 1.53 | 1.37 | 1.31 | 1.40 | 0.11 |
| | | % | 67% | 46% | 58% | 57% | 10% | 53% | 37% | 31% | 40% | 11% |
| 4 | h | RL | 1.08 | 1.09 | 1.05 | 1.07 | 0.02 | 1.09 | 1.05 | 1.04 | 1.06 | 0.03 |
| | | % | 9% | 9% | 5% | 8% | 2% | 9% | 5% | 4% | 6% | 3% |
| 5 | h | RL | 1.57 | 1.53 | 1.57 | 1.56 | 0.02 | 1.27 | 1.15 | 1.18 | 1.20 | 0.06 |
| | | % | 59% | 53% | 57% | 56% | 3% | 27% | 15% | 18% | 20% | 6% |
| 6 | h | RL | 1.87 | 2.10 | 2.03 | 2.00 | 0.12 | 1.42 | 1.32 | 1.23 | 1.32 | 0.10 |
| | | % | 89% | 110% | 103% | 101% | 11% | 42% | 32% | 23% | 32% | 10% |
| 6.5 | h | RL | 2.16 | 2.30 | 2.15 | 2.20 | 0.08 | 1.66 | 1.55 | 1.46 | 1.56 | 0.10 |
| | | % | 118% | 130% | 115% | 121% | 8% | 66% | 55% | 46% | 56% | 10% |
| 7 | h | RL | 2.20 | 2.21 | 2.23 | 2.21 | 0.02 | 1.54 | 1.41 | 1.34 | 1.43 | 0.10 |
| | | % | 122% | 121% | 123% | 122% | 1% | 54% | 41% | 34% | 43% | 10% |
| 7.5 | h | RL | 2.27 | 2.11 | 2.19 | 2.19 | 0.08 | 1.33 | 1.24 | 1.18 | 1.25 | 0.08 |
| | | % | 129% | 111% | 119% | 120% | 9% | 33% | 24% | 18% | 25% | 8% |
| 8 | h | RL | 2.10 | 2.06 | 2.14 | 2.10 | 0.04 | 1.25 | 1.16 | 1.23 | 1.21 | 0.05 |
| | | % | 112% | 106% | 114% | 111% | 4% | 25% | 16% | 23% | 21% | 5% |

TABLE 28
AHR results

| | BU Carbachol to Produce PC400 | |
|---|---|---|
| Sheep # | 24 h pre-ascaris | 24 h post Ascaris |

Control Trial

| | | |
|---|---|---|
| 2485 | 13 | 6 |
| 2515 | 22 | 12 |
| 2535 | 14 | 6 |

Drug Trial: Day 22

| | | |
|---|---|---|
| 2485 | 13 | 12 |
| 2515 | 24 | 24 |
| 2535 | 11 | 11 |

As shown in Table 27, treatment with AC000482 resulted in minimal attenuation of AR but robust attenuation of LR upon challenge on day 22. For example, untreated sheep display a mean LR increase of 1210% in R_{L} at 6.5 h compared to baseline, where AC000482 treated sheep on day 22 challenge show a more attenuated LR increase of 56% in R_{L} at 6.5 h compared to baseline. In addition, 24 h after the day 22 ascaris challenge all AC000482 treated sheep showed no signs of ascaris induced airway hyperresponsiveness, as shown by equivalent number of carbachol breath units required to produce PC₄₀₀. In contrast, in the control trial without AC000482 treatment, sheep required approximately half the amount of carbachol breath units to induce PC₄₀₀ post-ascaris challenge, signifying airway hyperresponsiveness.

### Example 12. Aerosolized Administration of MUC5AC RNAi Agents in Sheep

The sheep model of allergic asthma airway inflammation described in example 10, above, was used. Six (6) ascaris sensitive sheep with previously established responses to Ascaris suum challenge were administered either 0.5 mg/kg pulmonary deposited dose levels of AC000482 (n=3) of 0.25 mg/kg pulmonary deposited dose levels of AC000482 on days 1, 8 and 15. On day 21, AHR was assessed by determining the cumulative carbachol concentration (in breath units, BU) that increased R_{L} to 400% over the post-1×PBS value (PC₄₀₀). On day 22, sheep were challenged with Ascaris suum extract, and R_{L} was monitored out to 8 h post-challenge. On day 23, AHR was again assessed as on day 21.

TABLE 29
Airway mechanics results, 0.5 mg/kg dose level

| | | | Control (no treatment) | | | | | Drug Trial: Day 22 | | | | |
|---|---|---|---|---|---|---|---|---|---|---|---|---|
| | | | Animal # | | | Mean | | Animal # | | | Mean | |
| Timepoint | | | 2489 | 2497 | 2520 | Mean | S.D. | 2489 | 2497 | 2520 | Mean | S.D. |
| Baseline | | RL | 0.99 | 0.99 | 0.99 | 0.99 | 0.00 | 1.00 | 1.00 | 1.00 | 1.00 | 0.00 |
| Post- | | RL | 6.66 | 6.62 | 7.37 | 6.88 | 0.42 | 6.23 | 6.38 | 7.10 | 6.57 | 0.47 |
| Ascaris | | % | 573% | 569% | 644% | 595% | 43% | 523% | 538% | 610% | 557% | 47% |
| 1 | h | RL | 4.51 | 4.40 | 4.49 | 4.47 | 0.06 | 4.15 | 4.06 | 4.33 | 4.18 | 0.14 |
| | | % | 356% | 344% | 354% | 351% | 6% | 315% | 306% | 333% | 318% | 14% |
| 2 | h | RL | 2.53 | 2.57 | 2.37 | 2.49 | 0.11 | 2.25 | 2.31 | 2.10 | 2.22 | 0.11 |
| | | % | 156% | 160% | 139% | 152% | 11% | 125% | 131% | 110% | 122% | 11% |
| 3 | h | RL | 1.55 | 1.53 | 1.53 | 1.54 | 0.01 | 1.41 | 1.46 | 1.35 | 1.41 | 0.06 |
| | | % | 57% | 55% | 55% | 55% | 1% | 41% | 46% | 35% | 41% | 6% |
| 4 | h | RL | 1.06 | 1.21 | 1.12 | 1.13 | 0.08 | 1.07 | 1.02 | 1.04 | 1.04 | 0.03 |
| | | % | 7% | 22% | 13% | 14% | 8% | 7% | 2% | 4% | 4% | 3% |
| 5 | h | RL | 1.52 | 1.66 | 1.54 | 1.57 | 0.08 | 1.21 | 1.33 | 1.38 | 1.31 | 0.09 |
| | | % | 54% | 68% | 56% | 59% | 8% | 21% | 33% | 38% | 31% | 9% |
| 6 | h | RL | 2.13 | 2.09 | 2.03 | 2.08 | 0.05 | 1.46 | 1.56 | 1.52 | 1.51 | 0.05 |
| | | % | 115% | 111% | 105% | 110% | 5% | 46% | 56% | 52% | 51% | 5% |
| 6.5 | h | RL | 2.26 | 2.21 | 2.21 | 2.23 | 0.03 | 1.68 | 1.72 | 1.67 | 1.69 | 0.03 |
| | | % | 128% | 123% | 123% | 125% | 3% | 68% | 72% | 67% | 69% | 3% |
| 7 | h | RL | 2.32 | 2.26 | 2.30 | 2.29 | 0.03 | 1.62 | 1.68 | 1.61 | 1.64 | 0.04 |
| | | % | 134% | 128% | 132% | 132% | 3% | 62% | 68% | 61% | 64% | 4% |
| 7.5 | h | RL | 2.12 | 2.16 | 2.20 | 2.16 | 0.04 | 1.30 | 1.41 | 1.48 | 1.40 | 0.09 |
| | | % | 114% | 118% | 122% | 118% | 4% | 30% | 41% | 48% | 40% | 9% |
| 8 | h | RL | 2.08 | 2.03 | 2.12 | 2.08 | 0.05 | 1.25 | 1.26 | 1.13 | 1.21 | 0.07 |

| | | | | | | | | | | |
|---|---|---|---|---|---|---|---|---|---|---|
| % | 110% | 105% | 114% | 110% | 5% | 25% | 26% | 13% | 21% | 7% |

TABLE 30
Airway mechanics results, 0.25 mg/kg dose level

| | | | Control (no treatment) | | | | | Drug Trial: Day 22 | | | | |
|---|---|---|---|---|---|---|---|---|---|---|---|---|
| | | | Animal # | | | Mean | | Animal # | | | Mean | |
| Timepoint | | | 2457 | 2517 | 2539 | Mean | S.D. | 2457 | 2517 | 2539 | Mean | S.D. |
| Baseline | | RL | 1.00 | 1.01 | 1.00 | 1.00 | 0.01 | 1.00 | 1.00 | 1.00 | 1.00 | 0.00 |
| Post- | | RL | 6.63 | 7.29 | 6.03 | 6.65 | 0.63 | 6.50 | 7.03 | 6.26 | 6.60 | 0.39 |
| Ascaris | | % | 563% | 622% | 503% | 563% | 59% | 550% | 603% | 526% | 560% | 39% |
| 1 | h | RL | 4.15 | 4.26 | 4.50 | 4.30 | 0.18 | 4.09 | 4.10 | 4.11 | 4.10 | 0.01 |
| | | % | 315% | 322% | 350% | 329% | 19% | 309% | 310% | 311% | 310% | 1% |
| 2 | h | RL | 2.73 | 2.47 | 2.59 | 2.60 | 0.13 | 2.23 | 2.33 | 2.46 | 2.34 | 0.12 |
| | | % | 173% | 145% | 159% | 159% | 14% | 123% | 133% | 146% | 134% | 12% |
| 3 | h | RL | 1.54 | 1.37 | 1.62 | 1.51 | 0.13 | 1.45 | 1.27 | 1.54 | 1.42 | 0.14 |
| | | % | 54% | 36% | 62% | 51% | 14% | 45% | 27% | 54% | 42% | 14% |
| 4 | h | RL | 1.17 | 1.08 | 1.03 | 1.09 | 0.07 | 1.07 | 1.03 | 1.04 | 1.05 | 0.02 |
| | | % | 17% | 7% | 3% | 9% | 7% | 7% | 3% | 4% | 5% | 2% |
| 5 | h | RL | 1.69 | 1.61 | 1.29 | 1.53 | 0.21 | 1.43 | 1.21 | 1.32 | 1.32 | 0.11 |
| | | % | 69% | 59% | 29% | 52% | 21% | 43% | 21% | 32% | 32% | 11% |
| 6 | h | RL | 2.04 | 2.14 | 2.26 | 2.15 | 0.11 | 1.62 | 1.51 | 1.57 | 1.57 | 0.06 |
| | | % | 104% | 112% | 126% | 114% | 11% | 62% | 51% | 57% | 57% | 6% |
| 6.5 | h | RL | 2.18 | 2.36 | 2.14 | 2.23 | 0.12 | 1.87 | 1.78 | 1.83 | 1.83 | 0.05 |
| | | % | 118% | 134% | 114% | 122% | 10% | 87% | 78% | 83% | 83% | 5% |
| 7 | h | RL | 2.12 | 2.52 | 2.30 | 2.31 | 0.20 | 1.93 | 1.89 | 1.86 | 1.89 | 0.04 |
| | | % | 112% | 150% | 130% | 131% | 19% | 93% | 89% | 86% | 89% | 4% |
| 7.5 | h | RL | 2.17 | 2.35 | 2.21 | 2.24 | 0.09 | 1.74 | 1.67 | 1.68 | 1.70 | 0.04 |
| | | % | 117% | 133% | 121% | 124% | 8% | 74% | 67% | 68% | 70% | 4% |
| 8 | h | RL | 2.18 | 2.30 | 2.17 | 2.22 | 0.07 | 1.52 | 1.45 | 1.55 | 1.51 | 0.05 |
| | | % | 118% | 128% | 117% | 121% | 6% | 52% | 45% | 55% | 51% | 5% |

TABLE 31
AHR results, 0.5 mg/kg dose level

| | BU Carbachol to Produce PC400 | |
|---|---|---|
| Sheep # | 24 h pre-ascaris | 24 h post Ascaris |

Control Trial

| | | |
|---|---|---|
| 2489 | 24 | 13 |
| 2497 | 31 | 13 |
| 2520 | 26 | 13 |

Drug Trial: Day 22

| | | |
|---|---|---|
| 2489 | 26 | 25 |
| 2497 | 26 | 24 |
| 2520 | 27 | 25 |

TABLE 32
AHR results, 0.25 mg/kg dose level

| | BU Carbachol to Produce PC400 | |
|---|---|---|
| Sheep # | 24 h pre-ascaris | 24 h post Ascaris |

Control Trial

| | | |
|---|---|---|
| 2457 | 10 | 6 |
| 2517 | 28 | 13 |
| 2539 | 13 | 6 |

Drug Trial: Day 22

| | | |
|---|---|---|
| 2457 | 13 | 11 |
| 2517 | 26 | 25 |
| 2539 | 14 | 13 |

As shown in Table 29, treatment with AC000482 at 0.5 mg/kg dose level resulted in minimal attenuation of AR but robust attenuation of LR upon challenge on day 22. For example, untreated sheep display a mean LR increase of 125% in R_{L} at 6.5 h compared to baseline, where AC000482 treated sheep on day 22 challenge show a more attenuated LR increase of 69% in R_{L} at 6.5 h compared to baseline. In addition, 24 h after the day 22 ascaris challenge all AC000482 treated sheep showed no signs of ascaris induced airway hyperresponsiveness, as shown by similar number of carbachol breath units required to produce PC₄₀₀. In contrast, in the control trial without AC000482 treatment, sheep required approximately half the amount of carbachol breath units to induce PC₄₀₀ post-ascaris challenge, signifying airway hyperresponsiveness.

As shown in Table 30, treatment with AC000482 at 0.25 mg/kg dose level resulted in minimal attenuation of AR but robust attenuation of LR upon challenge on day 22. For example, untreated sheep display a mean LR increase of 122% in R_{L} at 6.5 h compared to baseline, where AC000482 treated sheep on day 22 challenge show a more attenuated LR increase of 83% in R_{L} at 6.5 h compared to baseline. In addition, 24 h after the day 22 ascaris challenge all AC000482 treated sheep showed no signs of ascaris induced airway hyperresponsiveness, as shown by similar number of carbachol breath units required to produce PC₄₀₀. In contrast, in the control trial without AC000482 treatment, sheep required approximately half the amount of carbachol breath units to induce PC₄₀₀ post-ascaris challenge, signifying airway hyperresponsiveness.

Collectively, the results demonstrate dose-responsive impacts of AC000482 treatment on airway mechanics following ascaris challenge. The results show that even at the lowest dose of AC000482, the impact on the late phase response is still substantial enough to block airway hyperresponsiveness 24 h post challenge.

### Example 13. Aerosolized Administration of MUC5AC RNAi Agents in Sheep

The sheep model of allergic asthma airway inflammation described in example 10, above, was used. Six (6) ascaris sensitive sheep with previously established responses to Ascaris suum challenge were administered, on days 1, 8 and 15, with either 1.0 mg/kg pulmonary deposited dose levels of AC000480 or 1.0 mg/kg pulmonary deposited dose of a negative control siRNA conjugate that included the same targeting ligand but is unable to load into the RISC complex and therefore is unable to mediate RNA interference gene silencing. On day 21, AHR was assessed by determining the cumulative carbachol concentration (in breath units, BU) that increased R_{L} to 400% over the post-1×PBS value (PC₄₀₀). On day 22, sheep were challenged with Ascaris suum extract, and R_{L} was monitored out to 8 h post-challenge. On day 23, AHR was again assessed as on day 21. Sheep dosed with AC000480 attenuated allergen-induced late-phase reaction and airway hyperresponsiveness in a dose dependent manner, while similar exposure of the negative control conjugate did not attenuate allergen-induced changes in airway mechanics.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An RNAi agent for inhibiting expression of a Mucin 5AC gene, comprising:
an antisense strand comprising at least 17 contiguous nucleotides differing by 0 or 1 nucleotides from any one of the sequences provided in Table 2 or Table 3; and
a sense strand comprising a nucleotide sequence that is at least partially complementary to the antisense strand.

2.-3. (canceled)

4. The RNAi agent of claim 1, wherein at least one nucleotide of the RNAi agent is a modified nucleotide or includes a modified internucleoside linkage.

5. (canceled)

6. The RNAi agent of claim 4, wherein the modified nucleotide is selected from the group consisting of: 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide.

7.-12. (canceled)

13. The RNAi agent of claim 1, wherein the sense strand and the antisense strand are each between 18 and 24 nucleotides in length.

14. The RNAi agent of claim 13, wherein the sense strand and the antisense strand are each 21 nucleotides in length.

15.-16. (canceled)

17. The RNAi agent of claim 1, wherein the sense strand comprises one or two inverted abasic residues.

18.-19. (canceled)

20. The RNAi agent of claim 1, wherein the antisense strand consists of, consists essentially of, or comprises a nucleotide sequence that differs by 0 or 1 nucleotides from one of the following nucleotide sequences 5′→3′):
(SEQ ID NO: 79)
UUGUAGUAGUCGCAGAACA;
or
(SEQ ID NO: 83)
UUCUUGUUCAGGCAAAUCA.

21. The RNAi agent of claim 1, wherein the antisense strand consists of, consists essentially of, or comprises a nucleotide sequence that differs by 0 or 1 nucleotides from one of the following nucleotide sequences (5′→3′):
(SEQ ID NO: 1525)
UUGUAGUAGUCGCAGAACAGC.

22. The RNAi agent of claim 1, wherein the sense strand consists of, consists essentially of, or comprises a nucleotide sequence that differs by 0 or 1 nucleotides from one of the following nucleotide sequences (5′→3′):
(SEQ ID NO: 568)
UGUUCUGCGACUACUACAA.

23. (canceled)

24. The RNAi agent of claim 1, wherein the antisense strand comprises, consists of, or consists essentially of a modified nucleotide sequence that differs by 0 or 1 nucleotides from one of the following nucleotide sequences (5′→3′):
(SEQ ID NO: 1127)
cPrpusUfsgsUfaGfuAfgUfcGfcAfgAfaCfaGfsc;
(SEQ ID NO: 1065)
usUfsgsUfaGfuAfgUfcGfcAfgAfaCfaGfsc;
wherein a, c, g, and u represent 2′-O-methyl adenosine, 2′-O-methyl cytidine, 2′-O-methyl guanosine, and 2′-O-methyl uridine, respectively; Af, Cf, Gf, and Uf represent 2′-fluoro adenosine, 2′-fluoro cytidine, 2′-fluoro guanosine, and 2′-fluoro uridine, respectively; cPrpu represents a 5′-cyclopropyl phosphonate-2′-O-methyl uridine; s represents a phosphorothioate linkage; and wherein all or substantially all of the nucleotides on the sense strand are modified nucleotides.

25. The RNAi agent of claim 1, wherein the sense strand comprises, consists of, or consists essentially of a modified nucleotide sequence that differs by 0 or 1 nucleotides from one of the following nucleotide sequences (5′→3′):
(SEQ ID NO: 1265)
gscuguucuGfCfGfacuacuacaa;
wherein a, c, g, and u represent 2′-O-methyl adenosine, 2′-O-methyl cytidine, 2′-O-methyl guanosine, and 2′-O-methyl uridine, respectively; Af, Cf, Gf, and Uf represent 2′-fluoro adenosine, 2′-fluoro cytidine, 2′-fluoro guanosine, and 2′-fluoro uridine, respectively; and s represents a phosphorothioate linkage; and wherein all or substantially all of the nucleotides on the antisense strand are modified nucleotides.

26. (canceled)

27. The RNAi agent of claim 1, wherein the RNAi agent is linked to a targeting ligand.

28.-30. (canceled)

31. The RNAi agent of claim 27, wherein the targeting ligand comprises the structure: 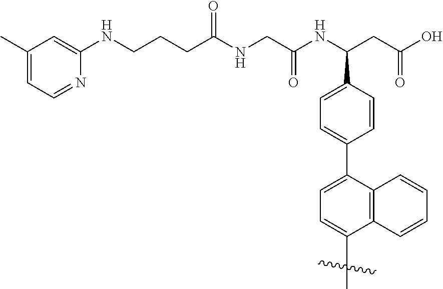 [SMILES: *c1ccc(-c2ccc([C@H](CC(=O)O)NC(=O)CNC(=O)CCCNc3cc(C)ccn3)cc2)c2ccccc12]
or a pharmaceutically acceptable salt thereof, or 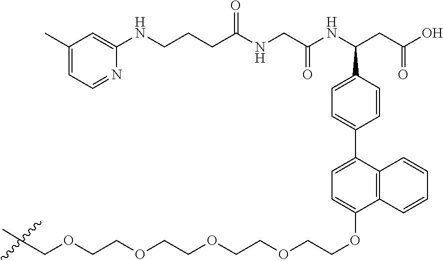 [SMILES: *COCCOCCOCCOCCOc1ccc(-c2ccc([C@H](CC(=O)O)NC(=O)CNC(=O)CCCNc3cc(C)ccn3)cc2)c2ccccc12]
or a pharmaceutically acceptable salt thereof, wherein [symbol] indicates the point of connection to the RNAi agent.

32. The RNAi agent of claim 27, wherein RNAi agent is conjugated to a targeting ligand having the following structure: 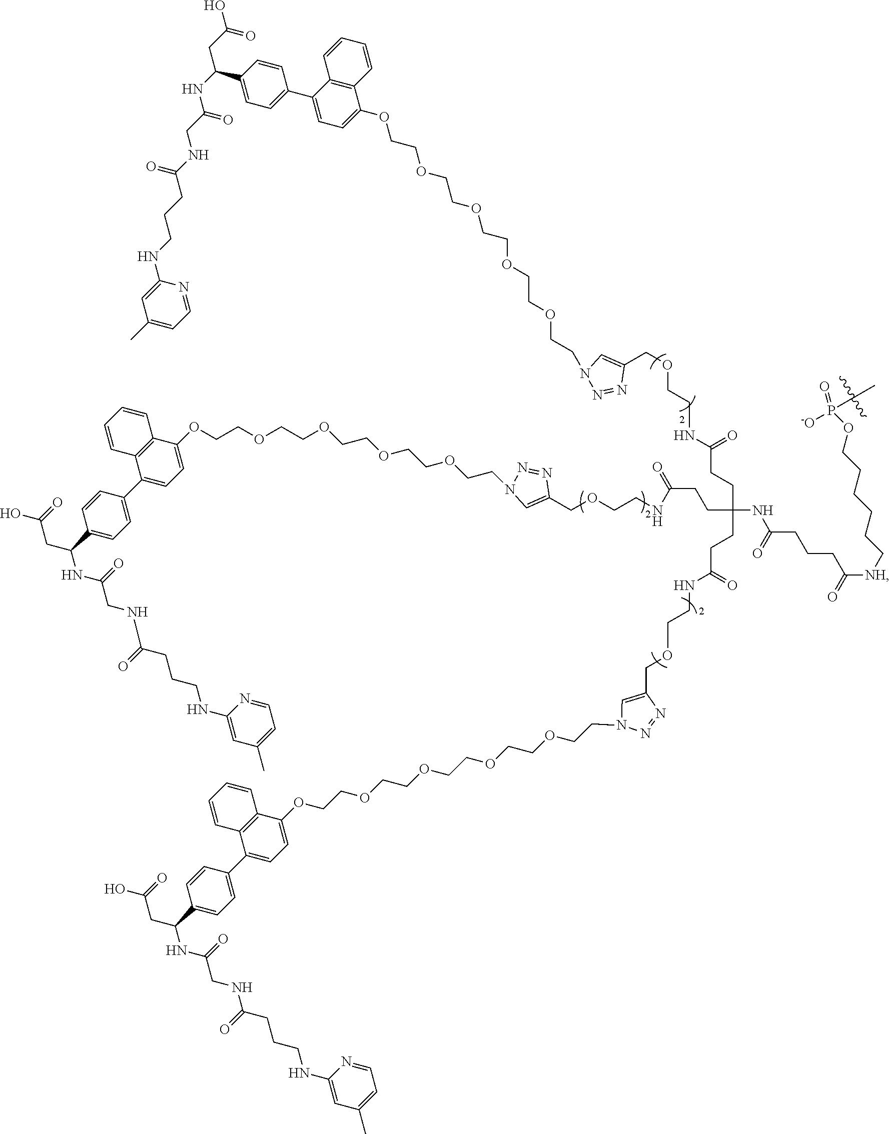
or a pharmaceutically acceptable salt thereof, wherein [symbol] indicates the point of connection to the RNAi agent.

33. (canceled)

34. The RNAi agent of claim 27, wherein the targeting ligand is conjugated to the sense strand.

35. (canceled)

36. A composition comprising the RNAi agent of claim 1, wherein the composition further comprises a pharmaceutically acceptable excipient.

37. The composition of claim 36, further comprising a second RNAi agent capable of inhibiting the expression of Mucin 5AC gene expression.

38.-40. (canceled)

41. The composition of claim 36, wherein the RNAi agent is a sodium salt.

42.-47. (canceled)

48. A method of treating one or more symptoms or diseases associated with MUC5AC protein levels, the method comprising administering to a human subject in need thereof a therapeutically effective amount of the composition of claim 36.

49.-62. (canceled)

63. A method of making an RNAi agent of claim 1, comprising annealing a sense strand and an antisense strand to form a double-stranded ribonucleic acid molecule.

64.-65. (canceled)
